SEQUENCE LISTING (342 sequences)

SEQ ID NO: 1 - protein, 9 aa, synthetic construct
  REGION 1..9: note = Synthetic
GGSISSSDY

SEQ ID NO: 2 - protein, 9 aa, synthetic construct
  REGION 1..9: note = Synthetic
GGSISSSST

SEQ ID NO: 3 - protein, 9 aa, synthetic construct
  REGION 1..9: note = Synthetic
GGSISSSDT

SEQ ID NO: 4 - protein, 9 aa, synthetic construct
  REGION 1..9: note = Synthetic
GGSISSADN

SEQ ID NO: 5 - protein, 9 aa, synthetic construct
  REGION 1..9: note = Synthetic
GGSVSSSST

SEQ ID NO: 6 - protein, 8 aa, synthetic construct
  REGION 1..8: note = Synthetic
GYSISSGY

SEQ ID NO: 7 - protein, 8 aa, synthetic construct
  REGION 1..8: note = Synthetic
GYSILSGY

SEQ ID NO: 8 - protein, 8 aa, synthetic construct
  REGION 1..8: note = Synthetic
GYSISSGH

SEQ ID NO: 9 - protein, 8 aa, synthetic construct
  REGION 1..8: note = Synthetic
GYSISSGF

SEQ ID NO: 10 - protein, 7 aa, synthetic construct
  REGION 1..7: note = Synthetic
GFTFDNY

SEQ ID NO: 11 - protein, 7 aa, synthetic construct
  REGION 1..7: note = Synthetic
GFTFSDY

SEQ ID NO: 12 - protein, 7 aa, synthetic construct
  REGION 1..7: note = Synthetic
GFTFSSS

SEQ ID NO: 13 - protein, 8 aa, synthetic construct
  REGION 1..8: note = Synthetic
GGSISSSN

SEQ ID NO: 14 - protein, 9 aa, synthetic construct
  REGION 1..9: note = Synthetic
GGSISSSSY

SEQ ID NO: 15-17 - skipped (3 entries)

SEQ ID NO: 18 - protein, 7 aa, synthetic construct
  REGION 1..7: note = Synthetic
SSDYYWG

SEQ ID NO: 19 - protein, 7 aa, synthetic construct
  REGION 1..7: note = Synthetic
SSSTYWA

SEQ ID NO: 20 - protein, 7 aa, synthetic construct
  REGION 1..7: note = Synthetic
SSSTYWG

SEQ ID NO: 21 - protein, 7 aa, synthetic construct
  REGION 1..7: note = Synthetic
SSDTYWG

SEQ ID NO: 22 - protein, 7 aa, synthetic construct
  REGION 1..7: note = Synthetic
SADNYWG

SEQ ID NO: 23 - protein, 7 aa, synthetic construct
  REGION 1..7: note = Synthetic
SSSTYWS

SEQ ID NO: 24 - protein, 6 aa, synthetic construct
  REGION 1..6: note = Synthetic
SGYYWG

SEQ ID NO: 25 - protein, 6 aa, synthetic construct
  REGION 1..6: note = Synthetic
SGYYWF

SEQ ID NO: 26 - protein, 6 aa, synthetic construct
  REGION 1..6: note = Synthetic
SGHYWI

SEQ ID NO: 27 - protein, 6 aa, synthetic construct
  REGION 1..6: note = Synthetic
SGFYWT

SEQ ID NO: 28 - protein, 6 aa, synthetic construct
  REGION 1..6: note = Synthetic
SGYYWL

SEQ ID NO: 29 - protein, 6 aa, synthetic construct
  REGION 1..6: note = Synthetic
SGHYWT

SEQ ID NO: 30 - protein, 5 aa, synthetic construct
  REGION 1..5: note = Synthetic
NYAMH

SEQ ID NO: 31 - protein, 5 aa, synthetic construct
  REGION 1..5: note = Synthetic
DYYMS

SEQ ID NO: 32 - protein, 5 aa, synthetic construct
  REGION 1..5: note = Synthetic
SSAMA

SEQ ID NO: 33 - protein, 6 aa, synthetic construct
  REGION 1..6: note = Synthetic
SSNWWS

SEQ ID NO: 34 - protein, 7 aa, synthetic construct
  REGION 1..7: note = Synthetic
SSSYYWG

SEQ ID NO: 35-37 - skipped (3 entries)

SEQ ID NO: 38 - protein, 5 aa, synthetic construct
  REGION 1..5: note = Synthetic
YYSGS

SEQ ID NO: 39 - protein, 5 aa, synthetic construct
  REGION 1..5: note = Synthetic
SSSGS

SEQ ID NO: 40 - protein, 5 aa, synthetic construct
  REGION 1..5: note = Synthetic
HHSGA

SEQ ID NO: 41 - protein, 5 aa, synthetic construct
  REGION 1..5: note = Synthetic
HYSGS

SEQ ID NO: 42 - protein, 5 aa, synthetic construct
  REGION 1..5: note = Synthetic
AYSGS

SEQ ID NO: 43 - protein, 5 aa, synthetic construct
  REGION 1..5: note = Synthetic
SYNAL

SEQ ID NO: 44 - protein, 5 aa, synthetic construct
  REGION 1..5: note = Synthetic
YHSGS

SEQ ID NO: 45 - protein, 5 aa, synthetic construct
  REGION 1..5: note = Synthetic
YHSAS

SEQ ID NO: 46 - protein, 6 aa, synthetic construct
  REGION 1..6: note = Synthetic
SARAGI

SEQ ID NO: 47 - protein, 6 aa, synthetic construct
  REGION 1..6: note = Synthetic
ASSGSV

SEQ ID NO: 48 - protein, 6 aa, synthetic construct
  REGION 1..6: note = Synthetic
SGSGIT

SEQ ID NO: 49 - protein, 5 aa, synthetic construct
  REGION 1..5: note = Synthetic
SYSGS

SEQ ID NO: 50 - protein, 6 aa, synthetic construct
  REGION 1..6: note = Synthetic
SSSGST

SEQ ID NO: 51-53 - skipped (3 entries)

SEQ ID NO: 54 - protein, 16 aa, synthetic construct
  REGION 1..16: note = Synthetic
SIYYSGSTYYNPSLKS

SEQ ID NO: 55 - protein, 16 aa, synthetic construct
  REGION 1..16: note = Synthetic
SISSSGSTYYNPSLKS

SEQ ID NO: 56 - protein, 16 aa, synthetic construct
  REGION 1..16: note = Synthetic
SIHHSGATYYNPSLKS

SEQ ID NO: 57 - protein, 16 aa, synthetic construct
  REGION 1..16: note = Synthetic
SIHYSGSTLYNPSLKS

SEQ ID NO: 58 - protein, 16 aa, synthetic construct
  REGION 1..16: note = Synthetic
SIHYSGSTYYNPSLKS

SEQ ID NO: 59 - protein, 16 aa, synthetic construct
  REGION 1..16: note = Synthetic
GIAYSGSTYYNPSLKS

SEQ ID NO: 60 - protein, 16 aa, synthetic construct
  REGION 1..16: note = Synthetic
SISYNALTYYNPSLKS

SEQ ID NO: 61 - protein, 16 aa, synthetic construct
  REGION 1..16: note = Synthetic
SIYHSGSTYYNPSLKS

SEQ ID NO: 62 - protein, 16 aa, synthetic construct
  REGION 1..16: note = Synthetic
GIYHSASTAYNPSLKS

SEQ ID NO: 63 - protein, 16 aa, synthetic construct
  REGION 1..16: note = Synthetic
GIYHSGSTYYNPSLKS

SEQ ID NO: 64 - protein, 16 aa, synthetic construct
  REGION 1..16: note = Synthetic
AIYHSGSTVYNPSLKS

SEQ ID NO: 65 - protein, 16 aa, synthetic construct
  REGION 1..16: note = Synthetic
GIYHSGSTAYNPSLKS

SEQ ID NO: 66 - protein, 17 aa, synthetic construct
  REGION 1..17: note = Synthetic
AISARAGITYYADSVKG

SEQ ID NO: 67 - protein, 17 aa, synthetic construct
  REGION 1..17: note = Synthetic
YIASSGSVIYYADSVKG

SEQ ID NO: 68 - protein, 17 aa, synthetic construct
  REGION 1..17: note = Synthetic
TISGSGITTWYADSVKG

SEQ ID NO: 69 - protein, 16 aa, synthetic construct
  REGION 1..16: note = Synthetic
EIYHSGSTNYNPSLKS

SEQ ID NO: 70 - protein, 16 aa, synthetic construct
  REGION 1..16: note = Synthetic
SISYSGSTYYNPSLKS

SEQ ID NO: 71 - protein, 17 aa, synthetic construct
  REGION 1..17: note = Synthetic
YISSSGSTIYYADSVKG

SEQ ID NO: 72-75 - skipped (4 entries)

SEQ ID NO: 76 - protein, 10 aa, synthetic construct
  REGION 1..10: note = Synthetic
GVRRAVPFDY

SEQ ID NO: 77 - protein, 10 aa, synthetic construct
  REGION 1..10: note = Synthetic
GIARAVPFDY

SEQ ID NO: 78 - protein, 10 aa, synthetic construct
  REGION 1..10: note = Synthetic
GPKRAVPFDY

SEQ ID NO: 79 - protein, 10 aa, synthetic construct
  REGION 1..10: note = Synthetic
GVRRAVPFVD

SEQ ID NO: 80 - protein, 10 aa, synthetic construct
  REGION 1..10: note = Synthetic
GVRRAVPFQR

SEQ ID NO: 81 - protein, 10 aa, synthetic construct
  REGION 1..10: note = Synthetic
GTRRAVPFDY

SEQ ID NO: 82 - protein, 10 aa, synthetic construct
  REGION 1..10: note = Synthetic
GVRRAVPFAD

SEQ ID NO: 83 - protein, 10 aa, synthetic construct
  REGION 1..10: note = Synthetic
GIRRAVPFDY

SEQ ID NO: 84 - protein, 10 aa, synthetic construct
  REGION 1..10: note = Synthetic
GQFRAVPFDY

SEQ ID NO: 85 - protein, 13 aa, synthetic construct
  REGION 1..13: note = Synthetic
GGTHTYSRGPMDV

SEQ ID NO: 86 - protein, 13 aa, synthetic construct
  REGION 1..13: note = Synthetic
GGTHTYSRGPFDV

SEQ ID NO: 87 - protein, 13 aa, synthetic construct
  REGION 1..13: note = Synthetic
GGTPIYSRGPLDV

SEQ ID NO: 88 - protein, 13 aa, synthetic construct
  REGION 1..13: note = Synthetic
GGGQTYSRGPLDV

SEQ ID NO: 89 - protein, 13 aa, synthetic construct
  REGION 1..13: note = Synthetic
GGGATYSRGPLDV

SEQ ID NO: 90 - protein, 13 aa, synthetic construct
  REGION 1..13: note = Synthetic
GGTHTYSRGPLDV

SEQ ID NO: 91 - protein, 13 aa, synthetic construct
  REGION 1..13: note = Synthetic
GGTVKYSRGPLDV

SEQ ID NO: 92 - protein, 13 aa, synthetic construct
  REGION 1..13: note = Synthetic
GGQVTYSRGPLDV

SEQ ID NO: 93 - protein, 13 aa, synthetic construct
  REGION 1..13: note = Synthetic
GGEVTYSRGPLDV

SEQ ID NO: 94 - protein, 14 aa, synthetic construct
  REGION 1..14: note = Synthetic
RIGYSYGTAPPFDV

SEQ ID NO: 95 - protein, 9 aa, synthetic construct
  REGION 1..9: note = Synthetic
HGTPRAFDI

SEQ ID NO: 96 - protein, 10 aa, synthetic construct
  REGION 1..10: note = Synthetic
GSRHLNAFNR

SEQ ID NO: 97 - protein, 12 aa, synthetic construct
  REGION 1..12: note = Synthetic
GVYHYDPYGMDV

SEQ ID NO: 98 - protein, 10 aa, synthetic construct
  REGION 1..10: note = Synthetic
TELGKMHFDY

SEQ ID NO: 99 - protein, 8 aa, synthetic construct
  REGION 1..8: note = Synthetic
GSPRYMQD

SEQ ID NO: 100 - protein, 12 aa, synthetic construct
  REGION 1..12: note = Synthetic
HSSLGTHNWFDP

SEQ ID NO: 101 - protein, 14 aa, synthetic construct
  REGION 1..14: note = Synthetic
EGALSYSWLAAFDI

SEQ ID NO: 102-104 - skipped (3 entries)

SEQ ID NO: 105 - protein, 12 aa, synthetic construct
  REGION 1..12: note = Synthetic
RASQSVSSSYLA

SEQ ID NO: 106 - protein, 12 aa, synthetic construct
  REGION 1..12: note = Synthetic
GASQSVSSDYLA

SEQ ID NO: 107 - protein, 12 aa, synthetic construct
  REGION 1..12: note = Synthetic
QASQAVSSNYLA

SEQ ID NO: 108 - protein, 12 aa, synthetic construct
  REGION 1..12: note = Synthetic
GASQSVSSAFLA

SEQ ID NO: 109 - protein, 12 aa, synthetic construct
  REGION 1..12: note = Synthetic
RASQSVSSTYLA

SEQ ID NO: 110 - protein, 12 aa, synthetic construct
  REGION 1..12: note = Synthetic
QASQSVSSSYLA

SEQ ID NO: 111 - protein, 12 aa, synthetic construct
  REGION 1..12: note = Synthetic
KASQAVSSSYLA

SEQ ID NO: 112 - protein, 12 aa, synthetic construct
  REGION 1..12: note = Synthetic
EASQSVSSSYLA

SEQ ID NO: 113 - protein, 12 aa, synthetic construct
  REGION 1..12: note = Synthetic
EASQSVSASYLA

SEQ ID NO: 114 - protein, 12 aa, synthetic construct
  REGION 1..12: note = Synthetic
EASQSVSSAYLA

SEQ ID NO: 115 - protein, 12 aa, synthetic construct
  REGION 1..12: note = Synthetic
RVSQSVSDAYLA

SEQ ID NO: 116 - protein, 12 aa, synthetic construct
  REGION 1..12: note = Synthetic
EVSQSVSASYLA

SEQ ID NO: 117 - protein, 12 aa, synthetic construct
  REGION 1..12: note = Synthetic
RASQSVSSAYLA

SEQ ID NO: 118 - protein, 12 aa, synthetic construct
  REGION 1..12: note = Synthetic
RASNAVSSSYLA

SEQ ID NO: 119 - protein, 11 aa, synthetic construct
  REGION 1..11: note = Synthetic
RASQSINSWLA

SEQ ID NO: 120 - protein, 11 aa, synthetic construct
  REGION 1..11: note = Synthetic
AASQGISSDLA

SEQ ID NO: 121 - protein, 11 aa, synthetic construct
  REGION 1..11: note = Synthetic
RASQDISTYLN

SEQ ID NO: 122 - protein, 16 aa, synthetic construct
  REGION 1..16: note = Synthetic
RSSQSLLHSNGYNYLD

SEQ ID NO: 123 - protein, 11 aa, synthetic construct
  REGION 1..11: note = Synthetic
RASQSISSYLN

SEQ ID NO: 124 - protein, 11 aa, synthetic construct
  REGION 1..11: note = Synthetic
RASQSVSSNLA

SEQ ID NO: 125-127 - skipped (3 entries)

SEQ ID NO: 128 - protein, 7 aa, synthetic construct
  REGION 1..7: note = Synthetic
GASSRAT

SEQ ID NO: 129 - protein, 7 aa, synthetic construct
  REGION 1..7: note = Synthetic
GAYSLAT

SEQ ID NO: 130 - protein, 7 aa, synthetic construct
  REGION 1..7: note = Synthetic
GASARAT

SEQ ID NO: 131 - protein, 7 aa, synthetic construct
  REGION 1..7: note = Synthetic
GASSREA

SEQ ID NO: 132 - protein, 7 aa, synthetic construct
  REGION 1..7: note = Synthetic
GASNRAA

SEQ ID NO: 133 - protein, 7 aa, synthetic construct
  REGION 1..7: note = Synthetic
GASSRQD

SEQ ID NO: 134 - protein, 7 aa, synthetic construct
  REGION 1..7: note = Synthetic
GASNRAT

SEQ ID NO: 135 - protein, 7 aa, synthetic construct
  REGION 1..7: note = Synthetic
DASSRAS

SEQ ID NO: 136 - protein, 7 aa, synthetic construct
  REGION 1..7: note = Synthetic
DASTRAT

SEQ ID NO: 137 - protein, 7 aa, synthetic construct
  REGION 1..7: note = Synthetic
GASDRAN

SEQ ID NO: 138 - protein, 7 aa, synthetic construct
  REGION 1..7: note = Synthetic
GASYRAT

SEQ ID NO: 139 - protein, 7 aa, synthetic construct
  REGION 1..7: note = Synthetic
DASSLES

SEQ ID NO: 140 - protein, 7 aa, synthetic construct
  REGION 1..7: note = Synthetic
SASSTQS

SEQ ID NO: 141 - protein, 7 aa, synthetic construct
  REGION 1..7: note = Synthetic
DAFNLET

SEQ ID NO: 142 - protein, 7 aa, synthetic construct
  REGION 1..7: note = Synthetic
LGSNRAS

SEQ ID NO: 143 - protein, 7 aa, synthetic construct
  REGION 1..7: note = Synthetic
GASRRAT

SEQ ID NO: 144 - protein, 7 aa, synthetic construct
  REGION 1..7: note = Synthetic
AASSLQS

SEQ ID NO: 145 - protein, 7 aa, synthetic construct
  REGION 1..7: note = Synthetic
GASTRAT

SEQ ID NO: 146-148 - skipped (3 entries)

SEQ ID NO: 149 - protein, 9 aa, synthetic construct
  REGION 1..9: note = Synthetic
QQAVHSPYT

SEQ ID NO: 150 - protein, 9 aa, synthetic construct
  REGION 1..9: note = Synthetic
QWAVHSPYT

SEQ ID NO: 151 - protein, 9 aa, synthetic construct
  REGION 1..9: note = Synthetic
QQVVHSPYT

SEQ ID NO: 152 - protein, 9 aa, synthetic construct
  REGION 1..9: note = Synthetic
QQTVHSPYT

SEQ ID NO: 153 - protein, 9 aa, synthetic construct
  REGION 1..9: note = Synthetic
QQAIHSPYT

SEQ ID NO: 154 - protein, 9 aa, synthetic construct
  REGION 1..9: note = Synthetic
QQHSSYPPT

SEQ ID NO: 155 - protein, 9 aa, synthetic construct
  REGION 1..9: note = Synthetic
QQHSLYPPT

SEQ ID NO: 156 - protein, 9 aa, synthetic construct
  REGION 1..9: note = Synthetic
QQFSSYPPT

SEQ ID NO: 157 - protein, 9 aa, synthetic construct
  REGION 1..9: note = Synthetic
QQVSSYPPT

SEQ ID NO: 158 - protein, 9 aa, synthetic construct
  REGION 1..9: note = Synthetic
QQHSIYPPT

SEQ ID NO: 159 - protein, 8 aa, synthetic construct
  REGION 1..8: note = Synthetic
QQYDSHIT

SEQ ID NO: 160 - protein, 9 aa, synthetic construct
  REGION 1..9: note = Synthetic
QQAYLYPIT

SEQ ID NO: 161 - protein, 9 aa, synthetic construct
  REGION 1..9: note = Synthetic
QQLPFLPIT

SEQ ID NO: 162 - protein, 9 aa, synthetic construct
  REGION 1..9: note = Synthetic
MQALGGPWT

SEQ ID NO: 163 - protein, 9 aa, synthetic construct
  REGION 1..9: note = Synthetic
QQYVSDPIT

SEQ ID NO: 164 - protein, 9 aa, synthetic construct
  REGION 1..9: note = Synthetic
QQVGSSPIT

SEQ ID NO: 165 - protein, 9 aa, synthetic construct
  REGION 1..9: note = Synthetic
QQSHLVPRT

SEQ ID NO: 166 - protein, 10 aa, synthetic construct
  REGION 1..10: note = Synthetic
QQANHHPPFT

SEQ ID NO: 167-169 - skipped (3 entries)

SEQ ID NO: 170 - protein, 120 aa, synthetic construct
  REGION 1..120: note = Synthetic
QLQLQESGPGLVKPSETLSLTCTVSGGSISSSDYYWGWIRQPPGKGLEWIGSIYYSGSTYYNPSLKSRVTISVDTSKNQFSLKLSSVTAADTAVYYCARGVRRAVPFDYWGQGTLVTVSS

SEQ ID NO: 171 - protein, 120 aa, synthetic construct
  REGION 1..120: note = Synthetic
QLQLQESGPGLVKPSETLSLTCTVSGGSISSSSTYWAWIRQPPGKGLEWIGSISSSGSTYYNPSLKSRVTISVDTSKNQFSLKLSSVTAADTAVYYCARGIARAVPFDYWGQGTLVTVSS

SEQ ID NO: 172 - protein, 120 aa, synthetic construct
  REGION 1..120: note = Synthetic
QLQLQESGPGLVKPSETLSLTCTVSGGSISSSSTYWGWIRQSPGKGLEWIGSIHHSGATYYNPSLKSRVTISVDTSKNQFSLKLSSVTAADTAVYYCARGPKRAVPFDYWGQGTLVTVSS

SEQ ID NO: 173 - protein, 120 aa, synthetic construct
  REGION 1..120: note = Synthetic
QLQLQESGPGLVKPSETLSLTCTVSGGSISSSDTYWGWIRQPPGKGLEWIGSIHYSGSTLYNPSLKSRVTISVDTSKNQFSLKLSSVTAADTAVYYCARGVRRAVPFVDWGQGTLVTVSS

SEQ ID NO: 174 - protein, 120 aa, synthetic construct
  REGION 1..120: note = Synthetic
QLQLQESGPGLVKPSETLSLTCTVSGGSISSADNYWGWIRQPPGKGLEWIGSIHYSGSTYYNPSLKSRVTISVDTSKNQFSLKLSSVTAADTAVYYCARGVRRAVPFQRWGQGTLVTVSS

SEQ ID NO: 175 - protein, 120 aa, synthetic construct
  REGION 1..120: note = Synthetic
QLQLQESGPGLVKPSETLSLTCTVSGGSISSSDTYWGWIRQPPGKGPEWIGSIHYSGSTLYNPSLKSRVTISVDTSKNQFSLKLSSVTAADTAVYYCARGVRRAVPFDYWGQGTLVTVSS

SEQ ID NO: 176 - protein, 120 aa, synthetic construct
  REGION 1..120: note = Synthetic
QLQLQESGPGLVKPSETLSLTCTVSGGSISSSSTYWSWIRQPPGKGLEWIGGIAYSGSTYYNPSLKSRVTISVDTSKNQFSLKLSSVTAADTAVYYCARGVRRAVPFDYWGQGTLVTVSS

SEQ ID NO: 177 - protein, 120 aa, synthetic construct
  REGION 1..120: note = Synthetic
QLQLQESGPGLVKPSETLSLTCTVSGGSISSSSTYWSWIRQPPGKGLEWIGSISYNALTYYNPSLKSRVTISVDTSKNQFSLKLSSVTAADTAVYYCARGTRRAVPFDYWGQGTLVTVSS

SEQ ID NO: 178 - protein, 120 aa, synthetic construct
  REGION 1..120: note = Synthetic
QLQLQESGPGLVKPSETLSLTCTVSGGSISSSSTYWSWIRQPPGKGLEWIGGIAYSGSTYYNPSLKSRVTISVDTSKNQFSLKLSSVTAADTAVYYCARGVRRAVPFADWGQGTLVTVSS

SEQ ID NO: 179 - protein, 120 aa, synthetic construct
  REGION 1..120: note = Synthetic
QLQLQESGPGLVKPSETLSLTCTVSGGSVSSSSTYWSWIRQPPGKGLEWIGGIAYSGSTYYNPSLKSRVTISVDTSKNQFSLKLSSVTAADTAVYYCARGVRRAVPFDYWGQGTLVTVSS

SEQ ID NO: 180 - protein, 120 aa, synthetic construct
  REGION 1..120: note = Synthetic
QLQLQESGPGLVKPSETLSLTCTVSGGSISSSSTYWSWIRQPPGKGLEWIGGIAYSGSTYYNPSLKSRVTISVDTSKNQFSLKLSSVTAADTAVYYCARGTRRAVPFDYWGQGTLVTVSS

SEQ ID NO: 181 - protein, 120 aa, synthetic construct
  REGION 1..120: note = Synthetic
QLQLQESGPGLVKPSETLSLTCTVSGGSISSSDTYWGWIRQPPGKGLEWIGSIHYSGSTLYNPSLKSRVTISVDTSKNQFSLKLSSVTAADTAVYYCARGIRRAVPFDYWGQGTLVTVSS

SEQ ID NO: 182 - protein, 120 aa, synthetic construct
  REGION 1..120: note = Synthetic
QLQLQESGPGLVKPSETLSLTCTVSGGSISSSDTYWGWIRQPPGKGLEWIGSIHYSGSTLYNPSLKSRVTISVDTSKNQFSLKLSSVTAADTAVYYCARGQFRAVPFDYWGQGTLVTVSS

SEQ ID NO: 183 - protein, 122 aa, synthetic construct
  REGION 1..122: note = Synthetic
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGGTHTYSRGPMDVWGQGTTVTVSS

SEQ ID NO: 184 - protein, 122 aa, synthetic construct
  REGION 1..122: note = Synthetic
QLQLQESGPRLVKPSETLSLTCAVSGYSISSGYYWGWIRQPPGKGLEWIGSIYHSGSTYYNPSLKSRVTISVDTSKNQFSLKLSSVTAADTAVYYCARGGTHTYSRGPFDVWGQGTTVTVSS

SEQ ID NO: 185 - protein, 122 aa, synthetic construct
  REGION 1..122: note = Synthetic
LVQLQESGPGLVKPSETLSLTCAVSGYSILSGYYWFWIRQPPGKGLEWIGGIYHSASTAYNPSLKSRVTISVDTSKNQFSLKLSSVTAADTAVYYCARGGTPIYSRGPLDVWGQGTTVTVSS

SEQ ID NO: 186 - protein, 122 aa, synthetic construct
  REGION 1..122: note = Synthetic
QVQLQESGPGLVKPSETLSLTCAVSGYSISSGHYWIWIRQPPGKGLEWIGGIYHSGSTYYNPSLKSRVTISVDTSKDQFSLKLSSVTAADTAVYYCARGGGQTYSRGPLDVWGQGTTVTVSS

SEQ ID NO: 187 - protein, 122 aa, synthetic construct
  REGION 1..122: note = Synthetic
QVQLQESGPGLVKPPETLSLTCAVSGYSISSGHYWIWIRQPPGKGLEWIGGIYHSGSTYYNPSLKSRVTISVDTSKNQFSLKLSSVTAADTAVYYCARGGGATYSRGPLDVWGQGTTVTVSS

SEQ ID NO: 188 - protein, 122 aa, synthetic construct
  REGION 1..122: note = Synthetic
QVQLQESGPGLVKPSETLSLTCAVSGYSILSGYYWFWIRQPPGKGLEWIGGIYHSGSTYYNPSLKSRVTISVDTSKNQFSLKLSSVTAADTAVYYCARGGTHTYSRGPLDVWGQGTTVTVSS

SEQ ID NO: 189 - protein, 122 aa, synthetic construct
  REGION 1..122: note = Synthetic
QVQLQESGPGLVKPSETLSLTCAVSGYSISSGFYWTWIRQPPGKGLEWIGAIYHSGSTVYNPSLKSRVTISVDTSKNQFSLKLSSVTAADTAVYYCARGGTHTYSRGPLDVWGQGTTVTVSS

SEQ ID NO: 190 - protein, 122 aa, synthetic construct
  REGION 1..122: note = Synthetic
QVQLQESGPGLVKPSETLSLTCAVSGYSISSGYYWLWIRQPPGKGLEWIGGIYHSASTAYNPSLKSRVTISVDTSKNQFSLKLSSVTAADTAVYYCARGGTVKYSRGPLDVWGQGTTVTVSS

SEQ ID NO: 191 - protein, 122 aa, synthetic construct
  REGION 1..122: note = Synthetic
QVQLQESGPGLVKPSETLSLTCAVSGYSISSGHYWTWIRQPPGKGLEWIGAIYHSGSTVYNPSLKSRVTISVDTSKNQFSLKLSSVTAADTAVYYCARGGQVTYSRGPLDVWGQGTTVTVSS

SEQ ID NO: 192 - protein, 122 aa, synthetic construct
  REGION 1..122: note = Synthetic
QVQLQESGPGLVKPSETLSLTCAVSGYSILSGYYWFWIRQPPGKGLEWIGGIYHSGSTAYNPSLKSRVTISVDTSKNQFSLKLSSVTAADTAVYYCARGGEVTYSRGPLDVWGQGTTVTVSS

SEQ ID NO: 193 - protein, 123 aa, synthetic construct
  REGION 1..123: note = Synthetic
EVQLLESGGGLVQPGGSLRLSCAASGFTFDNYAMHWVRQAPGKGLEWVSAISARAGITYYADSVKGRFTISRDNSKNTLYLQMNSLRAEDTAVYYCARRIGYSYGTAPPFDVWGQGTTVTVSS

SEQ ID NO: 194 - protein, 118 aa, synthetic construct
  REGION 1..118: note = Synthetic
QVQLVESGGGLVQPGGSLRLSCAASGFTFSDYYMSWIRQAPGKGLEWVSYIASSGSVIYYADSVKGRFTISRDNAKNSLYLQMNSLRAEDTAVYYCARHGTPRAFDIWGQGTTVTVSS

SEQ ID NO: 195 - protein, 119 aa, synthetic construct
  REGION 1..119: note = Synthetic
EVQLLESGGGLVQPGGSLRLSCAASGFTFSSSAMAWVRQAPGKGLEWVSTISGSGITTWYADSVKGRFTISRDNSKNTLYLQMNSLRAEDTAVYYCAKGSRHLNAFNRWGQGTTVTVSS

SEQ ID NO: 196 - protein, 121 aa, synthetic construct
  REGION 1..121: note = Synthetic
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARGVYHYDPYGMDVWGQGTTVTVSS

SEQ ID NO: 197 - protein, 120 aa, synthetic construct
  REGION 1..120: note = Synthetic
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARTELGKMHFDYWGQGTLVTVSS

SEQ ID NO: 198 - protein, 118 aa, synthetic construct
  REGION 1..118: note = Synthetic
QLQLQESGPGLVKPSETLSLTCTVSGGSISSSDYYWGWIRQPPGKGLEWIGSIYYSGSTYYNPSLKSRVTISVDTSKNQFSLKLSSVTAADTAVYYCARGSPRYMQDWGQGTLVTVSS

SEQ ID NO: 199 - protein, 121 aa, synthetic construct
  REGION 1..121: note = Synthetic
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARHSSLGTHNWFDPWGQGTLVTVSS

SEQ ID NO: 200 - protein, 123 aa, synthetic construct
  REGION 1..123: note = Synthetic
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCAREGALSYSWLAAFDIWGQGTMVTVSS

SEQ ID NO: 201-203 - skipped (3 entries)

SEQ ID NO: 204 - protein, 108 aa, synthetic construct
  REGION 1..108: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQAVHSPYTFGGGTKVEIK

SEQ ID NO: 205 - protein, 108 aa, synthetic construct
  REGION 1..108: note = Synthetic
EIVLTQSPGTLSLSPGERATLSCGASQSVSSDYLAWYQQKPGQAPRLLIYGAYSLATGIPDRFSGSGSGTDFTLTISRLEPEDFAVYYCQWAVHSPYTFGGGTKVEIK

SEQ ID NO: 206 - protein, 108 aa, synthetic construct
  REGION 1..108: note = Synthetic
EIVLTQSPGTLSLSPGERATLSCQASQAVSSNYLAWYQQKPGQAPRLLIYGASSRATGIPDRFSGSGSGTDFTLTISRLEPEDFAVYYCQQVVHSPYTFGGGTKVEIK

SEQ ID NO: 207 - protein, 108 aa, synthetic construct
  REGION 1..108: note = Synthetic
EIVLTQSPGTLSLSPGERATLSCGASQSVSSAFLAWYQQKPGQAPRLLIYGASARATGIPDRFSGSGSGTDFTLTISRLEPEDFAVYYCQQVVHSPYTFGGGTKVEIK

SEQ ID NO: 208 - protein, 108 aa, synthetic construct
  REGION 1..108: note = Synthetic
EIVLTQSPGTLSLSPGERATLSCRASQSVSSTYLAWYQQKPGQAPRLLIYGASSREAGIPDRFSGSGSGTDFTLTISRLEPEDFAVYYCQQTVHSPYTFGGGTKVEIK

SEQ ID NO: 209 - protein, 108 aa, synthetic construct
  REGION 1..108: note = Synthetic
EIVLTQSPGTLSLSPGERATLSCQASQAVSSNYLAWYQQKPGQAPRLLIYGASNRAAGIPDRFSGSGSGTDFTLTISRLEPEDFAVYYCQQAIHSPYTFGGGTKVEIK

SEQ ID NO: 210 - protein, 108 aa, synthetic construct
  REGION 1..108: note = Synthetic
EIVLTQSPGTLSLSPGERATLSCQASQSVSSSYLAWYQQKPGQAPRLLIYGASNRAAGIPDRFSGSGSGTDFTLTISRLEPEDFAVYYCQQVVHSPYTFGGGTKVEIK

SEQ ID NO: 211 - protein, 108 aa, synthetic construct
  REGION 1..108: note = Synthetic
EIVLTQSPGTLSLSPGERATLSCKASQAVSSSYLAWYQQKPGQAPRLLIYGASSRQDGIPDRFSGSGSGTDFTLTISRLEPEDFAVYYCQQVVHSPYTFGGGTKVEIK

SEQ ID NO: 212 - protein, 108 aa, synthetic construct
  REGION 1..108: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQHSSYPPTFGGGTKVEIK

SEQ ID NO: 213 - protein, 108 aa, synthetic construct
  REGION 1..108: note = Synthetic
EIVLTQSPGTLSLSPGERATLSCEASQSVSSSYLAWYQQKPGQAPRLLIYGASNRATGIPDRFSGSGSGTDFTLTISRLEPEDFAVYYCQQHSLYPPTFGGGTKVEIK

SEQ ID NO: 214 - protein, 108 aa, synthetic construct
  REGION 1..108: note = Synthetic
EIVLTQSPGTLSLSPGERATLSCEASQSVSASYLAWYQQKPGQAPRLLIYDASSRASGIPDRFSGSGSGTDFTLTISRLEPEDFAVYYCQQFSSYPPTFGGGTKVEIK

SEQ ID NO: 215 - protein, 108 aa, synthetic construct
  REGION 1..108: note = Synthetic
EIVLTQSPGTLSLSPGERATLSCEASQSVSSAYLAWYQQKPGQAPRLLIYDASTRATGIPDRFSGSGSGTDFTLTISRLEPEDFAVYYCQQVSSYPPTFGGGTKVEIK

SEQ ID NO: 216 - protein, 108 aa, synthetic construct
  REGION 1..108: note = Synthetic
EIVLTQSPGTLSLSPGERAALSCRVSQSVSDAYLAWYQQKPGQAPRLLIYDASSRASGIPDRFSGSGSGTDFTLTISRLEPEDFAVYYCQQVSSYPPTFGGGTKVEIK

SEQ ID NO: 217 - protein, 108 aa, synthetic construct
  REGION 1..108: note = Synthetic
EIVLTQSPGTLSLSPGERATLSCEVSQSVSASYLAWYQQKPGQAPRLLIYGASSRATGIPDRFSGSGSGTDFTLTISRLEPEDFAVYYCQQHSLYPPTFGGGTKVEIK

SEQ ID NO: 218 - protein, 108 aa, synthetic construct
  REGION 1..108: note = Synthetic
EIVLTQSPGTLSLSPGERATLSCRASQSVSSAYLAWYQQKPGQAPRLLIYGASDRANGIPDRFSGSGSGTDFTLTISRLEPEDFAVYYCQQHSLYPPTFGGGTKVEIK

SEQ ID NO: 219 - protein, 108 aa, synthetic construct
  REGION 1..108: note = Synthetic
EIVLTQSPGTLSLSPGERATLSCRASNAVSSSYLAWYQQKPGQAPRLLIYGASDRANGIPDRFSGSGSGTDFTLTISRLEPEDFAVYYCQQHSIYPPTFGGGTKVEIK

SEQ ID NO: 220 - protein, 108 aa, synthetic construct
  REGION 1..108: note = Synthetic
EIVLTQSPGTLSLSPGERATLSCRASNAVSSSYLAWYQQKPGQAPRLLIYGASYRATGIPDRFSGSGSGTDFTLTISRLEPEDFAVYYCQQHSLYPPTFGGGTKVEIK

SEQ ID NO: 221 - protein, 106 aa, synthetic construct
  REGION 1..106: note = Synthetic
DIQMTQSPSTLSASVGDRVTITCRASQSINSWLAWYQQKPGKAPKLLISDASSLESGVPSRFSGSGSGTEFTLTISSLQPDDFATYYCQQYDSHITFGGGTKVEIK

SEQ ID NO: 222 - protein, 107 aa, synthetic construct
  REGION 1..107: note = Synthetic
DIQMTQSPSSVSASVGDRVTITCAASQGISSDLAWYQQKPGKAPKLLIYSASSTQSGVPSRFSGSGSGTDFTLTISSLQPEDFATYYCQQAYLYPITFGGGTKVEIK

SEQ ID NO: 223 - protein, 107 aa, synthetic construct
  REGION 1..107: note = Synthetic
GVQMTQSPSSLSASVGDRVTITCRASQDISTYLNWYQQKPGKAPKLLIYDAFNLETGVPSRFSGSGSGTDFTFTISSLQPEDIATYYCQQLPFLPITFGGGTKVEIK

SEQ ID NO: 224 - protein, 112 aa, synthetic construct
  REGION 1..112: note = Synthetic
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALGGPWTFGGGTKVEIK

SEQ ID NO: 225 - protein, 108 aa, synthetic construct
  REGION 1..108: note = Synthetic
EIVLTQSPGTLSLSPGERATLSCRASQSVSSSYLAWYQQKPGQAPRLLIYGASRRATGIPDRFSGSGSGTDFTLTISRLEPEDFAVYYCQQYVSDPITFGGGTKVEIK

SEQ ID NO: 226 - protein, 108 aa, synthetic construct
  REGION 1..108: note = Synthetic
EIVLTQSPGTLSLSPGERATLSCRASQSVSSSYLAWYQQKPGQAPRLLIYGASRRATGIPDRFSGSGSGTDFTLTISRLEPEDFAVYYCQQVGSSPITFGGGTKVEIK

SEQ ID NO: 227 - protein, 107 aa, synthetic construct
  REGION 1..107: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSHLVPRTFGGGTKVEIK

SEQ ID NO: 228 - protein, 108 aa, synthetic construct
  REGION 1..108: note = Synthetic
EIVMTQSPATLSVSPGERATLSCRASQSVSSNLAWYQQKPGQAPRLLIYGASTRATGIPARFSGSGSGTEFTLTISSLQSEDFAVYYCQQANHHPPFTFGGGTKVEIK

SEQ ID NO: 229-231 - skipped (3 entries)

SEQ ID NO: 232 - protein, 450 aa, synthetic construct
  REGION 1..450: note = Synthetic
QLQLQESGPGLVKPSETLSLTCTVSGGSISSSDYYWGWIRQPPGKGLEWIGSIYYSGSTYYNPSLKSRVTISVDTSKNQFSLKLSSVTAADTAVYYCARGVRRAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - protein, 450 aa, synthetic construct
  REGION 1..450: note = Synthetic
QLQLQESGPGLVKPSETLSLTCTVSGGSISSSSTYWAWIRQPPGKGLEWIGSISSSGSTYYNPSLKSRVTISVDTSKNQFSLKLSSVTAADTAVYYCARGIARAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 234 - protein, 450 aa, synthetic construct
  REGION 1..450: note = Synthetic
QLQLQESGPGLVKPSETLSLTCTVSGGSISSSSTYWGWIRQSPGKGLEWIGSIHHSGATYYNPSLKSRVTISVDTSKNQFSLKLSSVTAADTAVYYCARGPKRAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 450 aa, synthetic construct
  REGION 1..450: note = Synthetic
QLQLQESGPGLVKPSETLSLTCTVSGGSISSSDTYWGWIRQPPGKGLEWIGSIHYSGSTLYNPSLKSRVTISVDTSKNQFSLKLSSVTAADTAVYYCARGVRRAVPFVDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - protein, 450 aa, synthetic construct
  REGION 1..450: note = Synthetic
QLQLQESGPGLVKPSETLSLTCTVSGGSISSADNYWGWIRQPPGKGLEWIGSIHYSGSTYYNPSLKSRVTISVDTSKNQFSLKLSSVTAADTAVYYCARGVRRAVPFQRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 450 aa, synthetic construct
  REGION 1..450: note = Synthetic
QLQLQESGPGLVKPSETLSLTCTVSGGSISSSDTYWGWIRQPPGKGPEWIGSIHYSGSTLYNPSLKSRVTISVDTSKNQFSLKLSSVTAADTAVYYCARGVRRAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 238 - protein, 450 aa, synthetic construct
  REGION 1..450: note = Synthetic
QLQLQESGPGLVKPSETLSLTCTVSGGSISSSSTYWSWIRQPPGKGLEWIGGIAYSGSTYYNPSLKSRVTISVDTSKNQFSLKLSSVTAADTAVYYCARGVRRAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 450 aa, synthetic construct
  REGION 1..450: note = Synthetic
QLQLQESGPGLVKPSETLSLTCTVSGGSISSSSTYWSWIRQPPGKGLEWIGSISYNALTYYNPSLKSRVTISVDTSKNQFSLKLSSVTAADTAVYYCARGTRRAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 240 - protein, 450 aa, synthetic construct
  REGION 1..450: note = Synthetic
QLQLQESGPGLVKPSETLSLTCTVSGGSISSSSTYWSWIRQPPGKGLEWIGGIAYSGSTYYNPSLKSRVTISVDTSKNQFSLKLSSVTAADTAVYYCARGVRRAVPFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 241 - protein, 450 aa, synthetic construct
  REGION 1..450: note = Synthetic
QLQLQESGPGLVKPSETLSLTCTVSGGSVSSSSTYWSWIRQPPGKGLEWIGGIAYSGSTYYNPSLKSRVTISVDTSKNQFSLKLSSVTAADTAVYYCARGVRRAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - protein, 450 aa, synthetic construct
  REGION 1..450: note = Synthetic
QLQLQESGPGLVKPSETLSLTCTVSGGSISSSSTYWSWIRQPPGKGLEWIGGIAYSGSTYYNPSLKSRVTISVDTSKNQFSLKLSSVTAADTAVYYCARGTRRAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 243 - protein, 450 aa, synthetic construct
  REGION 1..450: note = Synthetic
QLQLQESGPGLVKPSETLSLTCTVSGGSISSSDTYWGWIRQPPGKGLEWIGSIHYSGSTLYNPSLKSRVTISVDTSKNQFSLKLSSVTAADTAVYYCARGIRRAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 244 - protein, 450 aa, synthetic construct
  REGION 1..450: note = Synthetic
QLQLQESGPGLVKPSETLSLTCTVSGGSISSSDTYWGWIRQPPGKGLEWIGSIHYSGSTLYNPSLKSRVTISVDTSKNQFSLKLSSVTAADTAVYYCARGQFRAVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 245 - protein, 452 aa, synthetic construct
  REGION 1..452: note = Synthetic
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGGTHTYSRGPMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 246 - protein, 452 aa, synthetic construct
  REGION 1..452: note = Synthetic
QLQLQESGPRLVKPSETLSLTCAVSGYSISSGYYWGWIRQPPGKGLEWIGSIYHSGSTYYNPSLKSRVTISVDTSKNQFSLKLSSVTAADTAVYYCARGGTHTYSRGPFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 247 - protein, 452 aa, synthetic construct
  REGION 1..452: note = Synthetic
LVQLQESGPGLVKPSETLSLTCAVSGYSILSGYYWFWIRQPPGKGLEWIGGIYHSASTAYNPSLKSRVTISVDTSKNQFSLKLSSVTAADTAVYYCARGGTPIYSRGPLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 248 - protein, 452 aa, synthetic construct
  REGION 1..452: note = Synthetic
QVQLQESGPGLVKPSETLSLTCAVSGYSISSGHYWIWIRQPPGKGLEWIGGIYHSGSTYYNPSLKSRVTISVDTSKDQFSLKLSSVTAADTAVYYCARGGGQTYSRGPLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 249 - protein, 452 aa, synthetic construct
  REGION 1..452: note = Synthetic
QVQLQESGPGLVKPPETLSLTCAVSGYSISSGHYWIWIRQPPGKGLEWIGGIYHSGSTYYNPSLKSRVTISVDTSKNQFSLKLSSVTAADTAVYYCARGGGATYSRGPLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 250 - protein, 452 aa, synthetic construct
  REGION 1..452: note = Synthetic
QVQLQESGPGLVKPSETLSLTCAVSGYSILSGYYWFWIRQPPGKGLEWIGGIYHSGSTYYNPSLKSRVTISVDTSKNQFSLKLSSVTAADTAVYYCARGGTHTYSRGPLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 251 - protein, 452 aa, synthetic construct
  REGION 1..452: note = Synthetic
QVQLQESGPGLVKPSETLSLTCAVSGYSISSGFYWTWIRQPPGKGLEWIGAIYHSGSTVYNPSLKSRVTISVDTSKNQFSLKLSSVTAADTAVYYCARGGTHTYSRGPLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 252 - protein, 452 aa, synthetic construct
  REGION 1..452: note = Synthetic
QVQLQESGPGLVKPSETLSLTCAVSGYSISSGYYWLWIRQPPGKGLEWIGGIYHSASTAYNPSLKSRVTISVDTSKNQFSLKLSSVTAADTAVYYCARGGTVKYSRGPLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 253 - protein, 452 aa, synthetic construct
  REGION 1..452: note = Synthetic
QVQLQESGPGLVKPSETLSLTCAVSGYSISSGHYWTWIRQPPGKGLEWIGAIYHSGSTVYNPSLKSRVTISVDTSKNQFSLKLSSVTAADTAVYYCARGGQVTYSRGPLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 254 - protein, 452 aa, synthetic construct
  REGION 1..452: note = Synthetic
QVQLQESGPGLVKPSETLSLTCAVSGYSILSGYYWFWIRQPPGKGLEWIGGIYHSGSTAYNPSLKSRVTISVDTSKNQFSLKLSSVTAADTAVYYCARGGEVTYSRGPLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 255 - protein, 453 aa, synthetic construct
  REGION 1..453: note = Synthetic
EVQLLESGGGLVQPGGSLRLSCAASGFTFDNYAMHWVRQAPGKGLEWVSAISARAGITYYADSVKGRFTISRDNSKNTLYLQMNSLRAEDTAVYYCARRIGYSYGTAPPFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 256 - protein, 448 aa, synthetic construct
  REGION 1..448: note = Synthetic
QVQLVESGGGLVQPGGSLRLSCAASGFTFSDYYMSWIRQAPGKGLEWVSYIASSGSVIYYADSVKGRFTISRDNAKNSLYLQMNSLRAEDTAVYYCARHGTPRAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 257 - protein, 449 aa, synthetic construct
  REGION 1..449: note = Synthetic
EVQLLESGGGLVQPGGSLRLSCAASGFTFSSSAMAWVRQAPGKGLEWVSTISGSGITTWYADSVKGRFTISRDNSKNTLYLQMNSLRAEDTAVYYCAKGSRHLNAFNRWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 258 - protein, 451 aa, synthetic construct
  REGION 1..451: note = Synthetic
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARGVYHYDPYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 259 - protein, 450 aa, synthetic construct
  REGION 1..450: note = Synthetic
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARTELGKMHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 260 - protein, 448 aa, synthetic construct
  REGION 1..448: note = Synthetic
QLQLQESGPGLVKPSETLSLTCTVSGGSISSSDYYWGWIRQPPGKGLEWIGSIYYSGSTYYNPSLKSRVTISVDTSKNQFSLKLSSVTAADTAVYYCARGSPRYMQDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 261 - protein, 451 aa, synthetic construct
  REGION 1..451: note = Synthetic
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARHSSLGTHNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 262 - protein, 453 aa, synthetic construct
  REGION 1..453: note = Synthetic
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCAREGALSYSWLAAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 263-265 - skipped (3 entries)

SEQ ID NO: 266 - protein, 447 aa, synthetic construct
  REGION 1..447: note = Synthetic
QLQLQESGPGLVKPSETLSLTCTVSGGSISSSDYYWGWIRQPPGKGLEWIGSIYYSGSTYYNPSLKSRVTISVDTSKNQFSLKLSSVTAADTAVYYCARGVRRAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 267 - protein, 447 aa, synthetic construct
  REGION 1..447: note = Synthetic
QLQLQESGPGLVKPSETLSLTCTVSGGSISSSSTYWAWIRQPPGKGLEWIGSISSSGSTYYNPSLKSRVTISVDTSKNQFSLKLSSVTAADTAVYYCARGIARAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 268 - protein, 447 aa, synthetic construct
  REGION 1..447: note = Synthetic
QLQLQESGPGLVKPSETLSLTCTVSGGSISSSSTYWGWIRQSPGKGLEWIGSIHHSGATYYNPSLKSRVTISVDTSKNQFSLKLSSVTAADTAVYYCARGPKRAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 269 - protein, 447 aa, synthetic construct
  REGION 1..447: note = Synthetic
QLQLQESGPGLVKPSETLSLTCTVSGGSISSSDTYWGWIRQPPGKGLEWIGSIHYSGSTLYNPSLKSRVTISVDTSKNQFSLKLSSVTAADTAVYYCARGVRRAVPFVDWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 270 - protein, 447 aa, synthetic construct
  REGION 1..447: note = Synthetic
QLQLQESGPGLVKPSETLSLTCTVSGGSISSADNYWGWIRQPPGKGLEWIGSIHYSGSTYYNPSLKSRVTISVDTSKNQFSLKLSSVTAADTAVYYCARGVRRAVPFQRWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 271 - protein, 447 aa, synthetic construct
  REGION 1..447: note = Synthetic
QLQLQESGPGLVKPSETLSLTCTVSGGSISSSDTYWGWIRQPPGKGPEWIGSIHYSGSTLYNPSLKSRVTISVDTSKNQFSLKLSSVTAADTAVYYCARGVRRAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 272 - protein, 447 aa, synthetic construct
  REGION 1..447: note = Synthetic
QLQLQESGPGLVKPSETLSLTCTVSGGSISSSSTYWSWIRQPPGKGLEWIGGIAYSGSTYYNPSLKSRVTISVDTSKNQFSLKLSSVTAADTAVYYCARGVRRAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 273 - protein, 447 aa, synthetic construct
  REGION 1..447: note = Synthetic
QLQLQESGPGLVKPSETLSLTCTVSGGSISSSSTYWSWIRQPPGKGLEWIGSISYNALTYYNPSLKSRVTISVDTSKNQFSLKLSSVTAADTAVYYCARGTRRAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 274 - protein, 447 aa, synthetic construct
  REGION 1..447: note = Synthetic
QLQLQESGPGLVKPSETLSLTCTVSGGSISSSSTYWSWIRQPPGKGLEWIGGIAYSGSTYYNPSLKSRVTISVDTSKNQFSLKLSSVTAADTAVYYCARGVRRAVPFADWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 275 - protein, 447 aa, synthetic construct
  REGION 1..447: note = Synthetic
QLQLQESGPGLVKPSETLSLTCTVSGGSVSSSSTYWSWIRQPPGKGLEWIGGIAYSGSTYYNPSLKSRVTISVDTSKNQFSLKLSSVTAADTAVYYCARGVRRAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 276 - protein, 447 aa, synthetic construct
  REGION 1..447: note = Synthetic
QLQLQESGPGLVKPSETLSLTCTVSGGSISSSSTYWSWIRQPPGKGLEWIGGIAYSGSTYYNPSLKSRVTISVDTSKNQFSLKLSSVTAADTAVYYCARGTRRAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 277 - protein, 447 aa, synthetic construct
  REGION 1..447: note = Synthetic
QLQLQESGPGLVKPSETLSLTCTVSGGSISSSDTYWGWIRQPPGKGLEWIGSIHYSGSTLYNPSLKSRVTISVDTSKNQFSLKLSSVTAADTAVYYCARGIRRAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 278 - protein, 447 aa, synthetic construct
  REGION 1..447: note = Synthetic
QLQLQESGPGLVKPSETLSLTCTVSGGSISSSDTYWGWIRQPPGKGLEWIGSIHYSGSTLYNPSLKSRVTISVDTSKNQFSLKLSSVTAADTAVYYCARGQFRAVPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 279 - protein, 449 aa, synthetic construct
  REGION 1..449: note = Synthetic
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGGTHTYSRGPMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 280 - protein, 449 aa, synthetic construct
  REGION 1..449: note = Synthetic
QLQLQESGPRLVKPSETLSLTCAVSGYSISSGYYWGWIRQPPGKGLEWIGSIYHSGSTYYNPSLKSRVTISVDTSKNQFSLKLSSVTAADTAVYYCARGGTHTYSRGPFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 281 - protein, 449 aa, synthetic construct
  REGION 1..449: note = Synthetic
LVQLQESGPGLVKPSETLSLTCAVSGYSILSGYYWFWIRQPPGKGLEWIGGIYHSASTAYNPSLKSRVTISVDTSKNQFSLKLSSVTAADTAVYYCARGGTPIYSRGPLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 282 - protein, 449 aa, synthetic construct
  REGION 1..449: note = Synthetic
QVQLQESGPGLVKPSETLSLTCAVSGYSISSGHYWIWIRQPPGKGLEWIGGIYHSGSTYYNPSLKSRVTISVDTSKDQFSLKLSSVTAADTAVYYCARGGGQTYSRGPLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 283 - protein, 449 aa, synthetic construct
  REGION 1..449: note = Synthetic
QVQLQESGPGLVKPPETLSLTCAVSGYSISSGHYWIWIRQPPGKGLEWIGGIYHSGSTYYNPSLKSRVTISVDTSKNQFSLKLSSVTAADTAVYYCARGGGATYSRGPLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 284 - protein, 449 aa, synthetic construct
  REGION 1..449: note = Synthetic
QVQLQESGPGLVKPSETLSLTCAVSGYSILSGYYWFWIRQPPGKGLEWIGGIYHSGSTYYNPSLKSRVTISVDTSKNQFSLKLSSVTAADTAVYYCARGGTHTYSRGPLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 285 - protein, 449 aa, synthetic construct
  REGION 1..449: note = Synthetic
QVQLQESGPGLVKPSETLSLTCAVSGYSISSGFYWTWIRQPPGKGLEWIGAIYHSGSTVYNPSLKSRVTISVDTSKNQFSLKLSSVTAADTAVYYCARGGTHTYSRGPLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 286 - protein, 449 aa, synthetic construct
  REGION 1..449: note = Synthetic
QVQLQESGPGLVKPSETLSLTCAVSGYSISSGYYWLWIRQPPGKGLEWIGGIYHSASTAYNPSLKSRVTISVDTSKNQFSLKLSSVTAADTAVYYCARGGTVKYSRGPLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 287 - protein, 449 aa, synthetic construct
  REGION 1..449: note = Synthetic
QVQLQESGPGLVKPSETLSLTCAVSGYSISSGHYWTWIRQPPGKGLEWIGAIYHSGSTVYNPSLKSRVTISVDTSKNQFSLKLSSVTAADTAVYYCARGGQVTYSRGPLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 288 - protein, 449 aa, synthetic construct
  REGION 1..449: note = Synthetic
QVQLQESGPGLVKPSETLSLTCAVSGYSILSGYYWFWIRQPPGKGLEWIGGIYHSGSTAYNPSLKSRVTISVDTSKNQFSLKLSSVTAADTAVYYCARGGEVTYSRGPLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 289 - protein, 450 aa, synthetic construct
  REGION 1..450: note = Synthetic
EVQLLESGGGLVQPGGSLRLSCAASGFTFDNYAMHWVRQAPGKGLEWVSAISARAGITYYADSVKGRFTISRDNSKNTLYLQMNSLRAEDTAVYYCARRIGYSYGTAPPFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 290 - protein, 445 aa, synthetic construct
  REGION 1..445: note = Synthetic
QVQLVESGGGLVQPGGSLRLSCAASGFTFSDYYMSWIRQAPGKGLEWVSYIASSGSVIYYADSVKGRFTISRDNAKNSLYLQMNSLRAEDTAVYYCARHGTPRAFDIWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 291 - protein, 446 aa, synthetic construct
  REGION 1..446: note = Synthetic
EVQLLESGGGLVQPGGSLRLSCAASGFTFSSSAMAWVRQAPGKGLEWVSTISGSGITTWYADSVKGRFTISRDNSKNTLYLQMNSLRAEDTAVYYCAKGSRHLNAFNRWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 292 - protein, 448 aa, synthetic construct
  REGION 1..448: note = Synthetic
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARGVYHYDPYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 293 - protein, 447 aa, synthetic construct
  REGION 1..447: note = Synthetic
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARTELGKMHFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 294 - protein, 445 aa, synthetic construct
  REGION 1..445: note = Synthetic
QLQLQESGPGLVKPSETLSLTCTVSGGSISSSDYYWGWIRQPPGKGLEWIGSIYYSGSTYYNPSLKSRVTISVDTSKNQFSLKLSSVTAADTAVYYCARGSPRYMQDWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 295 - protein, 448 aa, synthetic construct
  REGION 1..448: note = Synthetic
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARHSSLGTHNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 296 - protein, 450 aa, synthetic construct
  REGION 1..450: note = Synthetic
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCAREGALSYSWLAAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 297-299 - skipped (3 entries)

SEQ ID NO: 300 - protein, 215 aa, synthetic construct
  REGION 1..215: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQAVHSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 301 - protein, 215 aa, synthetic construct; identical to SEQ ID NO: 300
  REGION 1..215: note = Synthetic

SEQ ID NO: 302 - protein, 215 aa, synthetic construct; identical to SEQ ID NO: 300
  REGION 1..215: note = Synthetic

SEQ ID NO: 303 - protein, 215 aa, synthetic construct; identical to SEQ ID NO: 300
  REGION 1..215: note = Synthetic

SEQ ID NO: 304 - protein, 215 aa, synthetic construct; identical to SEQ ID NO: 300
  REGION 1..215: note = Synthetic

SEQ ID NO: 305 - protein, 215 aa, synthetic construct
  REGION 1..215: note = Synthetic
EIVLTQSPGTLSLSPGERATLSCGASQSVSSDYLAWYQQKPGQAPRLLIYGAYSLATGIPDRFSGSGSGTDFTLTISRLEPEDFAVYYCQWAVHSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 306 - protein, 215 aa, synthetic construct
  REGION 1..215: note = Synthetic
EIVLTQSPGTLSLSPGERATLSCQASQAVSSNYLAWYQQKPGQAPRLLIYGASSRATGIPDRFSGSGSGTDFTLTISRLEPEDFAVYYCQQVVHSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 307 - protein, 215 aa, synthetic construct
  REGION 1..215: note = Synthetic
EIVLTQSPGTLSLSPGERATLSCGASQSVSSAFLAWYQQKPGQAPRLLIYGASARATGIPDRFSGSGSGTDFTLTISRLEPEDFAVYYCQQVVHSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 308 - protein, 215 aa, synthetic construct
  REGION 1..215: note = Synthetic
EIVLTQSPGTLSLSPGERATLSCRASQSVSSTYLAWYQQKPGQAPRLLIYGASSREAGIPDRFSGSGSGTDFTLTISRLEPEDFAVYYCQQTVHSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 309 - protein, 215 aa, synthetic construct
  REGION 1..215: note = Synthetic
EIVLTQSPGTLSLSPGERATLSCQASQAVSSNYLAWYQQKPGQAPRLLIYGASNRAAGIPDRFSGSGSGTDFTLTISRLEPEDFAVYYCQQAIHSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 310 - protein, 215 aa, synthetic construct
  REGION 1..215: note = Synthetic
EIVLTQSPGTLSLSPGERATLSCQASQSVSSSYLAWYQQKPGQAPRLLIYGASNRAAGIPDRFSGSGSGTDFTLTISRLEPEDFAVYYCQQVVHSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 311 - protein, 215 aa, synthetic construct
  REGION 1..215: note = Synthetic
EIVLTQSPGTLSLSPGERATLSCKASQAVSSSYLAWYQQKPGQAPRLLIYGASSRQDGIPDRFSGSGSGTDFTLTISRLEPEDFAVYYCQQVVHSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 312 - protein, 215 aa, synthetic construct; identical to SEQ ID NO: 300
  REGION 1..215: note = Synthetic

SEQ ID NO: 313 - protein, 215 aa, synthetic construct
  REGION 1..215: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQHSSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 314 - protein, 215 aa, synthetic construct; identical to SEQ ID NO: 313
  REGION 1..215: note = Synthetic

SEQ ID NO: 315 - protein, 215 aa, synthetic construct
  REGION 1..215: note = Synthetic
EIVLTQSPGTLSLSPGERATLSCEASQSVSSSYLAWYQQKPGQAPRLLIYGASNRATGIPDRFSGSGSGTDFTLTISRLEPEDFAVYYCQQHSLYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 316 - protein, 215 aa, synthetic construct
  REGION 1..215: note = Synthetic
EIVLTQSPGTLSLSPGERATLSCEASQSVSASYLAWYQQKPGQAPRLLIYDASSRASGIPDRFSGSGSGTDFTLTISRLEPEDFAVYYCQQFSSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 317 - protein, 215 aa, synthetic construct
  REGION 1..215: note = Synthetic
EIVLTQSPGTLSLSPGERATLSCEASQSVSSAYLAWYQQKPGQAPRLLIYDASTRATGIPDRFSGSGSGTDFTLTISRLEPEDFAVYYCQQVSSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 318 - protein, 215 aa, synthetic construct
  REGION 1..215: note = Synthetic
EIVLTQSPGTLSLSPGERAALSCRVSQSVSDAYLAWYQQKPGQAPRLLIYDASSRASGIPDRFSGSGSGTDFTLTISRLEPEDFAVYYCQQVSSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 319 - protein, 215 aa, synthetic construct
  REGION 1..215: note = Synthetic
EIVLTQSPGTLSLSPGERATLSCEVSQSVSASYLAWYQQKPGQAPRLLIYGASSRATGIPDRFSGSGSGTDFTLTISRLEPEDFAVYYCQQHSLYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 320 - protein, 215 aa, synthetic construct
  REGION 1..215: note = Synthetic
EIVLTQSPGTLSLSPGERATLSCRASQSVSSAYLAWYQQKPGQAPRLLIYGASDRANGIPDRFSGSGSGTDFTLTISRLEPEDFAVYYCQQHSLYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 321 - protein, 215 aa, synthetic construct
  REGION 1..215: note = Synthetic
EIVLTQSPGTLSLSPGERATLSCRASNAVSSSYLAWYQQKPGQAPRLLIYGASDRANGIPDRFSGSGSGTDFTLTISRLEPEDFAVYYCQQHSIYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 322 - protein, 215 aa, synthetic construct
  REGION 1..215: note = Synthetic
EIVLTQSPGTLSLSPGERATLSCRASNAVSSSYLAWYQQKPGQAPRLLIYGASYRATGIPDRFSGSGSGTDFTLTISRLEPEDFAVYYCQQHSLYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 323 - protein, 213 aa, synthetic construct
  REGION 1..213: note = Synthetic
DIQMTQSPSTLSASVGDRVTITCRASQSINSWLAWYQQKPGKAPKLLISDASSLESGVPSRFSGSGSGTEFTLTISSLQPDDFATYYCQQYDSHITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 324 - protein, 214 aa, synthetic construct
  REGION 1..214: note = Synthetic
DIQMTQSPSSVSASVGDRVTITCAASQGISSDLAWYQQKPGKAPKLLIYSASSTQSGVPSRFSGSGSGTDFTLTISSLQPEDFATYYCQQAYLYPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 325 - protein, 214 aa, synthetic construct
  REGION 1..214: note = Synthetic
GVQMTQSPSSLSASVGDRVTITCRASQDISTYLNWYQQKPGKAPKLLIYDAFNLETGVPSRFSGSGSGTDFTFTISSLQPEDIATYYCQQLPFLPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 326 - protein, 219 aa, synthetic construct
  REGION 1..219: note = Synthetic
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALGGPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 327 - protein, 215 aa, synthetic construct
  REGION 1..215: note = Synthetic
EIVLTQSPGTLSLSPGERATLSCRASQSVSSSYLAWYQQKPGQAPRLLIYGASRRATGIPDRFSGSGSGTDFTLTISRLEPEDFAVYYCQQYVSDPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 328 - protein, 215 aa, synthetic construct
  REGION 1..215: note = Synthetic
EIVLTQSPGTLSLSPGERATLSCRASQSVSSSYLAWYQQKPGQAPRLLIYGASRRATGIPDRFSGSGSGTDFTLTISRLEPEDFAVYYCQQVGSSPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 329 - protein, 214 aa, synthetic construct
  REGION 1..214: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSHLVPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 330 - protein, 215 aa, synthetic construct
  REGION 1..215: note = Synthetic
EIVMTQSPATLSVSPGERATLSCRASQSVSSNLAWYQQKPGQAPRLLIYGASTRATGIPARFSGSGSGTEFTLTISSLQSEDFAVYYCQQANHHPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 331-333 - skipped (3 entries)

SEQ ID NO: 334 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 335 - protein, 327 aa, synthetic construct
  REGION 1..327: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 336 - protein, 107 aa, synthetic construct [sequence omitted]

SEQ ID NO: 337 - protein, 338 aa, synthetic construct
  REGION 1..338: note = Synthetic
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 338 - protein, 319 aa, synthetic construct
  REGION 1..319: note = Synthetic
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDL

SEQ ID NO: 339 - protein, 336 aa, synthetic construct
  REGION 1..336: note = Synthetic
MAVMAPRTLLLVLSGVLALTQPRAGSHSMRYFYTAVSRPGRGQPRFIAVGYVDDTQFVRFDSDAESPRMEPRAPWVEQEGPEYWDRETQNMKTATQTYQANLRTLLRYYNQSEAGSHTFQKMYGCDLGPDGRLLRGYEQFAYDGRDYIILNEDLRSWTAADMAAQNTQRKWEAAGAAEQHRTYLEGECLEWLRRYLENGKETLQRADPPKTNVTHHPVSDYEATLRCWALGFYPAEITLTWQRDGEEQTEDTELVETRPTGDGTFQKWAAVVVPSGEEQRYTCHVQHEGLPKPLTLRWEPSSQSTILIVGIIAGLVLLGTVVTGAVVAAVMWRRKS

SEQ ID NO: 340 - protein, 331 aa, synthetic construct
  REGION 1..331: note = Synthetic
LLLVLSGVLALTQTRAGSHSMRYFYTSMSRPGRGQPRFIAVGYVDDTQFVRFDSDAESPRMEPRAPWVEQEGPEYWDRETQNMKTATQTYRENLRTLLRYYNQSEAGSHTIQKMYGCDLGPDGRLLRGYEQFAYDGRDYIALNEDLRSWTAADMAAQFTQRKWEAAGAAEQHRTYLEGECLEWLRRYLENGKETLQRADPPKTNVTHHPVSDYEATLRCWALGFYPAEITLTWQRDGEEQTEDTELVETRPTGDGTFQKWAAVVVPSGEEQRYTCHVQHEGLPEPLTLRWEPSSQSTILIVGIIAGLVLLGTVVTGAVVAAVMWRRKSSDR

SEQ ID NO: 341 - protein, 298 aa, synthetic construct
  REGION 1..298: note = Synthetic
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRW

SEQ ID NO: 342 - protein, 274 aa, synthetic construct
  REGION 1..274: note = Synthetic
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRW

(19) United States
(12) Patent Application Publication
(10) Pub. No.: US 2025/0353913 A1
(43) Pub. Date: Nov. 20, 2025

(54) ANTI-HLA-G ANTIBODIES, COMPOSITIONS COMPRISING ANTI-HLA-G ANTIBODIES AND METHODS OF USING ANTI-HLA-G ANTIBODIES

(71) Applicant: Tizona Therapeutics, South San Francisco, CA (US)

(72) Inventors: Courtney Beers, South San Francisco, CA (US); John Corbin, South San Francisco, CA (US); Doug Hodges, South San Francisco, CA (US); Achim Moesta, South San Francisco, CA (US); Vanessa Soros, South San Francisco, CA (US); Paul Fredrick Widboom, South San Francisco, CA (US); Joseph Robert Warfield, South San Francisco, CA (US)

(21) Appl. No.: 19/065,819

(22) Filed: Feb. 27, 2025

Related U.S. Application Data
(63) Continuation of application No. 17/530,072, filed on Nov. 18, 2021, now Pat. No. 12,264,199.
(63) Continuation of application No. 16/584,421, filed on Sep. 26, 2019, now Pat. No. 11,208,487.
(60) Provisional application No. 62/737,666, filed on Sep. 27, 2018.

(51) Int. Cl. C07K 16/28; A61K 45/06; A61P 35/00; C07K 16/32; G01N 33/569

(52) U.S. Cl. CPC C07K 16/2833; A61P 35/00; C07K 16/2818; C07K 16/2827; C07K 16/2896; C07K 16/32; G01N 33/56977; A61K 45/06; C07K 2317/24; C07K 2317/31; C07K 2317/54; C07K 2317/55; C07K 2317/565; C07K 2317/626; C07K 2317/732; C07K 2317/76; C07K 2317/92

(57) ABSTRACT

Provided herein are antibodies that selectively bind to HLA-G and compositions comprising the antibodies. Also provided are methods of using the antibodies, such as therapeutic and diagnostic methods.

### RELATED APPLICATION

This application claims priority to U.S. provisional application No. 62/737,666, filed Sep. 27, 2018, which is incorporated by reference herein in its entirety.

### FIELD

Provided herein are antibodies with binding specificity for HLA-G and compositions comprising the antibodies, including pharmaceutical compositions, diagnostic compositions, and kits. Also provided are methods of using anti-HLA-G antibodies for therapeutic and diagnostic purposes.

### BACKGROUND

HLA-G histocompatibility antigen, class I, G, also known as human leukocyte antigen G (HLA-G), is a protein that in humans is encoded by the HLA-G gene. HLA-G belongs to the HLA nonclassical class I heavy chain paralogues. HLA-G is a heterodimer consisting of a heavy chain and a light chain (beta-2 microglobulin). There are membrane bound and soluble forms of HLA-G.

HLA-G is normally expressed at the maternal-fetal interface and other immune-privileged sites. HLA-G may play a role in immune tolerance in pregnancy, being expressed in the placenta by extravillous trophoblast cells, while the classical MHC class I genes (HLA-A and HLA-B) are not. As HLA-G was first identified in placenta samples, many studies have evaluated its role in pregnancy disorders, such as preeclampsia and recurrent pregnancy loss. See, Michita, Rafael Tomoya et al., Human Immunology. 2016, 77 (10): 892-897, which is incorporated by reference herein in its entirety, including any drawings.

HLA-G has been shown to be immune-suppressive. By binding receptors expressed on various myeloid and lymphoid cells, HLA-G may directly inhibit the functions of NK cells, cytotoxic T-lymphocytes, B cells, neutrophils, monocytes, macrophages and dendritic cells. HLA-G also inhibits T and NK cell proliferation and cytolytic activities. HLA-G suppresses phagocytosis and induces the generation or expansion of regulatory T cells.

HLA-G mediates immune function through at least three ITIM-containing inhibitory receptors, ILT2, ILT4, and KIR2DL4. On lymphoid and myeloid cells, for example, HLA-G mediates function through ILT2. On myeloid cells, HLA-G mediates function through ILT4. On decidual NK cells, HLA-G mediates immune function through KIR2DL4 and ILT2.

HLA-G is an immune checkpoint target. HLA-G can directly inhibit immune cell function through receptor binding and/or trogocytosis and impairment of chemotaxis. HLA-G can lend tumor cells a higher invasive and metastatic potential. HLA-G promotes evasion of tumor immune surveillance, and enhances metastasis and the progression of malignancies. During tumor progression HLA-G has other effects, such as, inhibition of immune cell cytolysis, induction of immune cell apoptosis, and/or the generation of regulatory cells through receptor binding and/or trogocytosis.

HLA-G expression is upregulated on a broad spectrum of tumors and is associated with poor prognosis and disease progression. Serum HLA-G levels are elevated in breast, lung, colorectal cancer (CRC), gastric, esophageal, neuroblastoma, cervical, and hematological cancers. HLA-G has also been found to be correlated with clinical parameters in advanced disease, such as, tumor metastasis, poor prognosis, immune escape, and tumor invasiveness.

HLA-G is an attractive target for diseases, such as, for example, cancer.

### SUMMARY

Provided herein are antibodies that selectively bind HLA-G. In some embodiments, the antibodies bind human HLA-G. In some embodiments, the antibodies comprise at least one CDR sequence defined by a consensus sequence provided in this disclosure. In some embodiments, the antibodies comprise one or more of an illustrative CDR, V_{H}, or V_{L} sequence provided in this disclosure, or a variant thereof. In some aspects, the variant is a variant with one or more conservative amino acid substitutions.

Also provided are compositions and kits comprising the antibodies. In some embodiments, the compositions are pharmaceutical compositions. Any suitable pharmaceutical composition may be used. In some embodiments, the pharmaceutical composition is a composition for parenteral administration.

This disclosure also provides methods of using the anti-HLA-G antibodies provided herein. In some embodiments, the method is a method of treatment. In some embodiments, the method is an analytical method. In some embodiments, the method is a method of purifying and/or quantifying HLA-G.

In some embodiments, the method is a diagnostic method. In some embodiments, the diagnostic method comprises or consists of detecting tumor expressed HLA-G. In some embodiments, the diagnostic method comprises or consists of detecting soluble HLA-G. In some embodiments, the detection method comprises of consists of detecting HLA-G expression on immune cells.

In some embodiments, the antibodies are used to treat a disease or condition. In some aspects, the disease or condition is selected from a cancer, autoimmune disease, and infection. Some aspects provide for the use of any of the antibodies or pharmaceutical compositions provided herein to treat a disease or condition selected from a cancer, autoimmune disease, and infection.

### BRIEF DESCRIPTION OF THE DRAWINGS

FIG. 1 provides a table showing avid and monomeric affinities of anti-HLA-G antibodies to recombinant HLA-G protein.

FIG. 2A and FIG. 2B provide biolayer interferometry sensorgrams showing anti-HLA-G antibodies that bind to HLA-G and can be separated into three biochemical bins based on their ability to cross-block each other when tested for binding in pairwise fashion.

FIG. 3 provide biolayer interferometry sensorgrams showing antibodies that bind and block HLA-G interaction with ILT2 and ILT4 at varying levels of effectiveness.

FIG. 4 shows evaluation of anti-HLA-G antibodies binding to naturally expressed HLA-G found on JEG-3 tumor cells.

FIG. 5A, FIG. 5B, FIG. 5C, and FIG. 5D provide data showing anti-HLA-G Fabs and antibodies that restore NKL killing activity by blocking suppression mediated through interaction of HLA-G with ILT2 or ILT4.

FIG. 6 provides evaluation of anti-HLA-G antibodies to reverse HLA-G mediated suppression of primary human cells. FIG. 6A and FIG. 6B provide evaluation of phagocytosis in a human macrophage assay. FIG. 6C provides evaluation of primary human NK cell cytotoxic activity. FIG. 6D provides evaluation of primary human CD8+ T cell function.

FIG. 7A and FIG. 7B shows results representing values for binding of anti-HLA-G antibodies to individual recombinant HLA class Ia antigens immobilized on beads.

FIG. 8 shows the evaluation of anti-HLA-G antibodies binding to a panel of 28 HLA-typed B-LCL lines.

FIG. 9A and FIG. 9B show the evaluation of anti-HLA-G antibodies binding to various forms of HLA-G after site-directed mutagenesis.

FIG. 10 provides evidence for tumor growth inhibition by an anti-HLA-G antibody with and Fc effector function in a mouse tumor xenograft tumor model using 721.221 cells expressing HLA-G.

### DETAILED DESCRIPTION

### 1. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, NY. As appropriate, procedures involving the use of commercially available kits and reagents are generally carried out in accordance with manufacturer defined protocols and/or parameters unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value±10%, ±5%, or ±1%. In certain embodiments, the term “about” indicates the designated value±one standard deviation of that value.

The term “combinations thereof” includes every possible combination of elements to which the term refers.

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H}) and a heavy chain constant region (CH). The heavy chain constant region typically comprises three domains, C_{H1}, C_{H2}, and C_{H3}. Each light chain typically comprises a light chain variable region (V_{L}) and a light chain constant region. The light chain constant region typically comprises one domain, abbreviated CL.

The term “antibody” describes a type of immunoglobulin molecule and is used herein in its broadest sense. An antibody specifically includes intact antibodies (e.g., intact immunoglobulins), and antibody fragments and antigen binding proteins. Antibodies comprise at least one antigen-binding domain. One example of an antigen-binding domain is an antigen binding domain formed by a V_{H}-V_{L} dimer. An “HLA-G antibody,” “anti-HLA-G antibody,” “HLA-G Ab,” “HLA-G-specific antibody,” or “anti-HLA-G Ab” is an antibody, as described herein, which binds specifically to the antigen HLA-G.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and confer antigen specificity and binding affinity to the antibody. See Kabat et al., Sequences of Proteins of Immunological Interest 5th ed. (1991) Public Health Service, National Institutes of Health, Bethesda, MD, incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme), each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

Unless otherwise specified, the numbering scheme used for identification of a particular CDR herein is the Kabat/Chothia numbering scheme. Where the residues encompassed by these two numbering schemes diverge, the numbering scheme is specified as either Kabat or Chothia.

TABLE 1
Residues in CDRs according to Kabat
and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by papain digestion of a full-length antibody.

“F(ab′)₂” fragments contain two Fab′ fragments joined, near the hinge region, by disulfide bonds. F(ab′)₂ fragments may be generated, for example, by pepsin digestion of an intact antibody. The F(ab′) fragments can be dissociated, for example, by treatment with β-mercaptoethano1.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety. “scFv-Fc” fragments comprise an scFv attached to an Fc domain. For example, an Fc domain may be attached to the C-terminal of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated antibody” is one that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. An isolated antibody includes an antibody in situ within recombinant cells, since at least one component of the antibody's natural environment is not present. In some aspects, an isolated antibody is prepared by at least one purification step.

In some embodiments, an isolated antibody is purified to at least 80%, 85%, 90%, 95%, or 99% by weight. In some embodiments, an isolated antibody is provided as a solution comprising at least 85%, 90%, 95%, 98%, 99% to 100% by weight of an antibody, the remainder of the weight comprising the weight of other solutes dissolved in the solvent.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Affinity can be determined, for example, using surface plasmon resonance (SPR) technology, such as a Biacore® instrument, or using bio-layer interferometry technology, such as an Octet® instrument.

With regard to the binding of an antibody to a target molecule, the terms “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction. Specific binding can be measured, for example, by determining binding of a molecule compared to binding of a control molecule. Specific binding can also be determined by competition with a control molecule that is similar to the target, such as an excess of non-labeled target. In that case, specific binding is indicated if the binding of the labeled target to a probe is competitively inhibited by the excess non-labeled target.

The term “k_{d}” (sec⁻¹), as used herein, refers to the dissociation rate constant of a particular antibody-antigen interaction. This value is also referred to as the k_{off} value.

The term “kₐ” (M⁻¹×sec⁻¹), as used herein, refers to the association rate constant of a particular antibody-antigen interaction. This value is also referred to as the kₒₙ value.

The term “K_{D}” (M), as used herein, refers to the dissociation equilibrium constant of a particular antibody-antigen interaction. K_{D}=kₐ/k_{d}.

The term “K_{A}” (M⁻¹), as used herein, refers to the association equilibrium constant of a particular antibody-antigen interaction. K_{A}=kₐ/k_{d}.

An “affinity matured” antibody is one with one or more alterations in one or more CDRs or FRs that result in an improvement in the affinity of the antibody for its antigen, compared to a parent antibody which does not possess the alteration(s). In one embodiment, an affinity matured antibody has nanomolar or picomolar affinity for the target antigen. Affinity matured antibodies may be produced using a variety of methods known in the art. For example, Marks et al. (Bio/Technology, 1992, 10:779-783, incorporated by reference in its entirety) describes affinity maturation by V_{H} and V_{L} domain shuffling. Random mutagenesis of CDR and/or framework residues is described by, for example, Barbas et al. (Proc. Nat. Acad. Sci. U.S.A., 1994, 91:3809-3813); Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896, each of which is incorporated by reference in its entirety.

When used herein in the context of two or more antibodies, the term “competes with” or “cross-competes with” indicates that the two or more antibodies compete for binding to an antigen (e.g., HLA-G). In one exemplary assay, HLA-G is coated on a plate and allowed to bind a first antibody, after which a second, labeled antibody is added. If the presence of the first antibody reduces binding of the second antibody, then the antibodies compete. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 50%, at least 60%, at least 70%, at least 80%, or at least 90%.

The term “epitope” means a portion of an antigen capable of specific binding to an antibody. Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter is lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to HLA-G variants with different point-mutations.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, or CLUSTAL OMEGA software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution of one or more amino acids with one or more chemically or functionally similar amino acids. Conservative substitution tables providing similar amino acids are well known in the art. Polypeptide sequences having such substitutions are known as “conservatively modified variants.” By way of example, the following groups of amino acids are considered conservative substitutions for one another.

| | |
|---|---|
| Acidic Residues | D and E |
| Basic Residues | K, R, and H |
| Hydrophilic Uncharged Residues | S, T, N, and Q |
| Aliphatic Uncharged Residues | G,A, V, L, and I |
| Non polar Uncharged Residues | C, M, and P |
| Aromatic Residues | F, Y, and W |

| | |
|---|---|
| Alcohol Group-Containing Residues | S and T |
| Aliphatic Residues | I, L, V, and M |
| Cycloalkenyl-associated Residues | F, H, W, and Y |
| Hydrophobic Residues | A, C, F, G, H, I, L, M, V, W, and Y |
| Negatively Charged Residues | D and E |
| Polar Residues | C, D, E, H, K, N, Q, R, S, and T |
| Positively Charged Residues | H, K, and R |
| Small Residues | A, C, D, G, N, P, S, T, and V |
| Very Small Residues | A, G, and S |
| Residues Involved in Turn Formation | A, C, D, E, G, H, K, N, Q, R, S, P, and T |
| Flexible Residues | Q, T, K, S, G, P, D, E, and R |

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, NY. An antibody generated by making one or more conservative substitutions of amino acid residues in a parent antibody is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder.

As used herein, the term “therapeutically effective amount” or “effective amount” refers to an amount of an antibody or composition that when administered to a subject is effective to treat a disease or disorder.

As used herein, the term “subject” means a mammalian subject. Exemplary subjects include, but are not limited to humans, monkeys, dogs, cats, mice, rats, cows, horses, camels, avians, goats and sheep. In certain embodiments, the subject is a human. In some embodiments, the subject has cancer, an autoimmune disease or condition, and/or an infection that can be treated with an antibody provided herein. In some embodiments, the subject is a human that is suspected to have cancer, an autoimmune disease or condition, and/or an acute infection and chronic infection.

### 2. Antibodies

Provided herein are antibodies that selectively bind human HLA-G. In some aspects, the antibody selectively binds to the extracellular domain of human HLA-G.

In some embodiments, the antibody has one or more CDRs having particular lengths, in terms of the number of amino acid residues. In some embodiments, the Chothia CDR-H1 of the antibody is 6, 7, 8, or 9 residues in length. In some embodiments, the Kabat CDR-H1 of the antibody is 4, 5, 6, or 7 residues in length. In some embodiments, the Chothia CDR-H2 of the antibody is 5, 6, or 7 residues in length. In some embodiments, the Kabat CDR-H2 of the antibody is 15, 16, 17, or 18 residues in length. In some embodiments, the Kabat/Chothia CDR-H3 of the antibody is 5, 6, 7, 8, 9, 10, 11, 12, 13, 14 residues in length.

In some aspects, the Kabat/Chothia CDR-L1 of the antibody is 9, 10, 11, 12, 13, 14, 15, or 16 residues in length. In some aspects, the Kabat/Chothia CDR-L2 of the antibody is 6, 7, or 8 residues in length. In some aspects, the Kabat/Chothia CDR-L3 of the antibody is 8, 9, 10, 11, or 12 residues in length.

In some embodiments, the antibody comprises a light chain. In some aspects, the light chain is a kappa light chain. In some aspects, the light chain is a lambda light chain.

In some embodiments, the antibody comprises a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the antibody is an antibody fragment. In some aspects, the antibody fragment is an Fv fragment. In some aspects, the antibody fragment is a Fab fragment. In some aspects, the antibody fragment is a F (ab) 2 fragment. In some aspects, the antibody fragment is a Fab′ fragment. In some aspects, the antibody fragment is an scFv (sFv) fragment. In some aspects, the antibody fragment is an scFv-Fc fragment.

In some embodiments, the antibody is a monoclonal antibody. In some embodiments, the antibody is a polyclonal antibody.

In some embodiments, the antibody is a chimeric antibody. In some embodiments, the antibody is a humanized antibody. In some embodiments, the antibody is a human antibody.

In some embodiments, the antibody is an affinity matured antibody. In some aspects, the antibody is an affinity matured antibody derived from an illustrative sequence provided in this disclosure.

In some embodiments, the antibody blocks HLA-G interaction and/or binding to an ITIM inhibitory receptor. In some embodiments, the antibody blocks HLA-G interaction and/or binding to ILT2. In some embodiments, the antibody blocks HLA-G interaction and/or binding to ILT4. In some embodiments, the antibody blocks HLA-G interaction and/or binding to KIR2DL4. In embodiments, the antibody binds HLA-G but does not block the interaction between HLA-G and ILT2, ILT4, and/or KIRDL4. In some embodiments, the antibody disrupts HLA-G heterodimer and/or prevents dimerization of HLA-G.

In some embodiments, the antibody inhibits immune suppressive function. In some embodiments, the antibody inhibits HLA-G mediated suppression of NK cells. In some embodiments, the antibody inhibits HLA-G mediated suppression of cytotoxic T lymphocytes. In some embodiments, the antibody inhibits HLA-G mediated suppression of B cells. In some embodiments, the antibody inhibits HLA-G mediated suppression of neutrophils. In some embodiments, the antibody inhibits HLA-G mediated suppression of dendritic cells. In some embodiments, the antibody inhibits HLA-G mediated suppression of macrophages. In some embodiments, the antibody inhibits HLA-G mediated suppression of monocytes. In some embodiments, the antibody inhibits HLA-G mediated suppression of NK and/or T cell cytolysis and/or proliferation.

In some embodiments, the antibody prevents or inhibits HLA-G mediated suppression of phagocytosis. In some embodiments, the antibody mediates HLA-G mediated induction of T regulatory cells. In some embodiments, the antibody prevents or inhibits the generation or expansion of regulatory T cells.

The antibodies provided herein may be useful for the treatment of a variety of diseases and conditions, including cancers, autoimmune diseases, and infections. In some embodiments, the antibody inhibits HLA-G function on tumor cells. In some embodiments, the antibody inhibits HLA-G function on immune cells. In some embodiments, the antibody inhibits HLA-G function on myeloid cells. In some embodiments, the antibody inhibits HLA-G function on Tcell subsets. In some embodiments, the antibody inhibits metastasis. In some embodiments, the antibody inhibits angiogenesis.

In some embodiments, the antibody competes or is capable of competing for binding to human HLA-G with another antibody. In some embodiments, the antibody comprises or consists an antibody that is capable of competing for binding to human HLA-G with a reference antibody, wherein the reference antibody binds to an epitope comprising position 195, 197, and/or 198 of SEQ ID NO: 342 on a human HLA-G polypeptide. In some embodiments, the antibody and the reference antibody cross-compete or are capable of cross-competing for binding to human HLA-G with another antibody.

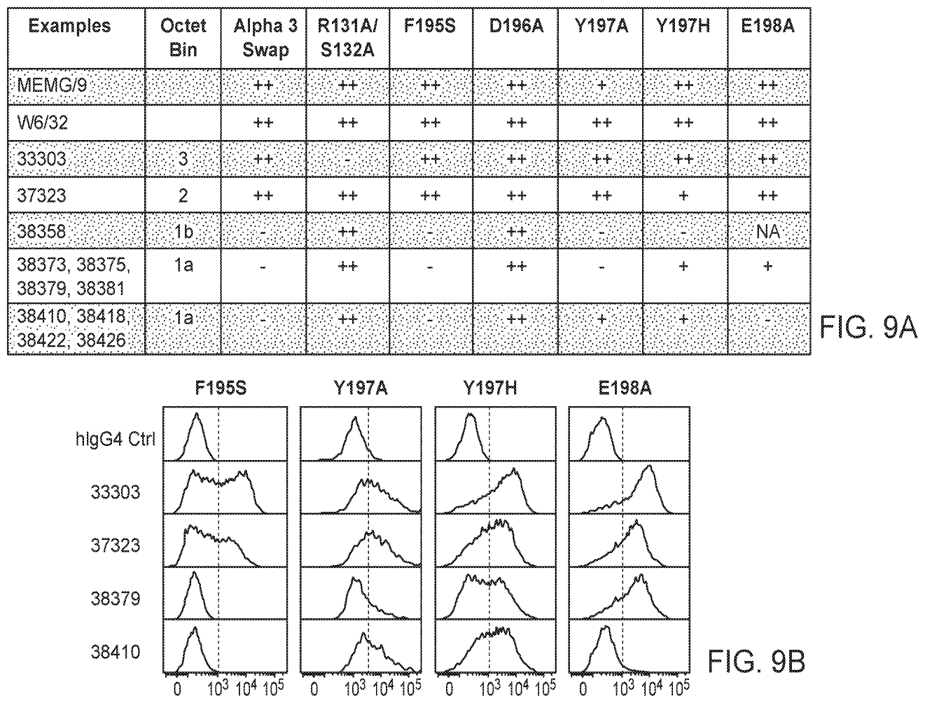

In some embodiments, the antibody binds to an epitope comprising position 195, 197, and/or 198 of SEQ ID NO: 342 on a human HLA-G polypeptide. In some aspects, the epitope comprises or consists of a contiguous or non-contiguous span of amino acids including residues 195, 197, and/or 198 of the sequence set forth in SEQ ID NO: 342. In some aspects, the epitope comprises a sequence that is identical or corresponds to residues 195, 197, and/or 198 of a sequence that is within the sequence set forth in SEQ ID NO: 342. In some aspects, the epitope has a sequence that has a 60%, 65%, 70%, 75%, 80%, 85%, 90%, or 95% identity to a sequence that is within the sequence set forth in SEQ ID NO: 342. In some aspects, the epitope has 1, 2, 3, 4, 5, 6, 7, 8, or 9 substitutions from a sequence that is within the sequence set forth in forth in SEQ ID NO: 342. In some aspects, the epitope has 1, 2, or 3 substitutions from residues a sequence that is within the sequence set forth in SEQ ID NO: 342. In some aspects, the antibody makes contact with any of the residues set forth in FIG. 9.

### 2.1. CDR-H3 Sequences

In some embodiments, the antibody comprises a CDR-H3 sequence comprising, consisting of, or consisting essentially of a sequence selected from SEQ ID NOS: 76-101. In some aspects, the antibody comprises a CDR-H3 sequence comprising, consisting of, or consisting essentially of SEQ ID NO: 76. In some aspects, the antibody comprises a CDR-H3 sequence comprising, consisting of, or consisting essentially of SEQ ID NO: 77. In some aspects, the antibody comprises a CDR-H3 sequence comprising, consisting of, or consisting essentially of SEQ ID NO: 78. In some aspects, the antibody comprises a CDR-H3 sequence comprising, consisting of, or consisting essentially of SEQ ID NO: 79. In some aspects, the antibody comprises a CDR-H3 sequence comprising, consisting of, or consisting essentially of SEQ ID NO: 80. In some aspects, the antibody comprises a CDR-H3 sequence comprising, consisting of, or consisting essentially of SEQ ID NO: 81. In some aspects, the antibody comprises a CDR-H3 sequence comprising, consisting of, or consisting essentially of SEQ ID NO: 82. In some aspects, the antibody comprises a CDR-H3 sequence comprising, consisting of, or consisting essentially of SEQ ID NO: 83. In some aspects, the antibody comprises a CDR-H3 sequence comprising, consisting of, or consisting essentially of SEQ ID NO: 84. In some aspects, the antibody comprises a CDR-H3 sequence comprising, consisting of, or consisting essentially of SEQ ID NO: 85. In some aspects, the antibody comprises a CDR-H3 sequence comprising, consisting of, or consisting essentially of SEQ ID NO: 86. In some aspects, the antibody comprises a CDR-H3 sequence comprising, consisting of, or consisting essentially of SEQ ID NO: 87. In some aspects, the antibody comprises a CDR-H3 sequence comprising, consisting of, or consisting essentially of SEQ ID NO: 88. In some aspects, the antibody comprises a CDR-H3 sequence comprising, consisting of, or consisting essentially of SEQ ID NO: 89. In some aspects, the antibody comprises a CDR-H3 sequence comprising, consisting of, or consisting essentially of SEQ ID NO: 90. In some aspects, the antibody comprises a CDR-H3 sequence comprising, consisting of, or consisting essentially of SEQ ID NO: 91. In some aspects, the antibody comprises a CDR-H3 sequence comprising, consisting of, or consisting essentially of SEQ ID NO: 92. In some aspects, the antibody comprises a CDR-H3 sequence comprising, consisting of, or consisting essentially of SEQ ID NO: 93. In some aspects, the antibody comprises a CDR-H3 sequence comprising, consisting of, or consisting essentially of SEQ ID NO: 94. In some aspects, the antibody comprises a CDR-H3 sequence comprising, consisting of, or consisting essentially of SEQ ID NO: 95. In some aspects, the antibody comprises a CDR-H3 sequence comprising, consisting of, or consisting essentially of SEQ ID NO: 96. In some aspects, the antibody comprises a CDR-H3 sequence comprising, consisting of, or consisting essentially of SEQ ID NO: 97. In some aspects, the antibody comprises a CDR-H3 sequence comprising, consisting of, or consisting essentially of SEQ ID NO: 98. In some aspects, the antibody comprises a CDR-H3 sequence comprising, consisting of, or consisting essentially of SEQ ID NO: 99. In some aspects, the antibody comprises a CDR-H3 sequence comprising, consisting of, or consisting essentially of SEQ ID NO: 100. In some aspects, the antibody comprises a CDR-H3 sequence comprising, consisting of, or consisting essentially of SEQ ID NO: 101.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

### 2.2. V_{H} Sequences Comprising Illustrative CDRs

In some embodiments, the antibody comprises a V_{H} sequence comprising one or more CDR-H sequences comprising, consisting of, or consisting essentially of one or more illustrative CDR-H sequences provided in this disclosure, and variants thereof.

### 2.2.1. V_{H} Sequences Comprising Illustrative Kabat CDRs

In some embodiments, the antibody comprises a V_{H} sequence comprising one or more Kabat CDR-H sequences comprising, consisting of, or consisting essentially of one or more illustrative Kabat CDR-H sequences provided in this disclosure, and variants thereof.

### 2.2.1.1.Kabat CDR-H3

In some embodiments, the antibody comprises a V_{H} sequence comprising a Kabat CDR-H3 sequence comprising, consisting of, or consisting essentially of a sequence selected from SEQ ID NOS: 76-101. In some aspects, the antibody comprises a V_{H} sequence comprising a Kabat CDR-H3 sequence comprising, consisting of, or consisting essentially of SEQ ID NO: 76. In some aspects, the antibody comprises a V_{H} sequence comprising a Kabat CDR-H3 sequence comprising, consisting of, or consisting essentially of SEQ ID NO: 77. In some aspects, the antibody comprises a V_{H} sequence comprising a Kabat CDR-H3 sequence comprising, consisting of, or consisting essentially of SEQ ID NO: 78. In some aspects, the antibody comprises a V_{H} sequence comprising a Kabat CDR-H3 sequence comprising, consisting of, or consisting essentially of SEQ ID NO: 79. In some aspects, the antibody comprises a V_{H} sequence comprising a Kabat CDR-H3 sequence comprising, consisting of, or consisting essentially of SEQ ID NO: 80. In some aspects, the antibody comprises a V_{H} sequence comprising a Kabat CDR-H3 sequence comprising, consisting of, or consisting essentially of SEQ ID NO: 81. In some aspects, the antibody comprises a V_{H} sequence comprising a Kabat CDR-H3 sequence comprising, consisting of, or consisting essentially of SEQ ID NO: 82. In some aspects, the antibody comprises a V_{H} sequence comprising a Kabat CDR-H3 sequence comprising, consisting of, or consisting essentially of SEQ ID NO: 83. In some aspects, the antibody comprises a V_{H} sequence comprising a Kabat CDR-H3 sequence comprising, consisting of, or consisting essentially of SEQ ID NO: 84. In some aspects, the antibody comprises a V_{H} sequence comprising a Kabat CDR-H3 sequence comprising, consisting of, or consisting essentially of SEQ ID NO: 85. In some aspects, the antibody comprises a V_{H} sequence comprising a Kabat CDR-H3 sequence comprising, consisting of, or consisting essentially of SEQ ID NO: 86. In some aspects, the antibody comprises a V_{H} sequence comprising a Kabat CDR-H3 sequence comprising, consisting of, or consisting essentially of SEQ ID NO: 87. In some aspects, the antibody comprises a V_{H} sequence comprising a Kabat CDR-H3 sequence comprising, consisting of, or consisting essentially of SEQ ID NO: 88. In some aspects, the antibody comprises a V_{H} sequence comprising a Kabat CDR-H3 sequence comprising, consisting of, or consisting essentially of SEQ ID NO: 89. In some aspects, the antibody comprises a V_{H} sequence comprising a Kabat CDR-H3 sequence comprising, consisting of, or consisting essentially of SEQ ID NO: 90. In some aspects, the antibody comprises a V_{H} sequence comprising a Kabat CDR-H3 sequence comprising, consisting of, or consisting essentially of SEQ ID NO: 91. In some aspects, the antibody comprises a V_{H} sequence comprising a Kabat CDR-H3 sequence comprising, consisting of, or consisting essentially of SEQ ID NO: 92. In some aspects, the antibody comprises a V_{H} sequence comprising a Kabat CDR-H3 sequence comprising, consisting of, or consisting essentially of SEQ ID NO: 94. In some aspects, the antibody comprises a V_{H} sequence comprising a Kabat CDR-H3 sequence comprising, consisting of, or consisting essentially of SEQ ID NO: 95. In some aspects, the antibody comprises a V_{H} sequence comprising a Kabat CDR-H3 sequence comprising, consisting of, or consisting essentially of SEQ ID NO: 96. In some aspects, the antibody comprises a V_{H} sequence comprising a Kabat CDR-H3 sequence comprising, consisting of, or consisting essentially of SEQ ID NO: 97. In some aspects, the antibody comprises a V_{H} sequence comprising a Kabat CDR-H3 sequence comprising, consisting of, or consisting essentially of SEQ ID NO: 98. In some aspects, the antibody comprises a V_{H} sequence comprising a Kabat CDR-H3 sequence comprising, consisting of, or consisting essentially of SEQ ID NO: 99. In some aspects, the antibody comprises a V_{H} sequence comprising a Kabat CDR-H3 sequence comprising, consisting of, or consisting essentially of SEQ ID NO: 100. In some aspects, the antibody comprises a V_{H} sequence comprising a Kabat CDR-H3 sequence comprising, consisting of, or consisting essentially of SEQ ID NO: 101.

### 2.2.1.2.Kabat CDR-H2

In some embodiments, the antibody comprises a V_{H} sequence comprising a Kabat CDR-H2 sequence comprising, consisting of, or consisting essentially of a sequence selected from SEQ ID NOS: 54-71. In some aspects, the antibody comprises a V_{H} sequence comprising a Kabat CDR-H2 sequence comprising, consisting of, or consisting essentially of SEQ ID NO: 54. In some aspects, the antibody comprises a V_{H} sequence comprising a Kabat CDR-H2 sequence comprising, consisting of, or consisting essentially of SEQ ID NO: 55. In some aspects, the antibody comprises a V_{H} sequence comprising a Kabat CDR-H2 sequence comprising, consisting of, or consisting essentially of SEQ ID NO: 56. In some aspects, the antibody comprises a V_{H} sequence comprising a Kabat CDR-H2 sequence comprising, consisting of, or consisting essentially of SEQ ID NO: 57. In some aspects, the antibody comprises a V_{H} sequence comprising a Kabat CDR-H2 sequence comprising, consisting of, or consisting essentially of SEQ ID NO: 58. In some aspects, the antibody comprises a V_{H} sequence comprising a Kabat CDR-H2 sequence comprising, consisting of, or consisting essentially of SEQ ID NO: 59. In some aspects, the antibody comprises a V_{H} sequence comprising a Kabat CDR-H2 sequence comprising, consisting of, or consisting essentially of SEQ ID NO: 60. In some aspects, the antibody comprises a V_{H} sequence comprising a Kabat CDR-H2 sequence comprising, consisting of, or consisting essentially of SEQ ID NO: 61. In some aspects, the antibody comprises a V_{H} sequence comprising a Kabat CDR-H2 sequence comprising, consisting of, or consisting essentially of SEQ ID NO: 62. In some aspects, the antibody comprises a V_{H} sequence comprising a Kabat CDR-H2 sequence comprising, consisting of, or consisting essentially of SEQ ID NO: 63. In some aspects, the antibody comprises a V_{H} sequence comprising a Kabat CDR-H2 sequence comprising, consisting of, or consisting essentially of SEQ ID NO: 64. In some aspects, the antibody comprises a V_{H} sequence comprising a Kabat CDR-H2 sequence comprising, consisting of, or consisting essentially of SEQ ID NO: 65. In some aspects, the antibody comprises a V_{H} sequence comprising a Kabat CDR-H2 sequence comprising, consisting of, or consisting essentially of SEQ ID NO: 66. In some aspects, the antibody comprises a V_{H} sequence comprising a Kabat CDR-H2 sequence comprising, consisting of, or consisting essentially of SEQ ID NO: 67. In some aspects, the antibody comprises a V_{H} sequence comprising a Kabat CDR-H2 sequence comprising, consisting of, or consisting essentially of SEQ ID NO: 68. In some aspects, the antibody comprises a V_{H} sequence comprising a Kabat CDR-H2 sequence comprising, consisting of, or consisting essentially of SEQ ID NO: 69. In some aspects, the antibody comprises a V_{H} sequence comprising a Kabat CDR-H2 sequence comprising, consisting of, or consisting essentially of SEQ ID NO: 70. In some aspects, the antibody comprises a V_{H} sequence comprising a Kabat CDR-H2 sequence comprising, consisting of, or consisting essentially of SEQ ID NO: 71.

### 2.2.1.3.Kabat CDR-H1

In some embodiments, the antibody comprises a V_{H} sequence comprising a Kabat CDR-H1 sequence comprising, consisting of, or consisting essentially of a sequence selected from SEQ ID NOS: 18-34. In some aspects, the antibody comprises a V_{H} sequence comprising a Kabat CDR-H1 sequence comprising, consisting of, or consisting essentially of SEQ ID NO: 18. In some aspects, the antibody comprises a V_{H} sequence comprising a Kabat CDR-H1 sequence comprising, consisting of, or consisting essentially of SEQ ID NO: 19. In some aspects, the antibody comprises a V_{H} sequence comprising a Kabat CDR-H1 sequence comprising, consisting of, or consisting essentially of SEQ ID NO: 20. In some aspects, the antibody comprises a V_{H} sequence comprising a Kabat CDR-H1 sequence comprising, consisting of, or consisting essentially of SEQ ID NO: 21. In some aspects, the antibody comprises a V_{H} sequence comprising a Kabat CDR-H1 sequence comprising, consisting of, or consisting essentially of SEQ ID NO: 22. In some aspects, the antibody comprises a V_{H} sequence comprising a Kabat CDR-H1 sequence comprising, consisting of, or consisting essentially of SEQ ID NO: 23. In some aspects, the antibody comprises a V_{H} sequence comprising a Kabat CDR-H1 sequence comprising, consisting of, or consisting essentially of SEQ ID NO: 24. In some aspects, the antibody comprises a V_{H} sequence comprising a Kabat CDR-H1 sequence comprising, consisting of, or consisting essentially of SEQ ID NO: 25. In some aspects, the antibody comprises a V_{H} sequence comprising a Kabat CDR-H1 sequence comprising, consisting of, or consisting essentially of SEQ ID NO: 26. In some aspects, the antibody comprises a V_{H} sequence comprising a Kabat CDR-H1 sequence comprising, consisting of, or consisting essentially of SEQ ID NO: 27. In some aspects, the antibody comprises a V_{H} sequence comprising a Kabat CDR-H1 sequence comprising, consisting of, or consisting essentially of SEQ ID NO: 28. In some aspects, the antibody comprises a V_{H} sequence comprising a Kabat CDR-H1 sequence comprising, consisting of, or consisting essentially of SEQ ID NO: 29. In some aspects, the antibody comprises a V_{H} sequence comprising a Kabat CDR-H1 sequence comprising, consisting of, or consisting essentially of SEQ ID NO: 30. In some aspects, the antibody comprises a V_{H} sequence comprising a Kabat CDR-H1 sequence comprising, consisting of, or consisting essentially of SEQ ID NO: 31. In some aspects, the antibody comprises a V_{H} sequence comprising a Kabat CDR-H1 sequence comprising, consisting of, or consisting essentially of SEQ ID NO: 32. In some aspects, the antibody comprises a V_{H} sequence comprising a Kabat CDR-H1 sequence comprising, consisting of, or consisting essentially of SEQ ID NO: 33. In some aspects, the antibody comprises a V_{H} sequence comprising a Kabat CDR-H1 sequence comprising, consisting of, or consisting essentially of SEQ ID NO: 34.

### 2.2.1.4.Kabat CDR-H3+Kabat CDR-H2

In some embodiments, the antibody comprises a V_{H} sequence comprising a Kabat CDR-H3 sequence comprising, consisting of, or consisting essentially of a sequence selected from SEQ ID NOS: 76-101, and a Kabat CDR-H2 sequence comprising, consisting of, or consisting essentially of a sequence selected from SEQ ID NOS: 54-71. In some aspects, the Kabat CDR-H3 sequence and the Kabat CDR-H2 sequence are both from a single illustrative V_{H} sequence provided in this disclosure. For example, in some aspects, the Kabat CDR-H3 and Kabat CDR-H2 are both from a single illustrative V_{H} sequence selected from SEQ ID NOS: 170-200.

### 2.2.1.5.Kabat CDR-H3+Kabat CDR-H1

In some embodiments, the antibody comprises a V_{H} sequence comprising a Kabat CDR-H3 sequence comprising, consisting of, or consisting essentially of a sequence selected from SEQ ID NOS: 76-101, and a Kabat CDR-H1 sequence comprising, consisting of, or consisting essentially of a sequence selected from SEQ ID NOS: 18-34. In some aspects, the Kabat CDR-H3 sequence and the Kabat CDR-H1 sequence are both from a single illustrative V_{H} sequence provided in this disclosure. For example, in some aspects, the Kabat CDR-H3 and Kabat CDR-H1 are both from a single illustrative V_{H} sequence selected from SEQ ID NOS: 170-200.

### 2.2.1.6.Kabat CDR-H1+Kabat CDR-H2

In some embodiments, the antibody comprises a V_{H} sequence comprising a Kabat CDR-H1 sequence comprising, consisting of, or consisting essentially of a sequence selected from SEQ ID NOS: 18-34 and a Kabat CDR-H2 sequence comprising, consisting of, or consisting essentially of a sequence selected from SEQ ID NOS: 54-71. In some aspects, the Kabat CDR-H1 sequence and the Kabat CDR-H2 sequence are both from a single illustrative V_{H} sequence provided in this disclosure. For example, in some aspects, the Kabat CDR-H1 and Kabat CDR-H2 are both from a single illustrative V_{H} sequence selected from SEQ ID NOS: 170-200.

### 2.2.1.7.Kabat CDR-H1+Kabat CDR-H2+Kabat CDR-H3

In some embodiments, the antibody comprises a V_{H} sequence comprising a Kabat CDR-H1 sequence comprising, consisting of, or consisting essentially of a sequence selected from SEQ ID NOS: 18-34, a Kabat CDR-H2 sequence comprising, consisting of, or consisting essentially of a sequence selected from SEQ ID NOS: 54-71, and a Kabat CDR-H3 sequence comprising, consisting of, or consisting essentially of a sequence selected from SEQ ID NOS: 76-101. In some aspects, the Kabat CDR-H1 sequence, Kabat CDR-H2 sequence, and Kabat CDR-H3 sequence are all from a single illustrative V_{H} sequence provided in this disclosure. For example, in some aspects, the Kabat CDR-H1, Kabat CDR-H2, and Kabat CDR-H3 are all from a single illustrative V_{H} sequence selected from SEQ ID NOS: 170-200.

In some aspects, the antibody comprises a V_{H} sequence comprising a Kabat CDR-H1 sequence comprising SEQ ID NO: 18, a Kabat CDR-H2 sequence comprising SEQ ID NO: 54, and a Kabat CDR-H3 sequence comprising SEQ ID NO: 76. In some aspects, the antibody comprises a V_{H} sequence comprising a Kabat CDR-H1 sequence comprising SEQ ID NO: 19, a Kabat CDR-H2 sequence comprising SEQ ED NO: 55, and a Kabat CDR-H3 sequence comprising SEQ ID NO: 77. In some aspects, the antibody comprises a V_{H} sequence comprising a Kabat CDR-H1 sequence comprising SEQ ID NO: 20, a Kabat CDR-H2 sequence comprising SEQ ID NO: 56, and a Kabat CDR-H3 sequence comprising SEQ ID NO: 78. In some aspects, the antibody comprises a V_{H} sequence comprising a Kabat CDR-H1 sequence comprising SEQ ID NO: 21, a Kabat CDR-H2 sequence comprising SEQ ID NO: 57, and a Kabat CDR-H3 sequence comprising SEQ ID NO: 79. In some aspects, the antibody comprises a V_{H} sequence comprising a Kabat CDR-H1 sequence comprising SEQ ID NO: 22, a Kabat CDR-H2 sequence comprising SEQ ID NO: 58, and a Kabat CDR-H3 sequence comprising SEQ ID NO: 80. In some aspects, the antibody comprises a V_{H} sequence comprising a Kabat CDR-H1 sequence comprising SEQ ID NO: 21, a Kabat CDR-H2 sequence comprising SEQ ID NO: 57, and a Kabat CDR-H3 sequence comprising SEQ ID NO: 76. In some aspects, the antibody comprises a V_{H} sequence comprising a Kabat CDR-H1 sequence comprising SEQ ID NO: 23, a Kabat CDR-H2 sequence comprising SEQ ID NO: 59, and a Kabat CDR-H3 sequence comprising SEQ ID NO: 76. In some aspects, the antibody comprises a V_{H} sequence comprising a Kabat CDR-H1 sequence comprising SEQ ID NO: 23, a Kabat CDR-H2 sequence comprising SEQ ID NO: 60, and a Kabat CDR-H3 sequence comprising SEQ ID NO: 81. In some aspects, the antibody comprises a V_{H} sequence comprising a Kabat CDR-H1 sequence comprising SEQ ID NO: 23, a Kabat CDR-H2 sequence comprising SEQ ID NO: 59, and a Kabat CDR-H3 sequence comprising SEQ ID NO: 82. In some aspects, the antibody comprises a V_{H} sequence comprising a Kabat CDR-H1 sequence comprising SEQ ID NO: 23, a Kabat CDR-H2 sequence comprising SEQ ID NO: 59, and a Kabat CDR-H3 sequence comprising SEQ ID NO: 76. In some aspects, the antibody comprises a V_{H} sequence comprising a Kabat CDR-H1 sequence comprising SEQ ID NO: 23, a Kabat CDR-H2 sequence comprising SEQ ID NO: 59, and a Kabat CDR-H3 sequence comprising SEQ ID NO: 81. In some aspects, the antibody comprises a V_{H} sequence comprising a Kabat CDR-H1 sequence comprising SEQ ID NO: 21, a Kabat CDR-H2 sequence comprising SEQ ID NO: 57, and a Kabat CDR-H3 sequence comprising SEQ ID NO: 83. In some aspects, the antibody comprises a V_{H} sequence comprising a Kabat CDR-H1 sequence comprising SEQ ID NO: 21, a Kabat CDR-H2 sequence comprising SEQ ID NO: 57, and a Kabat CDR-H3 sequence comprising SEQ ID NO: 84. In some aspects, the antibody comprises a V_{H} sequence comprising a Kabat CDR-H1 sequence comprising SEQ ID NO: 24, a Kabat CDR-H2 sequence comprising SEQ ID NO: 61, and a Kabat CDR-H3 sequence comprising SEQ ID NO: 85. In some aspects, the antibody comprises a V_{H} sequence comprising a Kabat CDR-H1 sequence comprising SEQ ID NO: 24, a Kabat CDR-H2 sequence comprising SEQ ID NO: 61, and a Kabat CDR-H3 sequence comprising SEQ ID NO: 86. In some aspects, the antibody comprises a V_{H} sequence comprising a Kabat CDR-H1 sequence comprising SEQ ID NO: 25, a Kabat CDR-H2 sequence comprising SEQ ID NO: 62, and a Kabat CDR-H3 sequence comprising SEQ ID NO: 87. In some aspects, the antibody comprises a V_{H} sequence comprising a Kabat CDR-H1 sequence comprising SEQ ID NO: 26, a Kabat CDR-H2 sequence comprising SEQ ID NO: 63, and a Kabat CDR-H3 sequence comprising SEQ ID NO: 88. In some aspects, the antibody comprises a V_{H} sequence comprising a Kabat CDR-H1 sequence comprising SEQ ID NO: 26, a Kabat CDR-H2 sequence comprising SEQ ID NO: 63, and a Kabat CDR-H3 sequence comprising SEQ ID NO: 89. In some aspects, the antibody comprises a V_{H} sequence comprising a Kabat CDR-H1 sequence comprising SEQ ID NO: 25, a Kabat CDR-H2 sequence comprising SEQ ID NO: 63, and a Kabat CDR-H3 sequence comprising SEQ ID NO: 90. In some aspects, the antibody comprises a V_{H} sequence comprising a Kabat CDR-H1 sequence comprising SEQ ID NO: 27, a Kabat CDR-H2 sequence comprising SEQ ID NO: 64, and a Kabat CDR-H3 sequence comprising SEQ ID NO: 90. In some aspects, the antibody comprises a V_{H} sequence comprising a Kabat CDR-H1 sequence comprising SEQ ID NO: 28, a Kabat CDR-H2 sequence comprising SEQ ID NO: 62, and a Kabat CDR-H3 sequence comprising SEQ ID NO: 91. In some aspects, the antibody comprises a V_{H} sequence comprising a Kabat CDR-H1 sequence comprising SEQ ID NO: 29, a Kabat CDR-H2 sequence comprising SEQ ID NO: 64, and a Kabat CDR-H3 sequence comprising SEQ ID NO: 92. In some aspects, the antibody comprises a V_{H} sequence comprising a Kabat CDR-H1 sequence comprising SEQ ID NO: 25, a Kabat CDR-H2 sequence comprising SEQ ID NO: 65, and a Kabat CDR-H3 sequence comprising SEQ ID NO: 93. In some aspects, the antibody comprises a V_{H} sequence comprising a Kabat CDR-H1 sequence comprising SEQ ID NO: 30, a Kabat CDR-H2 sequence comprising SEQ ID NO: 66, and a Kabat CDR-H3 sequence comprising SEQ ID NO: 94. In some aspects, the antibody comprises a V_{H} sequence comprising a Kabat CDR-H1 sequence comprising SEQ ID NO: 31, a Kabat CDR-H2 sequence comprising SEQ ID NO: 67, and a Kabat CDR-H3 sequence comprising SEQ ID NO: 95. In some aspects, the antibody comprises a V_{H} sequence comprising a Kabat CDR-H1 sequence comprising SEQ ID NO: 32, a Kabat CDR-H2 sequence comprising SEQ ID NO: 68, and a Kabat CDR-H3 sequence comprising SEQ ID NO: 96. In some aspects, the antibody comprises a V_{H} sequence comprising a Kabat CDR-H1 sequence comprising SEQ ID NO: 33, a Kabat CDR-H2 sequence comprising SEQ ID NO: 69, and a Kabat CDR-H3 sequence comprising SEQ ID NO: 97. In some aspects, the antibody comprises a V_{H} sequence comprising a Kabat CDR-H1 sequence comprising SEQ ID NO: 34, a Kabat CDR-H2 sequence comprising SEQ ID NO: 70, and a Kabat CDR-H3 sequence comprising SEQ ID NO: 98. In some aspects, the antibody comprises a V_{H} sequence comprising a Kabat CDR-H1 sequence comprising SEQ ID NO: 18, a Kabat CDR-H2 sequence comprising SEQ ID NO: 54, and a Kabat CDR-H3 sequence comprising SEQ ID NO: 99. In some aspects, the antibody comprises a V_{H} sequence comprising a Kabat CDR-H1 sequence comprising SEQ ID NO: 31, a Kabat CDR-H2 sequence comprising SEQ ID NO: 71, and a Kabat CDR-H3 sequence comprising SEQ ID NO: 100. In some aspects, the antibody comprises a V_{H} sequence comprising a Kabat CDR-H1 sequence comprising SEQ ID NO: 24, a Kabat CDR-H2 sequence comprising SEQ ID NO: 61, and a Kabat CDR-H3 sequence comprising SEQ ID NO: 101.

### 2.2.1.8. Variants of V_{H} Sequences Comprising Illustrative Kabat CDRs

In some embodiments, the V_{H} sequences provided herein comprise a variant of an illustrative Kabat CDR-H3, CDR-H2, and/or CDR-H1 sequence provided in this disclosure.

In some aspects, the Kabat CDR-H3 sequence comprises, consists of, or consists essentially of a variant of an illustrative Kabat CDR-H3 sequence provided in this disclosure. In some aspects, the Kabat CDR-H3 sequence comprises, consists of, or consists essentially of a sequence having at least 70%, 75%, 80%, 85%, 90%, or 95% identity with any of the illustrative Kabat CDR-H3 sequences provided in this disclosure. In some aspects, the Kabat CDR-H3 sequence comprises, consists of, or consists essentially of any of the illustrative Kabat CDR-H3 sequences provided in this disclosure, with 1, 2, or 3 amino acid substitutions. In some aspects, the amino acid substitutions are conservative amino acid substitutions.

In some aspects, the Kabat CDR-H2 sequence comprises, consists of, or consists essentially of a variant of an illustrative Kabat CDR-H2 sequence provided in this disclosure. In some aspects, the Kabat CDR-H2 sequence comprises, consists of, or consists essentially of a sequence having at least 70%, 75%, 80%, 85%, 90%, or 95% identity with any of the illustrative Kabat CDR-H2 sequences provided in this disclosure. In some aspects, the Kabat CDR-H2 sequence comprises, consists of, or consists essentially of any of the illustrative Kabat CDR-H2 sequences provided in this disclosure, with 1, 2, or 3 amino acid substitutions. In some aspects, the amino acid substitutions are conservative amino acid substitutions.

In some aspects, the Kabat CDR-H1 sequence comprises, consists of, or consists essentially of a variant of an illustrative Kabat CDR-H1 sequence provided in this disclosure. In some aspects, the Kabat CDR-H1 sequence comprises, consists of, or consists essentially of a sequence having at least 70%, 75%, 80%, 85%, 90%, or 95% identity with any of the illustrative Kabat CDR-H1 sequences provided in this disclosure. In some aspects, the Kabat CDR-H1 sequence comprises, consists of, or consists essentially of any of the illustrative Kabat CDR-H1 sequences provided in this disclosure, with 1, 2, or 3 amino acid substitutions. In some aspects, the amino acid substitutions are conservative amino acid substitutions.

### 2.2.2. V_{H} Sequences Comprising Illustrative Chothia CDRs

In some embodiments, the antibody comprises a V_{H} sequence comprising one or more Chothia CDR-H sequences comprising, consisting of, or consisting essentially of one or more illustrative Chothia CDR-H sequences provided in this disclosure, and variants thereof.

### 2.2.2.1.Chothia CDR-H3

In some embodiments, the antibody comprises a V_{H} sequence comprising a Chothia CDR-H3 sequence comprising, consisting of, or consisting essentially of a sequence selected from SEQ ID NOS: 76-101. In some aspects, the antibody comprises a V_{H} sequence comprising a Chothia CDR-H3 sequence comprising, consisting of, or consisting essentially of SEQ ID NO: 76. In some aspects, the antibody comprises a V_{H} sequence comprising a Chothia CDR-H3 sequence comprising, consisting of, or consisting essentially of SEQ ID NO: 77. In some aspects, the antibody comprises a V_{H} sequence comprising a Chothia CDR-H3 sequence comprising, consisting of, or consisting essentially of SEQ ID NO: 78. In some aspects, the antibody comprises a V_{H} sequence comprising a Chothia CDR-H3 sequence comprising, consisting of, or consisting essentially of SEQ ID NO: 79. In some aspects, the antibody comprises a V_{H} sequence comprising a Chothia CDR-H3 sequence comprising, consisting of, or consisting essentially of SEQ ID NO: 80. In some aspects, the antibody comprises a V_{H} sequence comprising a Chothia CDR-H3 sequence comprising, consisting of, or consisting essentially of SEQ ID NO: 81. In some aspects, the antibody comprises a V_{H} sequence comprising a Chothia CDR-H3 sequence comprising, consisting of, or consisting essentially of SEQ ID NO: 82. In some aspects, the antibody comprises a V_{H} sequence comprising a Chothia CDR-H3 sequence comprising, consisting of, or consisting essentially of SEQ ID NO: 83. In some aspects, the antibody comprises a V_{H} sequence comprising a Chothia CDR-H3 sequence comprising, consisting of, or consisting essentially of SEQ ID NO: 84. In some aspects, the antibody comprises a V_{H} sequence comprising a Chothia CDR-H3 sequence comprising, consisting of, or consisting essentially of SEQ ID NO: 85. In some aspects, the antibody comprises a V_{H} sequence comprising a Chothia CDR-H3 sequence comprising, consisting of, or consisting essentially of SEQ ID NO: 86. In some aspects, the antibody comprises a V_{H} sequence comprising a Chothia CDR-H3 sequence comprising, consisting of, or consisting essentially of SEQ ID NO: 87. In some aspects, the antibody comprises a V_{H} sequence comprising a Chothia CDR-H3 sequence comprising, consisting of, or consisting essentially of SEQ ID NO: 88. In some aspects, the antibody comprises a V_{H} sequence comprising a Chothia CDR-H3 sequence comprising, consisting of, or consisting essentially of SEQ ID NO: 89. In some aspects, the antibody comprises a V_{H} sequence comprising a Chothia CDR-H3 sequence comprising, consisting of, or consisting essentially of SEQ ID NO: 90. In some aspects, the antibody comprises a V_{H} sequence comprising a Chothia CDR-H3 sequence comprising, consisting of, or consisting essentially of SEQ ID NO: 91. In some aspects, the antibody comprises a V_{H} sequence comprising a Chothia CDR-H3 sequence comprising, consisting of, or consisting essentially of SEQ ID NO: 92. In some aspects, the antibody comprises a V_{H} sequence comprising a Chothia CDR-H3 sequence comprising, consisting of, or consisting essentially of SEQ ID NO: 93. In some aspects, the antibody comprises a V_{H} sequence comprising a Chothia CDR-H3 sequence comprising, consisting of, or consisting essentially of SEQ ID NO: 94. In some aspects, the antibody comprises a V_{H} sequence comprising a Chothia CDR-H3 sequence comprising, consisting of, or consisting essentially of SEQ ID NO: 95. In some aspects, the antibody comprises a V_{H} sequence comprising a Chothia CDR-H3 sequence comprising, consisting of, or consisting essentially of SEQ ID NO: 96. In some aspects, the antibody comprises a V_{H} sequence comprising a Chothia CDR-H3 sequence comprising, consisting of, or consisting essentially of SEQ ID NO: 97. In some aspects, the antibody comprises a V_{H} sequence comprising a Chothia CDR-H3 sequence comprising, consisting of, or consisting essentially of SEQ ID NO: 98. In some aspects, the antibody comprises a V_{H} sequence comprising a Chothia CDR-H3 sequence comprising, consisting of, or consisting essentially of SEQ ID NO: 99. In some aspects, the antibody comprises a V_{H} sequence comprising a Chothia CDR-H3 sequence comprising, consisting of, or consisting essentially of SEQ ID NO: 100. In some aspects, the antibody comprises a V_{H} sequence comprising a Chothia CDR-H3 sequence comprising, consisting of, or consisting essentially of SEQ ID NO: 101.

### 2.2.2.2.Chothia CDR-H2

In some embodiments, the antibody comprises a V_{H} sequence comprising a Chothia CDR-H2 sequence comprising, consisting of, or consisting essentially of a sequence selected from SEQ ID NOS: 38-50. In some aspects, the antibody comprises a V_{H} sequence comprising a Chothia CDR-H2 sequence comprising, consisting of, or consisting essentially of SEQ ID NO: 38. In some aspects, the antibody comprises a V_{H} sequence comprising a Chothia CDR-H2 sequence comprising, consisting of, or consisting essentially of SEQ ID NO: 39. In some aspects, the antibody comprises a V_{H} sequence comprising a Chothia CDR-H2 sequence comprising, consisting of, or consisting essentially of SEQ ID NO: 40. In some aspects, the antibody comprises a V_{H} sequence comprising a Chothia CDR-H2 sequence comprising, consisting of, or consisting essentially of SEQ ID NO: 41. In some aspects, the antibody comprises a V_{H} sequence comprising a Chothia CDR-H2 sequence comprising, consisting of, or consisting essentially of SEQ ID NO: 42. In some aspects, the antibody comprises a V_{H} sequence comprising a Chothia CDR-H2 sequence comprising, consisting of, or consisting essentially of SEQ ID NO: 43. In some aspects, the antibody comprises a V_{H} sequence comprising a Chothia CDR-H2 sequence comprising, consisting of, or consisting essentially of SEQ ID NO: 44. In some aspects, the antibody comprises a V_{H} sequence comprising a Chothia CDR-H2 sequence comprising, consisting of, or consisting essentially of SEQ ID NO: 45. In some aspects, the antibody comprises a V_{H} sequence comprising a Chothia CDR-H2 sequence comprising, consisting of, or consisting essentially of SEQ ID NO: 46. In some aspects, the antibody comprises a V_{H} sequence comprising a Chothia CDR-H2 sequence comprising, consisting of, or consisting essentially of SEQ ID NO: 47. In some aspects, the antibody comprises a V_{H} sequence comprising a Chothia CDR-H2 sequence comprising, consisting of, or consisting essentially of SEQ ID NO: 48. In some aspects, the antibody comprises a V_{H} sequence comprising a Chothia CDR-H2 sequence comprising, consisting of, or consisting essentially of SEQ ID NO: 49. In some aspects, the antibody comprises a V_{H} sequence comprising a Chothia CDR-H2 sequence comprising, consisting of, or consisting essentially of SEQ ID NO: 50.

### 2.2.2.3.Chothia CDR-H1

In some embodiments, the antibody comprises a V_{H} sequence comprising a Chothia CDR-H1 sequence comprising, consisting of, or consisting essentially of a sequence selected from SEQ ID NOS: 1-14. In some aspects, the antibody comprises a V_{H} sequence comprising a Chothia CDR-H1 sequence comprising, consisting of, or consisting essentially of SEQ ID NO: 1. In some aspects, the antibody comprises a V_{H} sequence comprising a Chothia CDR-H1 sequence comprising, consisting of, or consisting essentially of SEQ ID NO: 2. In some aspects, the antibody comprises a V_{H} sequence comprising a Chothia CDR-H1 sequence comprising, consisting of, or consisting essentially of SEQ ID NO: 3. In some aspects, the antibody comprises a V_{H} sequence comprising a Chothia CDR-H1 sequence comprising, consisting of, or consisting essentially of SEQ ID NO: 4. In some aspects, the antibody comprises a V_{H} sequence comprising a Chothia CDR-H1 sequence comprising, consisting of, or consisting essentially of SEQ ID NO: 5. In some aspects, the antibody comprises a V_{H} sequence comprising a Chothia CDR-H1 sequence comprising, consisting of, or consisting essentially of SEQ ID NO: 6. In some aspects, the antibody comprises a V_{H} sequence comprising a Chothia CDR-H1 sequence comprising, consisting of, or consisting essentially of SEQ ID NO: 7. In some aspects, the antibody comprises a V_{H} sequence comprising a Chothia CDR-H1 sequence comprising, consisting of, or consisting essentially of SEQ ID NO: 8. In some aspects, the antibody comprises a V_{H} sequence comprising a Chothia CDR-H1 sequence comprising, consisting of, or consisting essentially of SEQ ID NO: 9. In some aspects, the antibody comprises a V_{H} sequence comprising a Chothia CDR-H1 sequence comprising, consisting of, or consisting essentially of SEQ ID NO: 10. In some aspects, the antibody comprises a V_{H} sequence comprising a Chothia CDR-H1 sequence comprising, consisting of, or consisting essentially of SEQ ID NO: 11. In some aspects, the antibody comprises a V_{H} sequence comprising a Chothia CDR-H1 sequence comprising, consisting of, or consisting essentially of SEQ ID NO: 12. In some aspects, the antibody comprises a V_{H} sequence comprising a Chothia CDR-H1 sequence comprising, consisting of, or consisting essentially of SEQ ID NO: 13. In some aspects, the antibody comprises a V_{H} sequence comprising a Chothia CDR-H1 sequence comprising, consisting of, or consisting essentially of SEQ ID NO: 14.

### 2.2.2.4.Chothia CDR-H3+Chothia CDR-H2

In some embodiments, the antibody comprises a V_{H} sequence comprising a Chothia CDR-H3 sequence comprising, consisting of, or consisting essentially of a sequence selected from SEQ ID NOS: 76-101, and a Chothia CDR-H2 sequence comprising, consisting of, or consisting essentially of a sequence selected from SEQ ID NOS: 38-50. In some aspects, the Chothia CDR-H3 sequence and the Chothia CDR-H2 sequence are both from a single illustrative V_{H} sequence provided in this disclosure. For example, in some aspects, the Chothia CDR-H3 and Chothia CDR-H2 are both from a single illustrative V_{H} sequence selected from SEQ ID NOS: 170-200.

### 2.2.2.5.Chothia CDR-H3+Chothia CDR-H1

In some embodiments, the antibody comprises a V_{H} sequence comprising a Chothia CDR-H3 sequence comprising, consisting of, or consisting essentially of a sequence selected from SEQ ID NOS: 76-101, and a Chothia CDR-H1 sequence comprising, consisting of, or consisting essentially of a sequence selected from SEQ ID NOS: 1-14. In some aspects, the Chothia CDR-H3 sequence and the Chothia CDR-H1 sequence are both from a single illustrative V_{H} sequence provided in this disclosure. For example, in some aspects, the Chothia CDR-H3 and Chothia CDR-H1 are both from a single illustrative V_{H} sequence selected from SEQ ID NOS: 170-200.

### 2.2.2.6.Chothia CDR-H1+Chothia CDR-H2

In some embodiments, the antibody comprises a V_{H} sequence comprising a Chothia CDR-H1 sequence comprising, consisting of, or consisting essentially of a sequence selected from SEQ ID NOS: 1-14 and a Chothia CDR-H2 sequence comprising, consisting of, or consisting essentially of a sequence selected from SEQ ID NOS: 38-50. In some aspects, the Chothia CDR-H1 sequence and the Chothia CDR-H2 sequence are both from a single illustrative V_{H} sequence provided in this disclosure. For example, in some aspects, the Chothia CDR-H1 and Chothia CDR-H2 are both from a single illustrative V_{H} sequence selected from SEQ ID NOS: 170-200.

### 2.2.2.7.Chothia CDR-H1+Chothia CDR-H2+Chothia CDR-H3

In some embodiments, the antibody comprises a V_{H} sequence comprising a Chothia CDR-H1 sequence comprising, consisting of, or consisting essentially of a sequence selected from SEQ ID NOS: 1-14, a Chothia CDR-H2 sequence comprising, consisting of, or consisting essentially of a sequence selected from SEQ ID NOS: 38-50, and a Chothia CDR-H3 sequence comprising, consisting of, or consisting essentially of a sequence selected from SEQ ID NOS: 76-101. In some aspects, the Chothia CDR-H1 sequence, Chothia CDR-H2 sequence, and Chothia CDR-H3 sequence are all from a single illustrative V_{H} sequence provided in this disclosure. For example, in some aspects, the Chothia CDR-H1, Chothia CDR-H2, and Chothia CDR-H3 are all from a single illustrative V_{H} sequence selected from SEQ ID NOS: 170-200.

In some embodiments, the antibody comprises a V_{H} sequence comprising a Chothia CDR-H1 sequence comprising SEQ ID NO: 1, a Chothia CDR-H2 sequence comprising SEQ ID NO: 38, and a Chothia CDR-H3 sequence comprising SEQ ID NO: 76. In some embodiments, the antibody comprises a V_{H} sequence comprising a Chothia CDR-H1 sequence comprising SEQ ID NO: 2, a Chothia CDR-H2 sequence comprising SEQ ID NO: 39, and a Chothia CDR-H3 sequence comprising SEQ ID NO: 77. In some embodiments, the antibody comprises a V_{H} sequence comprising a Chothia CDR-H1 sequence comprising SEQ ID NO: 2, a Chothia CDR-H2 sequence comprising SEQ ID NO: 40, and a Chothia CDR-H3 sequence comprising SEQ ID NO: 78. In some embodiments, the antibody comprises a VHI sequence comprising a Chothia CDR-H1 sequence comprising SEQ ID NO: 3, a Chothia CDR-H2 sequence comprising SEQ ID NO: 41, and a Chothia CDR-H3 sequence comprising SEQ ID NO: 79. In some embodiments, the antibody comprises a V_{H} sequence comprising a Chothia CDR-H1 sequence comprising SEQ ID NO: 4, a Chothia CDR-H2 sequence comprising SEQ ID NO: 41, and a Chothia CDR-H3 sequence comprising SEQ ID NO: 80. In some embodiments, the antibody comprises a V_{H} sequence comprising a Chothia CDR-H1 sequence comprising SEQ ID NO: 3, a Chothia CDR-H2 sequence comprising SEQ ID NO: 41, and a Chothia CDR-H3 sequence comprising SEQ ID NO: 76. In some embodiments, the antibody comprises a V_{H} sequence comprising a Chothia CDR-H1 sequence comprising SEQ ID NO: 2, a Chothia CDR-H2 sequence comprising SEQ ID NO: 42, and a Chothia CDR-H3 sequence comprising SEQ ID NO: 76. In some embodiments, the antibody comprises a V_{H} sequence comprising a Chothia CDR-H1 sequence comprising SEQ ID NO: 2, a Chothia CDR-H2 sequence comprising SEQ ID NO: 43, and a Chothia CDR-H3 sequence comprising SEQ ID NO: 81. In some embodiments, the antibody comprises a V_{H} sequence comprising a Chothia CDR-H1 sequence comprising SEQ ID NO: 2, a Chothia CDR-H2 sequence comprising SEQ ID NO: 42, and a Chothia CDR-H3 sequence comprising SEQ ID NO: 82. In some embodiments, the antibody comprises a V_{H} sequence comprising a Chothia CDR-H1 sequence comprising SEQ ID NO: 5, a Chothia CDR-H2 sequence comprising SEQ ID NO: 42, and a Chothia CDR-H3 sequence comprising SEQ ID NO: 76. In some embodiments, the antibody comprises a V_{H} sequence comprising a Chothia CDR-H1 sequence comprising SEQ ID NO: 2, a Chothia CDR-H2 sequence comprising SEQ ID NO: 42, and a Chothia CDR-H3 sequence comprising SEQ ID NO: 81. In some embodiments, the antibody comprises a V_{H} sequence comprising a Chothia CDR-H1 sequence comprising SEQ ID NO: 3, a Chothia CDR-H2 sequence comprising SEQ ID NO: 41, and a Chothia CDR-H3 sequence comprising SEQ ID NO: 83. In some embodiments, the antibody comprises a V_{H} sequence comprising a Chothia CDR-H1 sequence comprising SEQ ID NO: 3, a Chothia CDR-H2 sequence comprising SEQ ID NO: 41, and a Chothia CDR-H3 sequence comprising SEQ ID NO: 84. In some embodiments, the antibody comprises a V_{H} sequence comprising a Chothia CDR-H1 sequence comprising SEQ ID NO: 6, a Chothia CDR-H2 sequence comprising SEQ ID NO: 44, and a Chothia CDR-H3 sequence comprising SEQ ID NO: 85. In some embodiments, the antibody comprises a V_{H} sequence comprising a Chothia CDR-H1 sequence comprising SEQ ID NO: 6, a Chothia CDR-H2 sequence comprising SEQ ID NO: 44, and a Chothia CDR-H3 sequence comprising SEQ ID NO: 86. In some embodiments, the antibody comprises a V_{H} sequence comprising a Chothia CDR-H1 sequence comprising SEQ ID NO: 7, a Chothia CDR-H2 sequence comprising SEQ ID NO: 45, and a Chothia CDR-H3 sequence comprising SEQ ID NO: 87. In some embodiments, the antibody comprises a V_{H} sequence comprising a Chothia CDR-H1 sequence comprising SEQ ID NO: 8, a Chothia CDR-H2 sequence comprising SEQ ID NO: 44, and a Chothia CDR-H3 sequence comprising SEQ ID NO: 88. In some embodiments, the antibody comprises a V_{H} sequence comprising a Chothia CDR-H1 sequence comprising SEQ ID NO: 8, a Chothia CDR-H2 sequence comprising SEQ ID NO: 44, and a Chothia CDR-H3 sequence comprising SEQ ID NO: 89. In some embodiments, the antibody comprises a V_{H} sequence comprising a Chothia CDR-H1 sequence comprising SEQ ID NO: 7, a Chothia CDR-H2 sequence comprising SEQ ID NO: 44, and a Chothia CDR-H3 sequence comprising SEQ ID NO: 90. In some embodiments, the antibody comprises a V_{H} sequence comprising a Chothia CDR-H1 sequence comprising SEQ ID NO: 9, a Chothia CDR-H2 sequence comprising SEQ ID NO: 44, and a Chothia CDR-H3 sequence comprising SEQ ID NO: 90. In some embodiments, the antibody comprises a V_{H} sequence comprising a Chothia CDR-H1 sequence comprising SEQ ID NO: 6, a Chothia CDR-H2 sequence comprising SEQ ID NO: 45, and a Chothia CDR-H3 sequence comprising SEQ ID NO: 91. In some embodiments, the antibody comprises a V_{H} sequence comprising a Chothia CDR-H1 sequence comprising SEQ ID NO: 8, a Chothia CDR-H2 sequence comprising SEQ ID NO: 44, and a Chothia CDR-H3 sequence comprising SEQ ID NO: 92. In some embodiments, the antibody comprises a V_{H} sequence comprising a Chothia CDR-H1 sequence comprising SEQ ID NO: 7, a Chothia CDR-H2 sequence comprising SEQ ID NO: 44, and a Chothia CDR-H3 sequence comprising SEQ ID NO: 93. In some embodiments, the antibody comprises a V_{H} sequence comprising a Chothia CDR-H1 sequence comprising SEQ ID NO: 10, a Chothia CDR-H2 sequence comprising SEQ ID NO: 46, and a Chothia CDR-H3 sequence comprising SEQ ID NO: 94. In some embodiments, the antibody comprises a V_{H} sequence comprising a Chothia CDR-H1 sequence comprising SEQ ID NO: 11, a Chothia CDR-H2 sequence comprising SEQ ID NO: 47, and a Chothia CDR-H3 sequence comprising SEQ ID NO: 95. In some embodiments, the antibody comprises a V_{H} sequence comprising a Chothia CDR-H1 sequence comprising SEQ ID NO: 12, a Chothia CDR-H2 sequence comprising SEQ ID NO: 48, and a Chothia CDR-H3 sequence comprising SEQ ID NO: 96. In some embodiments, the antibody comprises a V_{H} sequence comprising a Chothia CDR-H1 sequence comprising SEQ ID NO: 13, a Chothia CDR-H2 sequence comprising SEQ ID NO: 44, and a Chothia CDR-H3 sequence comprising SEQ ID NO: 97. In some embodiments, the antibody comprises a V_{H} sequence comprising a Chothia CDR-H1 sequence comprising SEQ ID NO: 14, a Chothia CDR-H2 sequence comprising SEQ ID NO: 49, and a Chothia CDR-H3 sequence comprising SEQ ID NO: 98. In some embodiments, the antibody comprises a V_{H} sequence comprising a Chothia CDR-H1 sequence comprising SEQ ID NO: 1, a Chothia CDR-H2 sequence comprising SEQ ID NO: 38, and a Chothia CDR-H3 sequence comprising SEQ ID NO: 99. In some embodiments, the antibody comprises a V_{H} sequence comprising a Chothia CDR-H1 sequence comprising SEQ ID NO: 11, a Chothia CDR-H2 sequence comprising SEQ ID NO: 50, and a Chothia CDR-H3 sequence comprising SEQ ID NO: 100. In some embodiments, the antibody comprises a V_{H} sequence comprising a Chothia CDR-H1 sequence comprising SEQ ID NO: 6, a Chothia CDR-H2 sequence comprising SEQ ID NO: 44, and a Chothia CDR-H3 sequence comprising SEQ ID NO: 101.

### 2.2.2.8. Variants of V_{H} Sequences Comprising Illustrative Chothia CDRs

In some embodiments, the V_{H} sequences provided herein comprise a variant of an illustrative Chothia CDR-H3, CDR-H2, and/or CDR-H1 sequence provided in this disclosure.

In some aspects, the Chothia CDR-H3 sequence comprises, consists of, or consists essentially of a variant of an illustrative Chothia CDR-H3 sequence provided in this disclosure. In some aspects, the Chothia CDR-H3 sequence comprises, consists of, or consists essentially of a sequence having at least 70%, 75%, 80%, 85%, 90%, or 95% identity with any of the illustrative Chothia CDR-H3 sequences provided in this disclosure. In some aspects, the Chothia CDR-H3 sequence comprises, consists of, or consists essentially of any of the illustrative Chothia CDR-H3 sequences provided in this disclosure, with 1, 2, or 3 amino acid substitutions. In some aspects, the amino acid substitutions are conservative amino acid substitutions.

In some aspects, the Chothia CDR-H2 sequence comprises, consists of, or consists essentially of a variant of an illustrative Chothia CDR-H2 sequence provided in this disclosure. In some aspects, the Chothia CDR-H2 sequence comprises, consists of, or consists essentially of a sequence having at least 70%, 75%, 80%, 85%, 90%, or 95% identity with any of the illustrative Chothia CDR-H2 sequences provided in this disclosure. In some aspects, the Chothia CDR-H2 sequence comprises, consists of, or consists essentially of any of the illustrative Chothia CDR-H2 sequences provided in this disclosure, with 1, 2, or 3 amino acid substitutions. In some aspects, the amino acid substitutions are conservative amino acid substitutions.

In some aspects, the Chothia CDR-H1 sequence comprises, consists of, or consists essentially of a variant of an illustrative Chothia CDR-H1 sequence provided in this disclosure. In some aspects, the Chothia CDR-H1 sequence comprises, consists of, or consists essentially of a sequence having at least 70%, 75%, 80%, 85%, 90%, or 95% identity with any of the illustrative Chothia CDR-H1 sequences provided in this disclosure. In some aspects, the Chothia CDR-H1 sequence comprises, consists of, or consists essentially of any of the illustrative Chothia CDR-H1 sequences provided in this disclosure, with 1, 2, or 3 amino acid substitutions. In some aspects, the amino acid substitutions are conservative amino acid substitutions.

### 2.3. V_{H} Sequences

In some embodiments, the antibody comprises a V_{H} sequence comprising, consisting of, or consisting essentially of a sequence selected from SEQ ID NOS: 170-200. In some aspects, the antibody comprises a V_{H} sequence comprising, consisting of, or consisting essentially of SEQ ID NO: 170. In some aspects, the antibody comprises a V_{H} sequence comprising, consisting of, or consisting essentially of SEQ ID NO: 171. In some aspects, the antibody comprises a V_{H} sequence comprising, consisting of, or consisting essentially of SEQ ID NO: 172. In some aspects, the antibody comprises a V_{H} sequence comprising, consisting of, or consisting essentially of SEQ ID NO: 173. In some aspects, the antibody comprises a V_{H} sequence comprising, consisting of, or consisting essentially of SEQ ID NO: 174. In some aspects, the antibody comprises a V_{H} sequence comprising, consisting of, or consisting essentially of SEQ ID NO: 175. In some aspects, the antibody comprises a V_{H} sequence comprising, consisting of, or consisting essentially of SEQ ID NO: 176. In some aspects, the antibody comprises a V_{H} sequence comprising, consisting of, or consisting essentially of SEQ ID NO: 177. In some aspects, the antibody comprises a V_{H} sequence comprising, consisting of, or consisting essentially of SEQ ID NO: 178. In some aspects, the antibody comprises a V_{H} sequence comprising, consisting of, or consisting essentially of SEQ ID NO: 179. In some aspects, the antibody comprises a V_{H} sequence comprising, consisting of, or consisting essentially of SEQ ID NO: 180. In some aspects, the antibody comprises a V_{H} sequence comprising, consisting of, or consisting essentially of SEQ ID NO: 181. In some aspects, the antibody comprises a V_{H} sequence comprising, consisting of, or consisting essentially of SEQ ID NO: 182. In some aspects, the antibody comprises a V_{H} sequence comprising, consisting of, or consisting essentially of SEQ ID NO: 183. In some aspects, the antibody comprises a V_{H} sequence comprising, consisting of, or consisting essentially of SEQ ID NO: 184. In some aspects, the antibody comprises a V_{H} sequence comprising, consisting of, or consisting essentially of SEQ ID NO: 185. In some aspects, the antibody comprises a V_{H} sequence comprising, consisting of, or consisting essentially of SEQ ID NO: 186. In some aspects, the antibody comprises a V_{H} sequence comprising, consisting of, or consisting essentially of SEQ ID NO: 187. In some aspects, the antibody comprises a V_{H} sequence comprising, consisting of, or consisting essentially of SEQ ID NO: 188. In some aspects, the antibody comprises a V_{H} sequence comprising, consisting of, or consisting essentially of SEQ ID NO: 189. In some aspects, the antibody comprises a V_{H} sequence comprising, consisting of, or consisting essentially of SEQ ID NO: 190. In some aspects, the antibody comprises a V_{H} sequence comprising, consisting of, or consisting essentially of SEQ ID NO: 191. In some aspects, the antibody comprises a V_{H} sequence comprising, consisting of, or consisting essentially of SEQ ID NO: 192. In some aspects, the antibody comprises a V_{H} sequence comprising, consisting of, or consisting essentially of SEQ ID NO: 193. In some aspects, the antibody comprises a V_{H} sequence comprising, consisting of, or consisting essentially of SEQ ID NO: 194. In some aspects, the antibody comprises a V_{H} sequence comprising, consisting of, or consisting essentially of SEQ ID NO: 195. In some aspects, the antibody comprises a V_{H} sequence comprising, consisting of, or consisting essentially of SEQ ID NO: 196. In some aspects, the antibody comprises a V_{H} sequence comprising, consisting of, or consisting essentially of SEQ ID NO: 197. In some aspects, the antibody comprises a V_{H} sequence comprising, consisting of, or consisting essentially of SEQ ID NO: 198. In some aspects, the antibody comprises a V_{H} sequence comprising, consisting of, or consisting essentially of SEQ ID NO: 199. In some aspects, the antibody comprises a V_{H} sequence comprising, consisting of, or consisting essentially of SEQ ID NO: 200.

### 2.3.1. Variants of V_{H} Sequences

In some embodiments, the V_{H} sequences provided herein comprise, consist of, or consist essentially of a variant of an illustrative V_{H} sequence provided in this disclosure.

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least 85%, 90%, 95%, 96%, 97%, 98%, 99%, or 99.5% identity with any of the illustrative V_{H} sequences provided in this disclosure.

In some embodiments, the V_{H} sequence comprises, consists of, or consists essentially of any of the illustrative V_{H} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 2.4. CDR-L3 Sequences

In some embodiments, the antibody comprises a CDR-L3 sequence comprising, consisting of, or consisting essentially of a sequence selected from SEQ ID NOS: 149-166. In some aspects, the antibody comprises a CDR-L3 sequence comprising, consisting of, or consisting essentially of SEQ ID NO: 149. In some aspects, the antibody comprises a CDR-L3 sequence comprising, consisting of, or consisting essentially of SEQ ID NO: 150. In some aspects, the antibody comprises a CDR-L3 sequence comprising, consisting of, or consisting essentially of SEQ ID NO: 151. In some aspects, the antibody comprises a CDR-L3 sequence comprising, consisting of, or consisting essentially of SEQ ID NO: 152. In some aspects, the antibody comprises a CDR-L3 sequence comprising, consisting of, or consisting essentially of SEQ ID NO: 153. In some aspects, the antibody comprises a CDR-L3 sequence comprising, consisting of, or consisting essentially of SEQ ID NO: 154. In some aspects, the antibody comprises a CDR-L3 sequence comprising, consisting of, or consisting essentially of SEQ ID NO: 155. In some aspects, the antibody comprises a CDR-L3 sequence comprising, consisting of, or consisting essentially of SEQ ID NO: 156. In some aspects, the antibody comprises a CDR-L3 sequence comprising, consisting of, or consisting essentially of SEQ ID NO: 157. In some aspects, the antibody comprises a CDR-L3 sequence comprising, consisting of, or consisting essentially of SEQ ID NO: 158. In some aspects, the antibody comprises a CDR-L3 sequence comprising, consisting of, or consisting essentially of SEQ ID NO: 159. In some aspects, the antibody comprises a CDR-L3 sequence comprising, consisting of, or consisting essentially of SEQ ID NO: 160. In some aspects, the antibody comprises a CDR-L3 sequence comprising, consisting of, or consisting essentially of SEQ ID NO: 161. In some aspects, the antibody comprises a CDR-L3 sequence comprising, consisting of, or consisting essentially of SEQ ID NO: 162. In some aspects, the antibody comprises a CDR-L3 sequence comprising, consisting of, or consisting essentially of SEQ ID NO: 163. In some aspects, the antibody comprises a CDR-L3 sequence comprising, consisting of, or consisting essentially of SEQ ID NO: 164. In some aspects, the antibody comprises a CDR-L3 sequence comprising, consisting of, or consisting essentially of SEQ ID NO: 165. In some aspects, the antibody comprises a CDR-L3 sequence comprising, consisting of, or consisting essentially of SEQ ID NO: 166.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

### 2.5. V_{L} Sequences Comprising Illustrative CDRs

In some embodiments, the antibody comprises a V_{L} sequence comprising one or more CDR-L sequences comprising, consisting of, or consisting essentially of one or more illustrative CDR-L sequences provided in this disclosure, and variants thereof.

### 2.5.1. CDR-L3

In some embodiments, the antibody comprises a V_{L} sequence comprising a CDR-L3 sequence comprising, consisting of, or consisting essentially of a sequence selected from SEQ ID NOS: 149-166. In some aspects, the antibody comprises a V_{L} sequence comprising a CDR-L3 sequence comprising, consisting of, or consisting essentially of SEQ ID NO: 149. In some aspects, the antibody comprises a V_{L} sequence comprising a CDR-L3 sequence comprising, consisting of, or consisting essentially of SEQ ID NO: 150. In some aspects, the antibody comprises a V_{L} sequence comprising a CDR-L3 sequence comprising, consisting of, or consisting essentially of SEQ ID NO: 151. In some aspects, the antibody comprises a V_{L} sequence comprising a CDR-L3 sequence comprising, consisting of, or consisting essentially of SEQ ID NO: 152. In some aspects, the antibody comprises a V_{L} sequence comprising a CDR-L3 sequence comprising, consisting of, or consisting essentially of SEQ ID NO: 153. In some aspects, the antibody comprises a V_{L} sequence comprising a CDR-L3 sequence comprising, consisting of, or consisting essentially of SEQ ID NO: 154. In some aspects, the antibody comprises a V_{L} sequence comprising a CDR-L3 sequence comprising, consisting of, or consisting essentially of SEQ ID NO: 155. In some aspects, the antibody comprises a V_{L} sequence comprising a CDR-L3 sequence comprising, consisting of, or consisting essentially of SEQ ID NO: 156. In some aspects, the antibody comprises a V_{L} sequence comprising a CDR-L3 sequence comprising, consisting of, or consisting essentially of SEQ ID NO: 157. In some aspects, the antibody comprises a V_{L} sequence comprising a CDR-L3 sequence comprising, consisting of, or consisting essentially of SEQ ID NO: 158. In some aspects, the antibody comprises a V_{L} sequence comprising a CDR-L3 sequence comprising, consisting of, or consisting essentially of SEQ ID NO: 159. In some aspects, the antibody comprises a V_{L} sequence comprising a CDR-L3 sequence comprising, consisting of, or consisting essentially of SEQ ID NO: 160. In some aspects, the antibody comprises a V_{L} sequence comprising a CDR-L3 sequence comprising, consisting of, or consisting essentially of SEQ ID NO: 161. In some aspects, the antibody comprises a V_{L} sequence comprising a CDR-L3 sequence comprising, consisting of, or consisting essentially of SEQ ID NO: 162. In some aspects, the antibody comprises a V_{L} sequence comprising a CDR-L3 sequence comprising, consisting of, or consisting essentially of SEQ ID NO: 163. In some aspects, the antibody comprises a V_{L} sequence comprising a CDR-L3 sequence comprising, consisting of, or consisting essentially of SEQ ID NO: 164. In some aspects, the antibody comprises a V_{L} sequence comprising a CDR-L3 sequence comprising, consisting of, or consisting essentially of SEQ ID NO: 165. In some aspects, the antibody comprises a V_{L} sequence comprising a CDR-L3 sequence comprising, consisting of, or consisting essentially of SEQ ID NO: 166.

### 2.5.2. CDR-L2

In some embodiments, the antibody comprises a V_{L} sequence comprising a CDR-L2 sequence comprising, consisting of, or consisting essentially of a sequence selected from SEQ ID NOS: 128-145. In some aspects, the antibody comprises a V_{L} sequence comprising a CDR-L2 sequence comprising, consisting of, or consisting essentially of SEQ ID NO: 128. In some aspects, the antibody comprises a V_{L} sequence comprising a CDR-L2 sequence comprising, consisting of, or consisting essentially of SEQ ID NO: 129. In some aspects, the antibody comprises a V_{L} sequence comprising a CDR-L2 sequence comprising, consisting of, or consisting essentially of SEQ ID NO: 130. In some aspects, the antibody comprises a V_{L} sequence comprising a CDR-L2 sequence comprising, consisting of, or consisting essentially of SEQ ID NO: 131. In some aspects, the antibody comprises a V_{L} sequence comprising a CDR-L2 sequence comprising, consisting of, or consisting essentially of SEQ ID NO: 132. In some aspects, the antibody comprises a V_{L} sequence comprising a CDR-L2 sequence comprising, consisting of, or consisting essentially of SEQ ID NO: 133. In some aspects, the antibody comprises a V_{L} sequence comprising a CDR-L2 sequence comprising, consisting of, or consisting essentially of SEQ ID NO: 134. In some aspects, the antibody comprises a V_{L} sequence comprising a CDR-L2 sequence comprising, consisting of, or consisting essentially of SEQ ID NO: 135. In some aspects, the antibody comprises a V_{L} sequence comprising a CDR-L2 sequence comprising, consisting of, or consisting essentially of SEQ ID NO: 136. In some aspects, the antibody comprises a V_{L} sequence comprising a CDR-L2 sequence comprising, consisting of, or consisting essentially of SEQ ID NO: 137. In some aspects, the antibody comprises a V_{L} sequence comprising a CDR-L2 sequence comprising, consisting of, or consisting essentially of SEQ ID NO: 138. In some aspects, the antibody comprises a V_{L} sequence comprising a CDR-L2 sequence comprising, consisting of, or consisting essentially of SEQ ID NO: 139. In some aspects, the antibody comprises a V_{L} sequence comprising a CDR-L2 sequence comprising, consisting of, or consisting essentially of SEQ ID NO: 140. In some aspects, the antibody comprises a V_{L} sequence comprising a CDR-L2 sequence comprising, consisting of, or consisting essentially of SEQ ID NO: 141. In some aspects, the antibody comprises a V_{L} sequence comprising a CDR-L2 sequence comprising, consisting of, or consisting essentially of SEQ ID NO: 142. In some aspects, the antibody comprises a V_{L} sequence comprising a CDR-L2 sequence comprising, consisting of, or consisting essentially of SEQ ID NO: 143. In some aspects, the antibody comprises a V_{L} sequence comprising a CDR-L2 sequence comprising, consisting of, or consisting essentially of SEQ ID NO: 144. In some aspects, the antibody comprises a V_{L} sequence comprising a CDR-L2 sequence comprising, consisting of, or consisting essentially of SEQ ID NO: 145.

### 2.5.3. CDR-L1

In some embodiments, the antibody comprises a V_{L} sequence comprising a CDR-L1 sequence comprising, consisting of, or consisting essentially of a sequence selected from SEQ ID NOS: 105-124. In some aspects, the antibody comprises a V_{L} sequence comprising a CDR-L1 sequence comprising, consisting of, or consisting essentially of SEQ ID NO: 105. In some aspects, the antibody comprises a V_{L} sequence comprising a CDR-L1 sequence comprising, consisting of, or consisting essentially of SEQ ID NO: 106. In some aspects, the antibody comprises a V_{L} sequence comprising a CDR-L1 sequence comprising, consisting of, or consisting essentially of SEQ ID NO: 107. In some aspects, the antibody comprises a V_{L} sequence comprising a CDR-L1 sequence comprising, consisting of, or consisting essentially of SEQ ID NO: 108. In some aspects, the antibody comprises a V_{L} sequence comprising a CDR-L1 sequence comprising, consisting of, or consisting essentially of SEQ ID NO: 109. In some aspects, the antibody comprises a V_{L} sequence comprising a CDR-L1 sequence comprising, consisting of, or consisting essentially of SEQ ID NO: 110. In some aspects, the antibody comprises a V_{L} sequence comprising a CDR-L1 sequence comprising, consisting of, or consisting essentially of SEQ ID NO: 111. In some aspects, the antibody comprises a V_{L} sequence comprising a CDR-L1 sequence comprising, consisting of, or consisting essentially of SEQ ID NO: 112. In some aspects, the antibody comprises a V_{L} sequence comprising a CDR-L1 sequence comprising, consisting of, or consisting essentially of SEQ ID NO: 113. In some aspects, the antibody comprises a V_{L} sequence comprising a CDR-L1 sequence comprising, consisting of, or consisting essentially of SEQ ID NO: 114. In some aspects, the antibody comprises a V_{L} sequence comprising a CDR-L1 sequence comprising, consisting of, or consisting essentially of SEQ ID NO: 115. In some aspects, the antibody comprises a V_{L} sequence comprising a CDR-L1 sequence comprising, consisting of, or consisting essentially of SEQ ID NO: 116. In some aspects, the antibody comprises a V_{L} sequence comprising a CDR-L1 sequence comprising, consisting of, or consisting essentially of SEQ ID NO: 117. In some aspects, the antibody comprises a V_{L} sequence comprising a CDR-L1 sequence comprising, consisting of, or consisting essentially of SEQ ID NO: 118. In some aspects, the antibody comprises a V_{L} sequence comprising a CDR-L1 sequence comprising, consisting of, or consisting essentially of SEQ ID NO: 119. In some aspects, the antibody comprises a V_{L} sequence comprising a CDR-L1 sequence comprising, consisting of, or consisting essentially of SEQ ID NO: 120. In some aspects, the antibody comprises a V_{L} sequence comprising a CDR-L1 sequence comprising, consisting of, or consisting essentially of SEQ ID NO: 121. In some aspects, the antibody comprises a V_{L} sequence comprising a CDR-L1 sequence comprising, consisting of, or consisting essentially of SEQ ID NO: 122. In some aspects, the antibody comprises a V_{L} sequence comprising a CDR-L1 sequence comprising, consisting of, or consisting essentially of SEQ ID NO: 123. In some aspects, the antibody comprises a V_{L} sequence comprising a CDR-L1 sequence comprising, consisting of, or consisting essentially of SEQ ID NO: 124.

### 2.5.4. CDR-L3+CDR-L2

In some embodiments, the antibody comprises a V_{L} sequence comprising a CDR-L3 sequence comprising, consisting of, or consisting essentially of a sequence selected from SEQ ID NOS: 149-166 and a CDR-L2 sequence comprising, consisting of, or consisting essentially of a sequence selected from SEQ ID NOS: 128-145. In some aspects, the CDR-L3 sequence and the CDR-L2 sequence are both from a single illustrative V_{L} sequence provided in this disclosure. For example, in some aspects, the CDR-L3 and CDR-L2 are both from a single illustrative V_{L} sequence selected from SEQ ID NOS: 204-228.

### 2.5.5. CDR-L3+CDR-L1

In some embodiments, the antibody comprises a V_{L} sequence comprising a CDR-L3 sequence comprising, consisting of, or consisting essentially of a sequence selected from SEQ ID NOS: 149-166 and a CDR-L1 sequence comprising, consisting of, or consisting essentially of a sequence selected from SEQ ID NOS: 105-124. In some aspects, the CDR-L3 sequence and the CDR-L1 sequence are both from a single illustrative V_{L} sequence provided in this disclosure. For example, in some aspects, the CDR-L3 and CDR-L1 are both from a single illustrative V_{L} sequence selected from SEQ ID NOS: 204-228.

### 2.5.6. CDR-L1+CDR-L2

In some embodiments, the antibody comprises a V_{L} sequence comprising a CDR-L1 sequence comprising, consisting of, or consisting essentially of a sequence selected from SEQ ID NOS: 105-124 and a CDR-L2 sequence comprising, consisting of, or consisting essentially of a sequence selected from SEQ ID NOS: 128-145. In some aspects, the CDR-L1 sequence and the CDR-L2 sequence are both from a single illustrative V_{L} sequence provided in this disclosure. For example, in some aspects, the CDR-L1 and CDR-L2 are both from a single illustrative V_{L} sequence selected from SEQ ID NOS: 204-228.

### 2.5.7. CDR-L1+CDR-L2+CDR-L3

In some embodiments, the antibody comprises a V_{L} sequence comprising a CDR-L1 sequence comprising, consisting of, or consisting essentially of a sequence selected from SEQ ID NOS: 105-124, a CDR-L2 sequence comprising, consisting of, or consisting essentially of a sequence selected from SEQ ID NOS: 128-145, and a CDR-L3 sequence comprising, consisting of, or consisting essentially of a sequence selected from SEQ ID NOS: 149-166. In some aspects, the CDR-L1 sequence, CDR-L2 sequence, and CDR-L3 sequence are all from a single illustrative V_{L} sequence provided in this disclosure. For example, in some aspects, the CDR-L1, CDR-L2, and CDR-L3 are all from a single illustrative V_{L} sequence selected from SEQ ID NOS: 204-228.

In some aspects, the antibody comprises a V_{L} sequence comprising a CDR-L1 sequence comprising SEQ ID NO: 105, a CDR-L2 sequence comprising SEQ ID NO: 128, and a CDR-L3 sequence SEQ ID NO: 149. In some aspects, the antibody comprises a V_{L} sequence comprising a CDR-L1 sequence comprising SEQ ID NO: 106, a CDR-L2 sequence comprising SEQ ID NO: 129, and a CDR-L3 sequence SEQ ID NO: 150. In some aspects, the antibody comprises a V_{L} sequence comprising a CDR-L1 sequence comprising SEQ ID NO: 107, a CDR-L2 sequence comprising SEQ ID NO: 128, and a CDR-L3 sequence SEQ ID NO: 151. In some aspects, the antibody comprises a V_{L} sequence comprising a CDR-L1 sequence comprising SEQ ID NO: 108, a CDR-L2 sequence comprising SEQ ID NO: 130, and a CDR-L3 sequence SEQ ID NO: 151. In some aspects, the antibody comprises a V_{L} sequence comprising a CDR-L1 sequence comprising SEQ ID NO: 109, a CDR-L2 sequence comprising SEQ ID NO: 131, and a CDR-L3 sequence SEQ ID NO: 152. In some aspects, the antibody comprises a V_{L} sequence comprising a CDR-L1 sequence comprising SEQ ID NO: 107, a CDR-L2 sequence comprising SEQ ID NO: 132, and a CDR-L3 sequence SEQ ID NO: 153. In some aspects, the antibody comprises a V_{L} sequence comprising a CDR-L1 sequence comprising SEQ ID NO: 110, a CDR-L2 sequence comprising SEQ ID NO: 132, and a CDR-L3 sequence SEQ ID NO: 151. In some aspects, the antibody comprises a V_{L} sequence comprising a CDR-L1 sequence comprising SEQ ID NO: 111, a CDR-L2 sequence comprising SEQ ID NO: 133, and a CDR-L3 sequence SEQ ID NO: 151. In some aspects, the antibody comprises a V_{L} sequence comprising a CDR-L1 sequence comprising SEQ ID NO: 105, a CDR-L2 sequence comprising SEQ ID NO: 128, and a CDR-L3 sequence SEQ ID NO: 154. In some aspects, the antibody comprises a V_{L} sequence comprising a CDR-L1 sequence comprising SEQ ID NO: 112, a CDR-L2 sequence comprising SEQ ID NO: 134, and a CDR-L3 sequence SEQ ID NO: 155. In some aspects, the antibody comprises a V_{L} sequence comprising a CDR-L1 sequence comprising SEQ ID NO: 113, a CDR-L2 sequence comprising SEQ ID NO: 135, and a CDR-L3 sequence SEQ ID NO: 156. In some aspects, the antibody comprises a V_{L} sequence comprising a CDR-L1 sequence comprising SEQ ID NO: 114, a CDR-L2 sequence comprising SEQ ID NO: 136, and a CDR-L3 sequence SEQ ID NO: 157. In some aspects, the antibody comprises a V_{L} sequence comprising a CDR-L1 sequence comprising SEQ ID NO: 115, a CDR-L2 sequence comprising SEQ ID NO: 135, and a CDR-L3 sequence SEQ ID NO: 157. In some aspects, the antibody comprises a V_{L} sequence comprising a CDR-L1 sequence comprising SEQ ID NO: 116, a CDR-L2 sequence comprising SEQ ID NO: 128, and a CDR-L3 sequence SEQ ID NO: 155. In some aspects, the antibody comprises a V_{L} sequence comprising a CDR-L1 sequence comprising SEQ ID NO: 117, a CDR-L2 sequence comprising SEQ ID NO: 137, and a CDR-L3 sequence SEQ ID NO: 155. In some aspects, the antibody comprises a V_{L} sequence comprising a CDR-L1 sequence comprising SEQ ID NO: 118, a CDR-L2 sequence comprising SEQ ID NO: 137, and a CDR-L3 sequence SEQ ID NO: 158. In some aspects, the antibody comprises a V_{L} sequence comprising a CDR-L1 sequence comprising SEQ ID NO: 118, a CDR-L2 sequence comprising SEQ ID NO: 138, and a CDR-L3 sequence SEQ ID NO: 155. In some aspects, the antibody comprises a V_{L} sequence comprising a CDR-L1 sequence comprising SEQ ID NO: 119, a CDR-L2 sequence comprising SEQ ID NO: 139, and a CDR-L3 sequence SEQ ID NO: 159. In some aspects, the antibody comprises a V_{L} sequence comprising a CDR-L1 sequence comprising SEQ ID NO: 120, a CDR-L2 sequence comprising SEQ ID NO: 140, and a CDR-L3 sequence SEQ ID NO: 160. In some aspects, the antibody comprises a V_{L} sequence comprising a CDR-L1 sequence comprising SEQ ID NO: 121, a CDR-L2 sequence comprising SEQ ID NO: 141, and a CDR-L3 sequence SEQ ID NO: 161. In some aspects, the antibody comprises a V_{L} sequence comprising a CDR-L1 sequence comprising SEQ ID NO: 122, a CDR-L2 sequence comprising SEQ ID NO: 142, and a CDR-L3 sequence SEQ ID NO: 162. In some aspects, the antibody comprises a V_{L} sequence comprising a CDR-L1 sequence comprising SEQ ID NO: 105, a CDR-L2 sequence comprising SEQ ID NO: 143, and a CDR-L3 sequence SEQ ID NO: 163. In some aspects, the antibody comprises a V_{L} sequence comprising a CDR-L1 sequence comprising SEQ ID NO: 105, a CDR-L2 sequence comprising SEQ ID NO: 143, and a CDR-L3 sequence SEQ ID NO: 164. In some aspects, the antibody comprises a V_{L} sequence comprising a CDR-L1 sequence comprising SEQ ID NO: 123, a CDR-L2 sequence comprising SEQ ID NO: 144, and a CDR-L3 sequence SEQ ID NO: 165. In some aspects, the antibody comprises a V_{L} sequence comprising a CDR-L1 sequence comprising SEQ ID NO: 124, a CDR-L2 sequence comprising SEQ ID NO: 145, and a CDR-L3 sequence SEQ ID NO: 166.

### 2.5.8. Variants of V_{L} Sequences Comprising Illustrative CDR-Ls

In some embodiments, the V_{L} sequences provided herein comprise a variant of an illustrative CDR-L3, CDR-L2, and/or CDR-L1 sequence provided in this disclosure.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

### 2.6. V_{L} Sequences

In some embodiments, the antibody comprises a V_{L} sequence comprising, consisting of, or consisting essentially of a sequence selected from SEQ ID NOS: 204-228. In some aspects, the antibody comprises a V_{L} sequence comprising, consisting of, or consisting essentially of SEQ ID NO: 204. In some aspects, the antibody comprises a V_{L} sequence comprising, consisting of, or consisting essentially of SEQ ID NO: 205. In some aspects, the antibody comprises a V_{L} sequence comprising, consisting of, or consisting essentially of SEQ ID NO: 206. In some aspects, the antibody comprises a V_{L} sequence comprising, consisting of, or consisting essentially of SEQ ID NO: 207. In some aspects, the antibody comprises a V_{L} sequence comprising, consisting of, or consisting essentially of SEQ ID NO: 208. In some aspects, the antibody comprises a V_{L} sequence comprising, consisting of, or consisting essentially of SEQ ID NO: 209. In some aspects, the antibody comprises a V_{L} sequence comprising, consisting of, or consisting essentially of SEQ ID NO: 210. In some aspects, the antibody comprises a V_{L} sequence comprising, consisting of, or consisting essentially of SEQ ID NO: 211. In some aspects, the antibody comprises a V_{L} sequence comprising, consisting of, or consisting essentially of SEQ ID NO: 212. In some aspects, the antibody comprises a V_{L} sequence comprising, consisting of, or consisting essentially of SEQ ID NO: 213. In some aspects, the antibody comprises a V_{L} sequence comprising, consisting of, or consisting essentially of SEQ ID NO: 214. In some aspects, the antibody comprises a V_{L} sequence comprising, consisting of, or consisting essentially of SEQ ID NO: 215. In some aspects, the antibody comprises a V_{L} sequence comprising, consisting of, or consisting essentially of SEQ ID NO: 216. In some aspects, the antibody comprises a V_{L} sequence comprising, consisting of, or consisting essentially of SEQ ID NO: 217. In some aspects, the antibody comprises a V_{L} sequence comprising, consisting of, or consisting essentially of SEQ ID NO: 218. In some aspects, the antibody comprises a V_{L} sequence comprising, consisting of, or consisting essentially of SEQ ID NO: 219. In some aspects, the antibody comprises a V_{L} sequence comprising, consisting of, or consisting essentially of SEQ ID NO: 220. In some aspects, the antibody comprises a V_{L} sequence comprising, consisting of, or consisting essentially of SEQ ID NO: 221. In some aspects, the antibody comprises a V_{L} sequence comprising, consisting of, or consisting essentially of SEQ ID NO: 222. In some aspects, the antibody comprises a V_{L} sequence comprising, consisting of, or consisting essentially of SEQ ID NO: 223. In some aspects, the antibody comprises a V_{L} sequence comprising, consisting of, or consisting essentially of SEQ ID NO: 224. In some aspects, the antibody comprises a V_{L} sequence comprising, consisting of, or consisting essentially of SEQ ID NO: 225. In some aspects, the antibody comprises a V_{L} sequence comprising, consisting of, or consisting essentially of SEQ ID NO: 226. In some aspects, the antibody comprises a V_{L} sequence comprising, consisting of, or consisting essentially of SEQ ID NO: 227. In some aspects, the antibody comprises a V_{L} sequence comprising, consisting of, or consisting essentially of SEQ ID NO: 228.

### 2.6.1. Variants of V_{L} Sequences

In some embodiments, the V_{L} sequences provided herein comprise, consist of, or consist essentially of a variant of an illustrative V_{L} sequence provided in this disclosure.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.05% identity with any of the illustrative V_{L} sequences provided in this disclosure.

In some embodiments, the V_{L} sequence comprises, consists of, or consists essentially of any of the illustrative V_{L} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 2.7. Pairs 2.7.1. CDR-H3—CDR-L3 Pairs

In some embodiments, the antibody comprises a CDR-H3 sequence and a CDR-L3 sequence. In some aspects, the CDR-H3 sequence is part of a V_{H} and the CDR-L3 sequence is part of a V_{L}.

In some aspects, the CDR-H3 sequence is a CDR-H3 sequence comprising, consisting of, or consisting essentially of SEQ ID NOS: 76-101, and the CDR-L3 sequence is a CDR-L3 sequence comprising, consisting of, or consisting essentially of SEQ ID NOS: 149-166.

In some aspects, the CDR-H3 sequence is SEQ ID NO: 76 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 77 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 78 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 79 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 80 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 81 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 82 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 83 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 84 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 85 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 86 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the. CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 87 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 88 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 89 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 90 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 91 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 92 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 93 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 94 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 95 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 96 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 97 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 98 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 99 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 100 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ED NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

In some aspects, the CDR-H3 sequence is SEQ ID NO: 101 and the CDR-L3 sequence is selected from SEQ ID NOS: 149-166. In some aspects, the CDR-L3 sequence is SEQ ID NO: 149. In some aspects, the CDR-L3 sequence is SEQ ID NO: 150. In some aspects, the CDR-L3 sequence is SEQ ID NO: 151. In some aspects, the CDR-L3 sequence is SEQ ID NO: 152. In some aspects, the CDR-L3 sequence is SEQ ID NO: 153. In some aspects, the CDR-L3 sequence is SEQ ID NO: 154. In some aspects, the CDR-L3 sequence is SEQ ID NO: 155. In some aspects, the CDR-L3 sequence is SEQ ID NO: 156. In some aspects, the CDR-L3 sequence is SEQ ID NO: 157. In some aspects, the CDR-L3 sequence is SEQ ID NO: 158. In some aspects, the CDR-L3 sequence is SEQ ID NO: 159. In some aspects, the CDR-L3 sequence is SEQ ID NO: 160. In some aspects, the CDR-L3 sequence is SEQ ID NO: 161. In some aspects, the CDR-L3 sequence is SEQ ID NO: 162. In some aspects, the CDR-L3 sequence is SEQ ID NO: 163. In some aspects, the CDR-L3 sequence is SEQ ID NO: 164. In some aspects, the CDR-L3 sequence is SEQ ID NO: 165. In some aspects, the CDR-L3 sequence is SEQ ID NO: 166.

### 2.7.1.1. Variants of CDR-H3-CDR-L3 Pairs

In some embodiments, the CDR-H3-CDR-L3 pairs provided herein comprise a variant of an illustrative CDR-H3 and/or CDR-L1 sequence provided in this disclosure.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

2.7.2. V_{H}-V_{L} Pairs

In some embodiments, the antibody comprises a V_{H} sequence and a V_{L} sequence.

In some aspects, the V_{H} sequence is a V_{H} sequence comprising, consisting of, or consisting essentially of SEQ ID NOS: 170-200 and the V_{L} sequence is a V_{L} sequence comprising, consisting of, or consisting essentially of SEQ ID NOS: 204-228.

In some aspects, the V_{H} sequence is SEQ ID NO: 170 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 171 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 172 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 173 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 174 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 175 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 176 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 177 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 178 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 179 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 180 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 181 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is. SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 182 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 183 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 184 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 185 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 186 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 187 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 188 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 189 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 190 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ED NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 191 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 192 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 193 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 194 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 195 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 196 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 197 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 198 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 199 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

In some aspects, the V_{H} sequence is SEQ ID NO: 200 and the V_{L} sequence is selected from SEQ ID NOS: 204-228. In some aspects, the V_{L} sequence is SEQ ID NO: 204. In some aspects, the V_{L} sequence is SEQ ID NO: 205. In some aspects, the V_{L} sequence is SEQ ID NO: 206. In some aspects, the V_{L} sequence is SEQ ID NO: 207. In some aspects, the V_{L} sequence is SEQ ID NO: 208. In some aspects, the V_{L} sequence is SEQ ID NO: 209. In some aspects, the V_{L} sequence is SEQ ID NO: 210. In some aspects, the V_{L} sequence is SEQ ID NO: 211. In some aspects, the V_{L} sequence is SEQ ID NO: 212. In some aspects, the V_{L} sequence is SEQ ID NO: 213. In some aspects, the V_{L} sequence is SEQ ID NO: 214. In some aspects, the V_{L} sequence is SEQ ID NO: 215. In some aspects, the V_{L} sequence is SEQ ID NO: 216. In some aspects, the V_{L} sequence is SEQ ID NO: 217. In some aspects, the V_{L} sequence is SEQ ID NO: 218. In some aspects, the V_{L} sequence is SEQ ID NO: 219. In some aspects, the V_{L} sequence is SEQ ID NO: 220. In some aspects, the V_{L} sequence is SEQ ID NO: 221. In some aspects, the V_{L} sequence is SEQ ID NO: 222. In some aspects, the V_{L} sequence is SEQ ID NO: 223. In some aspects, the V_{L} sequence is SEQ ID NO: 224. In some aspects, the V_{L} sequence is SEQ ID NO: 225. In some aspects, the V_{L} sequence is SEQ ID NO: 226. In some aspects, the V_{L} sequence is SEQ ID NO: 227. In some aspects, the V_{L} sequence is SEQ ID NO: 228.

### 2.7.3. CDR-H1+CDR-H2+CDR-H3+CDR-L1+CDR-L2+CDR−L3

In some aspects, the antibody comprises a V_{H} sequence comprising a Kabat CDR-H1 sequence comprising SEQ ID NO: 18, a Kabat CDR-H2 sequence comprising SEQ ID NO: 54, and a Kabat CDR-H3 sequence comprising SEQ ID NO: 76 and a V_{L} sequence comprising a CDR-L1 sequence comprising SEQ ID NO: 105, a CDR-L2 sequence comprising SEQ ID NO: 128, and a CDR-L3 sequence SEQ ID NO: 149. In some aspects, the antibody comprises a V_{H} sequence comprising a Kabat CDR-H1 sequence comprising SEQ ID NO: 19, a Kabat CDR-H2 sequence comprising SEQ ID NO: 55, and a Kabat CDR-H3 sequence comprising SEQ ID NO: 77 and a V_{L} sequence comprising a CDR-L1 sequence comprising SEQ ID NO: 105, a CDR-L2 sequence comprising SEQ ID NO: 128, and a CDR-L3 sequence SEQ ID NO: 149. In some aspects, the antibody comprises a V_{H} sequence comprising a Kabat CDR-H1 sequence comprising SEQ ID NO: 20, a Kabat CDR-H2 sequence comprising SEQ ID NO: 56, and a Kabat CDR-H3 sequence comprising SEQ ID NO: 78 and a V_{L} sequence comprising a CDR-L1 sequence comprising SEQ ID NO: 105, a CDR-L2 sequence comprising SEQ ID NO: 128, and a CDR-L3 sequence SEQ ID NO: 149. In some aspects, the antibody comprises a V_{H} sequence comprising a Kabat CDR-H1 sequence comprising SEQ ID NO: 21, a Kabat CDR-H2 sequence comprising SEQ ID NO: 57, and a Kabat CDR-H3 sequence comprising SEQ ID NO: 79 and a V_{L} sequence comprising a CDR-L1 sequence comprising SEQ ID NO: 105, a CDR-L2 sequence comprising SEQ ID NO: 128, and a CDR-L3 sequence SEQ ID NO: 149. In some aspects, the antibody comprises a V_{H} sequence comprising a Kabat CDR-H1 sequence comprising SEQ ID NO: 22, a Kabat CDR-H2 sequence comprising SEQ ID NO: 58, and a Kabat CDR-H3 sequence comprising SEQ ID NO: 80 and a V_{L} sequence comprising a CDR-L1 sequence comprising SEQ ID NO: 105, a CDR-L2 sequence comprising SEQ ID NO: 128, and a CDR-L3 sequence SEQ ID NO: 149. In some aspects, the antibody comprises a V_{H} sequence comprising a Kabat CDR-H1 sequence comprising SEQ ID NO: 21, a Kabat CDR-H2 sequence comprising SEQ ID NO: 57, and a Kabat CDR-H3 sequence comprising SEQ ID NO: 76 and a V_{L} sequence comprising a CDR-L1 sequence comprising SEQ ID NO: 106, a CDR-L2 sequence comprising SEQ ID NO: 129, and a CDR-L3 sequence SEQ ID NO: 150. In some aspects, the antibody comprises a V_{H} sequence comprising a Kabat CDR-H1 sequence comprising SEQ ID NO: 23, a Kabat CDR-H2 sequence comprising SEQ ID NO: 59, and a Kabat CDR-H3 sequence comprising SEQ ID NO: 76 and a V_{L} sequence comprising a CDR-L1 sequence comprising SEQ ID NO: 107, a CDR-L2 sequence comprising SEQ ID NO: 128, and a CDR-L3 sequence SEQ ID NO: 151. In some aspects, the antibody comprises a V_{H} sequence comprising a Kabat CDR-H1 sequence comprising SEQ ID NO: 23, a Kabat CDR-H2 sequence comprising SEQ ID NO: 60, and a Kabat CDR-H3 sequence comprising SEQ ID NO: 81 and a V_{L} sequence comprising a CDR-L1 sequence comprising SEQ ID NO: 108, a CDR-L2 sequence comprising SEQ ID NO: 130, and a CDR-L3 sequence SEQ ID NO: 151. In some aspects, the antibody comprises a V_{H} sequence comprising a Kabat CDR-H1 sequence comprising SEQ ID NO: 23, a Kabat CDR-H2 sequence comprising SEQ ID NO: 59, and a Kabat CDR-H3 sequence comprising SEQ ID NO: 82 and a V_{L} sequence comprising a CDR-L1 sequence comprising SEQ ID NO: 109, a CDR-L2 sequence comprising SEQ ID NO: 131, and a CDR-L3 sequence SEQ ID NO: 152. In some aspects, the antibody comprises a V_{H} sequence comprising a Kabat CDR-H1 sequence comprising SEQ ID NO: 23, a Kabat CDR-H2 sequence comprising SEQ ID NO: 59, and a Kabat CDR-H3 sequence comprising SEQ ID NO: 76 and a V_{L} sequence comprising a CDR-L1 sequence comprising SEQ ID NO: 107, a CDR-L2 sequence comprising SEQ ID NO: 132, and a CDR-L3 sequence SEQ ID NO:153. In some aspects, the antibody comprises a V_{H} sequence comprising a Kabat CDR-H1 sequence comprising SEQ ID NO: 23, a Kabat CDR-H2 sequence comprising SEQ ID NO: 59, and a Kabat CDR-H3 sequence comprising SEQ ID NO: 81 and a V_{L} sequence comprising a CDR-L1 sequence comprising SEQ ID NO: 110, a CDR-L2 sequence comprising SEQ ID NO: 132, and a CDR-L3 sequence SEQ ID NO: 151. In some aspects, the antibody comprises a V_{H} sequence comprising a Kabat CDR-H1 sequence comprising SEQ ID NO: 21, a Kabat CDR-H2 sequence comprising SEQ ID NO: 57, and a Kabat CDR-H3 sequence comprising SEQ ID NO: 83 and a V_{L} sequence comprising a CDR-L1 sequence comprising SEQ ID NO: 111, a CDR-L2 sequence comprising SEQ ID NO: 133, and a CDR-L3 sequence SEQ ID NO: 151. In some aspects, the antibody comprises a V_{H} sequence comprising a Kabat CDR-H1 sequence comprising SEQ ID NO: 21, a Kabat CDR-H2 sequence comprising SEQ ID NO: 57, and a Kabat CDR-H3 sequence comprising SEQ ID NO: 84 and a V_{L} sequence comprising a CDR-L1 sequence comprising SEQ ID NO: 105, a CDR-L2 sequence comprising SEQ ID NO: 128, and a CDR-L3 sequence SEQ ID NO: 149. In some aspects, the antibody comprises a V_{H} sequence comprising a Kabat CDR-H1 sequence comprising SEQ ID NO: 24, a Kabat CDR-H2 sequence comprising SEQ ID NO: 61, and a Kabat CDR-H3 sequence comprising SEQ ID NO: 85 and a V_{L} sequence comprising a CDR-L1 sequence comprising SEQ ID NO: 105, a CDR-L2 sequence comprising SEQ ID NO: 128, and a CDR-L3 sequence SEQ ID NO: 154. In some aspects, the antibody comprises a V_{H} sequence comprising a Kabat CDR-H1 sequence comprising SEQ ID NO: 24, a Kabat CDR-H2 sequence comprising SEQ ID NO: 61, and a Kabat CDR-H3 sequence comprising SEQ ID NO: 86 and a V_{L} sequence comprising a CDR-L1 sequence comprising SEQ ID NO: 105, a CDR-L2 sequence comprising SEQ ID NO: 128, and a CDR-L3 sequence SEQ ID NO: 154. In some aspects, the antibody comprises a V_{H} sequence comprising a Kabat CDR-H1 sequence comprising SEQ ID NO: 25, a Kabat CDR-H2 sequence comprising SEQ ID NO: 62, and a Kabat CDR-H3 sequence comprising SEQ ID NO: 87 and a V_{L} sequence comprising a CDR-L1 sequence comprising SEQ ID NO: 112, a CDR-L2 sequence comprising SEQ ID NO: 134, and a CDR-L3 sequence SEQ ID NO: 155. In some aspects, the antibody comprises a V_{H} sequence comprising a Kabat CDR-H1 sequence comprising SEQ ID NO: 26, a Kabat CDR-H2 sequence comprising SEQ ID NO: 63, and a Kabat CDR-H3 sequence comprising SEQ ID NO: 88 and a V_{L} sequence comprising a CDR-L1 sequence comprising SEQ ID NO: 113, a CDR-L2 sequence comprising SEQ ID NO: 135, and a CDR-L3 sequence SEQ ID NO: 156. In some aspects, the antibody comprises a V_{H} sequence comprising a Kabat CDR-H1 sequence comprising SEQ ID NO: 26, a Kabat CDR-H2 sequence comprising SEQ ID NO: 63, and a Kabat CDR-H3 sequence comprising SEQ ID NO: 89 and a V_{L} sequence comprising a CDR-L1 sequence comprising SEQ ID NO: 114, a CDR-L2 sequence comprising SEQ ID NO: 136, and a CDR-L3 sequence SEQ ID NO: 157. In some aspects, the antibody comprises a V_{H} sequence comprising a Kabat CDR-H1 sequence comprising SEQ ID NO: 25, a Kabat CDR-H2 sequence comprising SEQ ID NO: 63, and a Kabat CDR-H3 sequence comprising SEQ ID NO: 90 and a V_{L} sequence comprising a CDR-L1 sequence comprising SEQ ID NO: 115, a CDR-L2 sequence comprising SEQ ID NO: 135, and a CDR-L3 sequence SEQ ID NO: 157. In some aspects, the antibody comprises a V_{H} sequence comprising a Kabat CDR-H1 sequence comprising SEQ ID NO: 27, a Kabat CDR-H2 sequence comprising SEQ ID NO: 64, and a Kabat CDR-H3 sequence comprising SEQ ID NO: 90 and a V_{L} sequence comprising a CDR-L1 sequence comprising SEQ ID NO: 116, a CDR-L2 sequence comprising SEQ ID NO: 128, and a CDR-L3 sequence SEQ ID NO: 155. In some aspects, the antibody comprises a V_{H} sequence comprising a Kabat CDR-H1 sequence comprising SEQ ID NO: 28, a Kabat CDR-H2 sequence comprising SEQ ID NO: 62, and a Kabat CDR-H3 sequence comprising SEQ ID NO: 91 and a V_{L} sequence comprising a CDR-L1 sequence comprising SEQ ID NO: 117, a CDR-L2 sequence comprising SEQ ID NO: 137, and a CDR-L3 sequence SEQ ID NO: 155. In some aspects, the antibody comprises a V_{H} sequence comprising a Kabat CDR-H1 sequence comprising SEQ ID NO: 29, a Kabat CDR-H2 sequence comprising SEQ ID NO: 64, and a Kabat CDR-H3 sequence comprising SEQ ID NO: 92 and a V_{L} sequence comprising a CDR-L1 sequence comprising SEQ ID NO: 118, a CDR-L2 sequence comprising SEQ ID NO: 137, and a CDR-L3 sequence SEQ ID NO: 158. In some aspects, the antibody comprises a V_{H} sequence comprising a Kabat CDR-H1 sequence comprising SEQ ID NO: 25, a Kabat CDR-H2 sequence comprising SEQ ID NO: 65, and a Kabat CDR-H3 sequence comprising SEQ ID NO: 93 and a V_{L} sequence comprising a CDR-L1 sequence comprising SEQ ID NO: 118, a CDR-L2 sequence comprising SEQ ID NO: 138, and a CDR-L3 sequence SEQ ID NO: 155. In some aspects, the antibody comprises a V_{H} sequence comprising a Kabat CDR-H1 sequence comprising SEQ ID NO: 30, a Kabat CDR-H2 sequence comprising SEQ ID NO: 66, and a Kabat CDR-H3 sequence comprising SEQ ID NO: 94 and a V_{L} sequence comprising a CDR-L1 sequence comprising SEQ ID NO: 119, a CDR-L2 sequence comprising SEQ ID NO: 139, and a CDR-L3 sequence SEQ ID NO: 159. In some aspects, the antibody comprises a V_{H} sequence comprising a Kabat CDR-H1 sequence comprising SEQ ID NO: 31, a Kabat CDR-H2 sequence comprising SEQ ID NO: 67, and a Kabat CDR-H3 sequence comprising SEQ ID NO: 95 and a V_{L} sequence comprising a CDR-L1 sequence comprising SEQ ID NO: 120, a CDR-L2 sequence comprising SEQ ID NO: 140, and a CDR-L3 sequence SEQ ID NO: 160. In some aspects, the antibody comprises a V_{H} sequence comprising a Kabat CDR-H1 sequence comprising SEQ ID NO: 32, a Kabat CDR-H2 sequence comprising SEQ ID NO: 68, and a Kabat CDR-H3 sequence comprising SEQ ID NO: 96 and a V_{L} sequence comprising a CDR-L1 sequence comprising SEQ ID NO: 121, a CDR-L2 sequence comprising SEQ ID NO: 141, and a CDR-L3 sequence SEQ ID NO: 161. In some aspects, the antibody comprises a V_{H} sequence comprising a Kabat CDR-H1 sequence comprising SEQ ID NO: 33, a Kabat CDR-H2 sequence comprising SEQ ID NO: 69, and a Kabat CDR-H3 sequence comprising SEQ ID NO: 97 and a V_{L} sequence comprising a CDR-L1 sequence comprising SEQ ID NO: 122, a CDR-L2 sequence comprising SEQ ID NO: 142, and a CDR-L3 sequence SEQ ID NO: 162. In some aspects, the antibody comprises a V_{H} sequence comprising a Kabat CDR-H1 sequence comprising SEQ ID NO: 34, a Kabat CDR-H2 sequence comprising SEQ ID NO: 70, and a Kabat CDR-H3 sequence comprising SEQ ID NO: 98 and a V_{L} sequence comprising a CDR-L1 sequence comprising SEQ ID NO: 105, a CDR-L2 sequence comprising SEQ ID NO: 143, and a CDR-L3 sequence SEQ ID NO: 163. In some aspects, the antibody comprises a V_{H} sequence comprising a Kabat CDR-H1 sequence comprising SEQ ID NO: 18, a Kabat CDR-H2 sequence comprising SEQ ID NO: 54, and a Kabat CDR-H3 sequence comprising SEQ ID NO: 99 and a V_{L} sequence comprising a CDR-L1 sequence comprising SEQ ID NO: 105, a CDR-L2 sequence comprising SEQ ID NO: 143, and a CDR-L3 sequence SEQ ID NO: 164. In some aspects, the antibody comprises a V_{H} sequence comprising a Kabat CDR-H1 sequence comprising SEQ ID NO: 31, a Kabat CDR-H2 sequence comprising SEQ ID NO: 71, and a Kabat CDR-H3 sequence comprising SEQ ID NO: 100 and a V_{L} sequence comprising a CDR-L1 sequence comprising SEQ ID NO: 123, a CDR-L2 sequence comprising SEQ ID NO: 144, and a CDR-L3 sequence SEQ ID NO: 165. In some aspects, the antibody comprises a V_{H} sequence comprising a Kabat CDR-H1 sequence comprising SEQ ID NO: 24, a Kabat CDR-H2 sequence comprising SEQ ID NO: 61, and a Kabat CDR-H3 sequence comprising SEQ ID NO: 101 and a V_{L} sequence comprising a CDR-L1 sequence comprising SEQ ID NO: 124, a CDR-L2 sequence comprising SEQ ID NO: 145, and a CDR-L3 sequence SEQ ID NO: 166.

In some aspects, the antibody comprises a V_{H} sequence comprising a Chothia CDR-H1 sequence comprising SEQ ID NO: 1, a Chothia CDR-H2 sequence comprising SEQ ID NO: 38, and a Chothia CDR-H3 sequence comprising SEQ ID NO: 76 and a V_{L} sequence comprising a CDR-L1 sequence comprising SEQ ID NO: 105, a CDR-L2 sequence comprising SEQ ID NO: 128, and a CDR-L3 sequence SEQ ID NO: 149. In some aspects, the antibody comprises a V_{H} sequence comprising a Chothia CDR-H1 sequence comprising SEQ ID NO: 2, a Chothia CDR-H2 sequence comprising SEQ ID NO: 39, and a Chothia CDR-H3 sequence comprising SEQ ID NO: 77 and a V_{L} sequence comprising a CDR-L1 sequence comprising SEQ ID NO: 105, a CDR-L2 sequence comprising SEQ ID NO: 128, and a CDR-L3 sequence SEQ ID NO: 149. In some aspects, the antibody comprises a V_{H} sequence comprising a Chothia CDR-H1 sequence comprising SEQ ID NO: 2, a Chothia CDR-H2 sequence comprising SEQ ID NO: 40, and a Chothia CDR-H3 sequence comprising SEQ ID NO: 78 and a V_{L} sequence comprising a CDR-L1 sequence comprising SEQ ID NO: 105, a CDR-L2 sequence comprising SEQ ID NO: 128, and a CDR-L3 sequence SEQ ID NO: 149. In some aspects, the antibody comprises a V_{H} sequence comprising a Chothia CDR-H1 sequence comprising SEQ ID NO: 3, a Chothia CDR-H2 sequence comprising SEQ ID NO: 41, and a Chothia CDR-H3 sequence comprising SEQ ID NO: 79 and a V_{L} sequence comprising a CDR-L1 sequence comprising SEQ ID NO: 105, a CDR-L2 sequence comprising SEQ ID NO: 128, and a CDR-L3 sequence SEQ ID NO: 149. In some aspects, the antibody comprises a V_{H} sequence comprising a Chothia CDR-H1 sequence comprising SEQ ID NO: 4, a Chothia CDR-H2 sequence comprising SEQ ID NO: 41, and a Chothia CDR-H3 sequence comprising SEQ ID NO: 80 and a V_{L} sequence comprising a CDR-L1 sequence comprising SEQ ID NO: 105, a CDR-L2 sequence comprising SEQ ID NO: 128, and a CDR-L3 sequence SEQ ID NO: 149. In some aspects, the antibody comprises a V_{H} sequence comprising a Chothia CDR-H1 sequence comprising SEQ ID NO: 3, a Chothia CDR-H2 sequence comprising SEQ ID NO: 41, and a Chothia CDR-H3 sequence comprising SEQ ID NO: 76 and a V_{L} sequence comprising a CDR-L1 sequence comprising SEQ ID NO: 106, a CDR-L2 sequence comprising SEQ ID NO: 129, and a CDR-L3 sequence SEQ ID NO: 150. In some aspects, the antibody comprises a V_{H} sequence comprising a Chothia CDR-H1 sequence comprising SEQ ID NO: 2, a Chothia CDR-H2 sequence comprising SEQ ID NO: 42, and a Chothia CDR-H3 sequence comprising SEQ ID NO: 76 and a V_{L} sequence comprising a CDR-L1 sequence comprising SEQ ID NO: 107, a CDR-L2 sequence comprising SEQ ID NO: 128, and a CDR-L3 sequence SEQ ID NO: 151. In some aspects, the antibody comprises a V_{H} sequence comprising a Chothia CDR-H1 sequence comprising SEQ ID NO: 2, a Chothia CDR-H2 sequence comprising SEQ ID NO: 43, and a Chothia CDR-H3 sequence comprising SEQ ID NO: 81 and a V_{L} sequence comprising a CDR-L1 sequence comprising SEQ ID NO: 108, a CDR-L2 sequence comprising SEQ ID NO: 130, and a CDR-L3 sequence SEQ ID NO: 151. In some aspects, the antibody comprises a V_{H} sequence comprising a Chothia CDR-H1 sequence comprising SEQ ID NO: 2, a Chothia CDR-H2 sequence comprising SEQ ID NO: 42, and a Chothia CDR-H3 sequence comprising SEQ ID NO: 82 and a V_{L} sequence comprising a CDR-L1 sequence comprising SEQ ID NO: 109, a CDR-L2 sequence comprising SEQ ID NO: 131, and a CDR-L3 sequence SEQ ID NO: 152. In some aspects, the antibody comprises a V_{H} sequence comprising a Chothia CDR-H1 sequence comprising SEQ ID NO: 5, a Chothia CDR-H2 sequence comprising SEQ ID NO: 42, and a Chothia CDR-H3 sequence comprising SEQ ID NO: 76 and a V_{L} sequence comprising a CDR-L1 sequence comprising SEQ ID NO: 107, a CDR-L2 sequence comprising SEQ ID NO: 132, and a CDR-L3 sequence SEQ ID NO: 153. In some aspects, the antibody comprises a V_{H} sequence comprising a Chothia CDR-H1 sequence comprising SEQ ID NO: 2, a Chothia CDR-H2 sequence comprising SEQ ID NO: 42, and a Chothia CDR-H3 sequence comprising SEQ ID NO: 81 and a V_{L} sequence comprising a CDR-L1 sequence comprising SEQ ID NO: 110, a CDR-L2 sequence comprising SEQ ID NO: 132, and a CDR-L3 sequence SEQ ID NO: 151. In some aspects, the antibody comprises a V_{H} sequence comprising a Chothia CDR-H1 sequence comprising SEQ ID NO: 3, a Chothia CDR-H2 sequence comprising SEQ ID NO: 41, and a Chothia CDR-H3 sequence comprising SEQ ID NO: 83 and a V_{L} sequence comprising a CDR-L1 sequence comprising SEQ ID NO: 111, a CDR-L2 sequence comprising SEQ ID NO: 133, and a CDR-L3 sequence SEQ ID NO: 151. In some aspects, the antibody comprises a V_{H} sequence comprising a Chothia CDR-H1 sequence comprising SEQ ID NO: 3, a Chothia CDR-H2 sequence comprising SEQ ID NO: 41, and a Chothia CDR-H3 sequence comprising SEQ ID NO: 84 and a V_{L} sequence comprising a CDR-L1 sequence comprising SEQ ID NO: 105, a CDR-L2 sequence comprising SEQ ID NO: 128, and a CDR-L3 sequence SEQ ID NO: 149. In some aspects, the antibody comprises a V_{H} sequence comprising a Chothia CDR-H1 sequence comprising SEQ ID NO: 6, a Chothia CDR-H2 sequence comprising SEQ ID NO: 44, and a Chothia CDR-H3 sequence comprising SEQ ID NO: 85 and a V_{L} sequence comprising a CDR-L1 sequence comprising SEQ ID NO: 105, a CDR-L2 sequence comprising SEQ ID NO: 128, and a CDR-L3 sequence SEQ ID NO: 154. In some aspects, the antibody comprises a V_{H} sequence comprising a Chothia CDR-H1 sequence comprising SEQ ID NO: 6, a Chothia CDR-H2 sequence comprising SEQ ID NO: 44, and a Chothia CDR-H3 sequence comprising SEQ ID NO: 86 and a V_{L} sequence comprising a CDR-L1 sequence comprising SEQ ID NO: 105, a CDR-L2 sequence comprising SEQ ID NO: 128, and a CDR-L3 sequence SEQ ID NO: 154. In some aspects, the antibody comprises a V_{H} sequence comprising a Chothia CDR-H1 sequence comprising SEQ ID NO: 7, a Chothia CDR-H2 sequence comprising SEQ ID NO: 45, and a Chothia CDR-H3 sequence comprising SEQ ID NO: 87 and a V_{L} sequence comprising a CDR-L1 sequence comprising SEQ ID NO: 112, a CDR-L2 sequence comprising SEQ ID NO: 134, and a CDR-L3 sequence SEQ ID NO: 155. In some aspects, the antibody comprises a V_{H} sequence comprising a Chothia CDR-H1 sequence comprising SEQ ID NO: 8, a Chothia CDR-H2 sequence comprising SEQ ID NO: 44, and a Chothia CDR-H3 sequence comprising SEQ ID NO: 88 and a V_{L} sequence comprising a CDR-L1 sequence comprising SEQ ID NO: 113, a CDR-L2 sequence comprising SEQ ID NO: 135, and a CDR-L3 sequence SEQ ID NO: 156. In some aspects, the antibody comprises a V_{H} sequence comprising a Chothia CDR-H1 sequence comprising SEQ ID NO: 8, a Chothia CDR-H2 sequence comprising SEQ ID NO: 44, and a Chothia CDR-H3 sequence comprising SEQ ID NO: 89 and a V_{L} sequence comprising a CDR-L1 sequence comprising SEQ ID NO: 114, a CDR-L2 sequence comprising SEQ ID NO: 136, and a CDR-L3 sequence SEQ ID NO: 157. In some aspects, the antibody comprises a V_{H} sequence comprising a Chothia CDR-H1 sequence comprising SEQ ID NO: 7, a Chothia CDR-H2 sequence comprising SEQ ID NO: 44, and a Chothia CDR-H3 sequence comprising SEQ ID NO: 90 and a V_{L} sequence comprising a CDR-L1 sequence comprising SEQ ID NO: 115, a CDR-L2 sequence comprising SEQ ID NO: 135, and a CDR-L3 sequence SEQ ID NO: 157. In some aspects, the antibody comprises a V_{H} sequence comprising a Chothia CDR-H1 sequence comprising SEQ ID NO: 9, a Chothia CDR-H2 sequence comprising SEQ ID NO: 44, and a Chothia CDR-H3 sequence comprising SEQ ID NO: 90 and a V_{L} sequence comprising a CDR-L1 sequence comprising SEQ ID NO: 116, a CDR-L2 sequence comprising SEQ ID NO: 128, and a CDR-L3 sequence SEQ ID NO: 155. In some aspects, the antibody comprises a V_{H} sequence comprising a Chothia CDR-H1 sequence comprising SEQ ID NO: 6, a Chothia CDR-H2 sequence comprising SEQ ID NO: 45, and a Chothia CDR-H3 sequence comprising SEQ ID NO: 91 and a V_{L} sequence comprising a CDR-L1 sequence comprising SEQ ID NO: 117, a CDR-L2 sequence comprising SEQ ID NO: 137, and a CDR-L3 sequence SEQ ID NO: 155. In some aspects, the antibody comprises a V_{H} sequence comprising a Chothia CDR-H1 sequence comprising SEQ ID NO: 8, a Chothia CDR-H2 sequence comprising SEQ ID NO: 44, and a Chothia CDR-H3 sequence comprising SEQ ID NO: 92 and a V_{L} sequence comprising a CDR-L1 sequence comprising SEQ ID NO: 118, a CDR-L2 sequence comprising SEQ ID NO: 137, and a CDR-L3 sequence SEQ ID NO: 158. In some aspects, the antibody comprises a V_{H} sequence comprising a Chothia CDR-H1 sequence comprising SEQ ID NO: 7, a Chothia CDR-H2 sequence comprising SEQ ID NO: 44, and a Chothia CDR-H3 sequence comprising SEQ ID NO: 93 and a V_{L} sequence comprising a CDR-L1 sequence comprising SEQ ID NO: 118, a CDR-L2 sequence comprising SEQ ID NO: 138, and a CDR-L3 sequence SEQ ID NO: 155. In some aspects, the antibody comprises a V_{H} sequence comprising a Chothia CDR-H1 sequence comprising SEQ ID NO: 10, a Chothia CDR-H2 sequence comprising SEQ ID NO: 46, and a Chothia CDR-H3 sequence comprising SEQ ID NO: 94 and a V_{L} sequence comprising a CDR-L1 sequence comprising SEQ ID NO: 119, a CDR-L2 sequence comprising SEQ ID NO: 139, and a CDR-L3 sequence SEQ ID NO: 159. In some aspects, the antibody comprises a V_{H} sequence comprising a Chothia CDR-H1 sequence comprising SEQ ID NO: 11, a Chothia CDR-H2 sequence comprising SEQ ID NO: 47, and a Chothia CDR-H3 sequence comprising SEQ ID NO: 95 and a V_{L} sequence comprising a CDR-L1 sequence comprising SEQ ID NO: 120, a CDR-L2 sequence comprising SEQ ID NO: 140, and a CDR-L3 sequence SEQ ID NO: 160. In some aspects, the antibody comprises a V_{H} sequence comprising a Chothia CDR-H1 sequence comprising SEQ ID NO: 12, a Chothia CDR-H2 sequence comprising SEQ ID NO: 48, and a Chothia CDR-H3 sequence comprising SEQ ID NO: 96 and a V_{L} sequence comprising a CDR-L1 sequence comprising SEQ ID NO: 121, a CDR-L2 sequence comprising SEQ ID NO: 141, and a CDR-L3 sequence SEQ ID NO: 161. In some aspects, the antibody comprises a V_{H} sequence comprising a Chothia CDR-H1 sequence comprising SEQ ID NO: 13, a Chothia CDR-H2 sequence comprising SEQ ID NO: 44, and a Chothia CDR-H3 sequence comprising SEQ ID NO: 97 and a V_{L} sequence comprising a CDR-L1 sequence comprising SEQ ID NO: 122, a CDR-L2 sequence comprising SEQ ID NO: 142, and a CDR-L3 sequence SEQ ID NO: 162. In some aspects, the antibody comprises a V_{H} sequence comprising a Chothia CDR-H1 sequence comprising SEQ ID NO: 14, a Chothia CDR-H2 sequence comprising SEQ ID NO: 49, and a Chothia CDR-H3 sequence comprising SEQ ID NO: 98 and a V_{L} sequence comprising a CDR-L1 sequence comprising SEQ ID NO: 105, a CDR-L2 sequence comprising SEQ ID NO: 143, and a CDR-L3 sequence SEQ ID NO: 163. In some aspects, the antibody comprises a V_{H} sequence comprising a Chothia CDR-H1 sequence comprising SEQ ID NO: 1, a Chothia CDR-H2 sequence comprising SEQ ID NO: 38, and a Chothia CDR-H3 sequence comprising SEQ ID NO: 99 and a V_{L} sequence comprising a CDR-L1 sequence comprising SEQ ID NO: 105, a CDR-L2 sequence comprising SEQ ID NO: 143, and a CDR-L3 sequence SEQ ID NO: 164. In some aspects, the antibody comprises a V_{H} sequence comprising a Chothia CDR-H1 sequence comprising SEQ ID NO: 11, a Chothia CDR-H2 sequence comprising SEQ ID NO: 50, and a Chothia CDR-H3 sequence comprising SEQ ID NO: 100 and a V_{L} sequence comprising a CDR-L1 sequence comprising SEQ ID NO: 123, a CDR-L2 sequence comprising SEQ ID NO: 144, and a CDR-L3 sequence SEQ ID NO: 165. In some aspects, the antibody comprises a V_{H} sequence comprising a Chothia CDR-H1 sequence comprising SEQ ID NO: 6, a Chothia CDR-H2 sequence comprising SEQ ID NO: 44, and a Chothia CDR-H3 sequence comprising SEQ ID NO: 101 and a V_{L} sequence comprising a CDR-L1 sequence comprising SEQ ID NO: 124, a CDR-L2 sequence comprising SEQ ID NO: 145, and a CDR-L3 sequence SEQ ID NO: 166.

2.7.3.1. Variants of V_{H}-V_{L} Pairs

In some embodiments, the V_{H}-V_{L} pairs provided herein comprise a variant of an illustrative V_{H} and/or V_{L} sequence provided in this disclosure.

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least 85%, 90%, 95%, 96%, 97%, 98%, 99%, or 99.1% identity with any of the illustrative V_{H} sequences provided in this disclosure.

In some embodiments, the V_{H} sequence comprises, consists of, or consists essentially of any of the illustrative V_{H} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.05% identity with any of the illustrative V_{L} sequences provided in this disclosure.

In some embodiments, the V_{L} sequence comprises, consists of, or consists essentially of any of the illustrative V_{L} sequences provided in this disclosure, 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 2.7.4. HC+LC

In some aspects, the antibody comprises or consists of one or more heavy chains consisting of an HC sequence and one or more light chains consisting of an LC sequence. In some aspects, the antibody comprises or consists of two identical heavy chains consisting of an HC sequence and two identical light chains consisting of an LC sequence.

In some aspects, the HC sequence is an HC sequence comprising, consisting of, or consisting essentially of a sequence selected from SEQ ID NOS: 232-262 and the LC sequence is an LC sequence comprising, consisting of, or consisting essentially of SEQ ID NOS: 300-330. In some embodiments, the HC sequence is an HC sequence consisting of a sequence selected from SEQ ID NOS: 232-262 and the LC sequence is an LC sequence consisting of a sequence selected from SEQ ID NOS: 300-330. In some embodiments, the HC sequence is an HC sequence comprising, consisting of, or consisting essentially of a sequence selected from SEQ ID NOS: 266-296 and the LC sequence is an LC sequence comprising, consisting of, or consisting essentially of SEQ ID NOS: 300-330. In some embodiments, the HC sequence is an HC sequence consisting of a sequence selected from SEQ ID NOS: 266-296 and the LC sequence is an LC sequence consisting of a sequence selected from SEQ ID NOS: 300-330.

In some aspects, the HC sequence is an HC sequence consisting of SEQ ID NO: 232 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 233 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 234 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 235 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 236 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 237 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 238 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 239 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 240 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 241 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 242 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 243 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 244 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 245 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 246 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 247 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 248 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 249 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 250 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 251 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 252 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 253 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 254 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 255 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 256 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 257 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 258 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 259 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 260 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 261 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 262 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 266 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 267 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 268 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 269 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 270 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 271 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 272 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 273 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 274 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 275 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 276 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 277 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 278 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 279 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 280 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 281 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 282 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 283 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 284 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 285 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 286 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 287 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 288 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 289 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 290 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 291 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 292 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 293 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 294 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 295 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 296 and the LC sequence is an LC sequence consisting of a sequence selected from SEQ ID NOS: 300-330. In some aspects, the LC sequence is SEQ ID NO: 300. In some aspects, the LC sequence is SEQ ID NO: 301. In some aspects, the LC sequence is SEQ ID NO: 302. In some aspects, the LC sequence is SEQ ID NO: 303. In some aspects, the LC sequence is SEQ ID NO: 304. In some aspects, the LC sequence is SEQ ID NO: 305. In some aspects, the LC sequence is SEQ ID NO: 306. In some aspects, the LC sequence is SEQ ID NO: 307. In some aspects, the LC sequence is SEQ ID NO: 308. In some aspects, the LC sequence is SEQ ID NO: 309. In some aspects, the LC sequence is SEQ ID NO: 310. In some aspects, the LC sequence is SEQ ID NO: 311. In some aspects, the LC sequence is SEQ ID NO: 312. In some aspects, the LC sequence is SEQ ID NO: 313. In some aspects, the LC sequence is SEQ ID NO: 314. In some aspects, the LC sequence is SEQ ID NO: 315. In some aspects, the LC sequence is SEQ ID NO: 316. In some aspects, the LC sequence is SEQ ID NO: 317. In some aspects, the LC sequence is SEQ ID NO: 318. In some aspects, the LC sequence is SEQ ID NO: 319. In some aspects, the LC sequence is SEQ ID NO: 320. In some aspects, the LC sequence is SEQ ID NO: 321. In some aspects, the LC sequence is SEQ ID NO: 322. In some aspects, the LC sequence is SEQ ID NO: 323. In some aspects, the LC sequence is SEQ ID NO: 324. In some aspects, the LC sequence is SEQ ID NO: 325. In some aspects, the LC sequence is SEQ ID NO: 326. In some aspects, the LC sequence is SEQ ID NO: 327. In some aspects, the LC sequence is SEQ ID NO: 328. In some aspects, the LC sequence is SEQ ID NO: 329. In some aspects, the LC sequence is SEQ ID NO: 330.

In some aspects, the HC sequence is an HC sequence consisting of SEQ ID NO: 232 and the LC sequence is an LC sequence consisting of sequence SEQ ID NO: 300. In some aspects, the HC sequence is an HC sequence consisting of SEQ ID NO: 233 and the LC sequence is an LC sequence consisting of sequence SEQ ID NO: 301. In some aspects, the HC sequence is an HC sequence consisting of SEQ ID NO: 234 and the LC sequence is an LC sequence consisting of sequence SEQ ID NO: 302. In some aspects, the HC sequence is an HC sequence consisting of SEQ ID NO: 235 and the LC sequence is an LC sequence consisting of sequence SEQ ID NO: 303. In some aspects, the HC sequence is an HC sequence consisting of SEQ ID NO: 236 and the LC sequence is an LC sequence consisting of sequence SEQ ID NO: 304. In some aspects, the HC sequence is an HC sequence consisting of SEQ ID NO: 237 and the LC sequence is an LC sequence consisting of sequence SEQ ID NO: 305. In some aspects, the HC sequence is an HC sequence consisting of SEQ ID NO: 238 and the LC sequence is an LC sequence consisting of sequence SEQ ID NO: 306. In some aspects, the HC sequence is an HC sequence consisting of SEQ ID NO: 239 and the LC sequence is an LC sequence consisting of sequence SEQ ID NO: 307. In some aspects, the HC sequence is an HC sequence consisting of SEQ ID NO: 240 and the LC sequence is an LC sequence consisting of sequence SEQ ID NO: 308. In some aspects, the HC sequence is an HC sequence consisting of SEQ ID NO: 241 and the LC sequence is an LC sequence consisting of sequence SEQ ID NO: 309. In some aspects, the HC sequence is an HC sequence consisting of SEQ ID NO: 242 and the LC sequence is an LC sequence consisting of sequence SEQ ID NO: 310. In some aspects, the HC sequence is an HC sequence consisting of SEQ ID NO: 243 and the LC sequence is an LC sequence consisting of sequence SEQ ID NO: 311. In some aspects, the HC sequence is an HC sequence consisting of SEQ ID NO: 244 and the LC sequence is an LC sequence consisting of sequence SEQ ID NO: 312. In some aspects, the HC sequence is an HC sequence consisting of SEQ ID NO: 245 and the LC sequence is an LC sequence consisting of sequence SEQ ID NO: 313. In some aspects, the HC sequence is an HC sequence consisting of SEQ ID NO: 246 and the LC sequence is an LC sequence consisting of sequence SEQ ID NO: 314. In some aspects, the HC sequence is an HC sequence consisting of SEQ ID NO: 247 and the LC sequence is an LC sequence consisting of sequence SEQ ID NO: 315. In some aspects, the HC sequence is an HC sequence consisting of SEQ ID NO: 248 and the LC sequence is an LC sequence consisting of sequence SEQ ID NO: 316. In some aspects, the HC sequence is an HC sequence consisting of SEQ ID NO: 249 and the LC sequence is an LC sequence consisting of sequence SEQ ID NO: 317. In some aspects, the HC sequence is an HC sequence consisting of SEQ ID NO: 250 and the LC sequence is an LC sequence consisting of sequence SEQ ID NO: 318. In some aspects, the HC sequence is an HC sequence consisting of SEQ ID NO: 251 and the LC sequence is an LC sequence consisting of sequence SEQ ID NO: 319. In some aspects, the HC sequence is an HC sequence consisting of SEQ ID NO: 252 and the LC sequence is an LC sequence consisting of sequence SEQ ID NO: 320. In some aspects, the HC sequence is an HC sequence consisting of SEQ ID NO: 253 and the LC sequence is an LC sequence consisting of sequence SEQ ID NO: 321. In some aspects, the HC sequence is an HC sequence consisting of SEQ ID NO: 254 and the LC sequence is an LC sequence consisting of sequence SEQ ID NO: 322. In some aspects, the HC sequence is an HC sequence consisting of SEQ ID NO: 255 and the LC sequence is an LC sequence consisting of sequence SEQ ID NO: 323. In some aspects, the HC sequence is an HC sequence consisting of SEQ ID NO: 256 and the LC sequence is an LC sequence consisting of sequence SEQ ID NO: 324. In some aspects, the HC sequence is an HC sequence consisting of SEQ ID NO: 257 and the LC sequence is an LC sequence consisting of sequence SEQ ID NO: 325. In some aspects, the HC sequence is an HC sequence consisting of SEQ ID NO: 258 and the LC sequence is an LC sequence consisting of sequence SEQ ID NO: 326. In some aspects, the HC sequence is an HC sequence consisting of SEQ ID NO: 259 and the LC sequence is an LC sequence consisting of sequence SEQ ID NO: 327. In some aspects, the HC sequence is an HC sequence consisting of SEQ ID NO: 260 and the LC sequence is an LC sequence consisting of sequence SEQ ID NO: 328. In some aspects, the HC sequence is an HC sequence consisting of SEQ ID NO: 261 and the LC sequence is an LC sequence consisting of sequence SEQ ID NO: 329. In some aspects, the HC sequence is an HC sequence consisting of SEQ ID NO: 262 and the LC sequence is an LC sequence consisting of sequence SEQ ID NO: 330.

In some aspects, the HC sequence is an HC sequence consisting of SEQ ID NO: 266 and the LC sequence is an LC sequence consisting of a sequence SEQ ID NO: 300. In some aspects, the HC sequence is an HC sequence consisting of SEQ ID NO: 267 and the LC sequence is an LC sequence consisting of sequence SEQ ID NO: 301. In some aspects, the HC sequence is an HC sequence consisting of SEQ ID NO: 268 and the LC sequence is an LC sequence consisting of sequence SEQ ID NO: 302. In some aspects, the HC sequence is an HC sequence consisting of SEQ ID NO: 269 and the LC sequence is an LC sequence consisting of sequence SEQ ID NO: 303. In some aspects, the HC sequence is an HC sequence consisting of SEQ ID NO: 270 and the LC sequence is an LC sequence consisting of sequence SEQ ID NO: 304. In some aspects, the HC sequence is an HC sequence consisting of SEQ ID NO: 271 and the LC sequence is an LC sequence consisting of sequence SEQ ID NO: 305. In some aspects, the HC sequence is an HC sequence consisting of SEQ ID NO: 272 and the LC sequence is an LC sequence consisting of sequence SEQ ID NO: 306. In some aspects, the HC sequence is an HC sequence consisting of SEQ ID NO: 273 and the LC sequence is an LC sequence consisting of sequence SEQ ID NO: 307. In some aspects, the HC sequence is an HC sequence consisting of SEQ ID NO: 274 and the LC sequence is an LC sequence consisting of sequence SEQ ID NO: 308. In some aspects, the HC sequence is an HC sequence consisting of SEQ ID NO: 275 and the LC sequence is an LC sequence consisting of sequence SEQ ID NO: 309. In some aspects, the HC sequence is an HC sequence consisting of SEQ ID NO: 276 and the LC sequence is an LC sequence consisting of sequence SEQ ID NO: 310. In some aspects, the HC sequence is an HC sequence consisting of SEQ ID NO: 277 and the LC sequence is an LC sequence consisting of sequence SEQ ID NO: 311. In some aspects, the HC sequence is an HC sequence consisting of SEQ ID NO: 278 and the LC sequence is an LC sequence consisting of sequence SEQ ID NO: 312. In some aspects, the HC sequence is an HC sequence consisting of SEQ ID NO: 279 and the LC sequence is an LC sequence consisting of sequence SEQ ID NO: 313. In some aspects, the HC sequence is an HC sequence consisting of SEQ ID NO: 280 and the LC sequence is an LC sequence consisting of sequence SEQ ID NO: 314. In some aspects, the HC sequence is an HC sequence consisting of SEQ ID NO: 281 and the LC sequence is an LC sequence consisting of sequence SEQ ID NO: 315. In some aspects, the HC sequence is an HC sequence consisting of SEQ ID NO: 282 and the LC sequence is an LC sequence consisting of sequence SEQ ID NO: 316. In some aspects, the HC sequence is an HC sequence consisting of SEQ ID NO: 283 and the LC sequence is an LC sequence consisting of sequence SEQ ID NO: 317. In some aspects, the HC sequence is an HC sequence consisting of SEQ ID NO: 284 and the LC sequence is an LC sequence consisting of sequence SEQ ID NO: 318. In some aspects, the HC sequence is an HC sequence consisting of SEQ ID NO: 285 and the LC sequence is an LC sequence consisting of sequence SEQ ID NO: 319. In some aspects, the HC sequence is an HC sequence consisting of SEQ ID NO: 286 and the LC sequence is an LC sequence consisting of sequence SEQ ID NO: 320. In some aspects, the HC sequence is an HC sequence consisting of SEQ ID NO: 287 and the LC sequence is an LC sequence consisting of sequence SEQ ID NO: 321. In some aspects, the HC sequence is an HC sequence consisting of SEQ ID NO: 288 and the LC sequence is an LC sequence consisting of sequence SEQ ID NO: 322. In some aspects, the HC sequence is an HC sequence consisting of SEQ ID NO: 289 and the LC sequence is an LC sequence consisting of sequence SEQ ID NO: 323. In some aspects, the HC sequence is an HC sequence consisting of SEQ ID NO: 290 and the LC sequence is an LC sequence consisting of sequence SEQ ID NO: 324. In some aspects, the HC sequence is an HC sequence consisting of SEQ ID NO: 291 and the LC sequence is an LC sequence consisting of sequence SEQ ID NO: 325. In some aspects, the HC sequence is an HC sequence consisting of SEQ ID NO: 292 and the LC sequence is an LC sequence consisting of sequence SEQ ID NO: 326. In some aspects, the HC sequence is an HC sequence consisting of SEQ ID NO: 293 and the LC sequence is an LC sequence consisting of sequence SEQ ID NO: 327. In some aspects, the HC sequence is an HC sequence consisting of SEQ ID NO: 294 and the LC sequence is an LC sequence consisting of sequence SEQ ID NO: 328. In some aspects, the HC sequence is an HC sequence consisting of SEQ ID NO: 295 and the LC sequence is an LC sequence consisting of sequence SEQ ID NO: 329. In some aspects, the HC sequence is an HC sequence consisting of SEQ ID NO: 296 and the LC sequence is an LC sequence consisting of sequence SEQ ID NO: 330.

### 2.8. Consensus Sequences

In some embodiments, provided herein are anti-HLA-G antibodies comprising one or more sequences defined by consensus sequences. Each consensus sequence is based, at least in part, on one or more alignments of two or more useful anti-HLA-G CDR sequences provided in this disclosure. Based on such alignments, a person of skill in the art would recognize that different amino acid residues may useful in certain positions of the CDRs. Accordingly, each consensus sequence encompasses two or more useful anti-HLA-G CDR sequences.

### 2.8.1. CDR-H3 Consensus Sequences

In some embodiments, the antibody comprises a CDR-H3 sequence defined by the consensus sequence G-Y₂-Y₃-R-A-V-P-F-Y₉-Y₁₀ (SEQ ID NOS: 76-84), where y₂ is I, P, Q, T, or V; Y₃ is A, F, K, or R; Y₉ is A, D, Q, or V; Y₁₀ is D, R, or Y.

In some aspects, when y₂ is I; Y₃ is A or R; Y₉ is F; and Y₁₀ is Y. In some aspects, when Y₂ is V; Y₃ is R; Y₉ is A, D, Q, or V; and Y₁₀ is D, R, or Y. In some aspects, when Y₃ is R; Y₂ is I, T, or V; Y₉ is A, D, or Q; and y₁₀ is D, R, or Y. In some aspects, when Y₉ is D; Y₂ is I, P, Q, or V; Y₃ is A, F, K, or R; and y₁₀ is Y. In some aspects, when y₁₀ is Y; y₂ is I, P, R, or V; Y₃ is A, F, K, or R; and Y₉ is D. In some aspects, when Y₁₀ is D; y₂ is V; y₃ is R; and Y₉ is A or V.

In some aspects, when Y₂ is V; Y₃ is R; Y₉ is D; and Y₁₀ is Y. In some aspects, when Y₂ is I; Y₃ is A; Y₉ is D; and Y₁₀ is Y. In some aspects, when y₂ is P; Y₃ is K; y₉ is D; and y₁₀ is Y. In some aspects, when y₂ is V; Y₃ is R; Y₉ is V; and Y₁₀ is D. In some aspects, when Y₂ is V; Y₃ is R; Y₉ is Q; and Y₁₀ is R. In some aspects, when y₂ is T; Y₃ is R; Y₉ is D; and Y₁₀ is Y. In some aspects, when Y₂ is V; Y₃ is R; Y₉ is A; and Y₁₀ is D. In some aspects, when Y₂ is I; Y₃ is R; Y₉ is D; and Y₁₀ is Y. In some aspects, when y₂ is Q; y₃ is F; Y₉ is D; and y₁₀ is Y.

In some embodiments, the antibody comprises a CDR-H3 sequence defined by the consensus sequence G-G-Φ₃-Φ₄-Φ₅-Y-S-R-G-P-Φ₁₁-D-V (SEQ ID NOS: 85-93), where Φ₃ is E, G, Q, or T; Φ₄ is A, H, P, Q, or V; Φ₅ is K or T; and Oil is F, L, or M.

In some aspects, Φ₃ is G; when Φ₄ is A or Q; Φ₅ is T; and Qui is V. In some aspects, Φ₃ is T; when Φ₄ is H, P, or V; Φ₅ is I, T, or Y; and Φ₁₁ is V. In some aspects, Φ₄ is H; when Φ₃ is T; Φ₅ is T; and Φ₁₁ is F, L, or M. In some aspects, Φ₄ is V; when Φ₃ is E, T, or Q; Φ₅ is K or T; and Φ₁₁ is L. In some aspects, Φ₅ is T; when Φ₃ is E, G, T, or Q; Φ₄ is A, H, Q, or V; and Φ₁₁ is F, L, or M. In some aspects, Φ₁₁ is L; when Φ₃ is E, G, T, or Q; Φ₄ is A, H, P, Q, or V; and Φ₅ is I, K, or T.

In some aspects, when Φ₃ is T; when Φ₄ is H; Φ₅ is T; and Φ₁₁ is M. In some aspects, when Φ₃ is T; when Φ₄ is H; Φ₅ is T; and Φ₁₁ is F. In some aspects, when Φ₃ is T; when Φ₄ is P; 5 is I; and Φ₁₁ is L. In some aspects, when Φ₃ is G; when Φ₄ is Q; Φ₅ is T; and Φ₁₁ is L. In some aspects, when Φ₃ is G; when Φ₄ is A; Φs is T; and Φ₁₁ is L. In some aspects, when Φ₃ is T; when Φ₄ is H; Φ₅ is T; and Φ₁₁ is L. In some aspects, when Φ₃ is T; when Φ₄ is H; Φ₅ is T; and Φ₁₁ is L. In some aspects, when Φ₃ is T; when Φ₄ is V; Φ₅ is K; and Φ₁₁ is L. In some aspects, when Φ₃ is Q; when Φ₄ is V; Φs is T; and Φ₁₁ is L. In some aspects, when Φ₃ is E; when Φ₄ is V; Φ₅ is T; and Φ₁₁ is L.

### 2.8.2. Chothia CDR-H2 Consensus Sequences

In some embodiments, the antibody comprises a Chothia CDR-H2 sequence defined by the consensus sequence Y-ε₂-S-ε₄-S(SEQ ID NOS: 38 and 44-45), where 82 is H or Y and 84 is A or G.

In some aspects, when ε₂ is H; ε₄ is A or G. In some aspects, when ε₄ is G, ε₂ is H or Y.

In some aspects, when ε₂ is Y; ε₄ is G. In some aspects, when ε₂ is H; ε₄ is G. In some aspects, when ε₂ is H; ε₄ is A.

In some embodiments, the antibody comprises a Chothia CDR-H2 sequence defined by the consensus sequence α₁-α₂-S-G-S(SEQ ID NOS: 39, 41-42, and 49), where α₁ is A, H, or S; and α₂ is S or Y.

In some aspects, when α₁, is S; α₂ is S or Y.

In some aspects, when α₁ is S; α₂ is S. In some aspects, when al is S; α₂ is Y. In some aspects, when α₁ is H; α₂ is Y. In some aspects, when ai is A; α₂ is Y.

In some embodiments, the antibody comprises a Chothia CDR-H2 sequence defined by the consensus sequence β₁-β₂-S-G-β₅-β₆ (SEQ ID NOS: 56-60), where β₁ is A or S; β₂ is G or S; β₅ is I or S; and β₆ is T or V.

In some aspects, when β₁ is S, β₂ is G or S; β₅ is I or S; and β₆ is T or V. In some aspects, when β₂ is S, β₁ is A or S; β₅ S; and β₆ is T or V. In some aspects, when β₅ is S, β₁ is A or S; β₂ is S; and β₆ is T or V. In some aspects, when β₆ is T, β₁ is S; β₂ is G or S; and β₅ is I or T.

In some aspects, when β₁ is A; β₂ is S; β₅ is S; and β₆ is V. In some aspects, when β₁ is S; β₂ is G; Bs is I; and β₆ is T. In some aspects, when β₁ is S; β₂ is S; Bs is S; and β₆ is T.

### 2.8.3. Chothia CDR-H1 Consensus Sequences

In some embodiments, the antibody comprises a Chothia CDR-H1 sequence defined by the consensus sequence G-G-S-I-S-S-Ω₇-Ω₈-Ω₉ (SEQ ID NOS: 1-4 and 13-14), where Ω₇ is S or A; Ω₈ is D, S, or N; and Ω₉ is T, N, Y, or is absent.

In some aspects, when Ω₇ is S; Ω₈ is D, N, or S; and Ω₉ is T, Y, or is absent.

In some aspects, when Ω₈ is D; Ω₇ is S or A; and Ω₉ is N, T, or Y.

In some aspects, when Ω₉ is T; Ω₇ is S; and Ω₈ is D or S. In some aspects, when Ω₉ is Y; Ω₇ is S; and Ω₈ is D or S.

In some aspects, when Ω₇ is S; Ω₂₈ is D; and Ω₉ is Y. In some aspects, when Ω₇ is S; Ω₈ is S; and Ω₉ is T. In some aspects, when Ω₇ is S; Ω₈ is D; and Ω₉ is T. In some aspects, when Ω₇ is A; Ω₈ is D; and Ω₉ is N. In some aspects, when Ω₇ is S; 28 is N; and 09 is absent. In some aspects, when Ω₇ is S; Ω₈ is S; and Ω₉ is Y.

In some embodiments, the antibody comprises a Chothia CDR-H1 sequence defined by the consensus sequence G-Y-S-I-v₅-S-G-v₈ (SEQ ID NOS: 6-9), where v₅ is S or L and v₈ is F, H, or Y.

In some aspects, when vs is S, v& is F, H, or Y.

In some aspects, when vs is S, v₈ is F. In some aspects, when vs is S, v₈ is H. In some aspects, when vs is S, vs is Y. In some aspects, when vs is L, v₈ is Y.

In some embodiments, the antibody comprises a Chothia CDR-H1 sequence defined by the consensus sequence G-F-T-F-κ₅-κ₆-κ₇ (SEQ ID NOS: 10-12), where κ₅ is D or S; κ₆ is D, N, or S; and κ₇ is S or Y.

In some aspects, when κ₅ is S; κ₆ is D or S; and κ₇ is S or Y. In some aspects, when κ₇ is Y; κ₅ is D or S; and κ₆ is N or D.

In some aspects, when κ₅ is D; κ₆ is N; and κ₇ is Y. In some aspects, when κ₅ is S; κ₆ is D; and κ₇ is Y. In some aspects, when κ₅ is S; κ₆ is S; and κ₇ is S.

### 2.8.4. Kabat CDR-H2 Consensus Sequences

In some embodiments, the antibody comprises a Kabat CDR-H2 sequence defined by the consensus sequence β₁-I-β₃-β₄-β₅-β₆-β₇-T-β₉-Y-N-P-S-L-K-S(SEQ ID NOS: 54-65 and 69-70) where β₁ is A, E, G, or S; β₃ is A, H, S, or Y; β₄ is H, S, or Y; β₅ is N or S; β₆ is A or G; β₇ is A, L, or S; and β₉ A, N, L, V, or Y.

In some aspects, when β₁ is S; β₃ is A, H, S, or Y; β₄ is H, S, or Y; β₅ is N or S; β₆ is A or G; β₇ is A, L, or S; and β₉ is A, N, L, V, or Y. In some aspects, when β₁ is G; β₃ is A or Y; β₄ is H or Y; Bs is G or S; β₆ is A or G; β₇ is S; and β₉ is A or Y. In some aspects, when β₃ is Y; β₁ is A, E, G, or S; β₄ is H or Y; Bs is S; β₆ is A or G; Bris S; and β₉ is A, N, V, or Y. In some aspects, when β₃ is S; β₁ is S; β₄ is S or Y; Bs is N or S; β₆ is A or G; β₇ is L or S; and β₉ is Y. In some aspects, when β₃ is H; β₁ is S; β₄ is H or Y; Bs is S; β₆ is G; β₇ is A or S; and β₉ is L or Y. In some aspects, when β₄ is Y; β₁ is S or G; β₃ is A, H, S, or Y; β₅ is N or S; β₆ is A or G; β₇ is L or S; and β₉ is L or Y. In some aspects, when β₄ is H; β₁ is A, E, G, S; β₃ is H or Y; Bs is S; β₆ is A or G; β₇ is A or S; and β₉ is A, N, Y or V. In some aspects, when Bs is S; β₁ is A, E, G, or S; β₃ is A, H, S, or Y; β₄ is H, S, or Y; β₆ is A or G; β₇ is A or S; and β₉ is A, L, N, Y, or V. In some aspects, when β₆ is G; β₁ is A, E, G, or S; β₃ is A, H, S, or Y; β₄ is H, S, or Y; Bs is S; β₇ is S; and β₉ is A, N, L, V, or Y. In some aspects, when β₆ is A; β₁ is G or S; β₃ is S or Y; β₄ is H or Y; β₅ is N or S; β₇ is L or S; and β₉ is A or Y. In some aspects, when β₇ is S; β₁ is A, E, G, or S; β₃ is A, H, S, or Y; β₄ is H, S, or Y; β₅ is S; β₆ is A or G; and β₉ is A, L, N, V, or Y. In some aspects, when β₉ is Y; β₁ is G or S; β₃ is A, H, S, or Y; β₄ is H, S, or Y; β₅ is N or S; β₆ is A or G; and β₇ is A, L, or S. In some aspects, when β₉ is A; β₁ is G; β₃ is Y; β₄ is H; Bs is S; β₆ is A or G; and β₇ is S.

In some aspects, when β₁ is S; β₃ is Y; β₄ is Y; is S; β₆ is G; and β₇ is S; and β₉ is Y. In some aspects, when β₁ is S; β₃ is S; β₄ is S; β₅ is S; β₆ is G; and β₇ is S; and β₉ is Y. In some aspects, when β₁ is S; β₃ is H; β₄ is H; Bs is S; β₆ is G; and β₇ is A; and β₉ is Y. In some aspects, when β₁ is S; β₃ is H; β₄ is Y; β₅ is S; β₆ is G; and β₇ is S; and β₉ is L. In some aspects, when β₁ is S; β₃ is H; β₄ is Y; Bs is S; β₆ is G; and β₇ is S; and β₉ is Y. In some aspects, when β₁ is G; β₃ is A; β₄ is Y; β₅ is S; β₆ is G; and β₇ is S; and β₉ is Y. In some aspects, when β₁ is S; β₃ is S; β₄ is Y; β₅ is N; β₆ is A; and β₇ is L; and β₉ is Y. In some aspects, when β₁, is S; β₃ is Y; β₄ is H; β₅ is S; β₆ is G; and β₇ is S; and β₉ is Y. In some aspects, when β₁ is G; β₃ is Y; β₄ is H; β₅ is S; β₆ is A; and β₇ is S; and β₉ is A. In some aspects, when β₁ is G; β₃ is Y; β₄ is H; Bs is S; β₆ is G; and β₇ is S; and β₉ is Y. In some aspects, when β₁ is A; β₃ is Y; β₄ is H; Bs is S; β₆ is G; and β₇ is S; and β₉ is V. In some aspects, when β₁ is G; β₃ is Y; β₄ is H; β₅ is S; β₆ is G; and β₇ is S; and β₉ is A. In some aspects, when β₁ is E; β₃ is Y; β₄ is H; β₅ is S; β₆ is G; and β₇ is S; and β₉ is N. In some aspects, when β₁ is S; β₃ is S; β₄ is Y; β₅ is S; β₆ is G; and β₇ is S; and β₉ is Y.

### 2.8.5. Kabat CDR-H1 Consensus Sequences

In some embodiments, the antibody comprises a Kabat CDR-H1 sequence defined by the consensus sequence S-S-Δ₃-Δ₄-Y-W-Δ₇ (SEQ ID NOS: 18-21, 23, and 34), where Δ₃ is D or S; Δ₄ is T or Y; and Δ₇ is A, G, or S.

In some aspects, when Δ₃ is D; Δ₄ is T or Y; and Δ₇ is G. In some aspects, when Δ₃ is S; Δ₄ is T or Y; and Δ₇ is A, G, or S. In some aspects, when Δ₄ is T; Δ₃ is D or S; and Δ₇ is A, G, or S. In some aspects, when Δ₄ is Y; Δ₃ is D or S; and Δ₇ is G. In some aspects, when Δ₇ is G; Δ₃ is D or S; and Δ₄ is T or Y.

In some aspects, when Δ₃ is D; Δ₄ is Y; and Δ₇ is G. In some aspects, when Δ₃ is S; Δ₄ is T; and Δ₇ is A. In some aspects, when Δ₃ is S; Δ₄ is T; and Δ₇ is G. In some aspects, when Δ₃ is D; Δ₄ is T; and Δ₇ is G. In some aspects, when Δ₃ is S; Δ₄ is T; and Δ₇ is S. In some aspects, when Δ₃ is S; Δ₄ is Y; and Δ₇ is G.

In some embodiments, the antibody comprises a Kabat CDR-H1 sequence defined by the consensus sequence S-G-θ₃-Y-W-θ₆ (SEQ ID NOS: 24-29), where θ₃ is F, H, or Y; and θ₆ is F, G, I, L, or T.

In some aspects, when θ₃ is H, θ₆ is I or T. In some aspects, when θ₃ is Y, θ₆ is F, G, or L. In some aspects, when θ₆ is T, θ₃ is F or H.

In some aspects, when θ₃ is Y, θ₆ is G. In some aspects, when θ₃ is Y, θ₆ is F. In some aspects, when θ₃ is H, θ₆ is I. In some aspects, when θ₃ is F, θ₆ is T. In some aspects, when θ₃ is Y, θ₆ is L. In some aspects, when θ₃ is H, θ₆ is T.

### 2.8.6. CDR-L3 Consensus Sequences

In some embodiments, the antibody comprises a CDR-L3 sequence defined by the consensus sequence Q-π₂-π₃-π₄-H-S-P-Y-T (SEQ ID NOS: 149-153), where π₂ is Q or W; π₃ is A, T, or V; and π₄ is I or V.

In some aspects, when π₂ is Q; π₃ is A, T, or V; and π₄ is I or V. In some aspects, when π₃ is A; π₂ is Q or W; and π₄ is I or V. In some aspects, when π₄ is V; π₂ is Q or W; and π₃ is A, T, or V.

In some aspects, when π₂ is Q; π₃ is A; and π₄ is V. In some aspects, when π2 is W; π₃ is A; and π₄ is V. In some aspects, when π₂ is Q; π₃ is V; and π₄ is V. In some aspects, when π₂ is Q; π₃ is T; and π₄ is V. In some aspects, when π₂ is Q; π₃ is A; and π₄ is I.

In some embodiments, the antibody comprises a CDR-L3 sequence defined by the consensus sequence Q-Q-λ₃-S-λ₅-Y-P-P-T (SEQ ID NOS: 154-158), where λ₃ is F, H, or V; and λ₅ is I, L, or S.

In some aspects, when λ₃ is H; λ₅ is I, L, or S. In some aspects, when λ₅ is H; λ₃ is F, H, or V.

In some aspects, when λ₃ is H, λ₅ is S. In some aspects, when λ₃ is H, λ₅ is L. In some aspects, when λ₃ is F, λ₅ is S. In some aspects, when λ₃ is V, λ₅ is S. In some aspects, when λ₃ is H, λ₅ is I.

In some embodiments, the antibody comprises a CDR-L3 sequence defined by the consensus sequence Q-Q-ω₃-ω₄-ω₅-ω₆-P-I-T (SEQ ID NOS: 160-161 and 163-164), where ω₃ is A, L, V, or Y; ω₄ is G, P, V, or Y; ω₅ is S, L, or F; and ω₆ is D, L, S, or Y.

In some aspects, when ω₅ is S; ω₃ is V or Y; ω₄ is G or V; and ω₆ is D or S.

In some aspects, when ω₃ is A; ω₄ is Y; ω₅ is L; and ω₆ is Y. In some aspects, when ω₃ is L; ω₄ is P; ω₅ is F; and ω₆ is L. In some aspects, when ω₃ is Y; ω₄ is V; ω₅ is S; and ω₆ is D. In some aspects, when ω₃ is V; ω₄ is G; ω₅ is S; and ω₆ is S.

### 2.8.7. CDR-L2 Consensus Sequences

In some embodiments, the antibody comprises a CDR-L2 sequence defined by the consensus sequence ψ₁-A-S-4-R-A-ψ₇ (SEQ ID NOS: 128, 130, 132, 134-138, 143, and 145), where ψ₁ is D or G; ψ₄ is A, D, N, R, S, T, or Y; and ψ₇ is A, N, S, or T.

In some aspects, when ψ₁ is G; ψ₄ is A, D, N, R, S, T, or Y; and ψ₇ is A, N, or T. In some aspects, when ψ₁ is D; ψ₄ is S or T; and ψ₇ is S or T. In some aspects, when ψ₄ is S; ψ₁ is G or D; and ψ₇ is S or T. In some aspects, when ψ₄ is N; ψ₁ is G or D; and ψ₇ is A or T. In some aspects, when ψ₄ is T; ψ₁ is G or D; and wis T. In some aspects, when ψ₇ is T; ψ₁ is G or D; and ψ₄ is A, N, R, S, T, or Y.

In some aspects, when ψ₁ is G; ψ₄ is S; and ψ7 is T. In some aspects, when 4/1 is G; ψ₄ is A; and ψ₇ is T. In some aspects, when ψ₁ is N; ψ₄ is S; and ψ₇ is A. In some aspects, when ψ₁ is G; ψ₄ is N; and ψ₇ is T. In some aspects, when ψ₁ is D; ψ₄ is S; and ψ₇ is S. In some aspects, when ψ₁ is D; ψ₄ is S; and ψ7 is T. In some aspects, when ψ₁ is G; ψ₄ is D; and ψ₇ is N. In some aspects, when ψ₁ is G; ψ₄ is Y; and ψ₇ is T. In some aspects, when ψ₁ is G; ψ₄ is R; and ψ₇ is T. In some aspects, when ψ₁ is G; ψ₄ is T; and ψ₇ is T.

### 2.8.8. CDR-L1 Consensus Sequences

In some embodiments, the antibody comprises a CDR-L1 sequence defined by the consensus sequence ϕ₁-A-S-Q-ϕ₅-V-S-ϕ₉-ϕ₁₀-L-A (SEQ ID NOS: 105-112 and 117), where ϕ₁ is E, G, K, Q, or R; ϕ₅ is A or S; ϕ₉ is A, D, N, S, or T; and ϕ₁₀ is P or Y.

In some aspects, when di is R; ϕ₅ is Q or S; ϕ₉ is A, S, or T; and ϕ₁₀ is S or Y. In some aspects, when ϕ₁ is G; ϕ₅ is Q or S; ϕ₉ is A or D; and ϕ₁₀ is P or Y. In some aspects, when ϕ₁ is Q; ϕ₅ is A or S; ϕ₉ is N or S; and ϕ₁₀ is Y. In some aspects, when ϕ₅ is S; ϕ₁ is E, G, Q, or R; ϕ₉ is A, D, S, or T; and ϕ₁₀ is P or Y. In some aspects, when ϕ₅ is A; ϕ₁ is K or Q; ϕ₉ is N or S; and ϕ₁₀ is Y. In some aspects, when do is S; ϕ₁ is E, K, Q, or R; ϕ₅ is A or S; and Ω₁₀ is Y. In some aspects, when do is A; ϕ₁ is G or R; ϕ₅ is S; and ϕ₁₀ is P or Y. In some aspects, when ϕ₁₀ is Y; Ω₁ is E, G, K, Q, or R; ϕ₅ is A or S; and ϕ₉ is A, D, N, S, or T.

In some aspects, when is R; ϕ₅ is S; do is S; and ϕ₁₀ is Y. In some aspects, when di is G; ϕ₅ is S; do is D; and ϕ₁₀ is Y. In some aspects, when di is Q; ϕ₅ is A; ¢9 is N; and Ω₁₀ is Y. In some aspects, when di is G; ϕ₅ is S; ϕ₉ is A; and Ω₁₀ is P. In some aspects, when $1 is R; ϕ₅ is S; ϕ₉ is T; and ϕ₁₀ is Y. In some aspects, when di is Q; ϕ₅ is S; do is S; and ϕ₁₀ is Y. In some aspects, when di is K; ϕ₅ is A; ϕ₉ is S; and ϕ₁₀ is Y. In some aspects, when ϕ₁ is E; ϕ₅ is S; 49 is S; and ϕ₁₀ is Y. In some aspects, when di is R; ϕ₅ is S; ϕ₉ is A; and ϕ₁₀ is Y.

In some embodiments, the antibody comprises a CDR-L1 sequence defined by the consensus sequence R-A-S-Q-S-σ₆-σ₇-S-σ₉-L-σ₁₁ (SEQ ID NOS: 119 and 123-124), where σ₆ is I or V; σ₇ is N or S; σ₉ is N, W, or Y; σ₁₁ is A or N.

In some aspects, when σ₆ is I, σ₇ is N or S; σ₉ is W or Y; and σ₁₁ is A or N. In some aspects, when σ₇ is S, σ₆ is I or V; σ₉ is N or Y; and σ₁₁ is A or N. In some aspects, when σ₁₁ is A, σ₆ is I or V; σ₇ is N or S; and σ₉ is N or W.

In some aspects, when σ₆ is I; σ₇ is N; σ₉ is W; and σ₁₁ is A. In some aspects, when σ₆ is I; σ₇ is S; σ₉ is Y; and σ₁₁ is N. In some aspects, when σ₆ is V; σ₇ is S; σ₉ is N; and σ₁₁ is A.

In some embodiments, the antibody comprises a CDR-L1 sequence defined by the consensus sequence Γ₁-Γ₂-S-Q-S-V-S-Γ₈-Γ₉-Y-L-A (SEQ ID NOS: 113-116), where Ti is E or R; Γ₂ is A or V; Γ₈ is A, D, or S; and Γ₁₀ is A or S.

In some aspects, when Γ₁ is E; Γ₂ is A or V; Γ₈ A or S; and Γ₉ is A or S. In some aspects, when Γ₂ is A; Γ₁ is E; Γ₈ is A or S; and Γ₉ is A or S. In some aspects, when Γ₂ is V; Γ₁ is E or R; Γ₈ A or D; and Γ₉ is A or S. In some aspects, when Γ₈ is A; Ti is E; Γ₂ is A or V; and Γ₉ is S. In some aspects, when Γ₉ is S; Γ₁ is E; Γ₂ is A or V; and Γ₈ is A. In some aspects, when Γ₉ is A; Γ₁, is E or R; Γ₂ is A or V; and Γ₈ D or S.

In some aspects, when Γ₁ is E; Γ₂ is A; Γ₈ is A; and Γ₉ is S. In some aspects, when Γ₁ is E; Γ₂ is A; Γ₈ is S; and Γ₉ is A. In some aspects, when Γ₁ is R; Γ₂ is V; Γ₈ is A; and Γ₉ is A. In some aspects, when Ti is E; Γ₂ is V; Γ₈ is A; and Γ₉ is S.

### 3. Germline

In some embodiments, the antibody that specifically binds HLA-G is an antibody comprising a variable region that is encoded by a particular germline, gene, or a variant thereof. The illustrative antibodies provided herein comprise variable regions that are encoded by the heavy chain variable region germline genes VH3-23, VH4-39, VH3-11, and VH4-0B or variants thereof; and the light chain variable region germline genes VK1-33, VK3-20, VK1-12, and VK1-05 or variants thereof. One of skill in the art would recognize that the CDR sequences provided herein may also be useful when combined with variable regions encoded by other variable region germline genes or variants thereof. In particular, the CDR sequences provided herein may be useful when combined with variable regions encoded by variable region germline genes, or variants thereof, that are structurally similar to the variable region germline genes recited above. For example, in some embodiments, a CDR-H sequence provided herein may be combined with a variable region encoded by a variable region germline gene selected from the VH1 or VH3 family, or a variant thereof. In some embodiments, a CDR-L sequence provided herein may be combined with a variable region encoded by a variable region germline gene selected from the Vλ3, Wκ1, VK3, and VK4 families, or a variant thereof.

### 4. Affinity

In some embodiments, the affinity of the antibody for HLA-G, as indicated by KD, is less than about 10⁻⁵ M, less than about 10⁻⁶ M, less than about 10⁻⁷ M, less than about 10⁻⁸ M, less than about 10⁻⁹ M, less than about 10⁻¹⁰ M, less than about 10⁻¹¹ M, or less than about 10⁻¹² M. In some embodiments, the affinity of the antibody is between about 10⁻⁷ M and 10⁻¹¹ M. In some embodiments, the affinity of the antibody is between about 10⁻⁷ M and 10⁻¹⁰ M. In some embodiments, the affinity of the antibody is between about 10⁻⁷ M and 10⁻⁹ M. In some embodiments, the affinity of the antibody is between about 10⁻⁷ M and 10⁻⁸ M. In some embodiments, the affinity of the antibody is between about 10⁻⁸ M and 10⁻¹¹ M. In some embodiments, the affinity of the antibody is between about 10⁻⁸ M and 10⁻¹⁰ M. In some embodiments, the affinity of the antibody is between about 10⁻⁹ M and 10⁻¹¹ M. In some embodiments, the affinity of the antibody is between about 10⁻¹⁰ M and 10⁻¹¹ M.

In some embodiments, the affinity of the antibody for human HLA-G is between about 1.00×10⁻⁸ M and 9.43×10⁻¹⁰ M. In some embodiment, the affinity of the antibody for human HLA-G is about 1.00×10⁻⁸ M, about 1.08×10⁻⁸ M, about 1.10×10⁻⁸ M, about 1.13×10⁻⁸ M, about 1.14×10⁻⁸ M, about 1.16×10⁻⁸ M, about 1.29×10⁻⁸ M, about 1.40×10⁻⁸ M, about 1.41×10⁻⁸ M, about 1.46×10⁻⁸ M, about 1.67×10⁻⁸ M, about 1.79×10⁻⁸ M, about 1.81×10⁻⁸ M, about 2.04×10⁻⁸ M, about 2.30×10⁻⁸ M, about 2.49×10⁻⁸ M, about 2.59×10⁻⁸ M, about 2.94×10⁻⁸ M, about 2.95×10⁻⁸ M, about 3.11×10⁻⁸ M, about 3.98×10⁻⁹ M, about 4.11×10⁻⁹ M, about 4.20×10⁻⁹ M, about 4.33×10⁻⁹ M, about 4.39×10⁻⁹ M, about 4.42×10⁻⁹ M, about 4.72×10⁻⁹ M, about 5.24×10⁻⁹ M, about 5.30×10⁻⁹ M, about 5.35×10⁻⁹ M, about 5.40×10⁻⁹ M, about 5.55×10⁻⁹ M, about 5.56×10⁻⁹ M, about 5.80×10⁻⁹ M, about 5.89×10⁻⁹ M, about 5.92×10⁻⁹ M, about 5.98×10⁻⁹ M, about 5.99×10⁻⁹ M, about 6.10×10⁻⁹ M, about 6.34×10⁻⁹ M, about 6.66×10⁻⁹ M, about 6.75×10⁻⁹ M, about 7.19×10⁻⁹ M, about 7.69×10⁻⁹ M, about 7.93×10⁻⁹ M, about 8.23×10⁻⁹ M, about 8.34×10⁻⁹ M, about 8.37×10⁻⁹ M, about 8.62×10⁻⁹ M, about 8.82×10⁻⁹ M, about 9.21×10⁻⁹ M, about 9.51×10⁻⁹ M, about 1.62×10⁻¹⁰ M, about 1.63×10⁻¹⁰ M, 1.64×10⁻¹⁰ M about 1.65×10⁻¹⁰ M, about 1.66×10⁻¹⁰ M, about 1.71×10⁻¹⁰ M, about 1.72×10⁻¹⁰ M, about 1.86×10⁻¹⁰ M about 1.78×10⁻¹⁰ M, about 1.97×10⁻¹⁰ M, about 1.98×10⁻¹⁰ M, about 1.99×10⁻¹⁰ M, about 2.29×10⁻¹⁰ M, about 3.24×10⁻¹⁰ M M, about 6.47×10⁻¹⁰ M, about 6.96×10⁻¹⁰ M, about 7.84×10⁻¹⁰ M, about 9.41×10⁻¹⁰ M, or about 9.43×10⁻¹⁰ M.

In some embodiments the antibody has a kon when associating with human HLA-G of between about 1.41×10⁵ M⁻¹×sec⁻¹ and about 1.07×10⁶ M⁻¹×sec⁻¹. In some embodiments the antibody has a kon when associating with human HLA-G of about 1.41×10⁵ M⁻¹×sec⁻¹, about 1.49×10⁵ M⁻¹×sec⁻¹, about 1.66×10⁵ M⁻¹×sec⁻¹, about 2.90×10⁵ M⁻¹×sec⁻¹, about 3.60×10⁵ M⁻¹×sec⁻¹, about 3.74×10⁵ M⁻¹×sec⁻¹, about 3.78×10⁵ M⁻¹×sec⁻¹, about 4.03×10⁵ M⁻¹×sec⁻¹, about 4.30×10⁵ M⁻¹×sec⁻¹, about 4.32×10⁵ M⁻¹×sec⁻¹, about 4.34×10⁵ M⁻¹×sec⁻¹, about 4.60×10⁵ M⁻¹×sec⁻¹, about 4.64×10⁵ M⁻¹×sec⁻¹, about 4.80×10⁵ M⁻¹×sec⁻¹, about 4.84×10⁵ M⁻¹×sec⁻¹, about 4.87×10⁵ M⁻¹×sec⁻¹, about 4.91×10⁵ M⁻¹×sec⁻¹, about 4.96×10⁵ M⁻¹×sec⁻¹, about 4.97×10⁵ M⁻¹×sec⁻¹, about 4.98×10⁵ M⁻¹×sec⁻¹, about 5.01×10⁵ M⁻¹×sec⁻¹, about 5.14×10⁵ M⁻¹×sec⁻¹, about 5.19×10⁵ M⁻¹×sec⁻¹, about 5.20×10⁵ M⁻¹×sec⁻¹, about 5.26×10⁵ M⁻¹×sec⁻¹, about 5.27×10⁵ M⁻¹×sec⁻¹, about 5.30×10⁵ M⁻¹×sec⁻¹, about 5.61×10⁵ M⁻¹×sec⁻¹, about 5.90×10⁵ M⁻¹×sec⁻¹, about 6.31×10⁵ M⁻¹×sec⁻¹, about 6.32×10⁵ M⁻¹×sec⁻¹, about 6.36×10⁵ M⁻¹×sec⁻¹, about 6.46×10⁵ M⁻¹×sec⁻¹, about 6.53×10⁵ M⁻¹×sec⁻¹, about 6.66×10⁵ M⁻¹×sec⁻¹, about 6.84×10⁵ M⁻¹×sec⁻¹, about 7.20×10⁵ M⁻¹×sec⁻¹, about 7.24×10⁵ M⁻¹×sec⁻¹, about 7.48×10⁵ M⁻¹×sec⁻¹, about 7.36×10⁵ M⁻¹×sec⁻¹, about 7.58×10⁵ M⁻¹×sec⁻¹, about 7.77×10⁵ M⁻¹×sec⁻¹, about 7.92×10⁵ M⁻¹×sec⁻¹, about 7.94×10⁵ M⁻¹×sec⁻¹, about 7.96×10⁵ M⁻¹×sec⁻¹, about 8.03×10⁵ M⁻¹×sec⁻¹, about 8.24×10⁵ M⁻¹×sec⁻¹, about 8.26×10⁵ M⁻¹×sec⁻¹, about 8.55×10⁵ M⁻¹×sec⁻¹, about 8.63×10⁵ M⁻¹×sec⁻¹, about 8.66×10⁵ M⁻¹×sec⁻¹, about 8.74×10⁵ M⁻¹×sec⁻¹, about 8.79×10⁵ M⁻¹×sec⁻¹, about 8.92×10⁵ M⁻¹×sec⁻¹, about 8.96×10⁵ M⁻¹×sec⁻¹, about 9.09×10⁵ M⁻¹×sec⁻¹, about 9.31×10⁵ M⁻¹×sec⁻¹, about 9.35×10⁵ M⁻¹×sec⁻¹, about 9.38×10⁵ M⁻¹×sec⁻¹, about 9.46×10⁵ M⁻¹×sec⁻¹, about 9.54×10⁵ M⁻¹×sec⁻¹, about 9.73×10⁵ M⁻¹×sec⁻¹, about 9.83×10⁵ M⁻¹×sec⁻¹, about 9.84×10⁵ M⁻¹×sec⁻¹, about 9.91×10⁵ M⁻¹×sec⁻¹, about 1.05×10⁶ M⁻¹×sec⁻¹, or about 1.07×10⁶ M⁻¹×sec⁻¹.

In some embodiments the antibody has a koff when associating with human HLA-G of between about 1.06×10² sec-1 and about 8.55×10⁵ sec⁻¹. In some embodiments the antibody has a koff of about 1.06×10⁻² sec⁻¹, about 1.13×10⁻² sec⁻¹, about 1.87×10⁻² sec⁻¹, about 2.13×10⁻² sec⁻¹, about 3.62×10⁻³ sec⁻¹, about 3.66×10⁻³ sec⁻¹, about 3.75×10⁻³ sec⁻¹, about 3.78×10⁻³ sec⁻¹, about 3.83×10⁻³ sec⁻¹, about 3.96×10⁻³ sec⁻¹, about 4.17×10⁻³ sec⁻¹, about 4.27×10⁻³ sec⁻¹, about 4.29×10⁻³ sec⁻¹, about 4.41×10⁻³ sec⁻¹, about 4.42×10⁻³ sec⁻¹, about 4.46×10⁻³ sec⁻¹, about 4.54×10⁻³ sec⁻¹, about 4.65×10⁻³ sec⁻¹, about 4.85×10⁻³ sec⁻¹, about 4.88×10⁻³ sec⁻¹, about 4.89×10⁻³ sec⁻¹, about 4.98×10⁻³ sec⁻¹, about 5.26×10⁻³ sec⁻¹, about 5.44×10⁻³ sec⁻¹, about 5.47×10⁻³ sec⁻¹, about 5.71×10⁻³ sec⁻¹, about 5.72×10⁻³ sec⁻¹, about 5.84×10⁻³ sec⁻¹, about 5.90×10⁻³ sec⁻¹, about 5.92×10⁻³ sec⁻¹, about 5.93×10⁻³ sec⁻¹, about 6.24×10⁻³ sec⁻¹, about 6.25×10⁻³ sec⁻¹, about 6.28×10⁻³ sec⁻¹, about 6.49×10⁻³ sec⁻¹, about 6.50×10⁻³ sec⁻¹, about 6.71×10⁻³ sec⁻¹, about 6.78×10⁻³ sec⁻¹, about 6.83×10⁻³ sec⁻¹, about 6.98×10⁻³ sec⁻¹, about 7.17×10⁻³ sec⁻¹, about 7.42×10⁻³ sec⁻¹, about 8.12×10⁻³ sec⁻¹, about 8.16×10⁻³ sec⁻¹, about 8.26×10⁻³ sec⁻¹, about 8.64×10⁻³ sec⁻¹, about 8.76×10⁻³ sec⁻¹, about 8.91×10⁻³ sec⁻¹, about 9.31×10⁻³ sec⁻¹, about 9.32×10⁻³ sec⁻¹, about 9.87×10⁻³ sec⁻¹, about 1.82×10⁻⁴ sec⁻¹, about 4.38×10⁻⁴ sec⁻¹, about 4.59×10⁻⁴ sec⁻¹, about 4.99×10⁻⁴ sec⁻¹, about 5.73×10⁻⁴ sec⁻¹, about 6.03×10⁻⁴ sec⁻¹, or about 8.55×10⁻⁵ sec⁻¹.

In some aspects, the K_{D}, kₐ, and k_{d} are determined at 25° C. In some embodiments, the KD, ka, and kd are determined by surface plasmon resonance. In some embodiments, the KD, ka, and kd are determined according to the methods described in the examples.

### 5. Inhibition of HLA-G

In some aspects, the antibody decreases the affinity of HLA-G for its ligand(s). In some aspects, the antibody disrupts the association of HLA-G with beta-2-microglobulin and/or its cognate peptide. In some aspects the antibody prevents HLA-G dimerization or oligomizeration.

In some aspects, the antibody inhibits HLA-G function on tumor cells. In some aspects, the antibody inhibits HLA-G function on immune cells. In some embodiments, the antibody blocks HLA-G interaction and/or binding to an ITIM inhibitory receptor. In some embodiments, the antibody blocks HLA-G interaction and/or binding to ILT2. In some embodiments, the antibody blocks HLA-G interaction and/or binding to ILT4. In some embodiments, the antibody blocks HLA-G interaction and/or binding to KIR2DL4.

In some embodiments, the antibody inhibits immune suppressive function. In some embodiments, the antibody inhibits HLA-G mediated suppression of NK cells. In some embodiments, the antibody inhibits HLA-G mediated suppression of cytotoxic T lymphocytes. In some embodiments, the antibody inhibits HLA-G mediated suppression of B cells. In some embodiments, the antibody inhibits HLA-G mediated suppression of neutrophils. In some embodiments, the antibody inhibits HLA-G mediated suppression of dendritic cells. In some embodiments, the antibody inhibits HLA-G mediated suppression of macrophages. In some embodiments, the antibody inhibits HLA-G mediated suppression of monocytes. In some embodiments, the antibody inhibits HLA-G mediated suppression of NK and/or T cell cytolysis and/or proliferation.

In some embodiments, the antibody prevents or inhibits inhibits HLA-G mediated suppression of phagocytosis. In some embodiments, the antibody mediates HLA-G mediated induction of T regulatory cells. In some embodiments, the antibody prevents or inhibits the generation or expansion of regulatory T cells. In some embodiments, the antibody inhibits tumor growth by antibody-dependent cellular cytotoxicity (ADCC) or phagocytosis (ADCP).

In some aspects, the decrease is about or less than a 10% decrease, about or less than a 20% decrease, about or less than a 30% decrease, about or less than a 40% decrease, about or less than a 50% decrease, about or less than a 60% decrease, about or less than a 70% decrease, about or less than an 80% decrease, about or less than a 90% decrease, or about a complete decrease. In some aspects, the increase is about or greater than a 10% increase, about or greater than a 20% increase, about or greater than a 30% increase, about or greater than a 40% increase, about or greater than a 50% increase, about or greater than a 60% increase, about or greater than a 70% increase, about or greater than an 80% increase, about or greater than a 90% increase, or a complete increase.

### 6. HLA-G Assays

In some embodiments, the antibody binds to an epitope of HLA-G. An epitope often consists of a number of contiguous amino acids such as, for example, without limitation, 5-6 amino acids. In some embodiments, the epitope comprises or consists of contiguous or non-contiguous amino acids. In some embodiments, the contiguous or non-contiguous amino acids are within a domain of HLA-G. In some embodiments, the domain comprises or consists of an alpha three domain.

In some aspects, HLA-G has a sequence identical to the amino acid sequence set forth in SEQ ID NO: 342. In some aspects, the epitope has an amino acid sequence that is within the amino acid sequence set forth in SEQ ID NO: 342. In some aspects, the epitope has an amino acid sequence that is 60%, 65%, 70%, 75%, 80%, 85%, 90%, or 95% identical to a sequence that is within the sequence set forth in SEQ ID NO: SEQ ID NO: 342.

In some aspects, the epitope comprises or consists of a contiguous or non-contiguous span of amino acids including residues 195, 197, and/or 198 of the sequence set forth in SEQ ID NO: 342. In some aspects, the epitope comprises a sequence that is identical or corresponds to residues 195, 197, and/or 198 of a sequence that is within the sequence set forth in SEQ ID NO: 342. In some aspects, the epitope has a sequence that has a 60%, 65%, 70%, 75%, 80%, 85%, 90%, or 95% identity to a sequence that is within the sequence set forth in SEQ ID NO: 342. In some aspects, the epitope has 1, 2, 3, 4, 5, 6, 7, 8, or 9 substitutions from a sequence that is within the sequence set forth in forth in SEQ ID NO: 342. In some aspects, the epitope has 1, 2 or 3 substitutions from residues a sequence that is within the sequence set forth in SEQ ID NO: 342. In some aspects, the antibody makes contact with any of the residues set forth in FIG. 9.

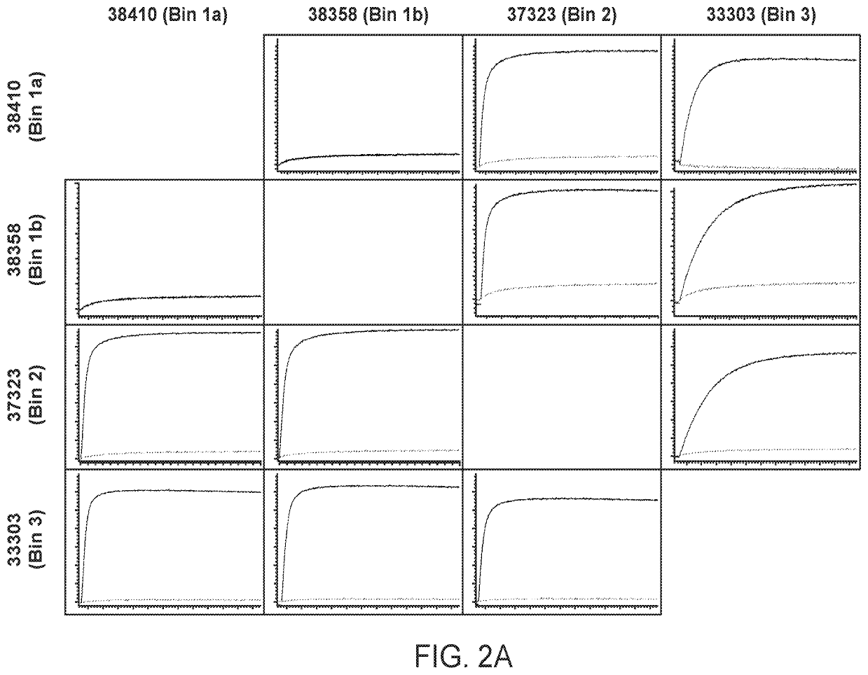

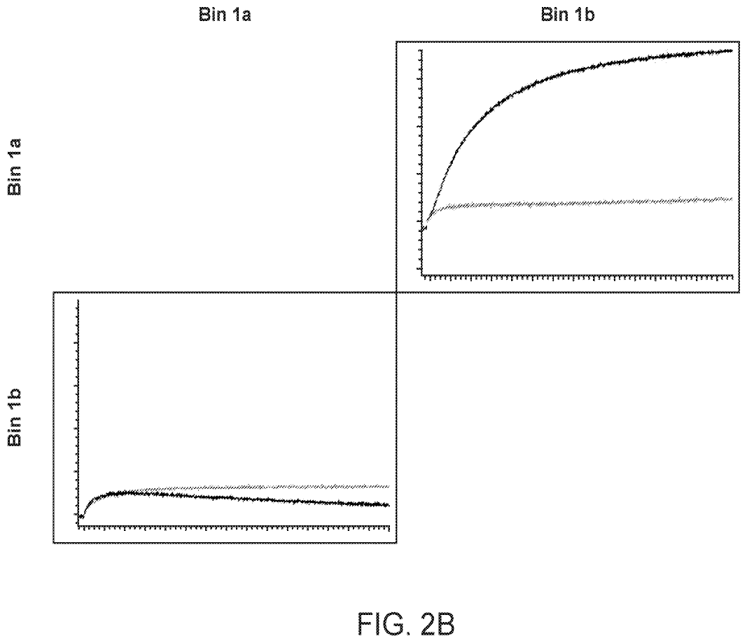

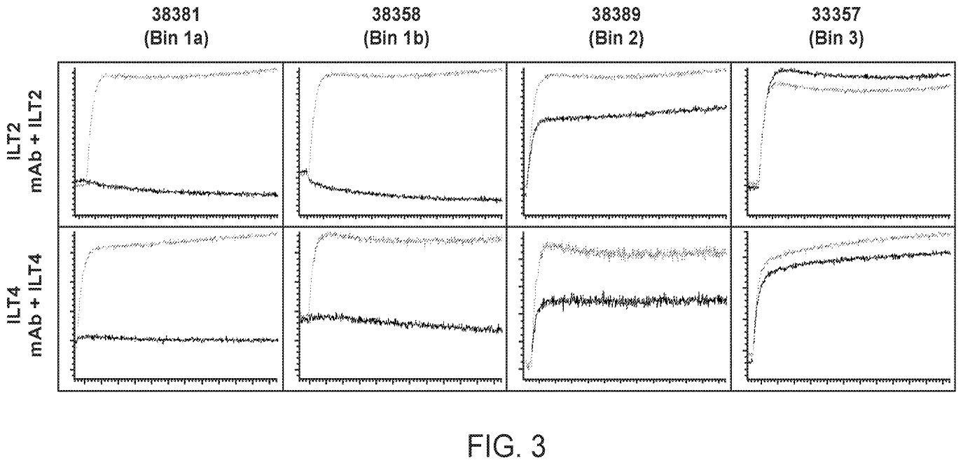

In some aspects, the antibody competes with any of the antibodies set forth herein. Competition could be, for example, without limitation, binding competition, inhibition competition, or any other form of competition. In some aspects, the antibody competes with 1, 2, 3, 4, 5, 6, 7, 8, 9, 10, 11, 12, or 13 of antibodies 38410, 38418, 38422, 38426, 38381, 38358, 37323, 38373, 38375, 38379, 38389, 33303, or 33357. In some aspects, the antibody competes with any of the antibodies set forth in FIG. 2 or FIG. 3. In some aspects, the antibody competes with 1, 2, 3, 4, 5, 6, 7, or 8 of 38373, 38375, 38379, 38418, 38422, 38426, 38410 or 38381. In some aspects, the antibody competes with 38358. In some aspects, the antibody competes with 1, 2, 3, 4, 5, 6, 7, 8, or 9 of 38373, 38375, 38379, 38418, 38422, 38426, 38410 or 38381, or 38358. In some aspects, the antibody competes with one or both of 37323 and/or 38389. In some aspects, the antibody competes with one or both of 33303 and/or 33357.

### 7. Glycosylation Variants

In some embodiments, an antibody may be altered to increase, decrease or eliminate the extent to which it is glycosylated. Glycosylation of polypeptides is typically either “N-linked” or “O-linked.”

“N-linked” glycosylation refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site.

“O-linked” glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition or deletion of N-linked glycosylation sites to the antibody may be accomplished by altering the amino acid sequence such that one or more of the above-described tripeptide sequences is created or removed. Addition or deletion of O-linked glycosylation sites may be accomplished by addition, deletion, or substitution of one or more serine or threonine residues in or to (as the case may be) the sequence of an antibody.

In certain embodiments, the antibody is glycosylated. In certain embodiments, the antibody is deglycosylated. Carbohydrates may be removed by standard techniques. In certain embodiments, the antibody is aglycosylated, for instance by expression in a system that does not glycosylate.

### 8. Fc Variants

In some embodiments, amino acid modifications may be introduced into the Pc region of an antibody provided herein to generate an Fc region variant. In some embodiments, the Fc region variant possesses some, but not all, effector functions. Such antibodies may be useful, for example, in applications in which the half-life of the antibody in vivo is important, yet certain effector functions are unnecessary or deleterious. Examples of effector functions include complement-dependent cytotoxicity (CDC) and antibody-directed complement-mediated cytotoxicity (ADCC). Numerous substitutions or substitutions or deletions with altered effector function are known in the art.

In some embodiments, the Fc is modified. In some embodiments, a hinge of an IgG1 or IgG4 antibody is modified. Modification of a hinge region stabilizes an antibody and prevents formation of unwanted bispecific antibodies. In some embodiments, the modification comprises an L234A, L235A, and/or G237A according to a Kabat numbering scheme or residues number 117, 118, and 120, respectively, wherein residues are numbered according to any of SEQ ID NO: 334. In some embodiments, the modification comprises an EU S228P or an S241P according to a Kabat numbering scheme or number 108 according to SEQ ID NO: 335. In some embodiments, an IgG Fc is engineered to modulate antibody effector function (See Wang et al., Protein Cell, 2018 January; 9 (1): 63-73), which is incorporated by reference herein in its entirety, including any drawings).

Non-limiting examples of in vitro assays to assess ADCC activity of a molecule of interest are provided in U.S. Pat. Nos. 5,500,362 and 5,821,337; Hellstrom et al., Proc. Natl. Acad. Sci. U.S.A., 1986, 83:7059-7063; Hellstrom et al., Proc. Natl. Acad. Sci. U.S.A., 1985, 82:1499-1502; and Bruggemann et al., J. Exp. Med., 1987, 166:1351-1361. Useful effector cells for such assays include peripheral blood mononuclear cells (PBMC) and Natural Killer (NK) cells. Alternatively, or additionally, ADCC activity of the molecule of interest may be assessed in vivo, using an animal model such as that disclosed in Clynes et al. Proc. Natl. Acad. Sci. U.S.A., 1998, 95:652-656.

Clq binding assays may also be carried out to confirm that the antibody is unable to bind Clq and hence lacks CDC activity. Examples of Clq binding assays include those described in WO 2006/029879 and WO 2005/100402.

Complement activation assays include those described, for example, in Gazzano-Santoro et al., J. Immunol. Methods, 1996, 202:163-171; Cragg et al., Blood, 2003, 101:1045-1052; and Cragg and Glennie, Blood, 2004, 103:2738-2743.

PcRn binding and in vivo clearance (half-life determination) can also be measured, for example, using the methods described in Petkova et al., Intl. Immunol., 2006, 18:1759-1769.

### 9. Preparation of Antibodies

### 9.1. Antigen Preparation

The HLA-G antigen to be used for production of antibodies may be intact HLA-G or a fragment of HLA-G. The intact HLA-G, or fragment of HLA-G, may be in the form of an isolated protein or expressed by a cell. Other forms of HLA-G useful for generating antibodies will be apparent to those skilled in the art.

### 9.2. Monoclonal Antibodies

Monoclonal antibodies may be obtained, for example, using the hybridoma method first described by Kohler et al., Nature, 1975, 256:495-497, and/or by recombinant DNA methods (see e.g., U.S. Pat. No. 4,816,567). Monoclonal antibodies may also be obtained, for example, using phage or yeast-based libraries. See e.g., U.S. Pat. Nos. 8,258,082 and 8,691,730.

In the hybridoma method, a mouse or other appropriate host animal is immunized to elicit lymphocytes that produce or are capable of producing antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal Antibodies: Principles and Practice 3^{rd} ed. (1986) Academic Press, San Diego, CA.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells; and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, CA), and SP-2 or X63-Ag8-653 cells (available from the American Type Culture Collection, Rockville, MD). Human myeloma and mouse-human heteromyeloma cell lines also have been described for the production of human monoclonal antibodies. See e.g., Kozbor, J. Immunol., 1984, 133:3001.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies.

### 9.3. Humanized Antibodies

Humanized antibodies may be generated by replacing most, or all, of the structural portions of a monoclonal antibody with corresponding human antibody sequences. Consequently, a hybrid molecule is generated in which only the antigen-specific variable, or CDR, is composed of non-human sequence. Methods to obtain humanized antibodies include those described in, for example, Winter and Milstein, Nature, 1991, 349:293-299; Rader et al., Proc. Nat. Acad. Sci. U.S.A., 1998, 95:8910-8915; Steinberger et al., J. Biol. Chem., 2000, 275:36073-36078; Queen et al., Proc. Natl. Acad. Sci. U.S.A., 1989, 86:10029-10033; and U.S. Pat. Nos. 5,585,089, 5,693,761, 5,693,762, and 6,180,370.

### 9.4. Human Antibodies

Human antibodies can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807. Human antibodies can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905). Human antibodies may also be generated by in vitro activated B cells (see e.g., U.S. Pat. Nos. 5,567,610 and 5,229,275). Human antibodies may also be derived from yeast-based libraries (see e.g., U.S. Pat. No. 8,691,730).

### 10. Vectors, Host Cells, and Recombinant Methods

The invention also provides isolated nucleic acids encoding anti-HLA-G antibodies, vectors and host cells comprising the nucleic acids and recombinant techniques for the production of the antibodies.

For recombinant production of the antibody, the nucleic acid encoding it may be isolated and inserted into a replicable vector for further cloning (i.e., amplification of the DNA) or expression. In some aspects, the nucleic acid may be produced by homologous recombination, for example as described in U.S. Pat. No. 5,204,244.

Many different vectors are known in the art. The vector components generally include, but are not limited to, one or more of the following: a signal sequence, an origin of replication, one or more marker genes, an enhancer element, a promoter, and a transcription termination sequence, for example as described in U.S. Pat. No. 5,534,615.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. licheniformis), Pseudomonas (P. aeruginosa), and Streptomyces. One useful E. coli cloning host is E. coli 294, although other strains such as E. coli B, E. coli X1776, and E. coli W3110 are suitable.

In addition to prokaryotes, eukaryotic microbes such as filamentous fungi or yeast are also suitable cloning or expression hosts for anti-HLA-G antibody-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K. lactis, K fragilis, K. bulgaricus K wickeramii, K. waltii, K. drosophilarum, K thermotolerans, and K marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the anti-HLA-G antibody of this invention may be cultured in a variety of media. Commercially available media such as, for example, Ham's P10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469, or WO 90/03430 and WO 87/00195 may be used.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. For example, Carter et al. (Bio/Technology, 1992, 10:163-167) describes a procedure for isolating antibodies which are secreted to the periplasmic space of E. coli. Briefly, cell paste is thawed in the presence of sodium acetate (pH 3.5), EDTA, and phenylmethylsulfonylfluoride (PMSP) over about 30 minutes. Cell debris can be removed by centrifugation.

In some embodiments, the antibody is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the antibody may be useful, for example, where the antibody accumulates in a cell as an insoluble aggregate, or where yields from periplasmic expression are low.

Where the antibody is secreted into the medium, supernatants from such expression systems are generally first concentrated using a commercially available protein concentration filter, for example, an Amicon® or Millipore® Pellcon® ultrafiltration unit. A protease inhibitor such as PMSP may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Pc domain that is present in the antibody. Protein A can be used to purify antibodies that are based on human γ1, γ2, or γ4 heavy chains (Lindmark et al., J. Immunol. Meth., 1983, 62:1-13). Protein G is useful for all mouse isotypes and for human γ3 (Guss et al., EMBO J., 1986, 5:1567-1575).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Pollowing any preliminary purification step(s), the mixture comprising the antibody of interest and contaminants may be subjected to low pH hydrophobic interaction chromatography using an elution buffer at a pH between about 2.5 to about 4.5, generally performed at low salt concentrations (e.g., from about 0 to about 0.25 M salt).

### 11. Pharmaceutical Compositions and Methods of Administration

Any of the antibodies provided herein can be provided in any appropriate pharmaceutical composition and be administered by any suitable route of administration. Suitable routes of administration include, but are not limited to, the inhalation, intra-arterial, intradermal, intramuscular, intraperitoneal, intravenous, nasal, parenteral, pulmonary, and subcutaneous routes.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

In some embodiments, the pharmaceutical composition comprises an anti-foaming agent. Any suitable anti-foaming agent may be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a cosolvent. Illustrative examples of cosolvents include ethanol, poly(ethylene) glycol, butylene glycol, dimethylacetamide, glycerin, and propylene glycol.

In some embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, and monosodium glutamate.

In some embodiments, the pharmaceutical composition comprises a carrier or filler. Illustrative examples of carriers or fillers include lactose, maltodextrin, mannitol, sorbitol, chitosan, stearic acid, xanthan gum, and guar gum.

In some embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene (glycol) succinate.

In some embodiments, the pharmaceutical composition comprises an anti-caking agent. Illustrative examples of anti-caking agents include calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6th Ed. (2009), The Pharmaceutical Press, incorporated by reference in its entirety.

In some embodiments, the pharmaceutical composition comprises a solvent. In some aspects, the solvent is saline solution, such as a sterile isotonic saline solution or dextrose solution. In some aspects, the solvent is water for injection.

In some embodiments, the pharmaceutical compositions are in a particulate form, such as a microparticle or a nanoparticle. Microparticles and nanoparticles may be formed from any suitable material, such as a polymer or a lipid. In some aspects, the microparticles or nanoparticles are micelles, liposomes, or polymersomes. In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibodies.

Purther encompassed herein are anhydrous pharmaceutical compositions and dosage forms comprising an antibody, since water can facilitate the degradation of some antibodies.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

In some embodiments, the pharmaceutical composition further comprises one or both of an antibody to an immune inhibitory receptor or ligand and/or an antibody to an immune stimulatory receptor and/or ligand. In some embodiments, the pharmaceutical composition further comprises an effective amount of at least one of the following: an anti-ILT2 antibody; an anti-ILT-4 antibody; an anti-ILT4 antibody; an anti-KIR2DL4 antibody; an anti-HLA-E antibody; an anti-NKG2A antibody; an anti-HLA-P antibody; an anti-PD-L1 antibody; an anti-PD-I antibody; an anti-CTLA4 antibody; an anti-CD38 antibody; an anti-CD73 antibody; an anti-A2A receptor antibody; an anti-A2B receptor antibody; an anti-A2A/A2B dual receptor antibody or a combination thereof; an anti-CD39 antibody; an anti-CD73 antibody; an anti-CD47 antibody; and/or a small molecule inhibitor. In some embodiments, the pharmaceutical composition further comprises an anti-Tim-3 antibody; an anti-TIGIT antibody; an anti-Vista antibody; an anti-CD94 antibody; an anti-ILT2 antibody, an anti-ILT4 antibody, an anti-PD-L1 antibody, and/or an anti-CD47 antibody; a small molecule inhibitor; a bi-specific T cell engager, CAR-T therapy, CAR-NK therapy, CAR-Macrophage therapy, engineered cell therapy, and/or adaptive T cell therapy; an oncolytic virus; a chemotherapy; and/or an ADCC capable therapy using effector competent antibodies such as anti-CD19, anti-CD20, anti-EGPR, anti-Her2, anti-SLAMF7, anti-CD52, anti-BCMA, anti-GD2, anti-CD38, and/or anti-CCR4. In some embodiments, ADCC capable therapy is enhanced ADCC capable therapy. In some embodiments, the effector is enhanced through afucosylation, point mutations, or another method.

### 11.1. Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intra-arterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the antibodies disclosed herein can also be incorporated into the parenteral dosage forms.

### 11.2. Dosage and Unit Dosage Forms

In human therapeutics, the doctor will determine the posology which he considers most appropriate according to a preventive or curative treatment and according to the age, weight, condition and other factors specific to the subject to be treated.

The amount of the antibody or composition which will be effective in the prevention or treatment of a disorder or one or more symptoms thereof will vary with the nature and severity of the disease or condition, and the route by which the antibody is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of a composition include milligram or microgram amounts of the antibody per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). In certain embodiment, the dosage of the antibody provided herein, based on weight of the antibody, administered to prevent, treat, manage, or ameliorate a disorder, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the composition or a composition provided herein administered to prevent, treat, manage, or ameliorate a disorder, or one or more symptoms thereof in a subject is 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.25 mg to 2.5 mg, 0.5 mg to 20 mg, 0.5 to 15 mg, 0.5 to 12 mg, 0.5 to 10 mg, 0.5 mg to 7.5 mg, 0.5 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

The dose can be administered according to a suitable schedule, for example, once, two times, three times, or for times weekly. It may be necessary to use dosages of the antibody outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the antibodies provided herein are also encompassed by the herein described dosage amounts and dose frequency schedules. Purther, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of an antibody or composition provided herein followed by one or more maintenance doses.

In certain embodiments, a dose of an antibody or composition provided herein can be administered to achieve a steady-state concentration of the antibody in blood or serum of the subject. The steady-state concentration can be determined by measurement according to techniques available to those of skill or can be based on the physical characteristics of the subject such as height, weight and age.

In certain embodiments, administration of the same composition may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least 1 day, 2 days, 3 days, 5 days, 10 days, 15 days, 30 days, 45 days, 2 months, 75 days, 3 months, or 6 months.

### 12. Therapeutic Applications

For therapeutic applications, the antibodies of the invention are administered to a mammal, generally a human, in a pharmaceutically acceptable dosage form such as those known in the art and those discussed above. For example, the antibodies of the invention may be administered to a human intravenously as a bolus or by continuous infusion over a period of time, by intramuscular, intraperitoneal, intra-cerebrospinal, subcutaneous, infra-articular, intrasynovial, intrathecal, or intratumoral routes. The antibodies also are suitably administered by peritumoral, intralesional, or perilesional routes, to exert local as well as systemic therapeutic effects. The intraperitoneal route may be particularly useful, for example, in the treatment of ovarian tumors.

The antibodies provided herein may be useful for the treatment of any disease or condition involving HLA-G, such as cancer, autoimmune disease, and infection.

Any suitable cancer may be treated with the antibodies provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor. In some embodiments, the cancer is selected from breast, lung, CRC, gastric, esophageal, neuroblastoma, cervical, and hematological cancers.

Any suitable autoimmune disease may be treated with the antibodies provided herein. Illustrative suitable autoimmune diseases, or diseases with an autoimmune component, include, for example, acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenia purpura (ATP), autoimmune thyroid disease, autoimmune urticarial, axonal & neuronal neuropathies, Balo disease, Behcet's disease, bullous pemphigoid, cardiomyopathy, Castleman disease, Celiac disease, Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogans syndrome, cold agglutinin disease, colitis, congenital heart block, coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis, Evans syndrome, fibromyalgia, fibrosing alveolitis, giant cell arteritis (temporal arteritis), giant cell myocarditis, glomerulonephritis, Goodpasture's syndrome, granulomatosis with polyangiitis (GPA) (formerly called Wegener's Granulomatosis), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenia purpura (ITP), IgA nephropathy, IgG4-related sclerosing disease, immunoregulatory lipoproteins, inclusion body myositis, inflammatory bowel disease, interstitial cystitis, juvenile arthritis, juvenile diabetes (Type 1 diabetes), juvenile myositis, Kawasaki syndrome, Lambert-Eaton syndrome, leukocytoclastic vasculitis, lichen planus, lichen sclerosus, ligneous conjunctivitis, linear IgA disease (LAD), lupus (SLE), Lyme disease (chronic), Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatic, polymyositis, postmyocardial infarction syndrome, postpericardiotomy syndrome, progesterone dermatitis, primary biliary cirrhosis, rimary sclerosing cholangitis, psoriasis, psoriatic arthritis, idiopathic pulmonary fibrosis, pyoderma gangrenosum, pure red cell aplasia, Raynauds phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/giant cell arteritis, thrombotic disease, thrombocytopeniac purpura (TTP), Tolosa-Hunt syndrome, transverse myelitis, type 1 diabetes, ulcerative colitis, undifferentiated connective tissue disease (UCTD), uveitis, vasculitis, vesiculobullous dermatosis, vitiligo, and Wegener's granulomatosis (now termed Granulomatosis with Polyangiitis (GPA).

Any suitable infection may be treated with the antibodies provided herein. Illustrative suitable infections include, for example, hepatitis A virus, hepatitis B virus, hepatitis C virus (HCV), human immunodeficiency virus (HIV), and other viral infections.

Some embodiments provide for treatment of a subject suffering from cancer, a chronic infection, or from an inflammatory disease, comprising the step of administering to the subject a pharmaceutical composition comprising an effective amount of any of the antibodies set forth herein. Some embodiments provide for treatment of a subject suffering from cancer, a chronic infection; or from an inflammatory disease, comprising the step of administering to the subject a pharmaceutical composition comprising an effective amount of any of the antibodies set forth herein in combination with an effective amount of another antibody set forth herein. In some embodiments, the cancer is a hematological cancer.

Some embodiments provide a method for modulating immune system function in a subject in need thereof, comprising the step of contacting a population of immune cells of the subject with a pharmaceutical composition comprising an effective amount of the antibody as set forth herein, under conditions such that the immune system is modulated. Some embodiments provide a method for modulating immune system function in a subject in need thereof, comprising the step of contacting a population of immune cells of the subject with an effective amount of any of the antibodies set forth herein in combination with an effective amount of another antibody set forth herein. In some embodiments, the antibody comprises a bispecific antibody or a complexing antibody.

In some embodiments, the bispecific antibody or the complexing antibody is administered in an amount sufficient to achieve 1, 2, 3, 4, 5, 6, 7, or 8 of the following in the subject:
a) inhibition of immune suppression;
b) reduction of levels of regulatory T cells;
c) increase an activity of myeloid cells;
d) increase in activity of cytotoxic T lymphocytes, NK cells, B cells, neutrophils, monocytes, macrophages, and/or dendritic cells;
e) increase in phagocytic activity;
f) inhibition of metastasis;
g) inhibition of tumor growth; and/or
h) induction of tumor regression.

In some embodiments, the bispecific antibody binds HLA-G and HLA-E.

In some embodiments, the method for modulating immune system function in a subject in need thereof further comprises administering chemotherapy, administering radiation therapy, and/or administering one or more additional therapeutic agents. In some embodiments, the one or more additional therapeutic agents comprise one or more immunostimulatory agents. In some embodiments, the one or more immunostimulatory agents comprise an antagonist to an inhibitory receptor of an immune cell. In some embodiments, the inhibitory receptor is at least one of ILT2, ILT3, ILT4, KIR2DL4, CTLA-4, PD-1, CD39, CD73, PD-L1, PD-L2, LAG-3, TIGIT, β₇-H3, β₇-H4, Tim3, neuritin, BTLA, CECAM-1, CECAM-5, VISTA, LAIR1, CD160, 2β₄, TGP-B, NKG2A, and/or a Killer-cell immunoglobulin-like receptor (KIR).

In some embodiments, the one or more immunostimulatory agents comprise an agonist of a co-stimulatory receptor of an immune cell. In some embodiments, the co-stimulatory receptor comprises one or more of OX40, CD2, CD27, ICAM-1, LPA-1, ICOS (CD278), 4-1BB (CD137), GITR, CD28, CD30, CD40, BAPPR, HVEM, CD7, LIGHT, NKG2C, SLAMP7, NKp30, NKp46, NKp80, CD160, and/or a CD83 ligand.

In some embodiments, the one or more immunostimulatory agents comprise a cytokine. In some embodiments, the cytokine is at least one of IL-1, IL-2, IL-5, IL-7, IL-10, IL-12, IL-15, IL-21, and/or IL-27. In some embodiments, the one or more immunostimulatory agents comprise an oncolytic virus. In some embodiments, the oncolytic virus comprises one or more of the oncolytic virus is a Herpes simplex virus, a Vesicular stomatitis virus, an adenovirus, a Newcastle disease virus, a vaccinia virus, or a maraba virus. In some embodiments, the one or more immunostimulatory agents comprise a chimeric antigen engineered T cell. In some embodiments, immunostimulatory agents comprise a bi- or multi-specific T cell directed antibody. In some embodiments, the one or more immunostimulatory agents comprises or consists of an ADCC competent antibody that may target CD19, CD20, EGPR, Her2, SLAMP7, CD52, BCMA, GD2, CD38, or CCR4. In some embodiments, the ADCC competent antibody is effector enhanced through afucosylation, point mutations, or otherwise.

In some embodiments, the one or more immunostimulatory agents comprise a bi-specific T cell engager and/or CART therapy, CAR-NK therapy, CAR-macrophage therapy, adoptive T cell therapy.

### 13. Diagnostic Applications and Diagnostic Methods

In some embodiments, the antibodies provided herein are used in diagnostic applications and/or diagnostic methods. For example, an anti-HLA-G antibody may be useful in assays for HLA-G protein. In some aspects, the antibody can be used to detect the expression of HLA-G in various cells and tissues. In some embodiments, the antibody can be used to detect the soluble HLA-G in fluids. In some embodiments, the fluids comprise or consist of blood, serum, ascites, and/or other fluids. The assays may be useful, for example, evaluating cancer and autoimmune disease.

In some embodiments, the diagnostic method comprises or consists of detecting tumor expressed HLA-G. In some embodiments, the diagnostic method comprises or consists of detecting soluble HLA-G. In some embodiments the detection method comprises or consists of detecting HLA-G expression on immune cells.

In some diagnostic applications, the antibody may be labeled with a detectable moiety. Suitable detectable moieties include, but are not limited to radioisotopes, fluorescent labels, and enzyme-substrate labels. In another embodiment of the invention, the anti-HLA-G antibody need not be labeled, and the presence thereof can be detected using a labeled antibody which specifically binds to the anti-HLA-G antibody.

### 14. Affinity Purification Reagents

The antibodies of the invention may be used as affinity purification agents. In this process, the antibodies may be immobilized on a solid phase such a resin or filter paper, using methods well known in the art. The immobilized antibody is contacted with a sample containing the HLA-G protein (or fragment thereof) to be purified, and thereafter the support is washed with a suitable solvent that will remove substantially all the material in the sample except the HLA-G protein, which is bound to the immobilized antibody. Finally, the support is washed with another suitable solvent, such as glycine buffer, pH 5.0, that will release the HLA-G protein from the antibody.

### 15. Kits

In some embodiments, an anti-HLA-G antibody provided herein is provided in the form of a kit, i.e., a packaged combination of reagents in predetermined amounts with instructions for performing a procedure. In some embodiments, the procedure is a diagnostic assay. In other embodiments, the procedure is a therapeutic procedure.

In some embodiments, the kit further comprises a solvent for the reconstitution of the anti-HLA-G antibody. In some embodiments, the anti-HLA-G antibody is provided in the form of a pharmaceutical composition.

### Examples

### Example 1: Selection of HLA-G Antibodies

HLA-G antibodies were selected from a synthetic library of human antibodies presented on the surface of yeast cells in IgG format, as generally described, e.g., in WO2009036379; WO2010105256; WO2012009568; and Xu et al., Protein Eng. Des. Sel., 2013, 26:663-670 (each incorporated by reference in its entirety), and more specifically as provided below. The sequences and characteristics of the antibodies isolated from the recombinant library are provided in Table S.

Eight naïve human synthetic yeast libraries each of ˜10E+09 diversity were propagated as described in WO2009036379; WO2010105256; WO2012009568; and Xu et al., Protein Eng. Des. Sel., 2013, 26:663-670; each incorporated by reference in its entirety. For the first two rounds of selection, a magnetic bead sorting technique utilizing the Miltenyi MACS® system was performed, as described in Siegel et al., J. Immunol. Meth., 2004, 286:141-153. The following rounds of selection were performed using flow cytometry-based sorting. For all rounds of selection, the antigen was biotinylated human HLA-G, decreasing concentrations of antigen were used in each subsequent round of selection. In addition to selection on antigen, some rounds of selection were employed to reduce the number of non-specific binders utilizing soluble membrane proteins from CHO cells (see WO2014179363 and Xu et al., Protein Eng. Des. Sel., 2013, 26:663-670, each incorporated by reference in its entirety). In addition to the CHO cell proteins, deselections against recombinant HLA-A/B/C proteins were performed to maintain specific binding to HLA-G. After the final round of sorting, yeast were plated and individual colonies were picked for characterization and for nomination of clones for affinity maturation.

Antibody variable domains of interest were synthesized, with codon optimization to maximize transient expression in host cells. The variable regions were cloned into expression vectors containing human immunoglobulin constant domains and their sequence confirmed. Antibody heavy and light chain vector pairings were transfected into Expi293 cells using the Expifectamine system (Invitrogen). Transient cultures were harvested on day 4 and clarified cell culture supernatant IgG titer was estimated using Bio-Layer Interferometry (BLI) using Octet (ForteBio) alongside standards. Antibodies were subsequently purified on a Protein A column and eluted using low pH glycine. Purified antibody samples were then buffer-exchanged or dialyzed into downstream assay-compatible buffers.

Antibody purity was assessed by running samples on SDS-PAGE and on an analytical size exclusion chromatography column.

Light Chain Shuffling: Heavy chain plasmids were extracted from naïve outputs (described herein) and transformed into a pre-made naïve light chain library with a diversity of 10E+06. Selections were performed as described above with one round of MACS sorting and three rounds of FACS sorting using decreasing amounts of biotinylated HLA-G antigen for respective rounds.

### Example 2: Affinity Maturation

Optimization of naïve clones was carried out utilizing four maturation strategies; diversification of CDR-H1 and CDR-H2; diversification of CDR-H3; diversification of CDR-L1, L2 and L3; shuffling of diversified heavy and light chains.

CDR-H1 and CDR-H2 Selection: The CDR-H3s from clones selected from light chain batch diversification, light chain diversification, and naive discovery efforts were independently recombined into premade libraries with CDR-H1 and CDR-H2 variants of a diversity of >10E+8. Selections were performed using HLA-G antigen. Affinity pressures were applied by using decreasing concentrations of antigen and HLA-G specificity was maintained with deselections against HLA-A/B/C antigens.

CDR-H3 Selection: After characterization of CDR-H1 and CDR-H2 variants, clones with binding to HLA antigens outside of HLA-G were removed. Chemical liabilities were also removed from the variable regions when applicable. The remaining clones obtained from the CDR-H1 and CDR-H2 selection procedure were subject to additional rounds of affinity maturation via walking dimer mutagenesis of the CDR-H3. Selections were performed using HLA-G as antigen generally as described above, except for employing FACS sorting for all selection rounds.

CDR-L1, L2, L3 Selection: Clones obtained from the CDR-H1 and CDR-H2 selection procedure were subject to additional rounds of affinity maturation via mutagenesis of the light chain. The CDR-L1 and CDR-L2 diversity was derived from a pre-made library while CDR-L3 diversity was derived from walking monomer mutagenesis. Selections were performed using HLA-G as antigen, starting with one round of MACS followed by three rounds of FACS in the CDR-L1, L2, L3 process described here.

Diversified Heavy Chain and Light Chain Shuffling: Outputs from CDR-H3 diversification and CDR-L1, L2, L3 diversification described above were recombined and selections were performed using HLA-G as antigen generally as described above, except for employing FACS sorting for all selection rounds.

### Example 3: Binding Affinity of Anti-HLA-G Antibodies to Recombinant HLA-G

Binding kinetics were measured using the Octet Red96 system (Pall ForteBio) at 30° C. in running buffer (1× Pall ForteBio Kinetics Buffer diluted into 1×PBS pH 7.4). In brief, 0.16 μg/ml of biotinylated recombinant HLA-G/β2m/peptide heterotrimer was loaded onto streptavidin (SA) biosensors to a binding response of approximately 0.25 nm. After a short baseline step in running buffer, the biosensors were exposed to varying concentrations (1.5, 3.0, or 30 nM) of full-length anti-HLA-G mAbs for the association step. Dissociation of the complex was monitored upon dipping the sensors to running buffer once again for up to 30 min. Data was processed using ForteBio Octet DataAnalysis software (version 10) with background subtraction of biosensors without HLA-G. A 1:1 Langmuir binding model was fit to each sensorgram to obtain association and dissociation rates via local-full or local-partial fitting. K_{D} was calculated from the ratio of k_{d} to kₐ. Monovalent affinities were obtain using identical methods but Fabs were used instead of IgGs.

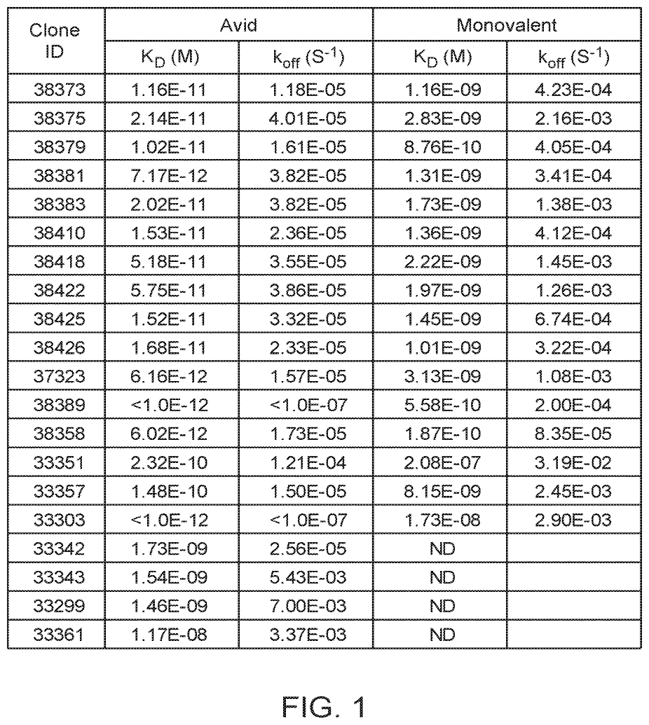

FIG. 1 provides a table showing avid and monovalent affinities of anti-HLA-G antibodies to recombinant HLA-G protein. Data shown in FIG. 1 had R²>0.98. ND=not determined using Fabs. The avid K_{D} values ranged from 11.7 nanomolar to sub picomolar with off-rates (k_{off}) ranging from 0.007 sec⁻¹ to the Octet off-rate limit (1.0×10⁻⁷ sec⁻¹). Monovalent K_{D} values ranged from 0.187 nM to 208 nM, but monovalent affinities for clones that had weaker avid affinities were not determined.

### Example 4: Antibodies that Bind to HLA-G and Bin Separately from Other Antibodies

Epitope binning assays to recombinant HLA-G heterotrimer were determined using the Octet Red96 system (Pall ForteBio) at 30° C. in running buffer (1×Pall ForteBio Kinetics Buffer diluted into 1×PBS pH 7.4). Briefly, 0.16 μg/ml of biotinylated recombinant HLA-G/β₂m/peptide heterotrimer was loaded onto streptavidin (SA) biosensors to a binding response of approximately 0.25 nm. After a short baseline step in running buffer, the biosensor was dipped into 20 μg/ml of each mAb and continued until the binding response to HLA-G reached saturation. For the competing association step, equimolar concentrations of each mAb to the saturating mAb was used.

Epitope binning was performed in each direction (every mAb was used as both the saturating mAb (association step 1) as well as the competing mAb (association step 2)). Self-blocking sensorgrams are shown in gray. Non-self sensograms are shown in black; black sensorgrams represent the pairwise binning mAb. Sensorgrams from only the second association step were shown in FIG. 2.

FIG. 2A provides biolayer interferometry sensorgrams showing tested antibodies that bind HLA-G can be divided into three distinct chemical bins, with one chemical bin being further divided. Gray sensorgrams indicate self-blocking; black sensorgrams indicate pairwise binning. The y-axis show saturating antibody; the x-axis shows competing antibody.

FIG. 2B provides biolayer sensorgrams showing antibodies (Fabs) that bind HLA-G and can be separated into two sub-bins from a broader bin 1. Bin 1a Fabs do not block competing bin 1b Fabs. However, bin 1b Fabs block bin 1a Fabs. As described above, gray sensorograms indicated self-blocking and black sensorograms represent pairwise binning. The y-axis represents the saturating Fab and the x-axis represents the competing Fab.

### Example 5: Anti-HLA-G Antibodies that Block HLA-G Interaction/Binding to Recombinant ILT2 and ILT4

Blocking assays between recombinant HLA-G heterotrimer and recombinant extracellular domain of ILT2 or ILT4 proteins was determined using the Octet Red96 system (Pall ForteBio) at 30° C. in running buffer (1× Pall ForteBio Kinetics Buffer diluted into 1×PBS pH 7.4). Briefly, 0.16 μg/ml of biotinylated recombinant HLA-G/β₂m/peptide heterotrimer was loaded onto streptavidin (SA) biosensors to a binding response of approximately 0.25 nm. After a short baseline step in running buffer, the biosensor was dipped into at least 200 nM of each mAb (up to 0.5 μM for weakly binding mAbs) and continued until the binding response to HLA-G reached saturation. A second association step immediately followed by dipping into wells containing dimerized ILT2 or ILT4. Dimers were obtained via pre-incubation of the His-tagged ILT2 or ILT4 with anti-His mAb (MAβ050, R&D Systems). ILT2/ILT4 binding response to mAb-saturated HLA-G (black sensorgrams) was compared to HLA-G in the absence of anti-HLA-G mAb (gray sensorgrams).

Sensorgrams from the second association step are shown in FIG. 3. The provided sensorgrams show antibodies that bind and fully block HLA-G interaction with ILT2 and ILT4, for example, the bin1a and bin1b antibodies 38381 and 38358, respectively. As indicated, gray sensorgrams indicate ILT2 or ILT4 binding in the absence of antibody; black sensorgrams represent ILT2 or ILT4 binding in the presence of antibody.

FIG. 3 also provides biolayer interferometry sensorgrams showing antibodies that bind but do not block HLA-G interaction with ILT2 or ILT4, for example, the bin 3 antibodies such as 33357. Gray sensorgrams indicate ILT2 or ILT4 binding response in the absence of antibody; black sensorgrams indicate ILT2 or ILT4 binding in the presence of antibody.

The provided sensorgrams also show antibodies that bind and block HLA-G interaction with ILT2 or ILT4 to an intermediate effect, such as the bin 2 antibody 38389.

### Example 6: Anti-HLA-G Antibodies Bind to HLA-G Naturally Expressed on JEG-3 Cells

JEG-3 parentals or HLA-G-knock out (JEG-3 KO) cells were incubated with 100 nM of anti-HLA-G antibodies for 2 hours at 4° C. in wash buffer (Phosphate Buffered Saline (PBS), 2% Fetal bovine serum (FBS) and 2 mM EDTA). Cells were then washed in wash buffer and incubated with an APC conjugated secondary antibody (goat anti-human IgG Kappa) at a 1:500 dilution in wash buffer for 30 minutes at 4° C. Cells were subsequently washed and resuspended in 1% paraformaldehyde/wash buffer followed by sample acquisition using a BD Fortessa X-20 flow cytometer (Becton Dickinson). Sample data was exported as PCS files and analyzed using PlowJo software v₁₀ (Tree Star, Inc.).

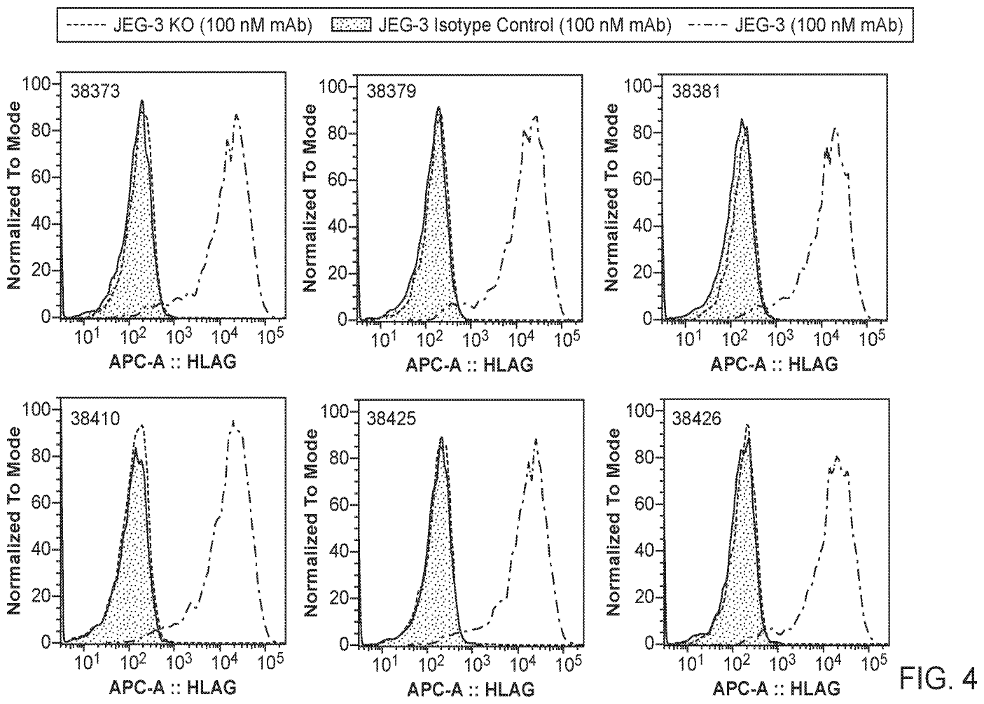

The results are shown in FIG. 4. The shaded histogram represents binding of an isotype control to JEG-3 cells at the same concentration.

### Example 7: Anti-HLA-G Antibodies Abrogate Suppression Mediated Through HLA-G-ILT2 or ILT4 Interaction in NKL Killing Assay

Inhibition of NKL cytotoxicity via the ILT2-HLA-G interaction or ILT4-HLA-G interaction is abrogated by anti-HLA-G antibodies. NKL (Effector) cells were co-incubated with 721.221 (Target) cells +/−HLA-G expression at an E:T ratio of 4:1 overnight at 37° C. 721.221s were pre-incubated with 200 nanomolar (nM) Fab fragments or full-length intact anti-HLA-G antibodies for 60 minutes at 37° C. Prior to antibody pre-treatment, 721.221 cells were labelled with 1:1000 CellTrace Violet (CTV) (Invitrogen) in 37° C. PBS.

After overnight co-incubation, cells were washed once in 4° C. wash buffer (Phosphate Buffeed Saline, 2% FBS, 2 mM EDTA) and then stained with 1:2000 Fixable Viability Dye eFluor™ 780 (Invitrogen) 30 minutes at 4° C. Cells were washed again in 4° C. wash buffer resuspended in wash buffer and analyzed for percent dead 721.221 cells by flow cytometry analysis on a BD Celesta flow cytometer. Percent of dead 721.221 cells was determined by gating on CTV positive cells, then dividing the Fixable Viability Dye positive portion of these cells by the total CTV count.

Antibody MEM-G/9 is a commercially available antibody (See, Fournel et al., Tissue Antigens 200:55: 510-518 (1999), which is incorporated by reference in its entirety herein, including any drawings).

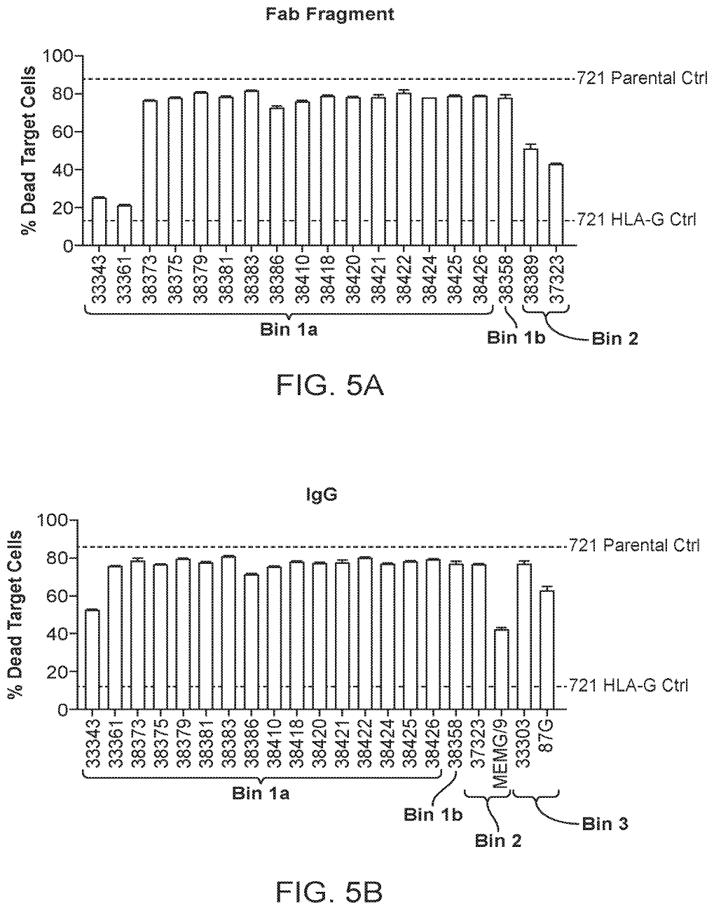

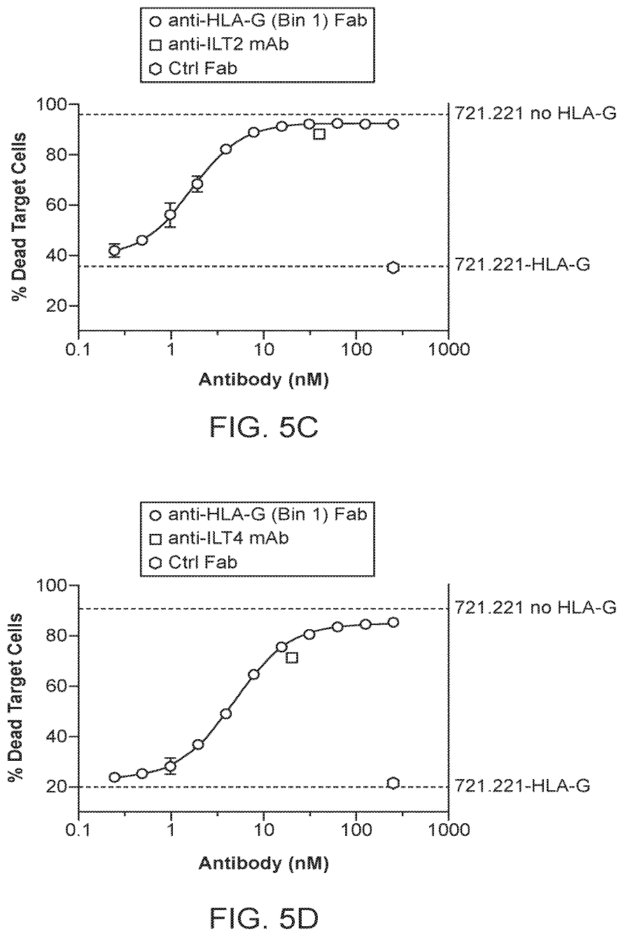

FIG. 5A (Fab fragments) and FIG. 5B (intact IgG) show the effect on NKL cells that endogenously express ILT2. The percent of dead target cells is shown for each antibody (indicated as a unique clone number in the figure) in addition to control values representing minimum and maximum killing in the presence and absence of HLA-G, respectively. As can be seen from FIG. 5, Bin 1 antibodies show the best blockade of all the antibodies tested.

FIG. 5C demonstrates the dose response of a specific Bin 1 anti-HLA-G Fab fragment for blockade of HLA-G-mediated suppression of NKL killing through endogenously expressed ILT2.

FIG. 5D demonstrates the dose response of a specific Bin 1 anti-HLA-G Fab fragment for blockade of HLA-G-mediated suppression of NKL killing through engineered expression of ILT4 (engineered cell line modified by knocking down ILT2 and retrovirally transduced to express ILT4).

### Example 8: Fab Fragments of Anti-HLA-G Antibodies Reverse HLA-G Mediated Suppression of Macrophage Phagocytosis, NK Cell Cytotoxicity, and CD8⁺ T Cell Activity

CD14+ enriched cells were differentiated into adherent macrophages by incubating at 37° C., 5% CO₂ for 7 days in complete RPMI (cRPMI) with recombinant human M-CSP. Cells were harvested after 7 days, washed, and resuspended in cRPMI. Cells were plated in a 96-well round bottom plate at 50,000 cells/well in 50 μl cRPMI and incubated at 37° C., 5% CO₂, before being combined with the target cells.

The target cells, consisting of either 721.221 parental cells or 721.221-HLA-G, were stained with CTV (1:1000) at 37° C. in PBS, washed and plated in cRPMI at 25,000 cells per well. Cells were subsequently incubated with anti-CD47 antibody with or without anti-HLA-G Fabs for 1 hour at 37° C., 5% CO₂. The mixture of 721.221 and antibody was then combined with macrophages and incubated for either 2 hours or overnight at 37° C., 5% CO₂. The final ratio of macrophage to 721.221 target cell was 2:1. After either the 2 hour or overnight incubation, cells were blocked with wash buffer/BD Fc Block™/2% mouse serum for 20 minutes and then stained with anti-CD11 b and live/dead discrimination dye for 20 minutes. Cells were then washed, resuspended in wash buffer and analyzed on the BD Fortessa flow cytometer. Sample data was exported as FCS files and analyzed using FlowJo software v10.

In the 2-hour format assay, macrophages were assayed for the presence of CTV as a readout of phagocytic uptake of CTV⁺ target cells. Live cells were gated on CD11b⁺/CTV⁺ and reported as a percent of total live macrophages. For the overnight assay, macrophage killing activity was assessed by measuring absolute counts of remaining live CTV⁺ cells per well.

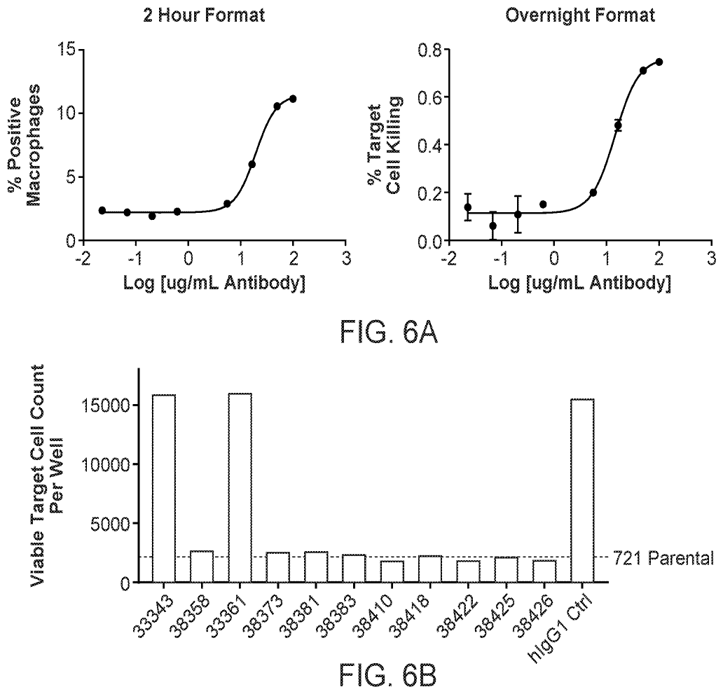

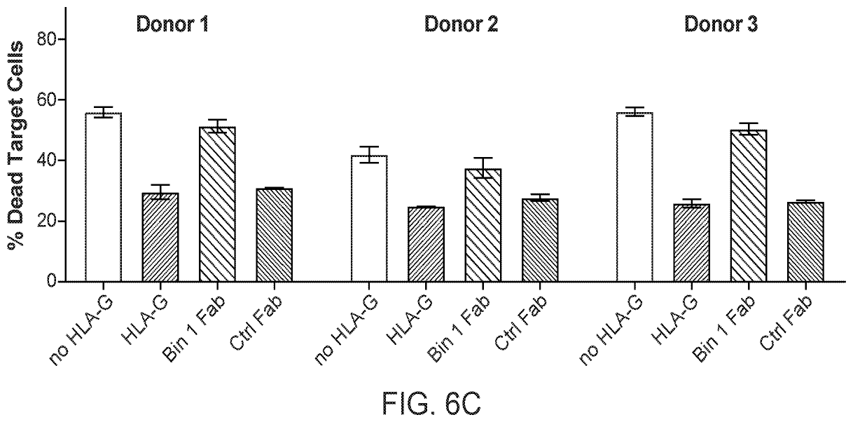

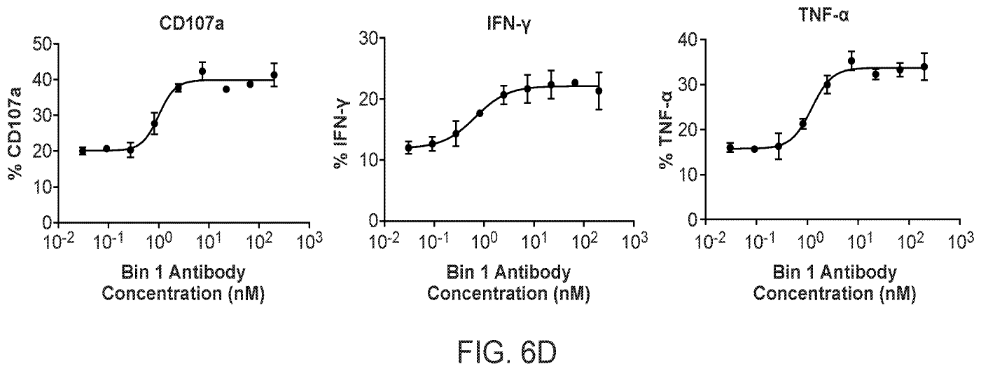

FIG. 6A compares results between the two hour and overnight assay formats using an anti-HLA-G antibody (Fab fragment). The overnight assay achieves similar results as the two hour assay that directly measures phagocytosis of target cells. FIG. 6B shows the results of Fab fragments of each anti-HLA-G antibody that were tested in the overnight assay to determine the level of restored phagocytic activity.

To demonstrate inhibition of primary human NK cell cytotoxicity is abrogated by an anti-HLA-G antibody, HLA-G expressing target cells (721.221-HLA-G) were pre-incubated with a representative Bin 1 antibody (Fab fragment) and then co-cultured with human primary NK (Effector) cells from 3 separate donors at an E:T ratio of 4:1 overnight at 37° C. Prior to antibody pre-treatment, 721.221 cells were labelled with 1:1000 CellTrace Violet (CTV) (Invitrogen) in 37° C. PBS.

After overnight co-incubation, cells were washed once in 4° C. wash buffer (Phosphate Buffered Saline, 2% PBS, 2 mM EDTA) and then stained with 1:2000 Fixable Viability Dye eFluor 780 (Invitrogen) for 30 minutes at 4° C. Cells were washed, resuspended in wash buffer, and analyzed for percent dead 721.221 cells by flow cytometry analysis on a BD Celesta flow cytometer. Percent of dead 721.221 cells was determined by gating on CTV positive cells, then dividing the Fixable Viability Dye positive portion of these cells by the total CTV count.

The results are shown in FIG. 6C. The percent of dead target cells is shown for the Bin 1 antibody (Fab) in addition to control values representing co-cultures with 721.221 target cells with and without HLA-G expression (noted as HLA-G and no HLA-G in figure, respectively). An isotype control Fab (Ctrl Fab) was used as a negative control.

To demonstrate an anti-HLA-G antibody reverses HLA-G mediated suppression of cytotoxic primary CD8⁺ T cell function, human CD8⁺ T cells treated with CD3/CD28 activator (ImmunoCult™, Stem Cell Technologies) for 1 hour were mixed with antibody pre-treated 721.221 cells (with and without expression of HLA-G) and co-cultured overnight at 37° C. After overnight co-incubation, Monensin Solution (ThermoFisher) and eFluor 450-CD107a (ThermoFisher) were added; the final amount of each reagent was 0.5 μg/mL antibody and 2 μM Monensin. The assay was incubated for 4 hours.

After the incubation, cells were washed and stained over several steps which included the following: (1) live/dead cell staining using Fixable Viability Dye eFluor 780 (ThermoFisher); (2) Fc blocking with staining buffer (Phosphate Buffered Saline, 2% FBS, 2 mM EDTA) containing 6% normal mouse serum and 5 μL Human TruStain FcX™ per sample (Biolegend) for 15 minutes at 4° C.; (3) CD8 and ILT2 staining with Alexa Fluor 700-CD8 (Biolegend, Clone SK1) and PE-CD85j/ILT2 (Biolegend, Clone HP-F1, ThermoFisher); Fixation and permeabilization using fix/perm reagent (ThermoFisher); (5) Intracellular staining with Brilliant Violet 711-IFN-γ (Biolegend, Clone 4S.B3) and APC-TNF-α (Biolegend, Clone MAb11) diluted in Permeabilization Buffer (ThermoFisher) for 20 minutes at 4° C. After the staining steps, cells were washed, centrifuged, and resuspended in staining buffer followed by sample acquisition on a BD Fortessa X-20 flow cytometer using the high throughput sampler.

FCS data was analyzed using FlowJo version 10 analysis software (FlowJo, LLC). Gates were made to select single cells, live cells, and the cell type of interest. CD8⁺ T cell populations were defined based on CD8⁺ILT2⁺or CD8⁺ILT2⁻. To determine the percent of cells positive for CD107a, IFN-γ, or TNP-α, gating was performed on each CD8⁺ T cell population and determined using fluorescence minus one cells and unstimulated cells.

Results depicted in FIG. 6D demonstrate that blockade of HLA-G with an anti-HLA-G antibody (representative Bin 1 clone) restored CD107a, IFN-γ, and TNP-α expression in CD8⁺ILT2⁺ T cells in a dose-dependent manner. There was no effect from HLA-G target cell expression or anti-HLA-G antibody treatment on CD8⁺ILT2⁻ cells (data not shown).

### Example 9: Anti-HLA-G Antibodies that are Selective Binders to HLA-G and do not Bind to Classical HLA Class I Antigens

Binding of antibodies to HLA antigens (97 different antigens spanning HLA-A, -B, and -C haplotypes) was determined using the LABScreen single antigen class I-combi assay (One Lambda). Antibodies at concentrations greater than 300 nM were incubated with single antigen LABScreen beads in binding buffer (Phosphate Buffered Saline, 10% FBS, 2 mM EDTA) for 45 minutes in the dark at room temperature with gentle shaking. Beads were washed twice with 200 μL of 1X LABScreen wash buffer in a 96-well V-bottom plate by centrifugation at 1500 g and removal of buffer by aspiration. Beads were then incubated with a PE-conjugated secondary antibody (polyclonal goat anti-human or anti-mouse in 1X wash buffer) for 30 minutes at room temperature in dark with gentle shaking. After incubation, beads were washed twice, resuspended in 90 μL of wash buffer, and then transferred to a 96-well flat bottom plate. Acquisition was performed on a Luminex 100 instrument and data output as a CSV file. Results for each single HLA antigen bead group were tabulated from median fluorescent intensity values.

Antibody 87G is a commercially available antibody and represents a blocking antibody (See, Odum et al., Eur. J. Immunol. 22:2121-2131 (1991) and Lee et al., Immunity, 591-600 Volume 3, Issue 5 (1995), each of which is incorporated by reference in its entirety herein, including any drawings).

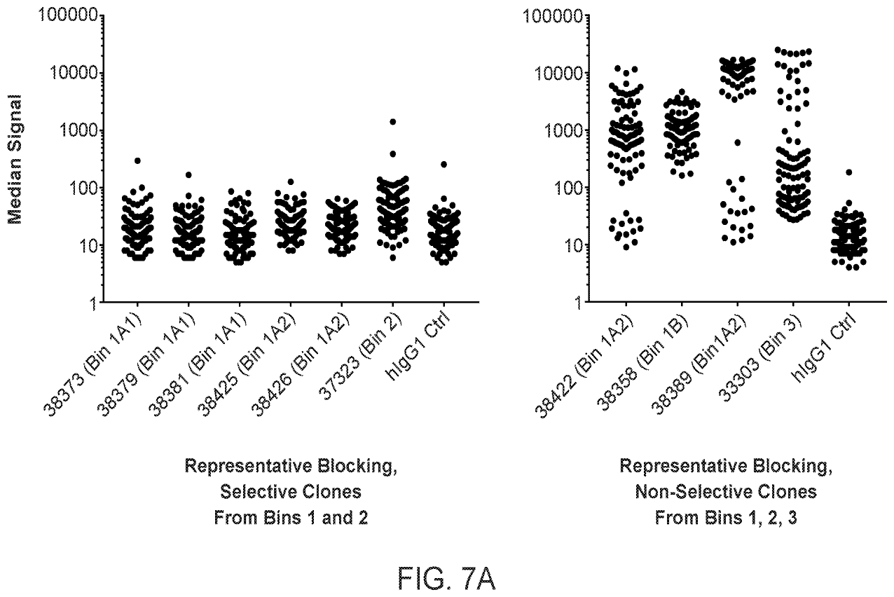

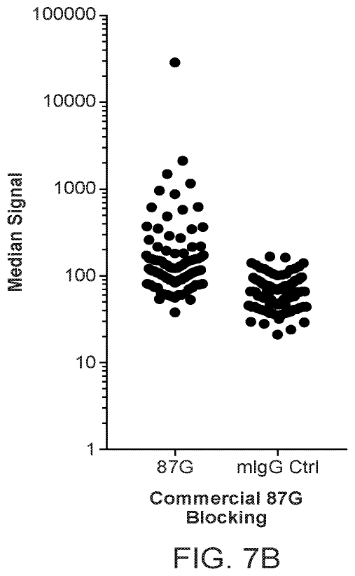

The results are shown in FIG. 7. Binding values (median fluorescent signal) to each single HLA antigen are shown for each anti-HLA-G antibody.

### Example 10: Anti-HLA-G Antibodies that do not Bind to Classical HLA Class Antigens Expressed at Physiological Levels on B-LCL Cells

Binding of antibodies to physiological levels of native HLA class Ia proteins was assessed by flow cytometry on a collection of twenty-eight B-LCL cell lines (shown in Table X). Cells were obtained from the International Histocompatibility Working Group cell line collection that holds a repository of B-LCL cell lines which have been HLA-typed. The cells were maintained in supplemented RPMI-1640 media containing 15% fetal bovine serum.

For antibody binding, anti-HLA antibodies were first conjugated to DyLight 650 (Thermo Fisher) by primary amine labeling via the NHS-ester moiety. Unconjugated dye was removed by flowing the antibody preparation through dye removal resin. Conjugation efficiency was between 1 to 3 moles of DyLight 650 per mole of antibody. B-LCL cell lines (ranging from 10,000 to 100,000 cells per well) were incubated with 300 nM of labeled antibodies in cold PBS containing 10% FBS and human Fc block (BD Biosciences). After 2 hours at 4° C., cells were washed twice in cold wash buffer (Phosphate Buffered Saline, 2% FBS, 2 mM EDTA) followed by sample acquisition on a BD FACS Celesta instrument. To assess overall HLA class I expression, a pan HLA class I antibody, clone W6/32, conjugated to FITC was incubated separately with each individual cell line. All cell lines were strongly positive for class I expression (data not shown). Data was exported as FCS files and analyzed using FlowJo software v10. Gating was performed on live singlet cells using DAPI for live-dead discrimination. Geometric MFI is shown for each B-LCL cell line.

TABLE X

| | Alternate | | Ethnic/ | Workshop | | | | | | |
|---|---|---|---|---|---|---|---|---|---|---|
| IHW ID | ID | Sex | Population Group | Involvement | HLA A* | | HLA B* | | HLA Cw* | |
| IHW09367 | LCK | | Asian | 13 | 0203 | 1102 | 4601 | 380201 | 0702 | 1202 |
| IHW09458 | FH70EY | M | | 13 | 3002 | 6802 | 0801 | 5301 | 0401 | 07 |
| IHW09383 | FH9 | M | Other | 13 | 2402 | 3303 | 4801 | 44032 | 0801 | 0701 |
| IHW09397 | DUG150 | | Unknown | 13 | 02 | 680101 | 5802 | 4501 | 0602 | 1601 |
| IHW09057 | TEM | M | Jewish | 10, 12 | 6601 | | 3801 | | 1203 | |
| IHW09010 | AMA | M | Algerian | 10, 12 | 6802 | | 5301 | | 0401 | |
| IHW09436 | FH48 | M | Caucasian | 13 | 2402 | 0201 | 4427 | 070201 | 070201 | 0704 |
| IHW01167 | 1413-1090 | M | Caucasian | CEPH | 680102 | 0201 | 0801 | 4402 | 0701 | 0501 |
| IHW01075 | 1344-8354 | F | Caucasian | CEPH | 2402 | 0301 | 4001 | 5101 | 030401 | 0102 |
| IHW01185 | 1416-1337 | M | Caucasian | CEPH | 0205 | 2501 | 4901 | 4402 | 0701 | 0501 |
| IHW09431 | FH43 | M | American Black | 13 | 3001 | 3301 | 5301 | 8101 | 04 | 08 |
| IHW01124 | 1362-8563 | M | Caucasian | CEPH | 0201 | 2902 | 3501 | 4403 | 0401 | 1601 |
| IHW01133 | 1362-8572 | F | Caucasian | CEPH | 2902 | 2601 | 4403 | 4402 | 1601 | 0501 |
| IHW01092 | 1346-8603 | M | Caucasian | CEPH | 0201 | 1101 | 0702 | 3501 | 0702 | 0401 |
| IHW09371 | ISH4 | | Asian | 13 | 0218 | 1101 | 1501 | 4601 | 0102 | 040101 |
| IHW09433 | FH45 | M | Asian | 13 | 1101 | 02 | 3802 | 4101 | 0702 | 17 |
| IHW09380 | FH6 | M | Hispanic | 13 | 2402 | 2901 | 2702 | 0705 | 1505 | 020202 |
| IHW09411 | FH29 | F | Hispanic | 13 | 0201 | 2902 | 1516 | 440301 | 1402 | 1601 |
| IHW09401 | TER-259 | | USA White/American Indian | 13 | 3201 | 6802 | 0801 | 44020101 | 0102 | 07 |
| IHW09398 | FH18 | F | American Black | 13 | 3601 | 7401 | 5301 | 5703 | 0401 | 0701 |
| IHW09382 | FH8 | F | Am. Black | 13 | 110101 | 3402 | 8201 | 270502 | 0302 | 0102 |
| IHW01026 | 1332-8257 | F | Caucasian | CEPH | 0301 | 3001 | 3501 | 0702 | 0401 | 0702 |
| IHW09409 | FH27 | M | Native American | 13 | 310102 | 3401 | 350101 | 380201 | 0401 | 0702 |
| IHW09452 | FH64 | | Unknown | 13 | 02 | 3201 | 44 | 1801 | 05 | 07 |
| IHW01028 | 1332-8259 | F | Caucasian | CEPH | 0301 | 0301 | 3501 | 2705 | 0401 | 020202 |
| IHW01061 | 1341-8465 | F | Caucasian | CEPH | 0201 | 680102 | 0702 | 0702 | 0702 | 0702 |
| IHW01099 | 1347-8434 | M | Caucasian | CEPH | 2501 | 0101 | 0801 | 1501 | 0701 | 0602 |
| IHW01136 | 1362-8575 | M | Caucasian | CEPH | 0101 | 1101 | 0702 | 5101 | 0702 | 1502 |

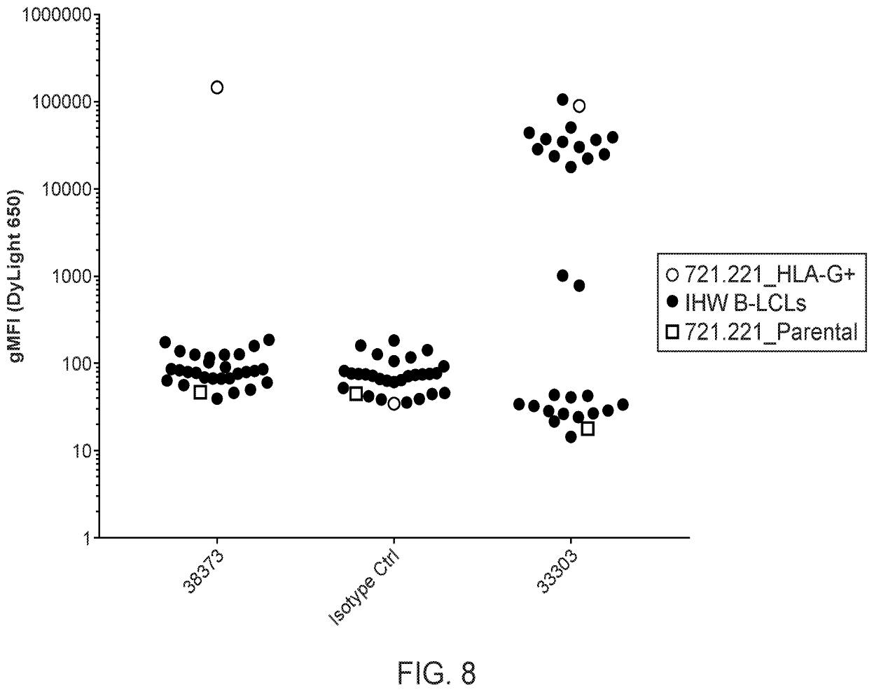

The results are shown in FIG. 8. Binding levels are provided as geometric MFI. Class I-negative 721.221 cell lines with and without expression of HLA-G were used as controls.

### Example 11: Critical HLA-G Amino Acid Determinants Required for HLA-G Antibody Binding

721.221 LCL cells transiently expressing various point mutants of HLA-G were established using the Neon® Transfection System (ThermoFisher Scientific). Cells were transfected with 1 μg of plasmid per point mutanin the 10 μL reaction format. Cells were cultured for 72 hours prior to staining with anti-HLA-G antibodies.

721.221 LCL cells (expressing various point mutants or a domain swap where the primary amino acid sequence encoding the alpha 3 domain was substituted with the analogous sequence from HLA-A*1101) were incubated with each antibody (100 nM) in wash buffer (Phosphate Buffered Saline, 2% FBS, 2 mM EDTA) for 60 minutes at 4° C. Cells were then washed with cold wash buffer and incubated with fluorescently labelled secondary antibody at 4° C. for 30 minutes. Numbered antibodies were incubated with 1:500 R-Phycoerythrin labelled goat-anti-human IgG (Jackson ImmunoResearch). W6/32 was incubated with 1:500 Alexa Fluor 488 goat-anti-mouse IgG (Jackson ImmunoResearch). MEMG/9 (Invitrogen) was pre-conjugated with Allophycocyanin. After incubation with secondary antibody, cells were washed and resuspended in cold wash buffer prior to analysis by flow cytometry on a BD Celesta instrument. Sample data was exported as FCS files and analyzed using FlowJo software v10. As shown in FIG. 9, mutation of residues 195, 197, and/or 198 results in loss of binding by bin 1 anti-HLA-G antibodies, revealing those residues are critical, though unlikely to be the only determinants for bin 1 antibody binding. Intriguingly, the biochemical bin defined by Octet as bin 1a (FIG. 2B) can be further distinguished based on either sensitivity to mutation at residue 197 (for example, 38379) or mutation at residue 198 (for example, 38410)

FIG. 9A shows the evaluation of HLA-G antibodies binding to various forms of HLA-G after site directed mutagenesis. FIG. 9B provides representative antibody binding histograms for each of the three point mutants (P195S, Y197A, Y197H, E198A).

### Example 12: Tumor Growth Inhibition for an Ant-HLA-G Antibody with and without Effector Function in a Mouse Xenograft Model Using 721.221 Cells Expressing HLA-G

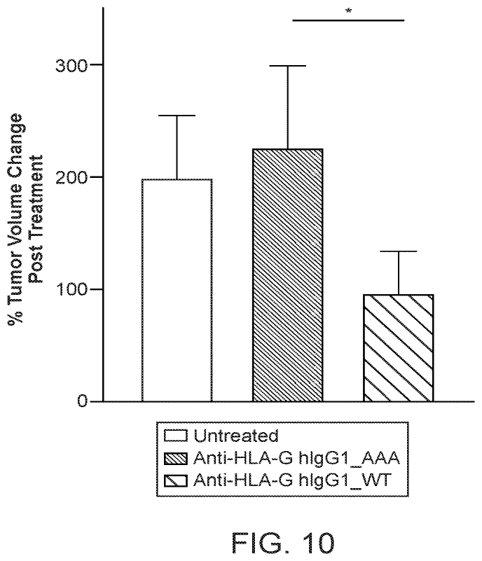

NOD/SCID mice were implanted subcutaneously with 10 million HLA-G expressing 721.221 cells in Matrigel on the right flank. On day 29 post-implantation, mice were randomized into groups based on tumor volume and dosed intraperitoneally with 300 μg of either an effector competent anti-HLA-G human IgG1 antibody (hIgG1 WT), the same anti-HLA-G antibody with mutations in the Fc domain to abrogate Fc receptor interactions (hIgG1_AAA), or left untreated. A second dose of antibody was given three days later. Data provided in FIG. 10 represents the percent change in tumor volume six days after initial treatment. These results demonstrate that an anti-HLA-G antibody can inhibit the growth of tumors expressing HLA-G by engaging Pc receptor dependent effector mechanisms, which may include antibody-dependent cellular cytotoxicity (ADCC) or phagocytosis (ADCP).

### Example S: Sequences

Table S provides sequences referred to herein.

TABLE S
Sequences.

| SEQ | | | |
|---|---|---|---|
| ID | | Scheme/ | |
| NO: | Region | Clone | Sequence |
| 1 | CDR-H1 | Chothia | GGSISSSDY |
| 2 | CDR-H1 | Chothia | GGSISSSST |
| 3 | CDR-H1 | Chothia | GGSISSSDT |
| 4 | CDR-H1 | Chothia | GGSISSADN |
| 5 | CDR-H1 | Chothia | GGSVSSSST |
| 6 | CDR-H1 | Chothia | GYSISSGY |
| 7 | CDR-H1 | Chothia | GYSILSGY |
| 8 | CDR-H1 | Chothia | GYSISSGH |
| 9 | CDR-H1 | Chothia | GYSISSGF |
| 10 | CDR-H1 | Chothia | GFTFDNY |
| 11 | CDR-H1 | Chothia | GFTFSDY |
| 12 | CDR-H1 | Chothia | GFTFSSS |
| 13 | CDR-H1 | Chothia | GGSISSSN |
| 14 | CDR-H1 | Chothia | GGSISSSSY |
| 15 | | | |
| 16 | | | |
| 17 | | | |
| 18 | CDR-H1 | Kabat | SSDYYWG |
| 19 | CDR-H1 | Kabat | SSSTYWA |
| 20 | CDR-H1 | Kabat | SSSTYWG |
| 21 | CDR-H1 | Kabat | SSDTYWG |
| 22 | CDR-H1 | Kabat | SADNYWG |
| 23 | CDR-H1 | Kabat | SSSTYWS |
| 24 | CDR-H1 | Kabat | SGYYWG |
| 25 | CDR-H1 | Kabat | SGYYWF |
| 26 | CDR-H1 | Kabat | SGHYWI |
| 27 | CDR-H1 | Kabat | SGFYWT |
| 28 | CDR-H1 | Kabat | SGYYWL |
| 29 | CDR-H1 | Kabat | SGHYWT |
| 30 | CDR-H1 | Kabat | NYAMH |
| 31 | CDR-H1 | Kabat | DYYMS |
| 32 | CDR-H1 | Kabat | SSAMA |
| 33 | CDR-H1 | Kabat | SSNWWS |
| 34 | CDR-H1 | Kabat | SSSYYWG |
| 35 | | | |
| 36 | | | |
| 37 | | | |
| 38 | CDR-H2 | Chothia | YYSGS |
| 39 | CDR-H2 | Chothia | SSSGS |
| 40 | CDR-H2 | Chothia | HHSGA |
| 41 | CDR-H2 | Chothia | HYSGS |
| 42 | CDR-H2 | Chothia | AYSGS |
| 43 | CDR-H2 | Chothia | SYNAL |
| 44 | CDR-H2 | Chothia | YHSGS |
| 45 | CDR-H2 | Chothia | YHSAS |
| 46 | CDR-H2 | Chothia | SARAGI |
| 47 | CDR-H2 | Chothia | ASSGSV |
| 48 | CDR-H2 | Chothia | SGSGIT |
| 49 | CDR-H2 | Chothia | SYSGS |
| 50 | CDR-H2 | Chothia | SSSGST |
| 51 | | | |
| 52 | | | |
| 53 | | | |
| 54 | CDR-H2 | Kabat | SIYYSGSTYYNPSLKS |
| 55 | CDR-H2 | Kabat | SISSSGSTYYNPSLKS |
| 56 | CDR-H2 | Kabat | SIHHSGATYYNPSLKS |
| 57 | CDR-H2 | Kabat | SIHYSGSTLYNPSLKS |
| 58 | CDR-H2 | Kabat | SIHYSGSTYYNPSLKS |
| 59 | CDR-H2 | Kabat | GIAYSGSTYYNPSLKS |
| 60 | CDR-H2 | Kabat | SISYNALTYYNPSLKS |
| 61 | CDR-H2 | Kabat | SIYHSGSTYYNPSLKS |
| 62 | CDR-H2 | Kabat | GIYHSASTAYNPSLKS |
| 63 | CDR-H2 | Kabat | GIYHSGSTYYNPSLKS |
| 64 | CDR-H2 | Kabat | AIYHSGSTVYNPSLKS |
| 65 | CDR-H2 | Kabat | GIYHSGSTAYNPSLKS |
| 66 | CDR-H2 | Kabat | AISARAGITYYADSVKG |
| 67 | CDR-H2 | Kabat | YIASSGSVIYYADSVKG |
| 68 | CDR-H2 | Kabat | TISGSGITTWYADSVKG |
| 69 | CDR-H2 | Kabat | EIYHSGSTNYNPSLKS |
| 70 | CDR-H2 | Kabat | SISYSGSTYYNPSLKS |
| 71 | CDR-H2 | Kabat | YISSSGSTIYYADSVKG |
| 72 | | | |
| 73 | | | |
| 74 | | | |
| 75 | | | |
| 76 | CDR-H3 | | GVRRAVPFDY |
| 77 | CDR-H3 | | GIARAVPFDY |
| 78 | CDR-H3 | | GPKRAVPFDY |
| 79 | CDR-H3 | | GVRRAVPFVD |
| 80 | CDR-H3 | | GVRRAVPFQR |
| 81 | CDR-H3 | | GTRRAVPFDY |
| 82 | CDR-H3 | | GVRRAVPFAD |
| 83 | CDR-H3 | | GIRRAVPFDY |
| 84 | CDR-H3 | | GQFRAVPFDY |
| 85 | CDR-H3 | | GGTHTYSRGPMDV |
| 86 | CDR-H3 | | GGTHTYSRGPFDV |
| 87 | CDR-H3 | | GGTPIYSRGPLDV |
| 88 | CDR-H3 | | GGGQTYSRGPLDV |
| 89 | CDR-H3 | | GGGATYSRGPLDV |
| 90 | CDR-H3 | | GGTHTYSRGPLDV |
| 91 | CDR-H3 | | GGTVKYSRGPLDV |
| 92 | CDR-H3 | | GGQVTYSRGPLDV |
| 93 | CDR-H3 | | GGEVTYSRGPLDV |
| 94 | CDR-H3 | | RIGYSYGTAPPFDV |
| 95 | CDR-H3 | | HGTPRAFDI |
| 96 | CDR-H3 | | GSRHLNAFNR |
| 97 | CDR-H3 | | GVYHYDPYGMDV |
| 98 | CDR-H3 | | TELGKMHFDY |
| 99 | CDR-H3 | | GSPRYMQD |
| 100 | CDR-H3 | | HSSLGTHNWFDP |
| 101 | CDR-H3 | | EGALSYSWLAAFDI |
| 102 | | | |
| 103 | | | |
| 104 | | | |
| 105 | CDR-L1 | | RASQSVSSSYLA |
| 106 | CDR-L1 | | GASQSVSSDYLA |
| 107 | CDR-L1 | | QASQAVSSNYLA |
| 108 | CDR-L1 | | GASQSVSSAFLA |
| 109 | CDR-L1 | | RASQSVSSTYLA |
| 110 | CDR-L1 | | QASQSVSSSYLA |
| 111 | CDR-L1 | | KASQAVSSSYLA |
| 112 | CDR-L1 | | EASQSVSSSYLA |
| 113 | CDR-L1 | | EASQSVSASYLA |
| 114 | CDR-L1 | | EASQSVSSAYLA |
| 115 | CDR-L1 | | RVSQSVSDAYLA |
| 116 | CDR-L1 | | EVSQSVSASYLA |
| 117 | CDR-L1 | | RASQSVSSAYLA |
| 118 | CDR-L1 | | RASNAVSSSYLA |
| 119 | CDR-L1 | | RASQSINSWLA |
| 120 | CDR-L1 | | AASQGISSDLA |
| 121 | CDR-L1 | | RASQDISTYLN |
| 122 | CDR-L1 | | RSSQSLLHSNG |
| | | | YNYLD |
| 123 | CDR-L1 | | RASQSISSYLN |
| 124 | CDR-L1 | | RASQSVSSNLA |
| 125 | | | |
| 126 | | | |
| 127 | | | |
| 128 | CDR-L2 | | GASSRAT |
| 129 | CDR-L2 | | GAYSLAT |
| 130 | CDR-L2 | | GASARAT |
| 131 | CDR-L2 | | GASSREA |
| 132 | CDR-L2 | | GASNRAA |
| 133 | CDR-L2 | | GASSRQD |
| 134 | CDR-L2 | | GASNRAT |
| 135 | CDR-L2 | | DASSRAS |
| 136 | CDR-L2 | | DASTRAT |
| 137 | CDR-L2 | | GASDRAN |
| 138 | CDR-L2 | | GASYRAT |
| 139 | CDR-L2 | | DASSLES |
| 140 | CDR-L2 | | SASSTQS |
| 141 | CDR-L2 | | DAFNLET |
| 142 | CDR-L2 | | LGSNRAS |
| 143 | CDR-L2 | | GASRRAT |
| 144 | CDR-L2 | | AASSLQS |
| 145 | CDR-L2 | | GASTRAT |
| 146 | | | |
| 147 | | | |
| 148 | | | |
| 149 | CDR-L3 | | QQAVHSPYT |
| 150 | CDR-L3 | | QWAVHSPYT |
| 151 | CDR-L3 | | QQVVHSPYT |
| 152 | CDR-L3 | | QQTVHSPYT |
| 153 | CDR-L3 | | QQAIHSPYT |
| 154 | CDR-L3 | | QQHSSYPPT |
| 155 | CDR-L3 | | QQHSLYPPT |
| 156 | CDR-L3 | | QQFSSYPPT |
| 157 | CDR-L3 | | QQVSSYPPT |
| 158 | CDR-L3 | | QQHSIYPPT |
| 159 | CDR-L3 | | QQYDSHIT |
| 160 | CDR-L3 | | QQAYLYPIT |
| 161 | CDR-L3 | | QQLPFLPIT |
| 162 | CDR-L3 | | MQALGGPWT |
| 163 | CDR-L3 | | QQYVSDPIT |
| 164 | CDR-L3 | | QQVGSSPIT |
| 165 | CDR-L3 | | QQSHLVPRT |
| 166 | CDR-L3 | | QQANHHPPFT |
| 167 | | | |
| 168 | | | |
| 169 | | | |
| 170 | VH | | QLQLQESGPGLVKPS |
| | | | ETLSLTCTVSGGSIS |
| | | | SSDYYWGWIRQPPGK |
| | | | GLEWIGSIYYSGSTY |
| | | | YNPSLKSRVTISVDT |
| | | | SKNQFSLKLSSVTAA |
| | | | DTAVYYCARGVRRAV |
| | | | PFDYWGQGTLVTVSS |
| 171 | VH | | QLQLQESGPGLVKPS |
| | | | ETLSLTCTVSGGSIS |
| | | | SSSTYWAWIRQPPGK |
| | | | GLEWIGSISSSGSTY |
| | | | YNPSLKSRVTISVDT |
| | | | SKNQFSLKLSSVTAA |
| | | | DTAVYYCARGIARAV |
| | | | PFDYWGQGTLVTVSS |
| 172 | VH | | QLQLQESGPGLVKPS |
| | | | ETLSLTCTVSGGSIS |
| | | | SSSTYWGWIRQSPGK |
| | | | GLEWIGSIHHSGATY |
| | | | YNPSLKSRVTISVDT |
| | | | SKNQFSLKLSSVTAA |
| | | | DTAVYYCARGPKRAV |
| | | | PFDYWGQGTLVTVSS |
| 173 | VH | | QLQLQESGPGLVKPS |
| | | | ETLSLTCTVSGGSIS |
| | | | SSDTYWGWIRQPPGK |
| | | | GLEWIGSIHYSGSTL |
| | | | YNPSLKSRVTISVDT |
| | | | SKNQFSLKLSSVTAA |
| | | | DTAVYYCARGVRRAV |
| | | | PFVDWGQGTLVTVSS |
| 174 | VH | | QLQLQESGPGLVKPS |
| | | | ETLSLTCTVSGGSIS |
| | | | SADNYWGWIRQPPGK |
| | | | GLEWIGSIHYSGSTY |
| | | | YNPSLKSRVTISVDT |
| | | | SKNQFSLKLSSVTAA |
| | | | DTAVYYCARGVRRAV |
| | | | PFQRWGQGTLVTVSS |
| 175 | VH | | QLQLQESGPGLVKPS |
| | | | ETLSLTCTVSGGSIS |
| | | | SSDTYWGWIRQPPGK |
| | | | GPEWIGSIHYSGSTL |
| | | | YNPSLKSRVTISVDT |
| | | | SKNQFSLKLSSVTAA |
| | | | DTAVYYCARGVRRAV |
| | | | PFDYWGQGTLVTVSS |
| 176 | VH | | QLQLQESGPGLVKPS |
| | | | ETLSLTCTVSGGSIS |
| | | | SSSTYWSWIRQPPGK |
| | | | GLEWIGGIAYSGSTY |
| | | | YNPSLKSRVTISVDT |
| | | | SKNQFSLKLSSVTAA |
| | | | DTAVYYCARGVRRAV |
| | | | PFDYWGQGTLVTVSS |
| 177 | VH | | QLQLQESGPGLVKPS |
| | | | ETLSLTCTVSGGSIS |
| | | | SSSTYWSWIRQPPGK |
| | | | GLEWIGSISYNALTY |
| | | | YNPSLKSRVTISVDT |
| | | | SKNQFSLKLSSVTAA |
| | | | DTAVYYCARGTRRAV |
| | | | PFDYWGQGTLVTVSS |
| 178 | VH | | QLQLQESGPGLVKPS |
| | | | ETLSLTCTVSGGSIS |
| | | | SSSTYWSWIRQPPGK |
| | | | GLEWIGGIAYSGSTY |
| | | | YNPSLKSRVTISVDT |
| | | | SKNQFSLKLSSVTAA |
| | | | DTAVYYCARGVRRAV |
| | | | PFADWGQGTLVTVSS |
| 179 | VH | | QLQLQESGPGLVKPS |
| | | | ETLSLTCTVSGGSVS |
| | | | SSSTYWSWIRQPPGK |
| | | | GLEWIGGIAYSGSTY |
| | | | YNPSLKSRVTISVDT |
| | | | SKNQFSLKLSSVTAA |
| | | | DTAVYYCARGVRRAV |
| | | | PFDYWGQGTLVTVSS |
| 180 | VH | | QLQLQESGPGLVKPS |
| | | | ETLSLTCTVSGGSIS |
| | | | SSSTYWSWIRQPPGK |
| | | | GLEWIGGIAYSGSTY |
| | | | YNPSLKSRVTISVDT |
| | | | SKNQFSLKLSSVTAA |
| | | | DTAVYYCARGTRRAV |
| | | | PFDYWGQGTLVTVSS |
| 181 | VH | | QLQLQESGPGLVKPS |
| | | | ETLSLTCTVSGGSIS |
| | | | SSDTYWGWIRQPPGK |
| | | | GLEWIGSIHYSGSTL |
| | | | YNPSLKSRVTISVDT |
| | | | SKNQFSLKLSSVTAA |
| | | | DTAVYYCARGIRRAV |
| | | | PFDYWGQGTLVTVSS |
| 182 | VH | | |
| 183 | VH | | QVQLQESGPGLVKPS |
| | | | ETLSLTCAVSGYSIS |
| | | | SGYYWGWIRQPPGKG |
| | | | LEWIGSIYHSGSTYY |
| | | | NPSLKSRVTISVDTS |
| | | | KNQFSLKLSSVTAAD |
| | | | TAVYYCARGGTHTYS |
| | | | RGPMDVWGQGTTVTV |
| | | | SS |
| 184 | VH | | QLQLQESGPRLVKPS |
| | | | ETLSLTCAVSGYSIS |
| | | | SGYYWGWIRQPPGKG |
| | | | LEWIGSIYHSGSTYY |
| | | | NPSLKSRVTISVDTS |
| | | | KNQFSLKLSSVTAAD |
| | | | TAVYYCARGGTHTYS |
| | | | RGPFDVWGQGTTVTV |
| | | | SS |
| 185 | VH | | LVQLQESGPGLVKPS |
| | | | ETLSLTCAVSGYSIL |
| | | | SGYYWFWIRQPPGKG |
| | | | LEWIGGIYHSASTAY |
| | | | NPSLKSRVTISVDTS |
| | | | KNQFSLKLSSVTAAD |
| | | | TAVYYCARGGTPIYS |
| | | | RGPLDVWGQGTTVTV |
| | | | SS |
| 186 | VH | | QVQLQESGPGLVKPS |
| | | | ETLSLTCAVSGYSIS |
| | | | SGHYWIWIRQPPGKG |
| | | | LEWIGGIYHSGSTYY |
| | | | NPSLKSRVTISVDTS |
| | | | KDQFSLKLSSVTAAD |
| | | | TAVYYCARGGGQTYS |
| | | | RGPLDVWGQGTTVTV |
| | | | SS |
| 187 | VH | | QVQLQESGPGLVKPPE |
| | | | TLSLTCAVSGYSISS |
| | | | GHYWIWIRQPPGKGL |
| | | | EWIGGIYHSGSTYYN |
| | | | PSLKSRVTISVDTSK |
| | | | NQFSLKLSSVTAADT |
| | | | AVYYCARGGGATYSR |
| | | | GPLDVWGQGTTVTVS |
| | | | S |
| 188 | VH | | QVQLQESGPGLVKPSE |
| | | | TLSLTCAVSGYSILSG |
| | | | YYWFWIRQPPGKGLEW |
| | | | IGGIYHSGSTYYNPS |
| | | | LKSRVTISVDTSKNQ |
| | | | FSLKLSSVTAADTAV |
| | | | YYCARGGTHTYSRGP |
| | | | LDVWGQGTTVTVSS |
| 189 | VH | | QVQLQESGPGLVKPSE |
| | | | TLSLTCAVSGYSISSG |
| | | | FYWTWIRQPPGKGLEW |
| | | | IGAIYHSGSTVYNPS |
| | | | LKSRVTISVDTSKNQ |
| | | | FSLKLSSVTAADTAV |
| | | | YYCARGGTHTYSRGP |
| | | | LDVWGQGTTVTVSS |
| 190 | VH | | QVQLQESGPGLVKPS |
| | | | ETLSLTCAVSGYSIS |
| | | | SGYYWLWIRQPPGKG |
| | | | LEWIGGIYHSASTAY |
| | | | NPSLKSRVTISVDTS |
| | | | KNQFSLKLSSVTAAD |
| | | | TAVYYCARGGTVKYS |
| | | | RGPLDVWGQGTTVTV |
| | | | SS |
| 191 | VH | | QVQLQESGPGLVKPS |
| | | | ETLSLTCAVSGYSIS |
| | | | SGHYWTWIRQPPGKG |
| | | | LEWIGAIYHSGSTVY |
| | | | NPSLKSRVTISVDTS |
| | | | KNQFSLKLSSVTAAD |
| | | | TAVYYCARGGQVTYS |
| | | | RGPLDVWGQGTTVTV |
| | | | SS |
| 192 | VH | | QVQLQESGPGLVKPS |
| | | | ETLSLTCAVSGYSIL |
| | | | SGYYWFWIRQPPGKG |
| | | | LEWIGGIYHSGSTAY |
| | | | NPSLKSRVTISVDTS |
| | | | KNQFSLKLSSVTAAD |
| | | | TAVYYCARGGEVTYS |
| | | | RGPLDVWGQGTTVTV |
| | | | SS |
| 193 | VH | | EVQLLESGGGLVQPG |
| | | | GSLRLSCAASGFTFD |
| | | | NYAMHWVRQAPGKGL |
| | | | EWVSAISARAGITYY |
| | | | ADSVKGRFTISRDNS |
| | | | KNTLYLQMNSLRAED |
| | | | TAVYYCARRIGYSYG |
| | | | TAPPFDVWGQGTTVT |
| | | | VSS |
| 194 | VH | | QVQLVESGGGLVQPG |
| | | | GSLRLSCAASGFTFS |
| | | | DYYMSWIRQAPGKGL |
| | | | EWVSYIASSGSVIYY |
| | | | ADSVKGRFTISRDNA |
| | | | KNSLYLQMNSLRAED |
| | | | TAVYYCARHGTPRAF |
| | | | DIWGQGTTVTVSS |
| 195 | VH | | EVQLLESGGGLVQPG |
| | | | GSLRLSCAASGFTFS |
| | | | SSAMAWVRQAPGKGL |
| | | | EWVSTISGSGITTWY |
| | | | ADSVKGRFTISRDNS |
| | | | KNTLYLQMNSLRAED |
| | | | TAVYYCAKGSRHLNA |
| | | | FNRWGQGTTVTVSS |
| 196 | VH | | QVQLQESGPGLVKPS |
| | | | GTLSLTCAVSGGSIS |
| | | | SSNWWSWVRQPPGKG |
| | | | LEWIGEIYHSGSTNY |
| | | | NPSLKSRVTISVDKS |
| | | | KNQFSLKLSSVTAAD |
| | | | TAVYYCARGVYHYDP |
| | | | YGMDVWGQGTTVTVS |
| | | | S |
| 197 | VH | | QLQLQESGPGLVKPS |
| | | | ETLSLTCTVSGGSIS |
| | | | SSSYYWGWIRQPPGK |
| | | | GLEWIGSISYSGSTY |
| | | | YNPSLKSRVTISVDT |
| | | | SKNQFSLKLSSVTAA |
| | | | DTAVYYCARTELGKM |
| | | | HFDYWGQGTLVTVSS |
| 198 | VH | | QLQLQESGPGLVKPS |
| | | | ETLSLTCTVSGGSIS |
| | | | SSDYYWGWIRQPPGK |
| | | | GLEWIGSIYYSGSTY |
| | | | YNPSLKSRVTISVDT |
| | | | SKNQFSLKLSSVTAA |
| | | | DTAVYYCARGSPRYM |
| | | | QDWGQGTLVTVSS |
| 199 | VH | | QVQLVESGGGLVKPG |
| | | | GSLRLSCAASGFTFS |
| | | | DYYMSWIRQAPGKGL |
| | | | EWVSYISSSGSTIYY |
| | | | ADSVKGRFTISRDNA |
| | | | KNSLYLQMNSLRAED |
| | | | TAVYYCARHSSLGTH |
| | | | NWFDPWGQGTLVTVS |
| | | | S |
| 200 | VH | | QVQLQESGPGLVKPS |
| | | | ETLSLTCAVSGYSIS |
| | | | SGYYWGWIRQPPGKG |
| | | | LEWIGSIYHSGSTYY |
| | | | NPSLKSRVTISVDTS |
| | | | KNQFSLKLSSVTAAD |
| | | | TAVYYCAREGALSYS |
| | | | WLAAFDIWGQGTMVT |
| | | | VSS |
| 201 | | | |
| 202 | | | |
| 203 | | | |
| 204 | VL | | EIVLTQSPGTLSLSP |
| | | | GERATLSCRASQSVS |
| | | | SSYLAWYQQKPGQAP |
| | | | RLLIYGASSRATGIP |
| | | | DRFSGSGSGTDFTLT |
| | | | ISRLEPEDFAVYYCQ |
| | | | QAVHSPYTFGGGTKV |
| | | | EIK |
| 205 | VL | | EIVLTQSPGTLSLSP |
| | | | GERATLSCGASQSVS |
| | | | SDYLAWYQQKPGQAP |
| | | | RLLIYGAYSLATGIP |
| | | | DRFSGSGSGTDFTLT |
| | | | ISRLEPEDFAVYYCQ |
| | | | WAVHSPYTFGGGTKV |
| | | | EIK |
| 206 | VL | | EIVLTQSPGTLSLSP |
| | | | GERATLSCQASQAVS |
| | | | SNYLAWYQQKPGQAP |
| | | | RLLIYGASSRATGIP |
| | | | DRFSGSGSGTDFTLT |
| | | | ISRLEPEDFAVYYCQ |
| | | | QVVHSPYTFGGGTKV |
| | | | EIK |
| 207 | VL | | EIVLTQSPGTLSLSP |
| | | | GERATLSCGASQSVS |
| | | | SAFLAWYQQKPGQAP |
| | | | RLLIYGASARATGIP |
| | | | DRFSGSGSGTDFTLT |
| | | | ISRLEPEDFAVYYCQ |
| | | | QVVHSPYTFGGGTKV |
| | | | EIK |
| 208 | VL | | EIVLTQSPGTLSLSP |
| | | | GERATLSCRASQSVS |
| | | | STYLAWYQQKPGQAP |
| | | | RLLIYGASSREAGIP |
| | | | DRFSGSGSGTDFTLT |
| | | | ISRLEPEDFAVYYCQ |
| | | | QTVHSPYTFGGGTKV |
| | | | EIK |
| 209 | VL | | EIVLTQSPGTLSLSP |
| | | | GERATLSCQASQAVS |
| | | | SNYLAWYQQKPGQAP |
| | | | RLLIYGASNRAAGIP |
| | | | DRFSGSGSGTDFTLT |
| | | | ISRLEPEDFAVYYCQ |
| | | | QAIHSPYTFGGGTKV |
| | | | EIK |
| 210 | VL | | EIVLTQSPGTLSLSP |
| | | | GERATLSCQASQSVS |
| | | | SSYLAWYQQKPGQAP |
| | | | RLLIYGASNRAAGIP |
| | | | DRFSGSGSGTDFTLT |
| | | | ISRLEPEDFAVYYCQ |
| | | | QVVHSPYTFGGGTKV |
| | | | EIK |
| 211 | VL | | EIVLTQSPGTLSLSP |
| | | | GERATLSCKASQAVS |
| | | | SSYLAWYQQKPGQAP |
| | | | RLLIYGASSRQDGIP |
| | | | DRFSGSGSGTDFTLT |
| | | | ISRLEPEDFAVYYCQ |
| | | | QVVHSPYTFGGGTKV |
| | | | EIK |
| 212 | VL | | EIVLTQSPGTLSLSP |
| | | | GERATLSCRASQSVS |
| | | | SSYLAWYQQKPGQAP |
| | | | RLLIYGASSRATGIP |
| | | | DRFSGSGSGTDFTLT |
| | | | ISRLEPEDFAVYYCQ |
| | | | QHSSYPPTFGGGTKV |
| | | | EIK |
| 213 | VL | | EIVLTQSPGTLSLSP |
| | | | GERATLSCEASQSVS |
| | | | SSYLAWYQQKPGQAP |
| | | | RLLIYGASNRATGIP |
| | | | DRFSGSGSGTDFTLT |
| | | | ISRLEPEDFAVYYCQ |
| | | | QHSLYPPTFGGGTKV |
| | | | EIK |
| 214 | VL | | EIVLTQSPGTLSLSP |
| | | | GERATLSCEASQSVS |
| | | | ASYLAWYQQKPGQAP |
| | | | RLLIYDASSRASGIP |
| | | | DRFSGSGSGTDFTLT |
| | | | ISRLEPEDFAVYYCQ |
| | | | QFSSYPPTFGGGTKV |
| | | | EIK |
| 215 | VL | | EIVLTQSPGTLSLSP |
| | | | GERATLSCEASQSVS |
| | | | SAYLAWYQQKPGQAP |
| | | | RLLIYDASTRATGIP |
| | | | DRFSGSGSGTDFTLT |
| | | | ISRLEPEDFAVYYCQ |
| | | | QVSSYPPTFGGGTKV |
| | | | EIK |
| 216 | VL | | EIVLTQSPGTLSLSP |
| | | | GERAALSCRVSQSVS |
| | | | DAYLAWYQQKPGQAP |
| | | | RLLIYDASSRASGIP |
| | | | DRFSGSGSGTDFTLT |
| | | | ISRLEPEDFAVYYCQ |
| | | | QVSSYPPTFGGGTKV |
| | | | EIK |
| 217 | VL | | EIVLTQSPGTLSLSP |
| | | | GERATLSCEVSQSVS |
| | | | ASYLAWYQQKPGQAP |
| | | | RLLIYGASSRATGIP |
| | | | DRFSGSGSGTDFTLT |
| | | | ISRLEPEDFAVYYCQ |
| | | | QHSLYPPTFGGGTKV |
| | | | EIK |
| 218 | VL | | EIVLTQSPGTLSLSP |
| | | | GERATLSCRASQSVS |
| | | | SAYLAWYQQKPGQAP |
| | | | RLLIYGASDRANGIP |
| | | | DRFSGSGSGTDFTLT |
| | | | ISRLEPEDFAVYYCQ |
| | | | QHSLYPPTFGGGTKV |
| | | | EIK |
| 219 | VL | | EIVLTQSPGTLSLSP |
| | | | GERATLSCRASNAVS |
| | | | SSYLAWYQQKPGQAP |
| | | | RLLIYGASDRANGIP |
| | | | DRFSGSGSGTDFTLT |
| | | | ISRLEPEDFAVYYCQ |
| | | | QHSIYPPTFGGGTKV |
| | | | EIK |
| 220 | VL | | EIVLTQSPGTLSLSP |
| | | | GERATLSCRASNAVS |
| | | | SSYLAWYQQKPGQAP |
| | | | RLLIYGASYRATGIP |
| | | | DRFSGSGSGTDFTLT |
| | | | ISRLEPEDFAVYYCQ |
| | | | QHSLYPPTFGGGTKV |
| | | | EIK |
| 221 | VL | | DIQMTQSPSTLSASV |
| | | | GDRVTITCRASQSIN |
| | | | SWLAWYQQKPGKAPK |
| | | | LLISDASSLESGVPS |
| | | | RFSGSGSGTEFTLTI |
| | | | SSLQPDDFATYYCQQ |
| | | | YDSHITFGGGTKVEI |
| | | | K |
| 222 | VL | | DIQMTQSPSSVSASV |
| | | | GDRVTITCAASQGIS |
| | | | SDLAWYQQKPGKAPK |
| | | | LLIYSASSTQSGVPS |
| | | | RFSGSGSGTDFTLTI |
| | | | SSLQPEDFATYYCQQ |
| | | | AYLYPITFGGGTKVE |
| | | | IK |
| 223 | VL | | GVQMTQSPSSLSASV |
| | | | GDRVTITCRASQDIS |
| | | | TYLNWYQQKPGKAPK |
| | | | LLIYDAFNLETGVPS |
| | | | RFSGSGSGTDFTFTI |
| | | | SSLQPEDIATYYCQQ |
| | | | LPFLPITFGGGTKVE |
| | | | IK |
| 224 | VL | | DIVMTQSPLSLPVTP |
| | | | GEPASISCRSSQSLL |
| | | | HSNGYNYLDWYLQKP |
| | | | GQSPQLLIYLGSNRA |
| | | | SGVPDRFSGSGSGTD |
| | | | FTLKISRVEAEDVGV |
| | | | YYCMQALGGPWTFGG |
| | | | GTKVEIK |
| 225 | VL | | EIVLTQSPGTLSLSP |
| | | | GERATLSCRASQSVS |
| | | | SSYLAWYQQKPGQAP |
| | | | RLLIYGASRRATGIP |
| | | | DRFSGSGSGTDFTLT |
| | | | ISRLEPEDFAVYYCQ |
| | | | QYVSDPITFGGGTKV |
| | | | EIK |
| 226 | VL | | EIVLTQSPGTLSLSP |
| | | | GERATLSCRASQSVS |
| | | | SSYLAWYQQKPGQAP |
| | | | RLLIYGASRRATGIP |
| | | | DRFSGSGSGTDFTLT |
| | | | ISRLEPEDFAVYYCQ |
| | | | QVGSSPITFGGGTKV |
| | | | EIK |
| 227 | VL | | DIQMTQSPSSLSASV |
| | | | GDRVTITCRASQSIS |
| | | | SYLNWYQQKPGKAPK |
| | | | LLIYAASSLQSGVPS |
| | | | RFSGSGSGTDFTLTI |
| | | | SSLQPEDFATYYCQQ |
| | | | SHLVPRTFGGGTKVE |
| | | | IK |
| 228 | VL | | EIVMTQSPATLSVSP |
| | | | GERATLSCRASQSVS |
| | | | SNLAWYQQKPGQAPR |
| | | | LLIYGASTRATGIPA |
| | | | RFSGSGSGTEFTLTI |
| | | | SSLQSEDFAVYYCQQ |
| | | | ANHHPPFTFGGGTKV |
| | | | EIK |
| 229 | | | |
| 230 | | | |
| 231 | | | |
| 232 | IGG1 AAA HC | 33343 | QLQLQESGPGLVKPS |
| | | | ETLSLTCTVSGGSIS |
| | | | SSDYYWGWIRQPPGK |
| | | | GLEWIGSIYYSGSTY |
| | | | YNPSLKSRVTISVDT |
| | | | SKNQFSLKLSSVTAA |
| | | | DTAVYYCARGVRRAV |
| | | | PFDYWGQGTLVTVSS |
| | | | ASTKGPSVFPLAPSS |
| | | | KSTSGGTAALGCLVK |
| | | | DYFPEPVTVSWNSGA |
| | | | LTSGVHTFPAVLQSS |
| | | | GLYSLSSVVTVPSSS |
| | | | LGTQTYICNVNHKPS |
| | | | NTKVDKKVEPKSCDK |
| | | | THTCPPCPAPEAAGA |
| | | | PSVFLFPPKPKDTLM |
| | | | ISRTPEVTCVVVDVS |
| | | | HEDPEVKFNWYVDGV |
| | | | EVHNAKTKPREEQYN |
| | | | STYRVVSVLTVLHQD |
| | | | WLNGKEYKCKVSNKA |
| | | | LPAPIEKTISKAKGQ |
| | | | PREPQVYTLPPSREE |
| | | | MTKNQVSLTCLVKGF |
| | | | YPSDIAVEWESNGQP |
| | | | ENNYKTTPPVLDSDG |
| | | | SFFLYSKLTVDKSRW |
| | | | QQGNVFSCSVMHEAL |
| | | | HNHYTQKSLSLSPGK |
| 233 | IGG1 AAA HC | 37268 | QLQLQESGPGLVKPS |
| | | | ETLSLTCTVSGGSIS |
| | | | SSSTYWAWIRQPPGK |
| | | | GLEWIGSISSSGSTY |
| | | | YNPSLKSRVTISVDT |
| | | | SKNQFSLKLSSVTAA |
| | | | DTAVYYCARGIARAV |
| | | | PFDYWGQGTLVTVSS |
| | | | ASTKGPSVFPLAPSS |
| | | | KSTSGGTAALGCLVK |
| | | | DYFPEPVTVSWNSGA |
| | | | LTSGVHTFPAVLQSS |
| | | | GLYSLSSVVTVPSSS |
| | | | LGTQTYICNVNHKPS |
| | | | NTKVDKKVEPKSCDK |
| | | | THTCPPCPAPEAAGA |
| | | | PSVFLFPPKPKDTLM |
| | | | ISRTPEVTCVVVDVS |
| | | | HEDPEVKFNWYVDGV |
| | | | EVHNAKTKPREEQYN |
| | | | STYRVVSVLTVLHQD |
| | | | WLNGKEYKCKVSNKA |
| | | | LPAPIEKTISKAKGQ |
| | | | PREPQVYTLPPSREE |
| | | | MTKNQVSLTCLVKGF |
| | | | YPSDIAVEWESNGQP |
| | | | ENNYKTTPPVLDSDG |
| | | | SFFLYSKLTVDKSRW |
| | | | QQGNVFSCSVMHEAL |
| | | | HNHYTQKSLSLSPGK |
| 234 | IGG1 AAA HC | 37269 | QLQLQESGPGLVKPS |
| | | | ETLSLTCTVSGGSIS |
| | | | SSSTYWGWIRQSPGK |
| | | | GLEWIGSIHHSGATY |
| | | | YNPSLKSRVTISVDT |
| | | | SKNQFSLKLSSVTAA |
| | | | DTAVYYCARGPKRAV |
| | | | PFDYWGQGTLVTVSS |
| | | | ASTKGPSVFPLAPSS |
| | | | KSTSGGTAALGCLVK |
| | | | DYFPEPVTVSWNSGA |
| | | | LTSGVHTFPAVLQSS |
| | | | GLYSLSSVVTVPSSS |
| | | | LGTQTYICNVNHKPS |
| | | | NTKVDKKVEPKSCDK |
| | | | THTCPPCPAPEAAGA |
| | | | PSVFLFPPKPKDTLM |
| | | | ISRTPEVTCVVVDVS |
| | | | HEDPEVKFNWYVDGV |
| | | | EVHNAKTKPREEQYN |
| | | | STYRVVSVLTVLHQD |
| | | | WLNGKEYKCKVSNKA |
| | | | LPAPIEKTISKAKGQ |
| | | | PREPQVYTLPPSREE |
| | | | MTKNQVSLTCLVKGF |
| | | | YPSDIAVEWESNGQP |
| | | | ENNYKTTPPVLDSDG |
| | | | SFFLYSKLTVDKSRW |
| | | | QQGNVFSCSVMHEAL |
| | | | HNHYTQKSLSLSPGK |
| 235 | IGG1 AAA HC | 37271 | QLQLQESGPGLVKPS |
| | | | ETLSLTCTVSGGSIS |
| | | | SSDTYWGWIRQPPGK |
| | | | GLEWIGSIHYSGSTL |
| | | | YNPSLKSRVTISVDT |
| | | | SKNQFSLKLSSVTAA |
| | | | DTAVYYCARGVRRAV |
| | | | PFVDWGQGTLVTVSS |
| | | | ASTKGPSVFPLAPSS |
| | | | KSTSGGTAALGCLVK |
| | | | DYFPEPVTVSWNSGA |
| | | | LTSGVHTFPAVLQSS |
| | | | GLYSLSSVVTVPSSS |
| | | | LGTQTYICNVNHKPS |
| | | | NTKVDKKVEPKSCDK |
| | | | THTCPPCPAPEAAGA |
| | | | PSVFLFPPKPKDTLM |
| | | | ISRTPEVTCVVVDVS |
| | | | HEDPEVKFNWYVDGV |
| | | | EVHNAKTKPREEQYN |
| | | | STYRVVSVLTVLHQD |
| | | | WLNGKEYKCKVSNKA |
| | | | LPAPIEKTISKAKGQ |
| | | | PREPQVYTLPPSREE |
| | | | MTKNQVSLTCLVKGF |
| | | | YPSDIAVEWESNGQP |
| | | | ENNYKTTPPVLDSDG |
| | | | SFFLYSKLTVDKSRW |
| | | | QQGNVFSCSVMHEAL |
| | | | HNHYTQKSLSLSPGK |
| 236 | IGG1 AAA HC | 37272 | QLQLQESGPGLVKPS |
| | | | ETLSLTCTVSGGSIS |
| | | | SADNYWGWIRQPPGK |
| | | | GLEWIGSIHYSGSTY |
| | | | YNPSLKSRVTISVDT |
| | | | SKNQFSLKLSSVTAA |
| | | | DTAVYYCARGVRRAV |
| | | | PFQRWGQGTLVTVSS |
| | | | ASTKGPSVFPLAPSS |
| | | | KSTSGGTAALGCLVK |
| | | | DYFPEPVTVSWNSGA |
| | | | LTSGVHTFPAVLQSS |
| | | | GLYSLSSVVTVPSSS |
| | | | LGTQTYICNVNHKPS |
| | | | NTKVDKKVEPKSCDK |
| | | | THTCPPCPAPEAAGA |
| | | | PSVFLFPPKPKDTLM |
| | | | ISRTPEVTCVVVDVS |
| | | | HEDPEVKFNWYVDGV |
| | | | EVHNAKTKPREEQYN |
| | | | STYRVVSVLTVLHQD |
| | | | WLNGKEYKCKVSNKA |
| | | | LPAPIEKTISKAKGQ |
| | | | PREPQVYTLPPSREE |
| | | | MTKNQVSLTCLVKGF |
| | | | YPSDIAVEWESNGQP |
| | | | ENNYKTTPPVLDSDG |
| | | | SFFLYSKLTVDKSRW |
| | | | QQGNVFSCSVMHEAL |
| | | | HNHYTQKSLSLSPGK |
| 237 | IGG1 AAA HC | 37277 | QLQLQESGPGLVKPS |
| | | | ETLSLTCTVSGGSIS |
| | | | SSDTYWGWIRQPPGK |
| | | | GPEWIGSIHYSGSTL |
| | | | YNPSLKSRVTISVDT |
| | | | SKNQFSLKLSSVTAA |
| | | | DTAVYYCARGVRRAV |
| | | | PFDYWGQGTLVTVSS |
| | | | ASTKGPSVFPLAPSS |
| | | | KSTSGGTAALGCLVK |
| | | | DYFPEPVTVSWNSGA |
| | | | LTSGVHTFPAVLQSS |
| | | | GLYSLSSVVTVPSSS |
| | | | LGTQTYICNVNHKPS |
| | | | NTKVDKKVEPKSCDK |
| | | | THTCPPCPAPEAAGA |
| | | | PSVFLFPPKPKDTLM |
| | | | ISRTPEVTCVVVDVS |
| | | | HEDPEVKFNWYVDGV |
| | | | EVHNAKTKPREEQYN |
| | | | STYRVVSVLTVLHQD |
| | | | WLNGKEYKCKVSNKA |
| | | | LPAPIEKTISKAKGQ |
| | | | PREPQVYTLPPSREE |
| | | | MTKNQVSLTCLVKGF |
| | | | YPSDIAVEWESNGQP |
| | | | ENNYKTTPPVLDSDG |
| | | | SFFLYSKLTVDKSRW |
| | | | QQGNVFSCSVMHEAL |
| | | | HNHYTQKSLSLSPGK |
| 238 | IGG1 AAA HC | 38373 | QLQLQESGPGLVKPS |
| | | | ETLSLTCTVSGGSIS |
| | | | SSSTYWSWIRQPPGK |
| | | | GLEWIGGIAYSGSTY |
| | | | YNPSLKSRVTISVDT |
| | | | SKNQFSLKLSSVTAA |
| | | | DTAVYYCARGVRRAV |
| | | | PFDYWGQGTLVTVSS |
| | | | ASTKGPSVFPLAPSS |
| | | | KSTSGGTAALGCLVK |
| | | | DYFPEPVTVSWNSGA |
| | | | LTSGVHTFPAVLQSS |
| | | | GLYSLSSVVTVPSSS |
| | | | LGTQTYICNVNHKPS |
| | | | NTKVDKKVEPKSCDK |
| | | | THTCPPCPAPEAAGA |
| | | | PSVFLFPPKPKDTLM |
| | | | ISRTPEVTCVVVDVS |
| | | | HEDPEVKFNWYVDGV |
| | | | EVHNAKTKPREEQYN |
| | | | STYRVVSVLTVLHQD |
| | | | WLNGKEYKCKVSNKA |
| | | | LPAPIEKTISKAKGQ |
| | | | PREPQVYTLPPSREE |
| | | | MTKNQVSLTCLVKGF |
| | | | YPSDIAVEWESNGQP |
| | | | ENNYKTTPPVLDSDG |
| | | | SFFLYSKLTVDKSRW |
| | | | QQGNVFSCSVMHEAL |
| | | | HNHYTQKSLSLSPGK |
| 239 | IGG1 AAA HC | 38375 | QLQLQESGPGLVKPS |
| | | | ETLSLTCTVSGGSIS |
| | | | SSSTYWSWIRQPPGK |
| | | | GLEWIGSISYNALTY |
| | | | YNPSLKSRVTISVDT |
| | | | SKNQFSLKLSSVTAA |
| | | | DTAVYYCARGTRRAV |
| | | | PFDYWGQGTLVTVSS |
| | | | ASTKGPSVFPLAPSS |
| | | | KSTSGGTAALGCLVK |
| | | | DYFPEPVTVSWNSGA |
| | | | LTSGVHTFPAVLQSS |
| | | | GLYSLSSVVTVPSSS |
| | | | LGTQTYICNVNHKPS |
| | | | NTKVDKKVEPKSCDK |
| | | | THTCPPCPAPEAAGA |
| | | | PSVFLFPPKPKDTLM |
| | | | ISRTPEVTCVVVDVS |
| | | | HEDPEVKFNWYVDGV |
| | | | EVHNAKTKPREEQYN |
| | | | STYRVVSVLTVLHQD |
| | | | WLNGKEYKCKVSNKA |
| | | | LPAPIEKTISKAKGQ |
| | | | PREPQVYTLPPSREE |
| | | | MTKNQVSLTCLVKGF |
| | | | YPSDIAVEWESNGQP |
| | | | ENNYKTTPPVLDSDG |
| | | | SFFLYSKLTVDKSRW |
| | | | QQGNVFSCSVMHEAL |
| | | | HNHYTQKSLSLSPGK |
| 240 | IGG1 AAA HC | 38379 | QLQLQESGPGLVKPS |
| | | | ETLSLTCTVSGGSIS |
| | | | SSSTYWSWIRQPPGK |
| | | | GLEWIGGIAYSGSTY |
| | | | YNPSLKSRVTISVDT |
| | | | SKNQFSLKLSSVTAA |
| | | | DTAVYYCARGVRRAV |
| | | | PFADWGQGTLVTVSS |
| | | | ASTKGPSVFPLAPSS |
| | | | KSTSGGTAALGCLVK |
| | | | DYFPEPVTVSWNSGA |
| | | | LTSGVHTFPAVLQSS |
| | | | GLYSLSSVVTVPSSS |
| | | | LGTQTYICNVNHKPS |
| | | | NTKVDKKVEPKSCDK |
| | | | THTCPPCPAPEAAGA |
| | | | PSVFLFPPKPKDTLM |
| | | | ISRTPEVTCVVVDVS |
| | | | HEDPEVKFNWYVDGV |
| | | | EVHNAKTKPREEQYN |
| | | | STYRVVSVLTVLHQD |
| | | | WLNGKEYKCKVSNKA |
| | | | LPAPIEKTISKAKGQ |
| | | | PREPQVYTLPPSREE |
| | | | MTKNQVSLTCLVKGF |
| | | | YPSDIAVEWESNGQP |
| | | | ENNYKTTPPVLDSDG |
| | | | SFFLYSKLTVDKSRW |
| | | | QQGNVFSCSVMHEAL |
| | | | HNHYTQKSLSLSPGK |
| 241 | IGG1 AAA HC | 38381 | QLQLQESGPGLVKPS |
| | | | ETLSLTCTVSGGSVS |
| | | | SSSTYWSWIRQPPGK |
| | | | GLEWIGGIAYSGSTY |
| | | | YNPSLKSRVTISVDT |
| | | | SKNQFSLKLSSVTAA |
| | | | DTAVYYCARGVRRAV |
| | | | PFDYWGQGTLVTVSS |
| | | | ASTKGPSVFPLAPSS |
| | | | KSTSGGTAALGCLVK |
| | | | DYFPEPVTVSWNSGA |
| | | | LTSGVHTFPAVLQSS |
| | | | GLYSLSSVVTVPSSS |
| | | | LGTQTYICNVNHKPS |
| | | | NTKVDKKVEPKSCDK |
| | | | THTCPPCPAPEAAGA |
| | | | PSVFLFPPKPKDTLM |
| | | | ISRTPEVTCVVVDVS |
| | | | HEDPEVKFNWYVDGV |
| | | | EVHNAKTKPREEQYN |
| | | | STYRVVSVLTVLHQD |
| | | | WLNGKEYKCKVSNKA |
| | | | LPAPIEKTISKAKGQ |
| | | | PREPQVYTLPPSREE |
| | | | MTKNQVSLTCLVKGF |
| | | | YPSDIAVEWESNGQP |
| | | | ENNYKTTPPVLDSDG |
| | | | SFFLYSKLTVDKSRW |
| | | | QQGNVFSCSVMHEAL |
| | | | HNHYTQKSLSLSPGK |
| 242 | IGG1 AAA HC | 38383 | QLQLQESGPGLVKPS |
| | | | ETLSLTCTVSGGSIS |
| | | | SSSTYWSWIRQPPGK |
| | | | GLEWIGGIAYSGSTY |
| | | | YNPSLKSRVTISVDT |
| | | | SKNQFSLKLSSVTAA |
| | | | DTAVYYCARGTRRAV |
| | | | PFDYWGQGTLVTVSS |
| | | | ASTKGPSVFPLAPSS |
| | | | KSTSGGTAALGCLVK |
| | | | DYFPEPVTVSWNSGA |
| | | | LTSGVHTFPAVLQSS |
| | | | GLYSLSSVVTVPSSS |
| | | | LGTQTYICNVNHKPS |
| | | | NTKVDKKVEPKSCDK |
| | | | THTCPPCPAPEAAGA |
| | | | PSVFLFPPKPKDTLM |
| | | | ISRTPEVTCVVVDVS |
| | | | HEDPEVKFNWYVDGV |
| | | | EVHNAKTKPREEQYN |
| | | | STYRVVSVLTVLHQD |
| | | | WLNGKEYKCKVSNKA |
| | | | LPAPIEKTISKAKGQ |
| | | | PREPQVYTLPPSREE |
| | | | MTKNQVSLTCLVKGF |
| | | | YPSDIAVEWESNGQP |
| | | | ENNYKTTPPVLDSDG |
| | | | SFFLYSKLTVDKSRW |
| | | | QQGNVFSCSVMHEAL |
| | | | HNHYTQKSLSLSPGK |
| 243 | IGG1 AAA HC | 38386 | QLQLQESGPGLVKPS |
| | | | ETLSLTCTVSGGSIS |
| | | | SSDTYWGWIRQPPGK |
| | | | GLEWIGSIHYSGSTL |
| | | | YNPSLKSRVTISVDT |
| | | | SKNQFSLKLSSVTAA |
| | | | DTAVYYCARGIRRAV |
| | | | PFDYWGQGTLVTVSS |
| | | | ASTKGPSVFPLAPSS |
| | | | KSTSGGTAALGCLVK |
| | | | DYFPEPVTVSWNSGA |
| | | | LTSGVHTFPAVLQSS |
| | | | GLYSLSSVVTVPSSS |
| | | | LGTQTYICNVNHKPS |
| | | | NTKVDKKVEPKSCDK |
| | | | THTCPPCPAPEAAGA |
| | | | PSVFLFPPKPKDTLM |
| | | | ISRTPEVTCVVVDVS |
| | | | HEDPEVKFNWYVDGV |
| | | | EVHNAKTKPREEQYN |
| | | | STYRVVSVLTVLHQD |
| | | | WLNGKEYKCKVSNKA |
| | | | LPAPIEKTISKAKGQ |
| | | | PREPQVYTLPPSREE |
| | | | MTKNQVSLTCLVKGF |
| | | | YPSDIAVEWESNGQP |
| | | | ENNYKTTPPVLDSDG |
| | | | SFFLYSKLTVDKSRW |
| | | | QQGNVFSCSVMHEAL |
| | | | HNHYTQKSLSLSPGK |
| 244 | IGG1 AAA HC | 37273 | QLQLQESGPGLVKPS |
| | | | ETLSLTCTVSGGSIS |
| | | | SSDTYWGWIRQPPGK |
| | | | GLEWIGSIHYSGSTL |
| | | | YNPSLKSRVTISVDT |
| | | | SKNQFSLKLSSVTAA |
| | | | DTAVYYCARGQFRAV |
| | | | PFDYWGQGTLVTVSS |
| | | | ASTKGPSVFPLAPSS |
| | | | KSTSGGTAALGCLVK |
| | | | DYFPEPVTVSWNSGA |
| | | | LTSGVHTFPAVLQSS |
| | | | GLYSLSSVVTVPSSS |
| | | | LGTQTYICNVNHKPS |
| | | | NTKVDKKVEPKSCDK |
| | | | THTCPPCPAPEAAGA |
| | | | PSVFLFPPKPKDTLM |
| | | | ISRTPEVTCVVVDVS |
| | | | HEDPEVKFNWYVDGV |
| | | | EVHNAKTKPREEQYN |
| | | | STYRVVSVLTVLHQD |
| | | | WLNGKEYKCKVSNKA |
| | | | LPAPIEKTISKAKGQ |
| | | | PREPQVYTLPPSREE |
| | | | MTKNQVSLTCLVKGF |
| | | | YPSDIAVEWESNGQP |
| | | | ENNYKTTPPVLDSDG |
| | | | SFFLYSKLTVDKSRW |
| | | | QQGNVFSCSVMHEAL |
| | | | HNHYTQKSLSLSPGK |
| 245 | IGG1 AAA HC | 33361 | QVQLQESGPGLVKPS |
| | | | ETLSLTCAVSGYSIS |
| | | | SGYYWGWIRQPPGKG |
| | | | LEWIGSIYHSGSTYY |
| | | | NPSLKSRVTISVDTS |
| | | | KNQFSLKLSSVTAAD |
| | | | TAVYYCARGGTHTYS |
| | | | RGPMDVWGQGTTVTV |
| | | | SSASTKGPSVFPLAP |
| | | | SSKSTSGGTAALGCL |
| | | | VKDYFPEPVTVSWNS |
| | | | GALTSGVHTFPAVLQ |
| | | | SSGLYSLSSVVTVPS |
| | | | SSLGTQTYICNVNHK |
| | | | PSNTKVDKKVEPKSC |
| | | | DKTHTCPPCPAPEAA |
| | | | GAPSVFLFPPKPKDT |
| | | | LMISRTPEVTCVVVD |
| | | | VSHEDPEVKFNWYVD |
| | | | GVEVHNAKTKPREEQ |
| | | | YNSTYRVVSVLTVLH |
| | | | QDWLNGKEYKCKVSN |
| | | | KALPAPIEKTISKAK |
| | | | GQPREPQVYTLPPSR |
| | | | EEMTKNQVSLTCLVK |
| | | | GFYPSDIAVEWESNG |
| | | | QPENNYKTTPPVLDS |
| | | | DGSFFLYSKLTVDKS |
| | | | RWQQGNVFSCSVMHE |
| | | | ALHNHYTQKSLSLSP |
| | | | GK |
| 246 | IGG1 AAA HC | 35624 | QLQLQESGPRLVKPS |
| | | | ETLSLTCAVSGYSIS |
| | | | SGYYWGWIRQPPGKG |
| | | | LEWIGSIYHSGSTYY |
| | | | NPSLKSRVTISVDTS |
| | | | KNQFSLKLSSVTAAD |
| | | | TAVYYCARGGTHTYS |
| | | | RGPFDVWGQGTTVTV |
| | | | SSASTKGPSVFPLAP |
| | | | SSKSTSGGTAALGCL |
| | | | VKDYFPEPVTVSWNS |
| | | | GALTSGVHTFPAVLQ |
| | | | SSGLYSLSSVVTVPS |
| | | | SSLGTQTYICNVNHK |
| | | | PSNTKVDKKVEPKSC |
| | | | DKTHTCPPCPAPEAA |
| | | | GAPSVFLFPPKPKDT |
| | | | LMISRTPEVTCVVVD |
| | | | VSHEDPEVKFNWYVD |
| | | | GVEVHNAKTKPREEQ |
| | | | YNSTYRVVSVLTVLH |
| | | | QDWLNGKEYKCKVSN |
| | | | KALPAPIEKTISKAK |
| | | | GQPREPQVYTLPPSR |
| | | | EEMTKNQVSLTCLVK |
| | | | GFYPSDIAVEWESNG |
| | | | QPENNYKTTPPVLDS |
| | | | DGSFFLYSKLTVDKS |
| | | | RWQQGNVFSCSVMHE |
| | | | ALHNHYTQKSLSLSP |
| | | | GK |
| 247 | IGG1 AAA HC | 38410 | LVQLQESGPGLVKPS |
| | | | ETLSLTCAVSGYSIL |
| | | | SGYYWFWIRQPPGKG |
| | | | LEWIGGIYHSASTAY |
| | | | NPSLKSRVTISVDTS |
| | | | KNQFSLKLSSVTAAD |
| | | | TAVYYCARGGTPIYS |
| | | | RGPLDVWGQGTTVTV |
| | | | SSASTKGPSVFPLAP |
| | | | SSKSTSGGTAALGCL |
| | | | VKDYFPEPVTVSWNS |
| | | | GALTSGVHTFPAVLQ |
| | | | SSGLYSLSSVVTVPS |
| | | | SSLGTQTYICNVNHK |
| | | | PSNTKVDKKVEPKSC |
| | | | DKTHTCPPCPAPEAA |
| | | | GAPSVFLFPPKPKDT |
| | | | LMISRTPEVTCVVVD |
| | | | VSHEDPEVKFNWYVD |
| | | | GVEVHNAKTKPREEQ |
| | | | YNSTYRVVSVLTVLH |
| | | | QDWLNGKEYKCKVSN |
| | | | KALPAPIEKTISKAK |
| | | | GQPREPQVYTLPPSR |
| | | | EEMTKNQVSLTCLVK |
| | | | GFYPSDIAVEWESNG |
| | | | QPENNYKTTPPVLDS |
| | | | DGSFFLYSKLTVDKS |
| | | | RWQQGNVFSCSVMHE |
| | | | ALENHYTQKSLSLSP |
| | | | GK |
| 248 | IGG1 AAA HC | 38418 | QVQLQESGPGLVKPS |
| | | | ETLSLTCAVSGYSIS |
| | | | SGHYWIWIRQPPGKG |
| | | | LEWIGGIYHSGSTYY |
| | | | NPSLKSRVTISVDTS |
| | | | KDQFSLKLSSVTAAD |
| | | | TAVYYCARGGGQTYS |
| | | | RGPLDVWGQGTTVTV |
| | | | SSASTKGPSVFPLAP |
| | | | SSKSTSGGTAALGCL |
| | | | VKDYFPEPVTVSWNS |
| | | | GALTSGVHTFPAVLQ |
| | | | SSGLYSLSSVVTVPS |
| | | | SSLGTQTYICNVNHK |
| | | | PSNTKVDKKVEPKSC |
| | | | DKTHTCPPCPAPEAA |
| | | | GAPSVFLFPPKPKDT |
| | | | LMISRTPEVTCVVVD |
| | | | VSHEDPEVKFNWYVD |
| | | | GVEVHNAKTKPREEQ |
| | | | YNSTYRVVSVLTVLH |
| | | | QDWLNGKEYKCKVSN |
| | | | KALPAPIEKTISKAK |
| | | | GQPREPQVYTLPPSR |
| | | | EEMTKNQVSLTCLVK |
| | | | GFYPSDIAVEWESNG |
| | | | QPENNYKTTPPVLDS |
| | | | DGSFFLYSKLTVDKS |
| | | | RWQQGNVFSCSVMHE |
| | | | ALENHYTQKSLSLSP |
| | | | GK |
| 249 | IGG1 AAA HC | 38420 | QVQLQESGPGLVKPP |
| | | | ETLSLTCAVSGYSIS |
| | | | SGHYWIWIRQPPGKG |
| | | | LEWIGGIYHSGSTYY |
| | | | NPSLKSRVTISVDTS |
| | | | KNQFSLKLSSVTAAD |
| | | | TAVYYCARGGGATYS |
| | | | RGPLDVWGQGTTVTV |
| | | | SSASTKGPSVFPLAP |
| | | | SSKSTSGGTAALGCL |
| | | | VKDYFPEPVTVSWNS |
| | | | GALTSGVHTFPAVLQ |
| | | | SSGLYSLSSVVTVPS |
| | | | SSLGTQTYICNVNHK |
| | | | PSNTKVDKKVEPKSC |
| | | | DKTHTCPPCPAPEAA |
| | | | GAPSVFLFPPKPKDT |
| | | | LMISRTPEVTCVVVD |
| | | | VSHEDPEVKFNWYVD |
| | | | GVEVHNAKTKPREEQ |
| | | | YNSTYRVVSVLTVLH |
| | | | QDWLNGKEYKCKVSN |
| | | | KALPAPIEKTISKAK |
| | | | GQPREPQVYTLPPSR |
| | | | EEMTKNQVSLTCLVK |
| | | | GFYPSDIAVEWESNG |
| | | | QPENNYKTTPPVLDS |
| | | | DGSFFLYSKLTVDKS |
| | | | RWQQGNVFSCSVMHE |
| | | | ALENHYTQKSLSLSP |
| | | | GK |
| 250 | IGG1 AAA HC | 38421 | QVQLQESGPGLVKPS |
| | | | ETLSLTCAVSGYSIL |
| | | | SGYYWFWIRQPPGKG |
| | | | LEWIGGIYHSGSTYY |
| | | | NPSLKSRVTISVDTS |
| | | | KNQFSLKLSSVTAAD |
| | | | TAVYYCARGGTHTYS |
| | | | RGPLDVWGQGTTVTV |
| | | | SSASTKGPSVFPLAP |
| | | | SSKSTSGGTAALGCL |
| | | | VKDYFPEPVTVSWNS |
| | | | GALTSGVHTFPAVLQ |
| | | | SSGLYSLSSVVTVPS |
| | | | SSLGTQTYICNVNHK |
| | | | PSNTKVDKKVEPKSC |
| | | | DKTHTCPPCPAPEAA |
| | | | GAPSVFLFPPKPKDT |
| | | | LMISRTPEVTCVVVD |
| | | | VSHEDPEVKFNWYVD |
| | | | GVEVHNAKTKPREEQ |
| | | | YNSTYRVVSVLTVLH |
| | | | QDWLNGKEYKCKVSN |
| | | | KALPAPIEKTISKAK |
| | | | GQPREPQVYTLPPSR |
| | | | EEMTKNQVSLTCLVK |
| | | | GFYPSDIAVEWESNG |
| | | | QPENNYKTTPPVLDS |
| | | | DGSFFLYSKLTVDKS |
| | | | RWQQGNVFSCSVMHE |
| | | | ALHNHYTQKSLSLSP |
| | | | GK |
| 251 | IGG1 AAA HC | 38422 | QVQLQESGPGLVKPS |
| | | | ETLSLTCAVSGYSIS |
| | | | SGFYWTWIRQPPGKG |
| | | | LEWIGAIYHSGSTVY |
| | | | NPSLKSRVTISVDTS |
| | | | KNQFSLKLSSVTAAD |
| | | | TAVYYCARGGTHTYS |
| | | | RGPLDVWGQGTTVTV |
| | | | SSASTKGPSVFPLAP |
| | | | SSKSTSGGTAALGCL |
| | | | VKDYFPEPVTVSWNS |
| | | | GALTSGVHTFPAVLQ |
| | | | SSGLYSLSSVVTVPS |
| | | | SSLGTQTYICNVNHK |
| | | | PSNTKVDKKVEPKSC |
| | | | DKTHTCPPCPAPEAA |
| | | | GAPSVFLFPPKPKDT |
| | | | LMISRTPEVTCVVVD |
| | | | VSHEDPEVKFNWYVD |
| | | | GVEVHNAKTKPREEQ |
| | | | YNSTYRVVSVLTVLH |
| | | | QDWLNGKEYKCKVSN |
| | | | KALPAPIEKTISKAK |
| | | | GQPREPQVYTLPPSR |
| | | | EEMTKNQVSLTCLVK |
| | | | GFYPSDIAVEWESNG |
| | | | QPENNYKTTPPVLDS |
| | | | DGSFFLYSKLTVDKS |
| | | | RWQQGNVFSCSVMHE |
| | | | ALHNHYTQKSLSLSP |
| | | | GK |
| 252 | IGG1 AAA HC | 38424 | QVQLQESGPGLVKPS |
| | | | ETLSLTCAVSGYSIS |
| | | | SGYYWLWIRQPPGKG |
| | | | LEWIGGIYHSASTAY |
| | | | NPSLKSRVTISVDTS |
| | | | KNQFSLKLSSVTAAD |
| | | | TAVYYCARGGTVKYS |
| | | | RGPLDVWGQGTTVTV |
| | | | SSASTKGPSVFPLAP |
| | | | SSKSTSGGTAALGCL |
| | | | VKDYFPEPVTVSWNS |
| | | | GALTSGVHTFPAVLQ |
| | | | SSGLYSLSSVVTVPS |
| | | | SSLGTQTYICNVNHK |
| | | | PSNTKVDKKVEPKSC |
| | | | DKTHTCPPCPAPEAA |
| | | | GAPSVFLFPPKPKDT |
| | | | LMISRTPEVTCVVVD |
| | | | VSHEDPEVKFNWYVD |
| | | | GVEVHNAKTKPREEQ |
| | | | YNSTYRVVSVLTVLH |
| | | | QDWLNGKEYKCKVSN |
| | | | KALPAPIEKTISKAK |
| | | | GQPREPQVYTLPPSR |
| | | | EEMTKNQVSLTCLVK |
| | | | GFYPSDIAVEWESNG |
| | | | QPENNYKTTPPVLDS |
| | | | DGSFFLYSKLTVDKS |
| | | | RWQQGNVFSCSVMHE |
| | | | ALHNHYTQKSLSLSP |
| | | | GK |
| 253 | IGG1 AAA HC | 38425 | QVQLQESGPGLVKPS |
| | | | ETLSLTCAVSGYSIS |
| | | | SGHYWTWIRQPPGKG |
| | | | LEWIGAIYHSGSTVY |
| | | | NPSLKSRVTISVDTS |
| | | | KNQFSLKLSSVTAAD |
| | | | TAVYYCARGGQVTYS |
| | | | RGPLDVWGQGTTVTV |
| | | | SSASTKGPSVFPLAP |
| | | | SSKSTSGGTAALGCL |
| | | | VKDYFPEPVTVSWNS |
| | | | GALTSGVHTFPAVLQ |
| | | | SSGLYSLSSVVTVPS |
| | | | SSLGTQTYICNVNHK |
| | | | PSNTKVDKKVEPKSC |
| | | | DKTHTCPPCPAPEAA |
| | | | GAPSVFLFPPKPKDT |
| | | | LMISRTPEVTCVVVD |
| | | | VSHEDPEVKFNWYVD |
| | | | GVEVHNAKTKPREEQ |
| | | | YNSTYRVVSVLTVLH |
| | | | QDWLNGKEYKCKVSN |
| | | | KALPAPIEKTISKAK |
| | | | GQPREPQVYTLPPSR |
| | | | EEMTKNQVSLTCLVK |
| | | | GFYPSDIAVEWESNG |
| | | | QPENNYKTTPPVLDS |
| | | | DGSFFLYSKLTVDKS |
| | | | RWQQGNVFSCSVMHE |
| | | | ALHNHYTQKSLSLSP |
| | | | GK |
| 254 | IGG1 AAA HC | 38426 | QVQLQESGPGLVKPS |
| | | | ETLSLTCAVSGYSIL |
| | | | SGYYWFWIRQPPGKG |
| | | | LEWIGGIYHSGSTAY |
| | | | NPSLKSRVTISVDTS |
| | | | KNQFSLKLSSVTAAD |
| | | | TAVYYCARGGEVTYS |
| | | | RGPLDVWGQGTTVTV |
| | | | SSASTKGPSVFPLAP |
| | | | SSKSTSGGTAALGCL |
| | | | VKDYFPEPVTVSWNS |
| | | | GALTSGVHTFPAVLQ |
| | | | SSGLYSLSSVVTVPS |
| | | | SSLGTQTYICNVNHK |
| | | | PSNTKVDKKVEPKSC |
| | | | DKTHTCPPCPAPEAA |
| | | | GAPSVFLFPPKPKDT |
| | | | LMISRTPEVTCVVVD |
| | | | VSHEDPEVKFNWYVD |
| | | | GVEVHNAKTKPREEQ |
| | | | YNSTYRVVSVLTVLH |
| | | | QDWLNGKEYKCKVSN |
| | | | KALPAPIEKTISKAK |
| | | | GQPREPQVYTLPPSR |
| | | | EEMTKNQVSLTCLVK |
| | | | GFYPSDIAVEWESNG |
| | | | QPENNYKTTPPVLDS |
| | | | DGSFFLYSKLTVDKS |
| | | | RWQQGNVFSCSVMHE |
| | | | ALHNHYTQKSLSLSP |
| | | | GK |
| 255 | IGG1 AAA HC | 37323 | EVQLLESGGGLVQPG |
| | | | GSLRLSCAASGFTFD |
| | | | NYAMHWVRQAPGKGL |
| | | | EWVSAISARAGITYY |
| | | | ADSVKGRFTISRDNS |
| | | | KNTLYLQMNSLRAED |
| | | | TAVYYCARRIGYSYG |
| | | | TAPPFDVWGQGTTVT |
| | | | VSSASTKGPSVFPLA |
| | | | PSSKSTSGGTAALGC |
| | | | LVKDYFPEPVTVSWN |
| | | | SGALTSGVHTFPAVL |
| | | | QSSGLYSLSSVVTVP |
| | | | SSSLGTQTYICNVNH |
| | | | KPSNTKVDKKVEPKS |
| | | | CDKTHTCPPCPAPEA |
| | | | AGAPSVFLFPPKPKD |
| | | | TLMISRTPEVTCVVV |
| | | | DVSHEDPEVKFNWYV |
| | | | DGVEVHNAKTKPREE |
| | | | QYNSTYRVVSVLTVL |
| | | | HQDWLNGKEYKCKVS |
| | | | NKALPAPIEKTISKA |
| | | | KGQPREPQVYTLPPS |
| | | | REEMTKNQVSLTCLV |
| | | | KGFYPSDIAVEWESN |
| | | | GQPENNYKTTPPVLD |
| | | | SDGSFFLYSKLTVDK |
| | | | SRWQQGNVFSCSVMH |
| | | | EALHNHYTQKSLSLS |
| | | | PGK |
| 256 | IGG1 AAA HC | 38389 | QVQLVESGGGLVQPG |
| | | | GSLRLSCAASGFTFS |
| | | | DYYMSWIRQAPGKGL |
| | | | EWVSYIASSGSVIYY |
| | | | ADSVKGRFTISRDNA |
| | | | KNSLYLQMNSLRAED |
| | | | TAVYYCARHGTPRAF |
| | | | DIWGQGTTVTVSSAS |
| | | | TKGPSVFPLAPSSKS |
| | | | TSGGTAALGCLVKDY |
| | | | FPEPVTVSWNSGALT |
| | | | SGVHTFPAVLQSSGL |
| | | | YSLSSVVTVPSSSLG |
| | | | TQTYICNVNHKPSNT |
| | | | KVDKKVEPKSCDKTH |
| | | | TCPPCPAPEAAGAPS |
| | | | VFLFPPKPKDTLMIS |
| | | | RTPEVTCVVVDVSHE |
| | | | DPEVKFNWYVDGVEV |
| | | | HNAKTKPREEQYNST |
| | | | YRVVSVLTVLHQDWL |
| | | | NGKEYKCKVSNKALP |
| | | | APIEKTISKAKGQPR |
| | | | EPQVYTLPPSREEMT |
| | | | KNQVSLTCLVKGFYP |
| | | | SDIAVEWESNGQPEN |
| | | | NYKTTPPVLDSDGSF |
| | | | FLYSKLTVDKSRWQQ |
| | | | GNVFSCSVMHEALHN |
| | | | HYTQKSLSLSPGK |
| 257 | IGG1 AAA HC | 38358 | EVQLLESGGGLVQPG |
| | | | GSLRLSCAASGFTFS |
| | | | SSAMAWVRQAPGKGL |
| | | | EWVSTISGSGITTWY |
| | | | ADSVKGRFTISRDNS |
| | | | KNTLYLQMNSLRAED |
| | | | TAVYYCAKGSRHLNA |
| | | | FNRWGQGTTVTVSSA |
| | | | STKGPSVFPLAPSSK |
| | | | STSGGTAALGCLVKD |
| | | | YFPEPVTVSWNSGAL |
| | | | TSGVHTFPAVLQSSG |
| | | | LYSLSSVVTVPSSSL |
| | | | GTQTYICNVNHKPSN |
| | | | TKVDKKVEPKSCDKT |
| | | | HTCPPCPAPEAAGAP |
| | | | SVFLFPPKPKDTLMI |
| | | | SRTPEVTCVVVDVSH |
| | | | EDPEVKFNWYVDGVE |
| | | | VHNAKTKPREEQYNS |
| | | | TYRVVSVLTVLHQDW |
| | | | LNGKEYKCKVSNKAL |
| | | | PAPIEKTISKAKGQP |
| | | | REPQVYTLPPSREEM |
| | | | TKNQVSLTCLVKGFY |
| | | | PSDIAVEWESNGQPE |
| | | | NNYKTTPPVLDSDGS |
| | | | FFLYSKLTVDKSRWQ |
| | | | QGNVFSCSVMHEALH |
| | | | NHYTQKSLSLSPGK |
| 258 | IGG1 AAA HC | 33303 | QVQLQESGPGLVKPS |
| | | | GTLSLTCAVSGGSIS |
| | | | SSNWWSWVRQPPGKG |
| | | | LEWIGEIYHSGSTNY |
| | | | NPSLKSRVTISVDKS |
| | | | KNQFSLKLSSVTAAD |
| | | | TAVYYCARGVYHYDP |
| | | | YGMDVWGQGTTVTVS |
| | | | SASTKGPSVFPLAPS |
| | | | SKSTSGGTAALGCLV |
| | | | KDYFPEPVTVSWNSG |
| | | | ALTSGVHTFPAVLQS |
| | | | SGLYSLSSVVTVPSS |
| | | | SLGTQTYICNVNHKP |
| | | | SNTKVDKKVEPKSCD |
| | | | KTHTCPPCPAPEAAG |
| | | | APSVFLFPPKPKDTL |
| | | | MISRTPEVTCVVVDV |
| | | | SHEDPEVKFNWYVDG |
| | | | VEVHNAKTKPREEQY |
| | | | NSTYRVVSVLTVLHQ |
| | | | DWLNGKEYKCKVSNK |
| | | | ALPAPIEKTISKAKG |
| | | | QPREPQVYTLPPSRE |
| | | | EMTKNQVSLTCLVKG |
| | | | FYPSDIAVEWESNGQ |
| | | | PENNYKTTPPVLDSD |
| | | | GSFFLYSKLTVDKSR |
| | | | WQQGNVFSCSVMHEA |
| | | | LHNHYTQKSLSLSPG |
| | | | K |
| 259 | IGG1 AAA HC | 33342 | QLQLQESGPGLVKPS |
| | | | ETLSLTCTVSGGSIS |
| | | | SSSYYWGWIRQPPGK |
| | | | GLEWIGSISYSGSTY |
| | | | YNPSLKSRVTISVDT |
| | | | SKNQFSLKLSSVTAA |
| | | | DTAVYYCARTELGKM |
| | | | HFDYWGQGTLVTVSS |
| | | | ASTKGPSVFPLAPSS |
| | | | KSTSGGTAALGCLVK |
| | | | DYFPEPVTVSWNSGA |
| | | | LTSGVHTFPAVLQSS |
| | | | GLYSLSSVVTVPSSS |
| | | | LGTQTYICNVNHKPS |
| | | | NTKVDKKVEPKSCDK |
| | | | THTCPPCPAPEAAGA |
| | | | PSVFLFPPKPKDTLM |
| | | | ISRTPEVTCVVVDVS |
| | | | HEDPEVKFNWYVDGV |
| | | | EVHNAKTKPREEQYN |
| | | | STYRVVSVLTVLHQD |
| | | | WLNGKEYKCKVSNKA |
| | | | LPAPIEKTISKAKGQ |
| | | | PREPQVYTLPPSREE |
| | | | MTKNQVSLTCLVKGF |
| | | | YPSDIAVEWESNGQP |
| | | | ENNYKTTPPVLDSDG |
| | | | SFFLYSKLTVDKSRW |
| | | | QQGNVFSCSVMHEAL |
| | | | HNHYTQKSLSLSPGK |
| 260 | IGG1 AAA HC | 33299 | QLQLQESGPGLVKPS |
| | | | ETLSLTCTVSGGSIS |
| | | | SSDYYWGWIRQPPGK |
| | | | GLEWIGSIYYSGSTY |
| | | | YNPSLKSRVTISVDT |
| | | | SKNQFSLKLSSVTAA |
| | | | DTAVYYCARGSPRYM |
| | | | QDWGQGTLVTVSSAS |
| | | | TKGPSVFPLAPSSKS |
| | | | TSGGTAALGCLVKDY |
| | | | FPEPVTVSWNSGALT |
| | | | SGVHTFPAVLQSSGL |
| | | | YSLSSVVTVPSSSLG |
| | | | TQTYICNVNHKPSNT |
| | | | KVDKKVEPKSCDKTH |
| | | | TCPPCPAPEAAGAPS |
| | | | VFLFPPKPKDTLMIS |
| | | | RTPEVTCVVVDVSHE |
| | | | DPEVKFNWYVDGVEV |
| | | | HNAKTKPREEQYNST |
| | | | YRVVSVLTVLHQDWL |
| | | | NGKEYKCKVSNKALP |
| | | | APIEKTISKAKGQPR |
| | | | EPQVYTLPPSREEMT |
| | | | KNQVSLTCLVKGFYP |
| | | | SDIAVEWESNGQPEN |
| | | | NYKTTPPVLDSDGSF |
| | | | FLYSKLTVDKSRWQQ |
| | | | GNVFSCSVMHEALHN |
| | | | HYTQKSLSLSPGK |
| 261 | IGG1 AAA HC | 33351 | QVQLVESGGGLVKPG |
| | | | GSLRLSCAASGFTFS |
| | | | DYYMSWIRQAPGKGL |
| | | | EWVSYISSSGSTIYY |
| | | | ADSVKGRFTISRDNA |
| | | | KNSLYLQMNSLRAED |
| | | | TAVYYCARHSSLGTH |
| | | | NWFDPWGQGTLVTVS |
| | | | SASTKGPSVFPLAPS |
| | | | SKSTSGGTAALGCLV |
| | | | KDYFPEPVTVSWNSG |
| | | | ALTSGVHTFPAVLQS |
| | | | SGLYSLSSVVTVPSS |
| | | | SLGTQTYICNVNHKP |
| | | | SNTKVDKKVEPKSCD |
| | | | KTHTCPPCPAPEAAG |
| | | | APSVFLFPPKPKDTL |
| | | | MISRTPEVTCVVVDV |
| | | | SHEDPEVKFNWYVDG |
| | | | VEVHNAKTKPREEQY |
| | | | NSTYRVVSVLTVLHQ |
| | | | DWLNGKEYKCKVSNK |
| | | | ALPAPIEKTISKAKG |
| | | | QPREPQVYTLPPSRE |
| | | | EMTKNQVSLTCLVKG |
| | | | FYPSDIAVEWESNGQ |
| | | | PENNYKTTPPVLDSD |
| | | | GSFFLYSKLTVDKSR |
| | | | WQQGNVFSCSVMHEA |
| | | | LHNHYTQKSLSLSPG |
| | | | K |
| 262 | IGG1 AAA HC | 33357 | QVQLQESGPGLVKPS |
| | | | ETLSLTCAVSGYSIS |
| | | | SGYYWGWIRQPPGKG |
| | | | LEWIGSIYHSGSTYY |
| | | | NPSLKSRVTISVDTS |
| | | | KNQFSLKLSSVTAAD |
| | | | TAVYYCAREGALSYS |
| | | | WLAAFDIWGQGTMVT |
| | | | VSSASTKGPSVFPLA |
| | | | PSSKSTSGGTAALGC |
| | | | LVKDYFPEPVTVSWN |
| | | | SGALTSGVHTFPAVL |
| | | | QSSGLYSLSSVVTVP |
| | | | SSSLGTQTYICNVNH |
| | | | KPSNTKVDKKVEPKS |
| | | | CDKTHTCPPCPAPEA |
| | | | AGAPSVFLFPPKPKD |
| | | | TLMISRTPEVTCVVV |
| | | | DVSHEDPEVKFNWYV |
| | | | DGVEVHNAKTKPREE |
| | | | QYNSTYRVVSVLTVL |
| | | | HQDWLNGKEYKCKVS |
| | | | NKALPAPIEKTISKA |
| | | | KGQPREPQVYTLPPS |
| | | | REEMTKNQVSLTCLV |
| | | | KGFYPSDIAVEWESN |
| | | | GQPENNYKTTPPVLD |
| | | | SDGSFFLYSKLTVDK |
| | | | SRWQQGNVFSCSVMH |
| | | | EALHNHYTQKSLSLS |
| | | | PGK |
| 263 | | | |
| 264 | | | |
| 265 | | | |
| 266 | IGG4 HC | 33343 | QLQLQESGPGLVKPS |
| | | | ETLSLTCTVSGGSIS |
| | | | SSDYYWGWIRQPPGK |
| | | | GLEWIGSIYYSGSTY |
| | | | YNPSLKSRVTISVDT |
| | | | SKNQFSLKLSSVTAA |
| | | | DTAVYYCARGVRRAV |
| | | | PFDYWGQGTLVTVSS |
| | | | ASTKGPSVFPLAPCS |
| | | | RSTSESTAALGCLVK |
| | | | DYFPEPVTVSWNSGA |
| | | | LTSGVHTFPAVLQSS |
| | | | GLYSLSSVVTVPSSS |
| | | | LGTKTYTCNVDHKPS |
| | | | NTKVDKRVESKYGPP |
| | | | CPPCPAPEFLGGPSV |
| | | | FLFPPKPKDTLMISR |
| | | | TPEVTCVVVDVSQED |
| | | | PEVQFNWYVDGVEVH |
| | | | NAKTKPREEQFNSTY |
| | | | RVVSVLTVLHQDWLN |
| | | | GKEYKCKVSNKGLPS |
| | | | SIEKTISKAKGQPRE |
| | | | PQVYTLPPSQEEMTK |
| | | | NQVSLTCLVKGFYPS |
| | | | DIAVEWESNGQPENN |
| | | | YKTTPPVLDSDGSFF |
| | | | LYSRLTVDKSRWQEG |
| | | | NVFSCSVMHEALHNH |
| | | | YTQKSLSLSPGK |
| 267 | IGG4 HC | 37268 | QLQLQESGPGLVKPS |
| | | | ETLSLTCTVSGGSIS |
| | | | SSSTYWAWIRQPPGK |
| | | | GLEWIGSISSSGSTY |
| | | | YNPSLKSRVTISVDT |
| | | | SKNQFSLKLSSVTAA |
| | | | DTAVYYCARGIARAV |
| | | | PFDYWGQGTLVTVSS |
| | | | ASTKGPSVFPLAPCS |
| | | | RSTSESTAALGCLVK |
| | | | DYFPEPVTVSWNSGA |
| | | | LTSGVHTFPAVLQSS |
| | | | GLYSLSSVVTVPSSS |
| | | | LGTKTYTCNVDHKPS |
| | | | NTKVDKRVESKYGPP |
| | | | CPPCPAPEFLGGPSV |
| | | | FLFPPKPKDTLMISR |
| | | | TPEVTCVVVDVSQED |
| | | | PEVQFNWYVDGVEVH |
| | | | NAKTKPREEQFNSTY |
| | | | RVVSVLTVLHQDWLN |
| | | | GKEYKCKVSNKGLPS |
| | | | SIEKTISKAKGQPRE |
| | | | PQVYTLPPSQEEMTK |
| | | | NQVSLTCLVKGFYPS |
| | | | DIAVEWESNGQPENN |
| | | | YKTTPPVLDSDGSFF |
| | | | LYSRLTVDKSRWQEG |
| | | | NVFSCSVMHEALHNH |
| | | | YTQKSLSLSPGK |
| 268 | IGG4 HC | 37269 | QLQLQESGPGLVKPS |
| | | | ETLSLTCTVSGGSIS |
| | | | SSSTYWGWIRQSPGK |
| | | | GLEWIGSIHHSGATY |
| | | | YNPSLKSRVTISVDT |
| | | | SKNQFSLKLSSVTAA |
| | | | DTAVYYCARGPKRAV |
| | | | PFDYWGQGTLVTVSS |
| | | | ASTKGPSVFPLAPCS |
| | | | RSTSESTAALGCLVK |
| | | | DYFPEPVTVSWNSGA |
| | | | LTSGVHTFPAVLQSS |
| | | | GLYSLSSVVTVPSSS |
| | | | LGTKTYTCNVDHKPS |
| | | | NTKVDKRVESKYGPP |
| | | | CPPCPAPEFLGGPSV |
| | | | FLFPPKPKDTLMISR |
| | | | TPEVTCVVVDVSQED |
| | | | PEVQFNWYVDGVEVH |
| | | | NAKTKPREEQFNSTY |
| | | | RVVSVLTVLHQDWLN |
| | | | GKEYKCKVSNKGLPS |
| | | | SIEKTISKAKGQPRE |
| | | | PQVYTLPPSQEEMTK |
| | | | NQVSLTCLVKGFYPS |
| | | | DIAVEWESNGQPENN |
| | | | YKTTPPVLDSDGSFF |
| | | | LYSRLTVDKSRWQEG |
| | | | NVFSCSVMHEALHNH |
| | | | YTQKSLSLSPGK |
| 269 | IGG4 HC | 37271 | QLQLQESGPGLVKPS |
| | | | ETLSLTCTVSGGSIS |
| | | | SSDTYWGWIRQPPGK |
| | | | GLEWIGSIHYSGSTL |
| | | | YNPSLKSRVTISVDT |
| | | | SKNQFSLKLSSVTAA |
| | | | DTAVYYCARGVRRAV |
| | | | PFVDWGQGTLVTVSS |
| | | | ASTKGPSVFPLAPCS |
| | | | RSTSESTAALGCLVK |
| | | | DYFPEPVTVSWNSGA |
| | | | LTSGVHTFPAVLQSS |
| | | | GLYSLSSVVTVPSSS |
| | | | LGTKTYTCNVDHKPS |
| | | | NTKVDKRVESKYGPP |
| | | | CPPCPAPEFLGGPSV |
| | | | FLFPPKPKDTLMISR |
| | | | TPEVTCVVVDVSQED |
| | | | PEVQFNWYVDGVEVH |
| | | | NAKTKPREEQFNSTY |
| | | | RVVSVLTVLHQDWLN |
| | | | GKEYKCKVSNKGLPS |
| | | | SIEKTISKAKGQPRE |
| | | | PQVYTLPPSQEEMTK |
| | | | NQVSLTCLVKGFYPS |
| | | | DIAVEWESNGQPENN |
| | | | YKTTPPVLDSDGSFF |
| | | | LYSRLTVDKSRWQEG |
| | | | NVFSCSVMHEALHNH |
| | | | YTQKSLSLSPGK |
| 270 | IGG4 HC | 37272 | QLQLQESGPGLVKPS |
| | | | ETLSLTCTVSGGSIS |
| | | | SADNYWGWIRQPPGK |
| | | | GLEWIGSIHYSGSTY |
| | | | YNPSLKSRVTISVDT |
| | | | SKNQFSLKLSSVTAA |
| | | | DTAVYYCARGVRRAV |
| | | | PFQRWGQGTLVTVSS |
| | | | ASTKGPSVFPLAPCS |
| | | | RSTSESTAALGCLVK |
| | | | DYFPEPVTVSWNSGA |
| | | | LTSGVHTFPAVLQSS |
| | | | GLYSLSSVVTVPSSS |
| | | | LGTKTYTCNVDHKPS |
| | | | NTKVDKRVESKYGPP |
| | | | CPPCPAPEFLGGPSV |
| | | | FLFPPKPKDTLMISR |
| | | | TPEVTCVVVDVSQED |
| | | | PEVQFNWYVDGVEVH |
| | | | NAKTKPREEQFNSTY |
| | | | RVVSVLTVLHQDWLN |
| | | | GKEYKCKVSNKGLPS |
| | | | SIEKTISKAKGQPRE |
| | | | PQVYTLPPSQEEMTK |
| | | | NQVSLTCLVKGFYPS |
| | | | DIAVEWESNGQPENN |
| | | | YKTTPPVLDSDGSFF |
| | | | LYSRLTVDKSRWQEG |
| | | | NVFSCSVMHEALHNH |
| | | | YTQKSLSLSPGK |
| 271 | IGG4 HC | 37277 | QLQLQESGPGLVKPS |
| | | | ETLSLTCTVSGGSIS |
| | | | SSDTYWGWIRQPPGK |
| | | | GPEWIGSIHYSGSTL |
| | | | YNPSLKSRVTISVDT |
| | | | SKNQFSLKLSSVTAA |
| | | | DTAVYYCARGVRRAV |
| | | | PFDYWGQGTLVTVSS |
| | | | ASTKGPSVFPLAPCS |
| | | | RSTSESTAALGCLVK |
| | | | DYFPEPVTVSWNSGA |
| | | | LTSGVHTFPAVLQSS |
| | | | GLYSLSSVVTVPSSS |
| | | | LGTKTYTCNVDHKPS |
| | | | NTKVDKRVESKYGPP |
| | | | CPPCPAPEFLGGPSV |
| | | | FLFPPKPKDTLMISR |
| | | | TPEVTCVVVDVSQED |
| | | | PEVQFNWYVDGVEVH |
| | | | NAKTKPREEQFNSTY |
| | | | RVVSVLTVLHQDWLN |
| | | | GKEYKCKVSNKGLPS |
| | | | SIEKTISKAKGQPRE |
| | | | PQVYTLPPSQEEMTK |
| | | | NQVSLTCLVKGFYPS |
| | | | DIAVEWESNGQPENN |
| | | | YKTTPPVLDSDGSFF |
| | | | LYSRLTVDKSRWQEG |
| | | | NVFSCSVMHEALHNH |
| | | | YTQKSLSLSPGK |
| 272 | IGG4 HC | 38373 | QLQLQESGPGLVKPS |
| | | | ETLSLTCTVSGGSIS |
| | | | SSSTYWSWIRQPPGK |
| | | | GLEWIGGIAYSGSTY |
| | | | YNPSLKSRVTISVDT |
| | | | SKNQFSLKLSSVTAA |
| | | | DTAVYYCARGVRRAV |
| | | | PFDYWGQGTLVTVSS |
| | | | ASTKGPSVFPLAPCS |
| | | | RSTSESTAALGCLVK |
| | | | DYFPEPVTVSWNSGA |
| | | | LTSGVHTFPAVLQSS |
| | | | GLYSLSSVVTVPSSS |
| | | | LGTKTYTCNVDHKPS |
| | | | NTKVDKRVESKYGPP |
| | | | CPPCPAPEFLGGPSV |
| | | | FLFPPKPKDTLMISR |
| | | | TPEVTCVVVDVSQED |
| | | | PEVQFNWYVDGVEVH |
| | | | NAKTKPREEQFNSTY |
| | | | RVVSVLTVLHQDWLN |
| | | | GKEYKCKVSNKGLPS |
| | | | SIEKTISKAKGQPRE |
| | | | PQVYTLPPSQEEMTK |
| | | | NQVSLTCLVKGFYPS |
| | | | DIAVEWESNGQPENN |
| | | | YKTTPPVLDSDGSFF |
| | | | LYSRLTVDKSRWQEG |
| | | | NVFSCSVMHEALHNH |
| | | | YTQKSLSLSPGK |
| 273 | IGG4 HC | 38375 | QLQLQESGPGLVKPS |
| | | | ETLSLTCTVSGGSIS |
| | | | SSSTYWSWIRQPPGK |
| | | | GLEWIGSISYNALTY |
| | | | YNPSLKSRVTISVDT |
| | | | SKNQFSLKLSSVTAA |
| | | | DTAVYYCARGTRRAV |
| | | | PFDYWGQGTLVTVSS |
| | | | ASTKGPSVFPLAPCS |
| | | | RSTSESTAALGCLVK |
| | | | DYFPEPVTVSWNSGA |
| | | | LTSGVHTFPAVLQSS |
| | | | GLYSLSSVVTVPSSS |
| | | | LGTKTYTCNVDHKPS |
| | | | NTKVDKRVESKYGPP |
| | | | CPPCPAPEFLGGPSV |
| | | | FLFPPKPKDTLMISR |
| | | | TPEVTCVVVDVSQED |
| | | | PEVQFNWYVDGVEVH |
| | | | NAKTKPREEQFNSTY |
| | | | RVVSVLTVLHQDWLN |
| | | | GKEYKCKVSNKGLPS |
| | | | SIEKTISKAKGQPRE |
| | | | PQVYTLPPSQEEMTK |
| | | | NQVSLTCLVKGFYPS |
| | | | DIAVEWESNGQPENN |
| | | | YKTTPPVLDSDGSFF |
| | | | LYSRLTVDKSRWQEG |
| | | | NVFSCSVMHEALHNH |
| | | | YTQKSLSLSPGK |
| 274 | IGG4 HC | 38379 | QLQLQESGPGLVKPS |
| | | | ETLSLTCTVSGGSIS |
| | | | SSSTYWSWIRQPPGK |
| | | | GLEWIGGIAYSGSTY |
| | | | YNPSLKSRVTISVDT |
| | | | SKNQFSLKLSSVTAA |
| | | | DTAVYYCARGVRRAV |
| | | | PFADWGQGTLVTVSS |
| | | | ASTKGPSVFPLAPCS |
| | | | RSTSESTAALGCLVK |
| | | | DYFPEPVTVSWNSGA |
| | | | LTSGVHTFPAVLQSS |
| | | | GLYSLSSVVTVPSSS |
| | | | LGTKTYTCNVDHKPS |
| | | | NTKVDKRVESKYGPP |
| | | | CPPCPAPEFLGGPSV |
| | | | FLFPPKPKDTLMISR |
| | | | TPEVTCVVVDVSQED |
| | | | PEVQFNWYVDGVEVH |
| | | | NAKTKPREEQFNSTY |
| | | | RVVSVLTVLHQDWLN |
| | | | GKEYKCKVSNKGLPS |
| | | | SIEKTISKAKGQPRE |
| | | | PQVYTLPPSQEEMTK |
| | | | NQVSLTCLVKGFYPS |
| | | | DIAVEWESNGQPENN |
| | | | YKTTPPVLDSDGSFF |
| | | | LYSRLTVDKSRWQEG |
| | | | NVFSCSVMHEALHNH |
| | | | YTQKSLSLSPGK |
| 275 | IGG4 HC | 38381 | QLQLQESGPGLVKPS |
| | | | ETLSLTCTVSGGSVS |
| | | | SSSTYWSWIRQPPGK |
| | | | GLEWIGGIAYSGSTY |
| | | | YNPSLKSRVTISVDT |
| | | | SKNQFSLKLSSVTAA |
| | | | DTAVYYCARGVRRAV |
| | | | PFDYWGQGTLVTVSS |
| | | | ASTKGPSVFPLAPCS |
| | | | RSTSESTAALGCLVK |
| | | | DYFPEPVTVSWNSGA |
| | | | LTSGVHTFPAVLQSS |
| | | | GLYSLSSVVTVPSSS |
| | | | LGTKTYTCNVDHKPS |
| | | | NTKVDKRVESKYGPP |
| | | | CPPCPAPEFLGGPSV |
| | | | FLFPPKPKDTLMISR |
| | | | TPEVTCVVVDVSQED |
| | | | PEVQFNWYVDGVEVH |
| | | | NAKTKPREEQFNSTY |
| | | | RVVSVLTVLHQDWLN |
| | | | GKEYKCKVSNKGLPS |
| | | | SIEKTISKAKGQPRE |
| | | | PQVYTLPPSQEEMTK |
| | | | NQVSLTCLVKGFYPS |
| | | | DIAVEWESNGQPENN |
| | | | YKTTPPVLDSDGSFF |
| | | | LYSRLTVDKSRWQEG |
| | | | NVFSCSVMHEALHNH |
| | | | YTQKSLSLSPGK |
| 276 | IGG4 HC | 38383 | QLQLQESGPGLVKPS |
| | | | ETLSLTCTVSGGSIS |
| | | | SSSTYWSWIRQPPGK |
| | | | GLEWIGGIAYSGSTY |
| | | | YNPSLKSRVTISVDT |
| | | | SKNQFSLKLSSVTAA |
| | | | DTAVYYCARGTRRAV |
| | | | PFDYWGQGTLVTVSS |
| | | | ASTKGPSVFPLAPCS |
| | | | RSTSESTAALGCLVK |
| | | | DYFPEPVTVSWNSGA |
| | | | LTSGVHTFPAVLQSS |
| | | | GLYSLSSVVTVPSSS |
| | | | LGTKTYTCNVDHKPS |
| | | | NTKVDKRVESKYGPP |
| | | | CPPCPAPEFLGGPSV |
| | | | FLFPPKPKDTLMISR |
| | | | TPEVTCVVVDVSQED |
| | | | PEVQFNWYVDGVEVH |
| | | | NAKTKPREEQFNSTY |
| | | | RVVSVLTVLHQDWLN |
| | | | GKEYKCKVSNKGLPS |
| | | | SIEKTISKAKGQPRE |
| | | | PQVYTLPPSQEEMTK |
| | | | NQVSLTCLVKGFYPS |
| | | | DIAVEWESNGQPENN |
| | | | YKTTPPVLDSDGSFF |
| | | | LYSRLTVDKSRWQEG |
| | | | NVFSCSVMHEALHNH |
| | | | YTQKSLSLSPGK |
| 277 | IGG4 HC | 38386 | QLQLQESGPGLVKPS |
| | | | ETLSLTCTVSGGSIS |
| | | | SSDTYWGWIRQPPGK |
| | | | GLEWIGSIHYSGSTL |
| | | | YNPSLKSRVTISVDT |
| | | | SKNQFSLKLSSVTAA |
| | | | DTAVYYCARGIRRAV |
| | | | PFDYWGQGTLVTVSS |
| | | | ASTKGPSVFPLAPCS |
| | | | RSTSESTAALGCLVK |
| | | | DYFPEPVTVSWNSGA |
| | | | LTSGVHTFPAVLQSS |
| | | | GLYSLSSVVTVPSSS |
| | | | LGTKTYTCNVDHKPS |
| | | | NTKVDKRVESKYGPP |
| | | | CPPCPAPEFLGGPSV |
| | | | FLFPPKPKDTLMISR |
| | | | TPEVTCVVVDVSQED |
| | | | PEVQFNWYVDGVEVH |
| | | | NAKTKPREEQFNSTY |
| | | | RVVSVLTVLHQDWLN |
| | | | GKEYKCKVSNKGLPS |
| | | | SIEKTISKAKGQPRE |
| | | | PQVYTLPPSQEEMTK |
| | | | NQVSLTCLVKGFYPS |
| | | | DIAVEWESNGQPENN |
| | | | YKTTPPVLDSDGSFF |
| | | | LYSRLTVDKSRWQEG |
| | | | NVFSCSVMHEALHNH |
| | | | YTQKSLSLSPGK |
| 278 | IGG4 HC | 37273 | QLQLQESGPGLVKPS |
| | | | ETLSLTCTVSGGSIS |
| | | | SSDTYWGWIRQPPGK |
| | | | GLEWIGSIHYSGSTL |
| | | | YNPSLKSRVTISVDT |
| | | | SKNQFSLKLSSVTAA |
| | | | DTAVYYCARGQFRAV |
| | | | PFDYWGQGTLVTVSS |
| | | | ASTKGPSVFPLAPCS |
| | | | RSTSESTAALGCLVK |
| | | | DYFPEPVTVSWNSGA |
| | | | LTSGVHTFPAVLQSS |
| | | | GLYSLSSVVTVPSSS |
| | | | LGTKTYTCNVDHKPS |
| | | | NTKVDKRVESKYGPP |
| | | | CPPCPAPEFLGGPSV |
| | | | FLFPPKPKDTLMISR |
| | | | TPEVTCVVVDVSQED |
| | | | PEVQFNWYVDGVEVH |
| | | | NAKTKPREEQFNSTY |
| | | | RVVSVLTVLHQDWLN |
| | | | GKEYKCKVSNKGLPS |
| | | | SIEKTISKAKGQPRE |
| | | | PQVYTLPPSQEEMTK |
| | | | NQVSLTCLVKGFYPS |
| | | | DIAVEWESNGQPENN |
| | | | YKTTPPVLDSDGSFF |
| | | | LYSRLTVDKSRWQEG |
| | | | NVFSCSVMHEALHNH |
| | | | YTQKSLSLSPGK |
| 279 | IGG4 HC | 33361 | QVQLQESGPGLVKPS |
| | | | ETLSLTCAVSGYSIS |
| | | | SGYYWGWIRQPPGKG |
| | | | LEWIGSIYHSGSTYY |
| | | | NPSLKSRVTISVDTS |
| | | | KNQFSLKLSSVTAAD |
| | | | TAVYYCARGGTHTYS |
| | | | RGPMDVWGQGTTVTV |
| | | | SSASTKGPSVFPLAP |
| | | | CSRSTSESTAALGCL |
| | | | VKDYFPEPVTVSWNS |
| | | | GALTSGVHTFPAVLQ |
| | | | SSGLYSLSSVVTVPS |
| | | | SSLGTKTYTCNVDHK |
| | | | PSNTKVDKRVESKYG |
| | | | PPCPPCPAPEFLGGP |
| | | | SVFLFPPKPKDTLMI |
| | | | SRTPEVTCVVVDVSQ |
| | | | EDPEVQFNWYVDGVE |
| | | | VHNAKTKPREEQFNS |
| | | | TYRVVSVLTVLHQDW |
| | | | LNGKEYKCKVSNKGL |
| | | | PSSIEKTISKAKGQP |
| | | | REPQVYTLPPSQEEM |
| | | | TKNQVSLTCLVKGFY |
| | | | PSDIAVEWESNGQPE |
| | | | NNYKTTPPVLDSDGS |
| | | | FFLYSRLTVDKSRWQ |
| | | | EGNVFSCSVMHEALH |
| | | | NHYTQKSLSLSPGK |
| 280 | IGG4 HC | 35624 | QLQLQESGPRLVKPS |
| | | | ETLSLTCAVSGYSIS |
| | | | SGYYWGWIRQPPGKG |
| | | | LEWIGSIYHSGSTYY |
| | | | NPSLKSRVTISVDTS |
| | | | KNQFSLKLSSVTAAD |
| | | | TAVYYCARGGTHTYS |
| | | | RGPFDVWGQGTTVTV |
| | | | SSASTKGPSVFPLAP |
| | | | CSRSTSESTAALGCL |
| | | | VKDYFPEPVTVSWNS |
| | | | GALTSGVHTFPAVLQ |
| | | | SSGLYSLSSVVTVPS |
| | | | SSLGTKTYTCNVDHK |
| | | | PSNTKVDKRVESKYG |
| | | | PPCPPCPAPEFLGGP |
| | | | SVFLFPPKPKDTLMI |
| | | | SRTPEVTCVVVDVSQ |
| | | | EDPEVQFNWYVDGVE |
| | | | VHNAKTKPREEQFNS |
| | | | TYRVVSVLTVLHQDW |
| | | | LNGKEYKCKVSNKGL |
| | | | PSSIEKTISKAKGQP |
| | | | REPQVYTLPPSQEEM |
| | | | TKNQVSLTCLVKGFY |
| | | | PSDIAVEWESNGQPE |
| | | | NNYKTTPPVLDSDGS |
| | | | FFLYSRLTVDKSRWQ |
| | | | EGNVFSCSVMHEALH |
| | | | NHYTQKSLSLSPGK |
| 281 | IGG4 HC | 38410 | LVQLQESGPGLVKPS |
| | | | ETLSLTCAVSGYSIL |
| | | | SGYYWFWIRQPPGKG |
| | | | LEWIGGIYHSASTAY |
| | | | NPSLKSRVTISVDTS |
| | | | KNQFSLKLSSVTAAD |
| | | | TAVYYCARGGTPIYS |
| | | | RGPLDVWGQGTTVTV |
| | | | SSASTKGPSVFPLAP |
| | | | CSRSTSESTAALGCL |
| | | | VKDYFPEPVTVSWNS |
| | | | GALTSGVHTFPAVLQ |
| | | | SSGLYSLSSVVTVPS |
| | | | SSLGTKTYTCNVDHK |
| | | | PSNTKVDKRVESKYG |
| | | | PPCPPCPAPEFLGGP |
| | | | SVFLFPPKPKDTLMI |
| | | | SRTPEVTCVVVDVSQ |
| | | | EDPEVQFNWYVDGVE |
| | | | VHNAKTKPREEQFNS |
| | | | TYRVVSVLTVLHQDW |
| | | | LNGKEYKCKVSNKGL |
| | | | PSSIEKTISKAKGQP |
| | | | REPQVYTLPPSQEEM |
| | | | TKNQVSLTCLVKGFY |
| | | | PSDIAVEWESNGQPE |
| | | | NNYKTTPPVLDSDGS |
| | | | FFLYSRLTVDKSRWQ |
| | | | EGNVFSCSVMHEALH |
| | | | NHYTQKSLSLSPGK |
| 282 | IGG4 HC | 38418 | QVQLQESGPGLVKPS |
| | | | ETLSLTCAVSGYSIS |
| | | | SGHYWIWIRQPPGKG |
| | | | LEWIGGIYHSGSTYY |
| | | | NPSLKSRVTISVDTS |
| | | | KDQFSLKLSSVTAAD |
| | | | TAVYYCARGGGQTYS |
| | | | RGPLDVWGQGTTVTV |
| | | | SSASTKGPSVFPLAP |
| | | | CSRSTSESTAALGCL |
| | | | VKDYFPEPVTVSWNS |
| | | | GALTSGVHTFPAVLQ |
| | | | SSGLYSLSSVVTVPS |
| | | | SSLGTKTYTCNVDHK |
| | | | PSNTKVDKRVESKYG |
| | | | PPCPPCPAPEFLGGP |
| | | | SVFLFPPKPKDTLMI |
| | | | SRTPEVTCVVVDVSQ |
| | | | EDPEVQFNWYVDGVE |
| | | | VHNAKTKPREEQFNS |
| | | | TYRVVSVLTVLHQDW |
| | | | LNGKEYKCKVSNKGL |
| | | | PSSIEKTISKAKGQP |
| | | | REPQVYTLPPSQEEM |
| | | | TKNQVSLTCLVKGFY |
| | | | PSDIAVEWESNGQPE |
| | | | NNYKTTPPVLDSDGS |
| | | | FFLYSRLTVDKSRWQ |
| | | | EGNVFSCSVMHEALH |
| | | | NHYTQKSLSLSPGK |
| 283 | IGG4 HC | 38420 | QVQLQESGPGLVKPP |
| | | | ETLSLTCAVSGYSIS |
| | | | SGHYWIWIRQPPGKG |
| | | | LEWIGGIYHSGSTYY |
| | | | NPSLKSRVTISVDTS |
| | | | KNQFSLKLSSVTAAD |
| | | | TAVYYCARGGGATYS |
| | | | RGPLDVWGQGTTVTV |
| | | | SSASTKGPSVFPLAP |
| | | | CSRSTSESTAALGCL |
| | | | VKDYFPEPVTVSWNS |
| | | | GALTSGVHTFPAVLQ |
| | | | SSGLYSLSSVVTVPS |
| | | | SSLGTKTYTCNVDHK |
| | | | PSNTKVDKRVESKYG |
| | | | PPCPPCPAPEFLGGP |
| | | | SVFLFPPKPKDTLMI |
| | | | SRTPEVTCVVVDVSQ |
| | | | EDPEVQFNWYVDGVE |
| | | | VHNAKTKPREEQFNS |
| | | | TYRVVSVLTVLHQDW |
| | | | LNGKEYKCKVSNKGL |
| | | | PSSIEKTISKAKGQP |
| | | | REPQVYTLPPSQEEM |
| | | | TKNQVSLTCLVKGFY |
| | | | PSDIAVEWESNGQPE |
| | | | NNYKTTPPVLDSDGS |
| | | | FFLYSRLTVDKSRWQ |
| | | | EGNVFSCSVMHEALH |
| | | | NHYTQKSLSLSPGK |
| 284 | IGG4 HC | 38421 | QVQLQESGPGLVKPS |
| | | | ETLSLTCAVSGYSIL |
| | | | SGYYWFWIRQPPGKG |
| | | | LEWIGGIYHSGSTYY |
| | | | NPSLKSRVTISVDTS |
| | | | KNQFSLKLSSVTAAD |
| | | | TAVYYCARGGTHTYS |
| | | | RGPLDVWGQGTTVTV |
| | | | SSASTKGPSVFPLAP |
| | | | CSRSTSESTAALGCL |
| | | | VKDYFPEPVTVSWNS |
| | | | GALTSGVHTFPAVLQ |
| | | | SSGLYSLSSVVTVPS |
| | | | SSLGTKTYTCNVDHK |
| | | | PSNTKVDKRVESKYG |
| | | | PPCPPCPAPEFLGGP |
| | | | SVFLFPPKPKDTLMI |
| | | | SRTPEVTCVVVDVSQ |
| | | | EDPEVQFNWYVDGVE |
| | | | VHNAKTKPREEQFNS |
| | | | TYRVVSVLTVLHQDW |
| | | | LNGKEYKCKVSNKGL |
| | | | PSSIEKTISKAKGQP |
| | | | REPQVYTLPPSQEEM |
| | | | TKNQVSLTCLVKGFY |
| | | | PSDIAVEWESNGQPE |
| | | | NNYKTTPPVLDSDGS |
| | | | FFLYSRLTVDKSRWQ |
| | | | EGNVFSCSVMHEALH |
| | | | NHYTQKSLSLSPGK |
| 285 | IGG4 HC | 38422 | QVQLQESGPGLVKPS |
| | | | ETLSLTCAVSGYSIS |
| | | | SGFYWTWIRQPPGKG |
| | | | LEWIGAIYHSGSTVY |
| | | | NPSLKSRVTISVDTS |
| | | | KNQFSLKLSSVTAAD |
| | | | TAVYYCARGGTHTYS |
| | | | RGPLDVWGQGTTVTV |
| | | | SSASTKGPSVFPLAP |
| | | | CSRSTSESTAALGCL |
| | | | VKDYFPEPVTVSWNS |
| | | | GALTSGVHTFPAVLQ |
| | | | SSGLYSLSSVVTVPS |
| | | | SSLGTKTYTCNVDHK |
| | | | PSNTKVDKRVESKYG |
| | | | PPCPPCPAPEFLGGP |
| | | | SVFLFPPKPKDTLMI |
| | | | SRTPEVTCVVVDVSQ |
| | | | EDPEVQFNWYVDGVE |
| | | | VHNAKTKPREEQFNS |
| | | | TYRVVSVLTVLHQDW |
| | | | LNGKEYKCKVSNKGL |
| | | | PSSIEKTISKAKGQP |
| | | | REPQVYTLPPSQEEM |
| | | | TKNQVSLTCLVKGFY |
| | | | PSDIAVEWESNGQPE |
| | | | NNYKTTPPVLDSDGS |
| | | | FFLYSRLTVDKSRWQ |
| | | | EGNVFSCSVMHEALH |
| | | | NHYTQKSLSLSPGK |
| 286 | IGG4 HC | 38424 | QVQLQESGPGLVKPS |
| | | | ETLSLTCAVSGYSIS |
| | | | SGYYWLWIRQPPGKG |
| | | | LEWIGGIYHSASTAY |
| | | | NPSLKSRVTISVDTS |
| | | | KNQFSLKLSSVTAAD |
| | | | TAVYYCARGGTVKYS |
| | | | RGPLDVWGQGTTVTV |
| | | | SSASTKGPSVFPLAP |
| | | | CSRSTSESTAALGCL |
| | | | VKDYFPEPVTVSWNS |
| | | | GALTSGVHTFPAVLQ |
| | | | SSGLYSLSSVVTVPS |
| | | | SSLGTKTYTCNVDHK |
| | | | PSNTKVDKRVESKYG |
| | | | PPCPPCPAPEFLGGP |
| | | | SVFLFPPKPKDTLMI |
| | | | SRTPEVTCVVVDVSQ |
| | | | EDPEVQFNWYVDGVE |
| | | | VHNAKTKPREEQFNS |
| | | | TYRVVSVLTVLHQDW |
| | | | LNGKEYKCKVSNKGL |
| | | | PSSIEKTISKAKGQP |
| | | | REPQVYTLPPSQEEM |
| | | | TKNQVSLTCLVKGFY |
| | | | PSDIAVEWESNGQPE |
| | | | NNYKTTPPVLDSDGS |
| | | | FFLYSRLTVDKSRWQ |
| | | | EGNVFSCSVMHEALH |
| | | | NHYTQKSLSLSPGK |
| 287 | IGG4 HC | 38425 | QVQLQESGPGLVKPS |
| | | | ETLSLTCAVSGYSIS |
| | | | SGHYWTWIRQPPGKG |
| | | | LEWIGAIYHSGSTVY |
| | | | NPSLKSRVTISVDTS |
| | | | KNQFSLKLSSVTAAD |
| | | | TAVYYCARGGQVTYS |
| | | | RGPLDVWGQGTTVTV |
| | | | SSASTKGPSVFPLAP |
| | | | CSRSTSESTAALGCL |
| | | | VKDYFPEPVTVSWNS |
| | | | GALTSGVHTFPAVLQ |
| | | | SSGLYSLSSVVTVPS |
| | | | SSLGTKTYTCNVDHK |
| | | | PSNTKVDKRVESKYG |
| | | | PPCPPCPAPEFLGGP |
| | | | SVFLFPPKPKDTLMI |
| | | | SRTPEVTCVVVDVSQ |
| | | | EDPEVQFNWYVDGVE |
| | | | VHNAKTKPREEQFNS |
| | | | TYRVVSVLTVLHQDW |
| | | | LNGKEYKCKVSNKGL |
| | | | PSSIEKTISKAKGQP |
| | | | REPQVYTLPPSQEEM |
| | | | TKNQVSLTCLVKGFY |
| | | | PSDIAVEWESNGQPE |
| | | | NNYKTTPPVLDSDGS |
| | | | FFLYSRLTVDKSRWQ |
| | | | EGNVFSCSVMHEALH |
| | | | NHYTQKSLSLSPGK |
| 288 | IGG4 HC | 38426 | QVQLQESGPGLVKPS |
| | | | ETLSLTCAVSGYSIL |
| | | | SGYYWFWIRQPPGKG |
| | | | LEWIGGIYHSGSTAY |
| | | | NPSLKSRVTISVDTS |
| | | | KNQFSLKLSSVTAAD |
| | | | TAVYYCARGGEVTYS |
| | | | RGPLDVWGQGTTVTV |
| | | | SSASTKGPSVFPLAP |
| | | | CSRSTSESTAALGCL |
| | | | VKDYFPEPVTVSWNS |
| | | | GALTSGVHTFPAVLQ |
| | | | SSGLYSLSSVVTVPS |
| | | | SSLGTKTYTCNVDHK |
| | | | PSNTKVDKRVESKYG |
| | | | PPCPPCPAPEFLGGP |
| | | | SVFLFPPKPKDTLMI |
| | | | SRTPEVTCVVVDVSQ |
| | | | EDPEVQFNWYVDGVE |
| | | | VHNAKTKPREEQFNS |
| | | | TYRVVSVLTVLHQDW |
| | | | LNGKEYKCKVSNKGL |
| | | | PSSIEKTISKAKGQP |
| | | | REPQVYTLPPSQEEM |
| | | | TKNQVSLTCLVKGFY |
| | | | PSDIAVEWESNGQPE |
| | | | NNYKTTPPVLDSDGS |
| | | | FFLYSRLTVDKSRWQ |
| | | | EGNVFSCSVMHEALH |
| | | | NHYTQKSLSLSPGK |
| 289 | IGG4 HC | 37323 | EVQLLESGGGLVQPG |
| | | | GSLRLSCAASGFTFD |
| | | | NYAMHWVRQAPGKGL |
| | | | EWVSAISARAGITYY |
| | | | ADSVKGRFTISRDNS |
| | | | KNTLYLQMNSLRAED |
| | | | TAVYYCARRIGYSYG |
| | | | TAPPFDVWGQGTTVT |
| | | | VSSASTKGPSVFPLA |
| | | | PCSRSTSESTAALGC |
| | | | LVKDYFPEPVTVSWN |
| | | | SGALTSGVHTFPAVL |
| | | | QSSGLYSLSSVVTVP |
| | | | SSSLGTKTYTCNVDH |
| | | | KPSNTKVDKRVESKY |
| | | | GPPCPPCPAPEFLGG |
| | | | PSVFLFPPKPKDTLM |
| | | | ISRTPEVTCVVVDVS |
| | | | QEDPEVQFNWYVDGV |
| | | | EVHNAKTKPREEQFN |
| | | | STYRVVSVLTVLHQD |
| | | | WLNGKEYKCKVSNKG |
| | | | LPSSIEKTISKAKGQ |
| | | | PREPQVYTLPPSQEE |
| | | | MTKNQVSLTCLVKGF |
| | | | YPSDIAVEWESNGQP |
| | | | ENNYKTTPPVLDSDG |
| | | | SFFLYSRLTVDKSRW |
| | | | QEGNVFSCSVMHEAL |
| | | | HNHYTQKSLSLSPGK |
| 290 | IGG4 HC | 38389 | QVQLVESGGGLVQPG |
| | | | GSLRLSCAASGFTFS |
| | | | DYYMSWIRQAPGKGL |
| | | | EWVSYIASSGSVIYY |
| | | | ADSVKGRFTISRDNA |
| | | | KNSLYLQMNSLRAED |
| | | | TAVYYCARHGTPRAF |
| | | | DIWGQGTTVTVSSAS |
| | | | TKGPSVFPLAPCSRS |
| | | | TSESTAALGCLVKDY |
| | | | FPEPVTVSWNSGALT |
| | | | SGVHTFPAVLQSSGL |
| | | | YSLSSVVTVPSSSLG |
| | | | TKTYTCNVDHKPSNT |
| | | | KVDKRVESKYGPPCP |
| | | | PCPAPEFLGGPSVFL |
| | | | FPPKPKDTLMISRTP |
| | | | EVTCVVVDVSQEDPE |
| | | | VQFNWYVDGVEVHNA |
| | | | KTKPREEQFNSTYRV |
| | | | VSVLTVLHQDWLNGK |
| | | | EYKCKVSNKGLPSSI |
| | | | EKTISKAKGQPREPQ |
| | | | VYTLPPSQEEMTKNQ |
| | | | VSLTCLVKGFYPSDI |
| | | | AVEWESNGQPENNYK |
| | | | TTPPVLDSDGSFFLY |
| | | | SRLTVDKSRWQEGNV |
| | | | FSCSVMHEALHNHYT |
| | | | QKSLSLSPGK |
| 291 | IGG4 HC | 38358 | EVQLLESGGGLVQPG |
| | | | GSLRLSCAASGFTFS |
| | | | SSAMAWVRQAPGKGL |
| | | | EWVSTISGSGITTWY |
| | | | ADSVKGRFTISRDNS |
| | | | KNTLYLQMNSLRAED |
| | | | TAVYYCAKGSRHLNA |
| | | | FNRWGQGTTVTVSSA |
| | | | STKGPSVFPLAPCSR |
| | | | STSESTAALGCLVKD |
| | | | YFPEPVTVSWNSGAL |
| | | | TSGVHTFPAVLQSSG |
| | | | LYSLSSVVTVPSSSL |
| | | | GTKTYTCNVDHKPSN |
| | | | TKVDKRVESKYGPPC |
| | | | PPCPAPEFLGGPSVF |
| | | | LFPPKPKDTLMISRT |
| | | | PEVTCVVVDVSQEDP |
| | | | EVQFNWYVDGVEVHN |
| | | | AKTKPREEQFNSTYR |
| | | | VVSVLTVLHQDWLNG |
| | | | KEYKCKVSNKGLPSS |
| | | | IEKTISKAKGQPREP |
| | | | QVYTLPPSQEEMTKN |
| | | | QVSLTCLVKGFYPSD |
| | | | IAVEWESNGQPENNY |
| | | | KTTPPVLDSDGSFFL |
| | | | YSRLTVDKSRWQEGN |
| | | | VFSCSVMHEALHNHY |
| | | | TQKSLSLSPGK |
| 292 | IGG4 HC | 33303 | QVQLQESGPGLVKPS |
| | | | GTLSLTCAVSGGSIS |
| | | | SSNWWSWVRQPPGKG |
| | | | LEWIGEIYHSGSTNY |
| | | | NPSLKSRVTISVDKS |
| | | | KNQFSLKLSSVTAAD |
| | | | TAVYYCARGVYHYDP |
| | | | YGMDVWGQGTTVTVS |
| | | | SASTKGPSVFPLAPC |
| | | | SRSTSESTAALGCLV |
| | | | KDYFPEPVTVSWNSG |
| | | | ALTSGVHTFPAVLQS |
| | | | SGLYSLSSVVTVPSS |
| | | | SLGTKTYTCNVDHKP |
| | | | SNTKVDKRVESKYGP |
| | | | PCPPCPAPEFLGGPS |
| | | | VFLFPPKPKDTLMIS |
| | | | RTPEVTCVVVDVSQE |
| | | | DPEVQFNWYVDGVEV |
| | | | HNAKTKPREEQFNST |
| | | | YRVVSVLTVLHQDWL |
| | | | NGKEYKCKVSNKGLP |
| | | | SSIEKTISKAKGQPR |
| | | | EPQVYTLPPSQEEMT |
| | | | KNQVSLTCLVKGFYP |
| | | | SDIAVEWESNGQPEN |
| | | | NYKTTPPVLDSDGSF |
| | | | FLYSRLTVDKSRWQE |
| | | | GNVFSCSVMHEALHN |
| | | | HYTQKSLSLSPGK |
| 293 | IGG4 HC | 33342 | QLQLQESGPGLVKPS |
| | | | ETLSLTCTVSGGSIS |
| | | | SSSYYWGWIRQPPGK |
| | | | GLEWIGSISYSGSTY |
| | | | YNPSLKSRVTISVDT |
| | | | SKNQFSLKLSSVTAA |
| | | | DTAVYYCARTELGKM |
| | | | HFDYWGQGTLVTVSS |
| | | | ASTKGPSVFPLAPCS |
| | | | RSTSESTAALGCLVK |
| | | | DYFPEPVTVSWNSGA |
| | | | LTSGVHTFPAVLQSS |
| | | | GLYSLSSVVTVPSSS |
| | | | LGTKTYTCNVDHKPS |
| | | | NTKVDKRVESKYGPP |
| | | | CPPCPAPEFLGGPSV |
| | | | FLFPPKPKDTLMISR |
| | | | TPEVTCVVVDVSQED |
| | | | PEVQFNWYVDGVEVH |
| | | | NAKTKPREEQFNSTY |
| | | | RVVSVLTVLHQDWLN |
| | | | GKEYKCKVSNKGLPS |
| | | | SIEKTISKAKGQPRE |
| | | | PQVYTLPPSQEEMTK |
| | | | NQVSLTCLVKGFYPS |
| | | | DIAVEWESNGQPENN |
| | | | YKTTPPVLDSDGSFF |
| | | | LYSRLTVDKSRWQEG |
| | | | NVFSCSVMHEALHNH |
| | | | YTQKSLSLSPGK |
| 294 | IGG4 HC | 33329 | QLQLQESGPGLVKPS |
| | | | ETLSLTCTVSGGSIS |
| | | | SSDYYWGWIRQPPGK |
| | | | GLEWIGSIYYSGSTY |
| | | | YNPSLKSRVTISVDT |
| | | | SKNQFSLKLSSVTAA |
| | | | DTAVYYCARGSPRYM |
| | | | QDWGQGTLVTVSSAS |
| | | | TKGPSVFPLAPCSRS |
| | | | TSESTAALGCLVKDY |
| | | | FPEPVTVSWNSGALT |
| | | | SGVHTFPAVLQSSGL |
| | | | YSLSSVVTVPSSSLG |
| | | | TKTYTCNVDHKPSNT |
| | | | KVDKRVESKYGPPCP |
| | | | PCPAPEFLGGPSVFL |
| | | | FPPKPKDTLMISRTP |
| | | | EVTCVVVDVSQEDPE |
| | | | VQFNWYVDGVEVHNA |
| | | | KTKPREEQFNSTYRV |
| | | | VSVLTVLHQDWLNGK |
| | | | EYKCKVSNKGLPSSI |
| | | | EKTISKAKGQPREPQ |
| | | | VYTLPPSQEEMTKNQ |
| | | | VSLTCLVKGFYPSDI |
| | | | AVEWESNGQPENNYK |
| | | | TTPPVLDSDGSFFLY |
| | | | SRLTVDKSRWQEGNV |
| | | | FSCSVMHEALHNHYT |
| | | | QKSLSLSPGK |
| 295 | IGG4 HC | 33351 | QVQLVESGGGLVKPG |
| | | | GSLRLSCAASGFTFS |
| | | | DYYMSWIRQAPGKGL |
| | | | EWVSYISSSGSTIYY |
| | | | ADSVKGRFTISRDNA |
| | | | KNSLYLQMNSLRAED |
| | | | TAVYYCARHSSLGTH |
| | | | NWFDPWGQGTLVTVS |
| | | | SASTKGPSVFPLAPC |
| | | | SRSTSESTAALGCLV |
| | | | KDYFPEPVTVSWNSG |
| | | | ALTSGVHTFPAVLQS |
| | | | SGLYSLSSVVTVPSS |
| | | | SLGTKTYTCNVDHKP |
| | | | SNTKVDKRVESKYGP |
| | | | PCPPCPAPEFLGGPS |
| | | | VFLFPPKPKDTLMIS |
| | | | RTPEVTCVVVDVSQE |
| | | | DPEVQFNWYVDGVEV |
| | | | HNAKTKPREEQFNST |
| | | | YRVVSVLTVLHQDWL |
| | | | NGKEYKCKVSNKGLP |
| | | | SSIEKTISKAKGQPR |
| | | | EPQVYTLPPSQEEMT |
| | | | KNQVSLTCLVKGFYP |
| | | | SDIAVEWESNGQPEN |
| | | | NYKTTPPVLDSDGSF |
| | | | FLYSRLTVDKSRWQE |
| | | | GNVFSCSVMHEALHN |
| | | | HYTQKSLSLSPGK |
| 296 | IGG4 HC | 33357 | QVQLQESGPGLVKPS |
| | | | ETLSLTCAVSGYSIS |
| | | | SGYYWGWIRQPPGKG |
| | | | LEWIGSIYHSGSTYY |
| | | | NPSLKSRVTISVDTS |
| | | | KNQFSLKLSSVTAAD |
| | | | TAVYYCAREGALSYS |
| | | | WLAAFDIWGQGTMVT |
| | | | VSSASTKGPSVFPLA |
| | | | PCSRSTSESTAALGC |
| | | | LVKDYFPEPVTVSWN |
| | | | SGALTSGVHTFPAVL |
| | | | QSSGLYSLSSVVTVP |
| | | | SSSLGTKTYTCNVDH |
| | | | KPSNTKVDKRVESKY |
| | | | GPPCPPCPAPEFLGG |
| | | | PSVFLFPPKPKDTLM |
| | | | ISRTPEVTCVVVDVS |
| | | | QEDPEVQFNWYVDGV |
| | | | EVHNAKTKPREEQFN |
| | | | STYRVVSVLTVLHQD |
| | | | WLNGKEYKCKVSNKG |
| | | | LPSSIEKTISKAKGQ |
| | | | PREPQVYTLPPSQEE |
| | | | MTKNQVSLTCLVKGF |
| | | | YPSDIAVEWESNGQP |
| | | | ENNYKTTPPVLDSDG |
| | | | SFFLYSRLTVDKSRW |
| | | | QEGNVFSCSVMHEAL |
| | | | HNHYTQKSLSLSPGK |
| 297 | | | |
| 298 | | | |
| 299 | | | |
| 300 | LC | 33343 | EIVLTQSPGTLSLSP |
| | | | GERATLSCRASQSVS |
| | | | SSYLAWYQQKPGQAP |
| | | | RLLIYGASSRATGIP |
| | | | DRFSGSGSGTDFTLT |
| | | | ISRLEPEDFAVYYCQ |
| | | | QAVHSPYTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 301 | LC | 37268 | EIVLTQSPGTLSLSP |
| | | | GERATLSCRASQSVS |
| | | | SSYLAWYQQKPGQAP |
| | | | RLLIYGASSRATGIP |
| | | | DRFSGSGSGTDFTLT |
| | | | ISRLEPEDFAVYYCQ |
| | | | QAVHSPYTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 302 | LC | 37269 | EIVLTQSPGTLSLSP |
| | | | GERATLSCRASQSVS |
| | | | SSYLAWYQQKPGQAP |
| | | | RLLIYGASSRATGIP |
| | | | DRFSGSGSGTDFTLT |
| | | | ISRLEPEDFAVYYCQ |
| | | | QAVHSPYTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 303 | LC | 37271 | EIVLTQSPGTLSLSP |
| | | | GERATLSCRASQSVS |
| | | | SSYLAWYQQKPGQAP |
| | | | RLLIYGASSRATGIP |
| | | | DRFSGSGSGTDFTLT |
| | | | ISRLEPEDFAVYYCQ |
| | | | QAVHSPYTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 304 | LC | 37272 | EIVLTQSPGTLSLSP |
| | | | GERATLSCRASQSVS |
| | | | SSYLAWYQQKPGQAP |
| | | | RLLIYGASSRATGIP |
| | | | DRFSGSGSGTDFTLT |
| | | | ISRLEPEDFAVYYCQ |
| | | | QAVHSPYTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 305 | LC | 37277 | EIVLTQSPGTLSLSP |
| | | | GERATLSCGASQSVS |
| | | | SDYLAWYQQKPGQAP |
| | | | RLLIYGAYSLATGIP |
| | | | DRFSGSGSGTDFTLT |
| | | | ISRLEPEDFAVYYCQ |
| | | | WAVHSPYTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 306 | LC | 38373 | EIVLTQSPGTLSLSP |
| | | | GERATLSCQASQAVS |
| | | | SNYLAWYQQKPGQAP |
| | | | RLLIYGASSRATGIP |
| | | | DRFSGSGSGTDFTLT |
| | | | ISRLEPEDFAVYYCQ |
| | | | QVVHSPYTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 307 | LC | 38375 | EIVLTQSPGTLSLSP |
| | | | GERATLSCGASQSVS |
| | | | SAFLAWYQQKPGQAP |
| | | | RLLIYGASARATGIP |
| | | | DRFSGSGSGTDFTLT |
| | | | ISRLEPEDFAVYYCQ |
| | | | QVVHSPYTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 308 | LC | 38379 | EIVLTQSPGTLSLSP |
| | | | GERATLSCRASQSVS |
| | | | STYLAWYQQKPGQAP |
| | | | RLLIYGASSREAGIP |
| | | | DRFSGSGSGTDFTLT |
| | | | ISRLEPEDFAVYYCQ |
| | | | QTVHSPYTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 309 | LC | 38381 | EIVLTQSPGTLSLSP |
| | | | GERATLSCQASQAVS |
| | | | SNYLAWYQQKPGQAP |
| | | | RLLIYGASNRAAGIP |
| | | | DRFSGSGSGTDFTLT |
| | | | ISRLEPEDFAVYYCQ |
| | | | QAIHSPYTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 310 | LC | 38383 | EIVLTQSPGTLSLSP |
| | | | GERATLSCQASQSVS |
| | | | SSYLAWYQQKPGQAP |
| | | | RLLIYGASNRAAGIP |
| | | | DRFSGSGSGTDFTLT |
| | | | ISRLEPEDFAVYYCQ |
| | | | QVVHSPYTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 311 | LC | 38386 | EIVLTQSPGTLSLSP |
| | | | GERATLSCKASQAVS |
| | | | SSYLAWYQQKPGQAP |
| | | | RLLIYGASSRQDGIP |
| | | | DRFSGSGSGTDFTLT |
| | | | ISRLEPEDFAVYYCQ |
| | | | QVVHSPYTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 312 | LC | 37273 | EIVLTQSPGTLSLSP |
| | | | GERATLSCRASQSVS |
| | | | SSYLAWYQQKPGQAP |
| | | | RLLIYGASSRATGIP |
| | | | DRFSGSGSGTDFTLT |
| | | | ISRLEPEDFAVYYCQ |
| | | | QAVHSPYTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 313 | LC | 33361 | EIVLTQSPGTLSLSP |
| | | | GERATLSCRASQSVS |
| | | | SSYLAWYQQKPGQAP |
| | | | RLLIYGASSRATGIP |
| | | | DRFSGSGSGTDFTLT |
| | | | ISRLEPEDFAVYYCQ |
| | | | QHSSYPPTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 314 | LC | 35624 | EIVLTQSPGTLSLSP |
| | | | GERATLSCRASQSVS |
| | | | SSYLAWYQQKPGQAP |
| | | | RLLIYGASSRATGIP |
| | | | DRFSGSGSGTDFTLT |
| | | | ISRLEPEDFAVYYCQ |
| | | | QHSSYPPTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 315 | LC | 38410 | EIVLTQSPGTLSLSP |
| | | | GERATLSCEASQSVS |
| | | | SSYLAWYQQKPGQAP |
| | | | RLLIYGASNRATGIP |
| | | | DRFSGSGSGTDFTLT |
| | | | ISRLEPEDFAVYYCQ |
| | | | QHSLYPPTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 316 | LC | 38418 | EIVLTQSPGTLSLSP |
| | | | GERATLSCEASQSVS |
| | | | ASYLAWYQQKPGQAP |
| | | | RLLIYDASSRASGIP |
| | | | DRFSGSGSGTDFTLT |
| | | | ISRLEPEDFAVYYCQ |
| | | | QFSSYPPTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 317 | LC | 38420 | EIVLTQSPGTLSLSP |
| | | | GERATLSCEASQSVS |
| | | | SAYLAWYQQKPGQAP |
| | | | RLLIYDASTRATGIP |
| | | | DRFSGSGSGTDFTLT |
| | | | ISRLEPEDFAVYYCQ |
| | | | QVSSYPPTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 318 | LC | 38421 | EIVLTQSPGTLSLSP |
| | | | GERAALSCRVSQSVS |
| | | | DAYLAWYQQKPGQAP |
| | | | RLLIYDASSRASGIP |
| | | | DRFSGSGSGTDFTLT |
| | | | ISRLEPEDFAVYYCQ |
| | | | QVSSYPPTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 319 | LC | 38422 | EIVLTQSPGTLSLSP |
| | | | GERATLSCEVSQSVS |
| | | | ASYLAWYQQKPGQAP |
| | | | RLLIYGASSRATGIP |
| | | | DRFSGSGSGTDFTLT |
| | | | ISRLEPEDFAVYYCQ |
| | | | QHSLYPPTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 320 | LC | 38424 | EIVLTQSPGTLSLSP |
| | | | GERATLSCRASQSVS |
| | | | SAYLAWYQQKPGQAP |
| | | | RLLIYGASDRANGIP |
| | | | DRFSGSGSGTDFTLT |
| | | | ISRLEPEDFAVYYCQ |
| | | | QHSLYPPTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 321 | LC | 38425 | EIVLTQSPGTLSLSP |
| | | | GERATLSCRASNAVS |
| | | | SSYLAWYQQKPGQAP |
| | | | RLLIYGASDRANGIP |
| | | | DRFSGSGSGTDFTLT |
| | | | ISRLEPEDFAVYYCQ |
| | | | QHSIYPPTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 322 | LC | 38426 | EIVLTQSPGTLSLSP |
| | | | GERATLSCRASNAVS |
| | | | SSYLAWYQQKPGQAP |
| | | | RLLIYGASYRATGIP |
| | | | DRFSGSGSGTDFTLT |
| | | | ISRLEPEDFAVYYCQ |
| | | | QHSLYPPTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 323 | LC | 37323 | DIQMTQSPSTLSASV |
| | | | GDRVTITCRASQSIN |
| | | | SWLAWYQQKPGKAPK |
| | | | LLISDASSLESGVPS |
| | | | RFSGSGSGTEFTLTI |
| | | | SSLQPDDFATYYCQQ |
| | | | YDSHITFGGGTKVEI |
| | | | KRTVAAPSVFIFPPS |
| | | | DEQLKSGTASVVCLL |
| | | | NNFYPREAKVQWKVD |
| | | | NALQSGNSQESVTEQ |
| | | | DSKDSTYSLSSTLTL |
| | | | SKADYEKHKVYACEV |
| | | | THQGLSSPVTKSFNR |
| | | | GEC |
| 324 | LC | 38389 | DIQMTQSPSSVSASV |
| | | | GDRVTITCAASQGIS |
| | | | SDLAWYQQKPGKAPK |
| | | | LLIYSASSTQSGVPS |
| | | | RFSGSGSGTDFTLTI |
| | | | SSLQPEDFATYYCQQ |
| | | | AYLYPITFGGGTKVE |
| | | | IKRTVAAPSVFIFPP |
| | | | SDEQLKSGTASVVCL |
| | | | LNNFYPREAKVQWKV |
| | | | DNALQSGNSQESVTE |
| | | | QDSKDSTYSLSSTLT |
| | | | LSKADYEKHKVYACE |
| | | | VTHQGLSSPVTKSFN |
| | | | RGEC |
| 325 | LC | 38358 | GVQMTQSPSSLSASV |
| | | | GDRVTITCRASQDIS |
| | | | TYLNWYQQKPGKAPK |
| | | | LLIYDAFNLETGVPS |
| | | | RFSGSGSGTDFTFTI |
| | | | SSLQPEDIATYYCQQ |
| | | | LPFLPITFGGGTKVE |
| | | | IKRTVAAPSVFIFPP |
| | | | SDEQLKSGTASVVCL |
| | | | LNNFYPREAKVQWKV |
| | | | DNALQSGNSQESVTE |
| | | | QDSKDSTYSLSSTLT |
| | | | LSKADYEKHKVYACE |
| | | | VTHQGLSSPVTKSFN |
| | | | RGEC |
| 326 | LC | 33303 | DIVMTQSPLSLPVTP |
| | | | GEPASISCRSSQSLL |
| | | | HSNGYNYLDWYLQKP |
| | | | GQSPQLLIYLGSNRA |
| | | | SGVPDRFSGSGSGTD |
| | | | FTLKISRVEAEDVGV |
| | | | YYCMQALGGPWTFGG |
| | | | GTKVEIKRTVAAPSV |
| | | | FIFPPSDEQLKSGTA |
| | | | SVVCLLNNFYPREAK |
| | | | VQWKVDNALQSGNSQ |
| | | | ESVTEQDSKDSTYSL |
| | | | SSTLTLSKADYEKHK |
| | | | VYACEVTHQGLSSPV |
| | | | TKSFNRGEC |
| 327 | LC | 33342 | EIVLTQSPGTLSLSP |
| | | | GERATLSCRASQSVS |
| | | | SSYLAWYQQKPGQAP |
| | | | RLLIYGASRRATGIP |
| | | | DRFSGSGSGTDFTLT |
| | | | ISRLEPEDFAVYYCQ |
| | | | QYVSDPITFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 328 | LC | 33299 | EIVLTQSPGTLSLSP |
| | | | GERATLSCRASQSVS |
| | | | SSYLAWYQQKPGQAP |
| | | | RLLIYGASRRATGIP |
| | | | DRFSGSGSGTDFTLT |
| | | | ISRLEPEDFAVYYCQ |
| | | | QVGSSPITFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 329 | LC | 33351 | DIQMTQSPSSLSASV |
| | | | GDRVTITCRASQSIS |
| | | | SYLNWYQQKPGKAPK |
| | | | LLIYAASSLQSGVPS |
| | | | RFSGSGSGTDFTLTI |
| | | | SSLQPEDFATYYCQQ |
| | | | SHLVPRTFGGGTKVE |
| | | | IKRTVAAPSVFIFPP |
| | | | SDEQLKSGTASVVCL |
| | | | LNNFYPREAKVQWKV |
| | | | DNALQSGNSQESVTE |
| | | | QDSKDSTYSLSSTLT |
| | | | LSKADYEKHKVYACE |
| | | | VTHQGLSSPVTKSFN |
| | | | RGEC |
| 330 | LC | 33357 | EIVMTQSPATLSVSP |
| | | | GERATLSCRASQSVS |
| | | | SNLAWYQQKPGQAPR |
| | | | LLIYGASTRATGIPA |
| | | | RFSGSGSGTEFTLTI |
| | | | SSLQSEDFAVYYCQQ |
| | | | ANHHPPFTFGGGTKV |
| | | | EIKRTVAAPSVFIFP |
| | | | PSDEQLKSGTASVVC |
| | | | LLNNFYPREAKVQWK |
| | | | VDNALQSGNSQESVT |
| | | | EQDSKDSTYSLSSTL |
| | | | TLSKADYEKHKVYAC |
| | | | EVTHQGLSSPVTKSF |
| | | | NRGEC |
| 331 | | | |
| 332 | | | |
| 333 | | | |
| 334 | Fc for | | ASTKGPSVFPLAPSS |
| | IGG1 | | KSTSGGTAALGCLVK |
| | | | DYFPEPVTVSWNSGA |
| | | | LTSGVHTFPAVLQSS |
| | | | GLYSLSSVVTVPSSS |
| | | | LGTQTYICNVNHKPS |
| | | | NTKVDKKVEPKSCDK |
| | | | THTCPPCPAPEAAGA |
| | | | PSVFLFPPKPKDTLM |
| | | | ISRTPEVTCVVVDVS |
| | | | HEDPEVKFNWYVDGV |
| | | | EVHNAKTKPREEQYN |
| | | | STYRVVSVLTVLHQD |
| | | | WLNGKEYKCKVSNKA |
| | | | LPAPIEKTISKAKGQ |
| | | | PREPQVYTLPPSREE |
| | | | MTKNQVSLTCLVKGF |
| | | | YPSDIAVEWESNGQP |
| | | | ENNYKTTPPVLDSDG |
| | | | SFFLYSKLTVDKSRW |
| | | | QQGNVFSCSVMHEAL |
| | | | HNHYTQKSLSLSPGK |
| 335 | Fc region | | ASTKGPSVFPLAPCS |
| | for IGG4 | | RSTSESTAALGCLVK |
| | | | DYFPEPVTVSWNSGA |
| | | | LTSGVHTFPAVLQSS |
| | | | GLYSLSSVVTVPSSS |
| | | | LGTKTYTCNVDHKPS |
| | | | NTKVDKRVESKYGPP |
| | | | CPPCPAPEFLGGPSV |
| | | | FLFPPKPKDTLMISR |
| | | | TPEVTCVVVDVSQED |
| | | | PEVQFNWYVDGVEVH |
| | | | NAKTKPREEQFNSTY |
| | | | RVVSVLTVLHQDWLN |
| | | | GKEYKCKVSNKGLPS |
| | | | SIEKTISKAKGQPRE |
| | | | PQVYTLPPSQEEMTK |
| | | | NQVSLTCLVKGFYPS |
| | | | DIAVEWESNGQPENN |
| | | | YKTTPPVLDSDGSFF |
| | | | LYSRLTVDKSRWQEG |
| | | | NVFSCSVMHEALHNH |
| | | | YTQKSLSLSPGK |
| 336 | Kappa | | RTVAAPSVFIFPPSD |
| | region | | EQLKSGTASVVCLLN |
| | for LC | | NFYPREAKVQWKVDN |
| | | | ALQSGNSQESVTEQD |
| | | | SKDSTYSLSSTLTLS |
| | | | KADYEKHKVYACEVT |
| | | | HQGLSSPVTKSFNRG |
| | | | EC |
| 337 | | hHLA-G1 | MVVMAPRTLFLLLSG |
| | | | ALTLTETWAGSHSMR |
| | | | YFSAAVSRPGRGEPR |
| | | | FIAMGYVDDTQFVRF |
| | | | DSDSACPRMEPRAPW |
| | | | VEQEGPEYWEEETRN |
| | | | TKAHAQTDRMNLQTL |
| | | | RGYYNQSEASSHTLQ |
| | | | WMIGCDLGSDGRLLR |
| | | | GYEQYAYDGKDYLAL |
| | | | NEDLRSWTAADTAAQ |
| | | | ISKRKCEAANVAEQR |
| | | | RAYLEGTCVEWLHRY |
| | | | LENGKEMLQRADPPK |
| | | | THVTHHPVFDYEATL |
| | | | RCWALGFYPAEIILT |
| | | | WQRDGEDQTQDVELV |
| | | | ETRPAGDGTFQKWAA |
| | | | VVVPSGEEQRYTCHV |
| | | | QHEGLPEPLMLRWKQ |
| | | | SSLPTIPIMGIVAGL |
| | | | VVLAAVVTGAAVAAV |
| | | | LWRKKSSD |
| 338 | | hHLA-G5 | MVVMAPRTLFLLLSG |
| | | | ALTLTETWAGSHSMR |
| | | | YFSAAVSRPGRGEPR |
| | | | FIAMGYVDDTQFVRF |
| | | | DSDSACPRMEPRAPW |
| | | | VEQEGPEYWEEETRN |
| | | | TKAHAQTDRMNLQTL |
| | | | RGYYNQSEASSHTLQ |
| | | | WMIGCDLGSDGRLLR |
| | | | GYEQYAYDGKDYLAL |
| | | | NEDLRSWTAADTAAQ |
| | | | ISKRKCEAANVAEQR |
| | | | RAYLEGTCVEWLHRY |
| | | | LENGKEMLQRADPPK |
| | | | THVTHHPVFDYEATL |
| | | | RCWALGFYPAEIILT |
| | | | WQRDGEDQTQDVELV |
| | | | ETRPAGDGTFQKWAA |
| | | | VVVPSGEEQRYTCHV |
| | | | QHEGLPEPLMLRWSK |
| | | | EGDGGIMSVRESRSL |
| | | | SEDL |
| 339 | | Cyno HLA-AG | MAVMAPRTLLLVLSG |
| | | | VLALTQPRAGSHSMR |
| | | | YFYTAVSRPGRGQPR |
| | | | FIAVGYVDDTQFVRF |
| | | | DSDAESPRMEPRAPW |
| | | | VEQEGPEYWDRETQN |
| | | | MKTATQTYQANLRTL |
| | | | LRYYNQSEAGSHTFQ |
| | | | KMYGCDLGPDGRLLR |
| | | | GYEQFAYDGRDYIIL |
| | | | NEDLRSWTAADMAAQ |
| | | | NTQRKWEAAGAAEQH |
| | | | RTYLEGECLEWLRRY |
| | | | LENGKETLQRADPPK |
| | | | TNVTHHPVSDYEATL |
| | | | RCWALGFYPAEITLT |
| | | | WQRDGEEQTEDTELV |
| | | | ETRPTGDGTFQKWAA |
| | | | VVVPSGEEQRYTCHV |
| | | | QHEGLPKPLTLRWEP |
| | | | SSQSTILIVGIIAGL |
| | | | VLLGTVVTGAVVAAV |
| | | | MWRRKS |
| 340 | | Rhesus | MAVMAPRTLLLVLSG |
| | | HLA-AG | VLALTQTRAGSHSMR |
| | | | YFYTSMSRPGRGQPR |
| | | | FIAVGYVDDTQFVRF |
| | | | DSDAESPRMEPRAPW |
| | | | VEQEGPEYWDRETQN |
| | | | MKTATQTYRENLRTL |
| | | | LRYYNQSEAGSHTIQ |
| | | | KMYGCDLGPDGRLLR |
| | | | GYEQFAYDGRDYIAL |
| | | | NEDLRSWTAADMAAQ |
| | | | FTQRKWEAAGAAEQH |
| | | | RTYLEGECLEWLRRY |
| | | | LENGKETLQRADPPK |
| | | | TNVTHHPVSDYEATL |
| | | | RCWALGFYPAEITLT |
| | | | WQRDGEEQTEDTELV |
| | | | ETRPTGDGTFQKWAA |
| | | | VVVPSGEEQRYTCHV |
| | | | QHEGLPEPLTLRWEP |
| | | | SSQSTILIVGIIAGL |
| | | | VLLGTVVTGAVVAAV |
| | | | MWRRKSSDR |
| 341 | | hHLA-G ECD | MVVMAPRTLFLLLSGA |
| | | with | LTLTETWAGSHSMRFS |
| | | signal | AAVSRPGRGEPRFYIA |
| | | peptide | MGYVDDTQFVRFDSDS |
| | | | ACPRMEPRAPWVEQEG |
| | | | PEYWEEETRNTKAHAQ |
| | | | TDRMNLQTLRGYYNQ |
| | | | SEASSHTLQWMIGCD |
| | | | LGSDGRLLRGYEQYA |
| | | | YDGKDYLALNEDLRS |
| | | | WTAADTAAQISKRKC |
| | | | EAANVAEQRRAYLEG |
| | | | TCVEWLHRYLENGKE |
| | | | MLQRADPPKTHVTHH |
| | | | PVFDYEATLRCWALG |
| | | | FYPAEIILTWQRDGE |
| | | | DQTQDVELVETRPAG |
| | | | DGTFQKWAAVVVPSG |
| | | | EEQRYTCHVQHEGLP |
| | | | EPLMLRW |
| 342 | | hHLA-G ECD | GSHSMRYFSAAVSRP |
| | | without | GRGEPRFIAMGYVDD |
| | | signal | TQFVRFDSDSACPRM |
| | | peptide | EPRAPWVEQEGPEYW |
| | | | EEETRNTKAHAQTDR |
| | | | MNLQTLRGYYNQSEA |
| | | | SSHTLQWMIGCDLGS |
| | | | DGRLLRGYEQYAYDG |
| | | | KDYLALNEDLRSWTA |
| | | | ADTAAQISKRKCEAA |
| | | | NVAEQRRAYLEGTCV |
| | | | EWLHRYLENGKEMLQ |
| | | | RADPPKTHVTHHPVF |
| | | | DYEATLRCWALGFYP |
| | | | AEIILTWQRDGEDQT |
| | | | QDVELVETRPAGDGT |
| | | | FQKWAAVVVPSGEEQ |
| | | | RYTCHVQHEGLPEPL |
| | | | MLRW |

### EQUIVALENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

1-10. (canceled)

11. An isolated antibody molecule capable of binding to human HLA-G (hHLA-G), comprising a heavy chain variable region (VH) and a light chain variable region (VL), the V_{H} and/or V_{L} comprising 1, 2, 3, 4, 5, or 6 of:
a) a VHCDR 1 having the sequence set forth in SEQ ID NOS: 1-14 or SEQ ID NOS: 18-34,
b) a VHCDR2 having the sequence set forth in SEQ ID NOS: 38-50 or SEQ ID NOS: 54-71,
c) a VHCDR3 having the sequence set forth in SEQ ID NOS: 76-101,
d) a VLCDR1 having the sequence set forth in SEQ ID NOS: 105-124,
e) a VLCDR2 having the sequence set forth in SEQ ID NOS: 128-145, and
f) a VLCDR3 having the sequence set forth in SEQ ID NOS: 149-166.

12. An isolated antibody molecule capable of binding to human HLA-G (hHLA-G) according to claim 11
the VH comprising:
a) a VHCDR1 having a sequence set forth in SEQ ID NOS: 1-14 or SEQ ID NOS: 18-34,
b) a VHCDR2 having a sequence set forth in SEQ ID NOS: 38-50 or SEQ ID NOS: 54-71, and
c) a VHCDR3 having a sequence set forth in SEQ ID NOS: 76-101; and
the VL comprising:
a) a VLCDR1 having a sequence set forth in SEQ ID NO: 105-124,
b) a VLCDR2 having a sequence set forth in SEQ ID NO: 128-145, and
c) a VLCDR3 having a sequence set forth in SEQ ID NO: 149-166.

13-39. (canceled)

40. An isolated antibody molecule capable of binding to human HLA-G (hHLA-G) according to claim 11
the VH comprising 1, 2, or 3 of:
a) a VHCDR1 having an amino acid sequence that is at least 90% identical to the sequence set forth in SEQ ID NOS: 1-14 or 18-34,
b) a VHCDR2 having an amino acid sequence that is at least 90% identical to the sequence set forth in SEQ ID NOS: 54-71, and
c) a VHCDR3 having an amino acid sequence that is at least 90% identical to the sequence set forth in SEQ ID NOS: 76-101; and
the VL comprising 1, 2, or 3 of:
a) a VLCDR1 having an amino acid sequence that is at least 90% identical to the sequence set forth in SEQ ID NOS: 105-124,
b) a VLCDR2 having an amino acid sequence that is at least 90% identical to the sequence set forth in SEQ ID NOS: 128-145, and
c) a VLCDR3 having an amino acid sequence that is at least 90% identical to the sequence set forth in SEQ ID NOs 149-166.

41. An isolated antibody molecule capable of binding to human HLA-G (hHLA-G) according to claim 40
VH comprising 1, 2, or 3 of:
a) a VHCDR1 having an amino acid sequence that is homologous to the sequence set forth in SEQ ID NOS: 1-14 or SEQ ID NOS: 18-34,
b) a VHCDR2 having an amino acid sequence that is homologous to the sequence set forth in SEQ ID NOS: 38-50 or SEQ ID NOS: 54-71, and
c) a VHCDR3 having an amino acid sequence that is homologous to the sequence set forth in SEQ ID NOS: 76-101; and
VL comprising 1, 2, or 3 of:
a) a VLCDR1 having an amino acid sequence that is homologous to the sequence set forth in SEQ ID NOS: 105-124,
b) a VLCDR2 having an amino acid sequence that is homologous to the sequence set forth in SEQ ID NOS: 128-145, and
c) a VLCDR3 having an amino acid sequence that is homologous to the sequence set forth in SEQ ID NOS: 149-166.

42. An isolated antibody molecule capable of binding to human HLA-G (hHLA-G) according to claim 41, further comprising a heavy chain and a light chain, the heavy chain comprising one or more molecules having a sequence consisting of one of SEQ ID NOS: 232-262 or SEQ ID NOS: 266-296 and the light chain comprising one or more molecules having a sequence consisting of one of SEQ ID NOS: 300-330.

43. An isolated antibody molecule capable of binding to human HLA-G (HLA-G) according to claim 42, further comprising a heavy chain and a light chain,
a) the heavy chain comprising one or more molecules, each molecule having a sequence consisting of SEQ ID NO: 232 and the light chain comprising one or more, each molecule having a sequence consisting of SEQ ID NO: 300;
b) the heavy chain comprising one or more molecules, each molecule having a sequence consisting of SEQ ID NO: 233 and the light chain comprising one or more, each molecule having a sequence consisting of SEQ ID NO: 301;
c) the heavy chain comprising one or more molecules, each molecule having a sequence consisting of SEQ ID NO: 234 and the light chain comprising one or more, each molecule having a sequence consisting of SEQ ID NO: 302;
d) the heavy chain comprising one or more molecules, each molecule having a sequence consisting of SEQ ID NO: 235 and the light chain comprising one or more, each molecule having a sequence consisting of SEQ ID NO: 303;
e) the heavy chain comprising one or more molecules, each molecule having a sequence consisting of SEQ ID NO: 236 and the light chain comprising one or more, each molecule having a sequence consisting of SEQ ID NO: 304;
f) the heavy chain comprising one or more molecules, each molecule having a sequence consisting of SEQ ID NO: 237 and the light chain comprising one or more, each molecule having a sequence consisting of SEQ ID NO: 305;
g) the heavy chain comprising one or more molecules, each molecule having a sequence consisting of SEQ ID NO: 238 and the light chain comprising one or more, each molecule having a sequence consisting of SEQ ID NO: 306;
h) the heavy chain comprising one or more molecules, each molecule having a sequence consisting of SEQ ID NO: 239 and the light chain comprising one or more, each molecule having a sequence consisting of SEQ ID NO: 307;
i) the heavy chain comprising one or more molecules, each molecule having a sequence consisting of SEQ ID NO: 240 and the light chain comprising one or more, each molecule having a sequence consisting of SEQ ID NO: 308;
j) the heavy chain comprising one or more molecules, each molecule having a sequence consisting of SEQ ID NO: 241 and the light chain comprising one or more, each molecule having a sequence consisting of SEQ ID NO: 309;
k) the heavy chain comprising one or more molecules, each molecule having a sequence consisting of SEQ ID NO: 242 and the light chain comprising one or more, each molecule having a sequence consisting of SEQ ID NO: 310;
l) the heavy chain comprising one or more molecules, each molecule having a sequence consisting of SEQ ID NO: 243 and the light chain comprising one or more, each molecule having a sequence consisting of SEQ ID NO: 311;
m) the heavy chain comprising one or more molecules, each molecule having a sequence consisting of SEQ ID NO: 244 and the light chain comprising one or more, each molecule having a sequence consisting of SEQ ID NO: 312;
n) the heavy chain comprising one or more molecules, each molecule having a sequence consisting of SEQ ID NO: 245 and the light chain comprising one or more, each molecule having a sequence consisting of SEQ ID NO: 313;
o) the heavy chain comprising one or more molecules, each molecule having a sequence consisting of SEQ ID NO: 246 and the light chain comprising one or more, each molecule having a sequence consisting of SEQ ID NO: 314;
p) the heavy chain comprising one or more molecules, each molecule having a sequence consisting of SEQ ID NO: 247 and the light chain comprising one or more, each molecule having a sequence consisting of SEQ ID NO: 315;
q) the heavy chain comprising one or more molecules, each molecule having a sequence consisting of SEQ ID NO: 248 and the light chain comprising one or more, each molecule having a sequence consisting of SEQ ID NO: 316;
r) the heavy chain comprising one or more molecules, each molecule having a sequence consisting of SEQ ID NO: 249 and the light chain comprising one or more, each molecule having a sequence consisting of SEQ ID NO: 317;
s) the heavy chain comprising one or more molecules, each molecule having a sequence consisting of SEQ ID NO: 250 and the light chain comprising one or more, each molecule having a sequence consisting of SEQ ID NO: 318;
t) the heavy chain comprising one or more molecules, each molecule having a sequence consisting of SEQ ID NO: 251 and the light chain comprising one or more, each molecule having a sequence consisting of SEQ ID NO: 319;
u) the heavy chain comprising one or more molecules, each molecule having a sequence consisting of SEQ ID NO: 252 and the light chain comprising one or more, each molecule having a sequence consisting of SEQ ID NO: 320;
v) the heavy chain comprising one or more molecules, each molecule having a sequence consisting of SEQ ID NO: 253 and the light chain comprising one or more, each molecule having a sequence consisting of SEQ ID NO: 321;
w) the heavy chain comprising one or more molecules, each molecule having a sequence consisting of SEQ ID NO: 254 and the light chain comprising one or more, each molecule having a sequence consisting of SEQ ID NO: 322;
x) the heavy chain comprising one or more molecules, each molecule having a sequence consisting of SEQ ID NO: 255 and the light chain comprising one or more, each molecule having a sequence consisting of SEQ ID NO: 323;
y) the heavy chain comprising one or more molecules, each molecule having a sequence consisting of SEQ ID NO: 256 and the light chain comprising one or more, each molecule having a sequence consisting of SEQ ID NO: 324;
z) the heavy chain comprising one or more molecules, each molecule having a sequence consisting of SEQ ID NO: 257 and the light chain comprising one or more, each molecule having a sequence consisting of SEQ ID NO: 325;
aa) the heavy chain comprising one or more molecules, each molecule having a sequence consisting of SEQ ID NO: 258 and the light chain comprising one or more, each molecule having a sequence consisting of SEQ ID NO: 326;
ab) the heavy chain comprising one or more molecules, each molecule having a sequence consisting of SEQ ID NO: 259 and the light chain comprising one or more, each molecule having a sequence consisting of SEQ ID NO: 327;
ac) the heavy chain comprising one or more molecules, each molecule having a sequence consisting of SEQ ID NO: 260 and the light chain comprising one or more, each molecule having a sequence consisting of SEQ ID NO: 328;
ad) the heavy chain comprising one or more molecules, each molecule having a sequence consisting of SEQ ID NO: 261 and the light chain comprising one or more, each molecule having a sequence consisting of SEQ ID NO: 329;
ae) the heavy chain comprising one or more molecules, each molecule having a sequence consisting of SEQ ID NO: 262 and the light chain comprising one or more, each molecule having a sequence consisting of SEQ ID NO: 330;
af) the heavy chain comprising one or more molecules, each molecule having a sequence consisting of SEQ ID NO: 266 and the light chain comprising one or more, each molecule having a sequence consisting of SEQ ID NO: 300;
ag) the heavy chain comprising one or more molecules, each molecule having a sequence consisting of SEQ ID NO: 267 and the light chain comprising one or more, each molecule having a sequence consisting of SEQ ID NO: 301;
ah) the heavy chain comprising one or more molecules, each molecule having a sequence consisting of SEQ ID NO: 268 and the light chain comprising one or more, each molecule having a sequence consisting of SEQ ID NO: 302;
ai) the heavy chain comprising one or more molecules, each molecule having a sequence consisting of SEQ ID NO: 269 and the light chain comprising one or more, each molecule having a sequence consisting of SEQ ID NO: 303;
aj) the heavy chain comprising one or more molecules, each molecule having a sequence consisting of SEQ ID NO: 270 and the light chain comprising one or more, each molecule having a sequence consisting of SEQ ID NO: 304;
ak) the heavy chain comprising one or more molecules, each molecule having a sequence consisting of SEQ ID NO: 271 and the light chain comprising one or more, each molecule having a sequence consisting of SEQ ID NO: 305;
al) the heavy chain comprising one or more molecules, each molecule having a sequence consisting of SEQ ID NO: 272 and the light chain comprising one or more, each molecule having a sequence consisting of SEQ ID NO: 306;
am) the heavy chain comprising one or more molecules, each molecule having a sequence consisting of SEQ ID NO: 273 and the light chain comprising one or more, each molecule having a sequence consisting of SEQ ID NO: 307;
an) the heavy chain comprising one or more molecules, each molecule having a sequence consisting of SEQ ID NO: 274 and the light chain comprising one or more, each molecule having a sequence consisting of SEQ ID NO: 308;
ao) the heavy chain comprising one or more molecules, each molecule having a sequence consisting of SEQ ID NO: 275 and the light chain comprising one or more, each molecule having a sequence consisting of SEQ ID NO: 309;
ap) the heavy chain comprising one or more molecules, each molecule having a sequence consisting of SEQ ID NO: 276 and the light chain comprising one or more, each molecule having a sequence consisting of SEQ ID NO: 310;
aq) the heavy chain comprising one or more molecules, each molecule having a sequence consisting of SEQ ID NO: 277 and the light chain comprising one or more, each molecule having a sequence consisting of SEQ ID NO: 311;
ar) the heavy chain comprising one or more molecules, each molecule having a sequence consisting of SEQ ID NO: 278 and the light chain comprising one or more, each molecule having a sequence consisting of SEQ ID NO: 312;
as) the heavy chain comprising one or more molecules, each molecule having a sequence consisting of SEQ ID NO: 279 and the light chain comprising one or more, each molecule having a sequence consisting of SEQ ID NO: 313;
at) the heavy chain comprising one or more molecules, each molecule having a sequence consisting of SEQ ID NO: 280 and the light chain comprising one or more, each molecule having a sequence consisting of SEQ ID NO: 314;
au) the heavy chain comprising one or more molecules, each molecule having a sequence consisting of SEQ ID NO: 281 and the light chain comprising one or more, each molecule having a sequence consisting of SEQ ID NO: 315;
av) the heavy chain comprising one or more molecules, each molecule having a sequence consisting of SEQ ID NO: 282 and the light chain comprising one or more, each molecule having a sequence consisting of SEQ ID NO: 316;
aw) the heavy chain comprising one or more molecules, each molecule having a sequence consisting of SEQ ID NO: 283 and the light chain comprising one or more, each molecule having a sequence consisting of SEQ ID NO: 317;
ax) the heavy chain comprising one or more molecules, each molecule having a sequence consisting of SEQ ID NO: 284 and the light chain comprising one or more, each molecule having a sequence consisting of SEQ ID NO: 318;
ay) the heavy chain comprising one or more molecules, each molecule having a sequence consisting of SEQ ID NO: 285 and the light chain comprising one or more, each molecule having a sequence consisting of SEQ ID NO: 319;
az) the heavy chain comprising one or more molecules, each molecule having a sequence consisting of SEQ ID NO: 286 and the light chain comprising one or more, each molecule having a sequence consisting of SEQ ID NO: 320;
ba) the heavy chain comprising one or more molecules, each molecule having a sequence consisting of SEQ ID NO: 287 and the light chain comprising one or more, each molecule having a sequence consisting of SEQ ID NO: 321;
bb) the heavy chain comprising one or more molecules, each molecule having a sequence consisting of SEQ ID NO: 288 and the light chain comprising one or more, each molecule having a sequence consisting of SEQ ID NO: 322;
bc) the heavy chain comprising one or more molecules, each molecule having a sequence consisting of SEQ ID NO: 289 and the light chain comprising one or more, each molecule having a sequence consisting of SEQ ID NO: 323;
bd) the heavy chain comprising one or more molecules, each molecule having a sequence consisting of SEQ ID NO: 290 and the light chain comprising one or more, each molecule having a sequence consisting of SEQ ID NO: 324;
be) the heavy chain comprising one or more molecules, each molecule having a sequence consisting of SEQ ID NO: 291 and the light chain comprising one or more, each molecule having a sequence consisting of SEQ ID NO: 325;
bf) the heavy chain comprising one or more molecules, each molecule having a sequence consisting of SEQ ID NO: 292 and the light chain comprising one or more, each molecule having a sequence consisting of SEQ ID NO: 326;
bg) the heavy chain comprising one or more molecules, each molecule having a sequence consisting of SEQ ID NO: 293 and the light chain comprising one or more, each molecule having a sequence consisting of SEQ ID NO: 327;
bh) the heavy chain comprising one or more molecules, each molecule having a sequence consisting of SEQ ID NO: 294 and the light chain comprising one or more, each molecule having a sequence consisting of SEQ ID NO: 328;
bi) the heavy chain comprising one or more molecules, each molecule having a sequence consisting of SEQ ID NO: 295 and the light chain comprising one or more, each molecule having a sequence consisting of SEQ ID NO: 329; or
bj) the heavy chain comprising one or more molecules, each molecule having a sequence consisting of SEQ ID NO: 296 and the light chain comprising one or more, each molecule having a sequence consisting of SEQ ID NO: 330.

44. An isolated nucleic acid encoding an antibody according to claim 40.

45. An isolated antibody molecule capable of binding to human HLA-G (hHLA-G) according to claim 11, further comprising a heavy chain variable region (VH) and a light chain variable region (VL), VH and/or VL comprising 1, 2, 3, 4, 5, or 6 of:
a) a VHCDR1 sequence comprising:
(i) Kabat CDR-H1 sequence defined by the consensus sequence S-S-Δ₃-Δ₄-Y-W-Δ₇ (SEQ ID NOS: 18-21, 23, and 34), where Δ₃ is D or S; Δ₄ is T or Y; and Δ₇ is A, G, or S;
(ii) a Kabat CDR-H1 sequence defined by the consensus sequence S-G-θ₃-Y-W-θ₆ (SEQ ID NOS: 24-29), where θ₃ is F, H, or Y; and θ₆ is F, G, I, L, or T;
(iii) a Chothia CDR-H1 sequence defined by the consensus sequence G-G-S-I-S-S-Ω₇-Ω₈-Ω₉ (SEQ ID NOS: 1-4 and 13-14), where Ω₇ is S or A; Ω₈ is D, S, or N; and Ω₉ is T, N, Y, or is absent; or
(iv) a Chothia CDR-H1 sequence defined by the consensus sequence G-F-T-F-κ₅-κ₆-κ₇ (SEQ ID NOS: 10-12), where κ₅ is D or s; κ₆ is D, N, or S; and κ₇ is S or Y,
b) a VHCDR2 sequence comprising:
(i) a Kabat CDR-H2 sequence defined by the consensus sequence β₁-I-β₃-β₄-β₅-β₆-β₇-T-β₉-Y-N-P-S-L-K-S(SEQ ID NOS: 54-65 and 69-70) where β₁ is A, E, G, or S; β₃ is A, H, S, or Y; β₄ is H, S, or Y; Bs is N or S; β₆ is A or G; β₇ is A, L, or S; and β₉ A, N, L, V, or Y;
(ii) a Chothia CDR-H2 sequence defined by the consensus sequence Y-ε₂-S-ε₄-S(SEQ ID NOS: 38 and 44-45), where ε₂ is H or Y and ε₄ is A or G;
(iii) a Chothia CDR-H2 sequence defined by the consensus sequence α₁-α₂-S-G-S(SEQ ID NOS: 39, 41-42, and 49), where ai is A, H, or S; and az is S or Y; or
(iv) a Chothia CDR-H2 sequence defined by the consensus sequence β₁-β₂-S-G-β₅-β₆ (SEQ ID NOS: 56-60), where β₁ is A or S; β₂ is G or S; β₅ is I or S; and β₆ is T or V,
c) a VHCDR3 sequence comprising:
(i) a CDR-H3 sequence defined by the consensus sequence G-Y₂-Y₃-R-A-V-P-F-Y₉-y₁₀ (SEQ ID NOS: 76-84), where y₂ is I, P, Q, T, or V; y₃ is A, F, K, or R; Y₉ is A, D, Q, or V; y₁₀ is D, R, or Y; or
(ii) a CDR-H3 sequence defined by the consensus sequence G-G-Φ₃-Φ₄-Φ₅-Y-S-R-G-P-Φ₁₁-D-V (SEQ ID NOS: 85-93), where Φ₃ is E, G, Q, or T; Φ₄ is A, H, P, Q, or V; Φs is K or T; and Φ₁₁ is F, L, or M,
d) a VLCDR1 sequence comprising:
(i) a CDR-L1 sequence defined by the consensus sequence ϕ₁-A-S-Q-ϕ₅-V-S-S-ϕ₉-ϕ₁₀-L-A (SEQ ID NOS: 105-112 and 117), where ϕ₁ is E, G, K, Q, or R; ϕ₅ is A or S; ϕ₉ is A, D, N, S, or T; and ϕ₁₀ is F or Y;
(ii) a CDR-L1 sequence defined by the consensus sequence R-A-S-Q-S-σ₆-σ₇-S-σ₉-L-σ₁₁ (SEQ ID NOS: 119 and 123-124), where σ₆ is I or V; σ₇ is N or S; σ₉ is N, W, or Y; σ₁₁ is A or N; or
(iii) a CDR-L1 sequence defined by the consensus sequence Γ₁-Γ₂-S-Q-S-V-S-Γ₈-Γ₉-Y-L-A (SEQ ID NOS: 113-116), where Γ₁ is E or R; Γ₂ is A or V; Γ₈ is A, D, or S; and Γ₁₀ is A or S,
e) a VLCDR2 sequence comprising:
(i) a CDR-L2 sequence defined by the consensus sequence ψ₁-A-S-ψ₄-R-A-ψ₇ (SEQ ID NOS: 128, 130, 132, 134-138, 143, and 145), where ψ₁ is D or G; ψ₄ is A, D, N, R, S, T, or Y; and ψ₇ is A, N, S, or T, and
f) a VLCDR3 sequence comprising:
(i) a CDR-L3 sequence defined by the consensus sequence Q-π₂-π₃-π₄-H-S-P-Y-T (SEQ ID NOS: 149-153), where π₂ is Q or W; π₃ is A, T, or V; and π₄ is I or V;
(ii) a CDR-L3 sequence defined by the consensus sequence Q-Q-λ₃-S-λ₅-Y-P-P-T (SEQ ID NOS: 154-158), where λ₃ is F, H, or V; and λ₅ is I, L, or S; or
(iii) a CDR-L3 sequence defined by the consensus sequence Q-Q-ω₃-ω₄-ω₅-ω₆-P-I-T (SEQ ID NOS: 160-161 and 163-164), where ω₃ is A, L, V, or Y; Φ₄ is G, P, V, or Y; ω₅ is S, L, or F; and ω₆ is D, L, S, or Y.
